(19) United States
(12) Patent Application Publication
(10) Pub. No.: US 2022/0259668 A1
(43) Pub. Date: Aug. 18, 2022

(54) RAPID ANEUPLOIDY DETECTION

(71) Applicant: The Johns Hopkins University, Baltimore, MD (US)

(72) Inventors: Bert Vogelstein, Baltimore, MD (US); Kenneth W. Kinzler, Baltimore, MD (US); Christopher Douville, Baltimore, MD (US); Nickolas Papadopoulos, Towson, MD (US); Cristian Tomasetti, Baltimore, MD (US)

(21) Appl. No.: 17/611,788

(22) PCT Filed: May 15, 2020
(86) PCT No.: PCT/US2020/033209

§ 371 Date: Nov. 16, 2021

Related U.S. Application Data
(60) Provisional application No. 62/971,050, filed on Feb. 6, 2020.
(60) Provisional application No. 62/905,327, filed on Sep. 24, 2019.
(60) Provisional application No. 62/849,662, filed on May 17, 2019.

(51) Int. Cl. C12Q 1/6886; G16B 40/20; G01N 33/574

(52) U.S. Cl. CPC C12Q 1/6886; G16B 40/20; G01N 2800/50; C12Q 2600/156; C12Q 2600/118; G01N 33/57484

(57) ABSTRACT

This document provides methods and materials for identifying chromosomal anomalies that can be used to identify a mammal as having a disease (e.g., cancer or congenital abnormality). For example, this document provides methods and materials for evaluating sequencing data to identify a mammal as having a disease associated with one or more chromosomal anomalies (e.g., cancer or congenital abnormalities). For example, this document provides methods and materials for evaluating sequencing data that can be used in cancer diagnostics, non-invasive prenatal testing (NIPT), preimplantation genetic diagnosis and evaluation of congenital abnormalities.

### CROSS-REFERENCE TO RELATED APPLICATIONS

This application claims the benefit of U.S. Provisional Application Ser. No. 62/849,662, filed on May 17, 2019; U.S. Provisional Application Ser. No. 62/905,327, filed on Sep. 24, 2019 and U.S. Provisional Application Ser. No. 62/971,050, filed on Feb. 6, 2020. The disclosures of the prior applications are considered part of (and are incorporated by reference herein) the disclosure of this application.

### STATEMENT REGARDING FEDERAL FUNDING

This invention was made with government support under grants CA230691 and CA230400 awarded by the National Institutes of Health. The government has certain rights in the invention.

### BACKGROUND

### 1. Technical Field

This document provides methods and materials for identifying chromosomal anomalies that can be used in cancer diagnostics, non-invasive prenatal testing (NIPT), preimplantation genetic diagnosis, and evaluation of congenital abnormalities. For example, this document provides methods and materials for evaluating sequencing data to identify a mammal as having a disease associated with one or more chromosomal anomalies (e.g., cancer or congenital abnormality). Additionally or alternatively, this document provides methods and materials for evaluating sequencing data that can be used in cancer diagnostics, non-invasive prenatal testing (NIPT), preimplantation genetic diagnosis, and evaluation of congenital abnormalities.

### 2. Background Information

Aneuploidy is defined as an abnormal chromosome number. It was the first genomic abnormality identified in cancers (Boveri 2008 Journal of cell science 121 (Supplement 1):1-84; and Nowell 1976 Science 194(4260):23-28), and it has been estimated to be present in >90% of cancers of most histopathologic types (Knouse et al. 2017 Annual Review of Cancer Biology 1:335-354). Aneuploidy in cancers was first detected by karyotypic studies, later evaluated through microarrays, Sanger sequencing, and most recently, massively parallel sequencing methods (Wang et al. 2002 Proceedings of the National Academy of Sciences 99(25):16156-16161). Recent sequencing methods include those employing circular binary segmentation, hidden Markov models, expectation maximization and mean-shift (as reviewed in (Zhao et al. 2013 BMC bioinformatics 14(11):S1)). In addition to their application to cancer genomes, these technologies form the basis for the non-invasive prenatal detection of fetuses with Downs' Syndrome and other trisomies (Bianchi et al. 2015 JAMA 314(2):162-169; Zhao et al. 2015 Clinical chemistry 61(4):608-616).

### SUMMARY

This disclosure relates to methods and materials for identifying one or more chromosomal anomalies (e.g., aneuploidy). In some embodiments, this disclosure provides methods and materials for using amplicon-based sequencing data to identify a mammal as having a disease or disorder associated with one or more chromosomal anomalies. For example, methods and materials described herein can be applied to a sample obtained from a mammal to identify the mammal as having one or more chromosomal anomalies. For example, a mammal can be identified as having a disease or disorder based, at least in part, on the presence of one or more aneuploidies. In some embodiments, a single primer pair is used to amplify genomic elements throughout the genome. For example, a single primer pair described herein can be used to amplify ˜1,000,000 unique repetitive elements (e.g., amplicons). In some embodiments, the amplified unique repetitive elements average less than 100 basepairs (bp) in size. In some embodiments, an approach (called WALDO for Within-Sample-AneupLoidy-DetectiOn) can be used to evaluate the sequencing data obtained from amplicons to identify the presence of one or more chromosomal anomalies (e.g., aneuploidy). As described herein, assessment of aneuploidy in 1,348 plasma samples from healthy people and 883 plasma samples from cancer patients detected aneuploidy in 49% of the plasma samples from cancer patients.

In one aspect, provided herein is a method of testing for the presence of aneuploidy in a genome of a mammal. The method comprises amplifying a plurality of chromosomal sequences in a DNA sample with a pair of primers complementary to the chromosomal sequences to form a plurality of amplicons; determining at least a portion of the nucleic acid sequence of one or more of the plurality of amplicons; mapping the sequenced amplicons to a reference genome; dividing the DNA sample into a plurality of genomic intervals; quantifying a plurality of features for the amplicons mapped to the genomic intervals; comparing the plurality of features of amplicons in a first genomic interval with the plurality of features of amplicons in one or more different genomic intervals; and wherein at least 100,000 amplicons are formed in the step of amplifying (e.g., the plurality of amplicons can include ˜745,000 amplicons).

In some embodiments, the method is performed in vitro. In some embodiments, the plurality of amplicons comprise about 1,000,000 amplicons, e.g., about 1,000,000-10,000 amplicons; about 1,000,000-50,000 amplicons; about 1,000,000-100,000 amplicons; about 1,000,000-200,000 amplicons; about 1,000,000-300,000 amplicons; about 1,000,000-400,000 amplicons; about 1,000,000-500,000 amplicons; about 1,000,000-600,000 amplicons; about 1,000,000-700,000 amplicons; about 1,000,000-800,000 amplicons; about 1,000,000-900,000 amplicons; about 900,000-10,000 amplicons; about 800,000-10,000 amplicons; about 700,000-10,000 amplicons; about 600,000-10,000 amplicons; about 500,000-10,000 amplicons; about 400,000-10,000 amplicons; about 300,000-10,000 amplicons; about 200,000-10,000 amplicons; about 100,000-10,000 amplicons or about 50,000-10,000 amplicons.

In some embodiments, the plurality of amplicons comprises about 50,000 amplicons; about 100,000 amplicons; about 150,000 amplicons; about 200,000 amplicons; about 250,000 amplicons; about 300,000 amplicons; about 350,000 amplicons; about 400,000 amplicons; about 450,000 amplicons; about 500,00 amplicons; about 550,000 amplicons; about 600,000 amplicons; about 650,000 amplicons; about 700,000 amplicons; about 750,000 amplicons; about 800,000 amplicons; about 850,000 amplicons; about 900,000 amplicons; about 950,000 amplicons; or about 1,000,000 amplicons.

In some embodiments, the plurality of amplicons comprises about 750,000 amplicons.

In some embodiments, the plurality of amplicons comprises about 350,000 amplicons.

In some embodiments, the number of repetitive elements, e.g., amplicons, amplified by the single primer pair disclosed herein is a function of: the number of repetitive elements present in a sample and/or the length of a repetitive element present in a sample. For example, in some samples, the number of repetitive elements, e.g., amplicons, that can be detected with the single primer pair is about ˜750,000 amplicons. In some embodiments, in other samples, the number of repetitive elements, e.g., amplicons, that can be detected with the single primer pair is about ˜350,000 amplicons.

In some embodiments, the DNA sample is a plurality of euploid DNA samples. In some embodiments, the DNA sample is a plurality of test DNA samples. In some embodiments, the DNA sample is a plurality of test DNA samples. In some embodiments, the DNA sample is from plasma. In some embodiments, the DNA sample is from serum. In some embodiments, the DNA sample comprises cell fetal DNA. In some embodiments, the DNA sample comprises at least 3 picograms of DNA. In some embodiments, the mammal is a human. In some embodiments the pair of primers comprises a first primer comprising SEQ ID NO: 1 and a second primer comprising SEQ ID NO: 10. In some embodiments, the methods provide herein include one or more additional pairs of primers. In some embodiments, the amplicons include repetitive elements (e.g., one or more types of repetitive elements shown in Table 1). In some embodiments, the amplicons include unique short interspersed nucleotide elements (SINEs). In some embodiments, the amplicons include unique long interspersed nucleotide elements (LINEs).

In some embodiments, the average length of the amplicons is about 100 basepairs or less. In some embodiments, the average length of the amplicons is less than about 110 bp, e.g., about 10-110 bp, about 10-105 bp, about 10-100 bp, about 10-99 bp, about 10-98 bp, about 10-97 bp, about 10-96 bp, about 10-95 bp, about 10-94 bp, about 10-93 bp, about 10-92 bp, about 10-91 bp, about 10-90 bp, about 10-89 bp, about 10-87 bp, about 10-86 bp, about 10-85 bp, about 10-84 bp, about 10-83 bp, about 10-82 bp, about 10-81 bp, about 10-80 bp, about 10-79 bp, about 10-78 bp, about 10-77 bp, about 10-76 bp, about 10-75 bp, about 10-74 bp, about 10-73 bp, about 10-72 bp, about 10-71 bp, about 10-70 bp, about 10-65 bp, about 10-60 bp, about 10-55 bp, about 10-50 bp, about 10-40 bp, about 10-30 bp, about 10-20 bp, about 15-110 bp, about 20-110 bp, about 25-110 bp, about 30-110 bp, about 35-110 bp, about 40-110 bp, about 45-110 bp, about 50-110 bp, about 55-110 bp about 60-110 bp, about 65-110 bp, about 70-110 bp, about 75-110 bp, about 80-110 bp, about 85-110 bp, about 90-110 bp, about 95-110 bp, about 100-110 bp, or about 105-110 bp.

In some embodiments, the average length of the amplicons is about 10 bp; about 20 bp; about 30 bp; about 40 bp; about 45 bp; about 50 bp; about 60 bp; about 65 bp; about 70 bp; about 75 bp; about 80 bp; about 85 bp; about 90 bp; about 95 bp; about 100 bp; about 105 bp or about 110 bp.

In some embodiments, the amplicons comprise one or more long amplicons where the average length is 1000 basepairs or greater. In some embodiments, the long amplicons comprise DNA from a contaminating cell. In some embodiments, the contaminating cell is a leukocyte. In some embodiments, the genomic intervals comprise from about 100 nucleotides to about 125,000,000 nucleotides (e.g., the genomic intervals can include about 500,000 nucleotides).

In another aspect, the disclosure provides a method of evaluating a subject for the presence of, or the risk of developing, any of a plurality of, e.g., any of at least four, cancers in the subject comprising:

(i) acquiring, e.g., directly acquiring or indirectly acquiring, a value for, e.g., detecting, the presence of one or more genetic biomarkers, e.g., one or more mutations (e.g., one or more driver gene mutations), in each of one or more genes (e.g., one or more driver genes, e.g., in at least four driver genes), and optionally wherein, each gene, e.g., driver gene, is associated with the presence, or risk, of a cancer of the plurality of cancers;

(ii) acquiring, e.g., directly acquiring or indirectly acquiring, a value for, e.g., detecting, the level of each of a plurality of, e.g., at least four, protein biomarkers, and optionally wherein, the level of each protein biomarker of the plurality is associated with the presence, or risk, of a cancer of the plurality of cancers; or

(iii) acquiring, e.g., directly acquiring or indirectly acquiring, a value for, e.g., detecting, aneuploidy, wherein the aneuploidy value is a function of the copy number or length of a genomic sequence disposed between at least two terminal repeated elements of a repeated element family (RE Family), wherein the RE family comprises:

(a) a RE Family other than a long interspersed nucleotide element (LINE);

(b) a RE Family which when amplified with a primer moiety complementary to its repeated terminal elements, provides amplicons having an average length of less than X nts, wherein X is 100, 105, or 110,

(c) a RE family which is less than about 700 bp long; or

(d) a RE family which is present in at least 100 copies per genome;

and optionally wherein, the aneuploidy is associated with the presence, or risk, of a cancer of the plurality of cancers;

thereby evaluating the subject for the presence of or risk of developing, any of the plurality of, e.g., any of at least four, cancers.

In an embodiment, one of (i), (ii) and (iii) is directly acquired. In an embodiment, (i) and (ii) are directly acquired. In an embodiment, (i) and (iii) are directly acquired. In an embodiment, (ii) and (iii) are directly acquired. In an embodiment, all of (i), (ii) and (iii) are directly acquired.

In an embodiment, one of (i), (ii) and (iii) is indirectly acquired. In an embodiment, (i) and (ii) are indirectly acquired. In an embodiment, (i) and (iii) are indirectly acquired. In an embodiment, (ii) and (iii) are indirectly acquired. In an embodiment, all of (i), (ii) and (iii) are indirectly acquired.

In an embodiment, the method comprises sequencing one or more subgenomic intervals or amplicons comprising the genetic biomarkers. In an embodiment, the method comprises analyzing one or more genomic sequences for aneuploidy. In an embodiment, the method comprises, contacting a protein biomarker with a detection reagent. In an embodiment, the method comprises: (1) sequencing one or more subgenomic intervals or amplicons comprising the genetic biomarkers; (2) analyzing one or more genomic sequences for aneuploidy, and/or (3) contacting a protein biomarker with a detection reagent.

In an embodiment, the aneuploidy value is a function of the copy number of the genomic sequence disposed between at least two terminal repeated elements of a RE Family. In an embodiment, the aneuploidy value is a function of the length of the genomic sequence disposed between at least two terminal repeated elements of a repeated element family (RE Family).

In some embodiments, the method is performed in vitro.

In an embodiment, a sample, e.g., a biological sample, obtained from the subject is evaluated for one, two or all of (i)-(iii). In an embodiment, the biological sample comprises a liquid sample, e.g., a blood sample. In an embodiment, the biological sample comprises a cell-free DNA sample, a plasma sample or a serum sample. In an embodiment, the biological sample comprises cell-free DNA, e.g., circulating tumor DNA. In an embodiment, the biological sample comprises cells and/or tissue. In an embodiment, the biological sample comprises cells (e.g., normal or cancer cells) and cell-free DNA.

In an embodiment of any of the methods disclosed herein, specificity of detection of the cancer in the plurality of cancers with (i), (ii) and (iii) is substantially the same as, e.g., not substantially lower than, the specificity of detection of the cancer in the plurality of cancers with: (i); (ii); (iii); (i) and (ii); (i) and (iii); or (ii) and (iii).

In an embodiment of any of the methods disclosed herein, sensitivity of detection of the cancer in the plurality of cancers with (i), (ii) and (iii) is higher, e.g., about 1.1, 1.2, 1.3, 1.4, 1.5, 2, 2.5, 3, 3.5, 4, 4.5, 5, 5.5, 6, 6.5, 7, 7.5, 8, 8.5, 9, 9.5, or 10 fold higher, than the sensitivity of detection of the cancer in the plurality of cancers with: (i); (ii); (iii); (i) and (ii); (i) and (iii); or (ii) and (iii). In an embodiment, an increased sensitivity of detection, e.g., about 1.1, 1.2, 1.3, 1.4, 1.5, 2, 2.5, 3, 3.5, 4, 4.5, 5, 5.5, 6, 6.5, 7, 7.5, 8, 8.5, 9, 9.5, or 10 fold increase in sensitivity of detection at a specified specificity, e.g., at a predetermined specificity, e.g., at least about 90%, 91%, 92%, 93%, 94%, 95%, 96%, 97%, 98%, 99% or 100% specificity.

In some embodiments, the plurality of amplicons comprise about 1,000,000 amplicons, e.g., about 1,000,000-10,000 amplicons; about 1,000,000-50,000 amplicons; about 1,000,000-100,000 amplicons; about 1,000,000-200,000 amplicons; about 1,000,000-300,000 amplicons; about 1,000,000-400,000 amplicons; about 1,000,000-500,000 amplicons; about 1,000,000-600,000 amplicons; about 1,000,000-700,000 amplicons; about 1,000,000-800,000 amplicons; about 1,000,000-900,000 amplicons; about 900,000-10,000 amplicons; about 800,000-10,000 amplicons; about 700,000-10,000 amplicons; about 600,000-10,000 amplicons; about 500,000-10,000 amplicons; about 400,000-10,000 amplicons; about 300,000-10,000 amplicons; about 200,000-10,000 amplicons; about 100,000-10,000 amplicons or about 50,000-10,000 amplicons.

In some embodiments, the plurality of amplicons comprises about 50,000 amplicons; about 100,000 amplicons; about 150,000 amplicons; about 200,000 amplicons; about 250,000 amplicons; about 300,000 amplicons; about 350,000 amplicons; about 400,000 amplicons; about 450,000 amplicons; about 500,00 amplicons; about 550,000 amplicons; about 600,000 amplicons; about 650,000 amplicons; about 700,000 amplicons; about 750,000 amplicons; about 800,000 amplicons; about 850,000 amplicons; about 900,000 amplicons; about 950,000 amplicons; or about 1,000,000 amplicons.

In some embodiments, the plurality of amplicons comprises about 750,000 amplicons.

In some embodiments, the plurality of amplicons comprises about 350,000 amplicons.

In some embodiments, the number of repetitive elements, e.g., amplicons, amplified by the single primer pair disclosed herein is a function of: the number of repetitive elements present in a sample and/or the length of a repetitive element present in a sample. For example, in some samples, the number of repetitive elements, e.g., amplicons, that can be detected with the single primer pair is about ˜750,000 amplicons. In some embodiments, in other samples, the number of repetitive elements, e.g., amplicons, that can be detected with the single primer pair is about ˜350,000 amplicons.

In some embodiments, the average length of the amplicons is about 100 basepairs or less. In some embodiments, the average length of the amplicons is less than about 110 bp, e.g., about 10-110 bp, about 10-105 bp, about 10-100 bp, about 10-99 bp, about 10-98 bp, about 10-97 bp, about 10-96 bp, about 10-95 bp, about 10-94 bp, about 10-93 bp, about 10-92 bp, about 10-91 bp, about 10-90 bp, about 10-89 bp, about 10-87 bp, about 10-86 bp, about 10-85 bp, about 10-84 bp, about 10-83 bp, about 10-82 bp, about 10-81 bp, about 10-80 bp, about 10-79 bp, about 10-78 bp, about 10-77 bp, about 10-76 bp, about 10-75 bp, about 10-74 bp, about 10-73 bp, about 10-72 bp, about 10-71 bp, about 10-70 bp, about 10-65 bp, about 10-60 bp, about 10-55 bp, about 10-50 bp, about 10-40 bp, about 10-30 bp, about 10-20 bp, about 15-110 bp, about 20-110 bp, about 25-110 bp, about 30-110 bp, about 35-110 bp, about 40-110 bp, about 45-110 bp, about 50-110 bp, about 55-110 bp about 60-110 bp, about 65-110 bp, about 70-110 bp, about 75-110 bp, about 80-110 bp, about 85-110 bp, about 90-110 bp, about 95-110 bp, about 100-110 bp, or about 105-110 bp.

In some embodiments, the average length of the amplicons is about 10 bp; about 20 bp; about 30 bp; about 40 bp; about 45 bp; about 50 bp; about 60 bp; about 65 bp; about 70 bp; about 75 bp; about 80 bp; about 85 bp; about 90 bp; about 95 bp; about 100 bp; about 105 bp or about 110 bp.

In some embodiments, the method further comprises subjecting the subject to a radiologic scan, e.g., a PET-CT scan, of an organ or body region. In some embodiments, the radiologic scanning of an organ or body region characterizes the cancer. In some embodiments, the radiologic scanning of an organ or body region identifies the location of the cancer. In some embodiments, the radiologic scan is a PET-CT scan. In some embodiments, the radiologic scanning is performed after the subject is evaluated for the presence of each of a plurality of cancers.

In another aspect, the disclosure provides a method of testing for the presence of aneuploidy in a genome of a mammal. The method comprises:
a) amplifying a plurality of chromosomal sequences in a DNA sample with a primer moiety, e.g., a primer or pair of primers complementary to the chromosomal sequences to form a plurality of amplicons, e.g., wherein the primer moiety amplifies a sufficient number of sequences to allow aneuploidy detection;
b) determining at least a portion of the nucleic acid sequence of one or more of the plurality of amplicons;
c) mapping the sequenced amplicons to a reference genome;
d) dividing the DNA sample into a plurality of genomic intervals;
e) quantifying a plurality of features for the amplicons mapped to the genomic intervals;
f) comparing the plurality of features of amplicons in a first genomic interval with the plurality of features of amplicons in one or more different genomic intervals; and
   wherein a number of amplicons sufficient to detect aneuploidy, e.g., at least 10,000, 20,000, 50,000, or 100,000 amplicons are formed in the step of amplifying.

In some embodiments, the method is performed in vitro.

In an embodiment of any of the methods disclosed herein, increase in sensitivity of detection of the cancer in the plurality of cancers does not affect, e.g., reduce or substantially reduce, the specificity of detection of the cancer in the plurality of cancer. In an embodiment, the specificity of detection of the cancer in the plurality of cancers is at a plateau, e.g., the specificity of detection is not altered by detection of additional biomarkers.

In another aspect, provided herein is a method of detecting aneuploidy in a sample comprising low input DNA, using any of the methods disclosed herein.

In some embodiments, the sample comprises about 0.01 picogram (pg) to 500 pg of DNA. In some embodiments, the sample comprises about 0.01-500 pg, 0.05-400 pg, 0.1-300 pg, 0.5-200 pg, 1-100 pg, 10-90 pg, or 20-50 pg DNA. In some embodiments, the sample comprises at least 0.01 pg, at least 0.01 pg, at least 0.1 pg, at least 1 pg, at least 2 pg, at least 3 pg, at least 4 pg, at least 5 pg, at least 6 pg, at least 7 pg, at least 8 pg, at least 9 pg at least 10 pg, at least 11 pg, at least 12 pg, at least 13 pg, at least 14 pg, at least 15 pg, at least 16 pg, at least 17 pg, at least 18 pg, at least 19 pg, at least 20 pg, at least 21 pg, at least 22 pg, at least 23 pg, at least 24 pg, at least 25 pg, at least 26 pg, at least 27 pg, at least 28 pg, at least 29 pg, at least 30 pg, at least 31 pg, at least 32 pg, at least 33 pg, at least 34 pg, at least 35 pg, at least 36 pg, at least 37 pg, at least 38 pg, at least 39 pg, at least 40 pg, at least 50 pg, at least 60 pg, at least 70 pg, at least 80 pg, at least 90 pg, at least 100 pg, at least 150 pg, at least 200 pg, at least 300 pg, at least 350 pg, at least 400 pg, at least 450 pg, or at least 500 pg DNA.

In some embodiments, the sample comprises 1 pg DNA. In some embodiments, the sample comprises 2 pg DNA. In some embodiments, the sample comprises 3 pg DNA. In some embodiments, the sample comprises 4 pg DNA. In some embodiments, the sample comprises 5 pg DNA. In some embodiments, the sample comprises 10 pg DNA.

In some embodiments, the sample is a biological sample from a subject. In an embodiment, the biological sample comprises a liquid sample, e.g., a blood sample. In an embodiment, the biological sample comprises a cell-free DNA sample, a plasma sample or a serum sample. In an embodiment, the biological sample comprises cell-free DNA, e.g., circulating tumor DNA. In an embodiment, the biological sample comprises cells and/or tissue. In an embodiment, the biological sample comprises cells (e.g., normal or cancer cells) and cell-free DNA.

In some embodiments, the sample is a trisomy 21 sample. In some embodiments, the sample is a forensic sample. In some embodiments, the sample is from an embryo, e.g., preimplantation embryo.

In some embodiments, the sample is a biobank sample, e.g., as described in Example 3.

In some embodiments, the method is used for diagnostics, e.g., preimplantation diagnostics.

In some embodiments, the method is used for forensics.

In some embodiments, the method is an in vitro method.

In another aspect, provided herein is a method of identifying or distinguishing a sample using any of the methods disclosed herein.

In some embodiments, the sample, e.g., first sample, from a subject (e.g., first subject) is distinguished from a second sample from a second subject. In some embodiments, the sample, e.g., first sample, is identified as being from the first subject based on a polymorphism (e.g., a plurality of polymorphisms, e.g., common polymorphisms). In some embodiments, the second sample is identified as being from the second subject based on a polymorphism (e.g., a plurality of polymorphisms, e.g., common polymorphisms). In some embodiments, a common polymorphism is present in a repetitive element, e.g., as described herein. In some embodiments, methods disclosed in Example 8 can be used to identify and/or distinguish the sample.

In another aspect, provided herein is a reaction mixture comprising: at least 2, 3, 4, 5, 6, 7, 8, 9 or 10 detection reagents, wherein a detection reagent mediates a readout that is a value of the level or presence of: (i) one or more genetic biomarkers referred to herein; (ii) one or more protein biomarkers referred to herein; and/or (iii) the copy number or length, e.g., aneuploidy, of a genomic sequence disposed between at least two terminal repeated elements of a repeated element family (RE Family) referred to herein.

In yet another aspect, the disclosure provides a kit comprising: (a) at least 2, 3, 4, 5, 6, 7, 8, 9 or 10 detection reagents, wherein a detection reagent mediates a readout that is a value of the level or presence of: (i) one or more genetic biomarkers referred to herein; (ii) one or more protein biomarkers referred to herein; and/or (iii) the copy number or length, e.g., aneuploidy, of a genomic sequence disposed between at least two terminal repeated elements of a repeated element family (RE Family) referred to herein; and (b) instructions for using said kit.

In some embodiments of any of the methods disclosed herein, quantifying amplicons mapped to genomic intervals comprises identifying a plurality of genomic intervals with one or more shared amplicon features. In some embodiments, the shared amplicon feature is the number of the mapped amplicons.

In some embodiments of any of the methods disclosed herein, the shared amplicon feature is the average length of the mapped amplicons. In some embodiments, the plurality of genomic intervals with shared amplicon features are grouped into clusters. In some embodiments, each cluster includes about two hundred genomic intervals. In some embodiments, the clusters comprise predefined clusters. In some embodiments, the comparison of the genomic intervals further comprises matching one or more genomic intervals from test samples to predefined clusters. In some embodiments, matching genomic intervals from test samples to predefined clusters further comprises identifying one or more genomic intervals with shared amplicon features outside a predetermined significance threshold for a predefined cluster. In some embodiments, the method comprises supervised machine learning. In some embodiments, the supervised machine learning employs a support vector machine model.

In some embodiments of any of the methods disclosed herein, a single pair of primers is used for the amplification of a plurality of amplicons from a DNA sample comprising a first primer comprising a sequence that is at least 80% identical to SEQ ID NO: 1 and a second primer comprising a sequence that is at least 80% identical to SEQ ID NO: 10. In some embodiments, the sequence of the first primer is at least 90% identical to SEQ ID NO. 1. In some embodiments, the sequence of the first primer is at least 95% identical to SEQ ID NO. 1. In some embodiments, the sequence of the first primer is 100% identical to SEQ ID NO. 1. In some embodiments, the sequence of the second primer is at least 90% identical to SEQ ID NO. 10. In some embodiments, the sequence of the second primer is at least 95% identical to SEQ ID NO. 10. In some embodiments, the sequence of the second primer is 100% identical to SEQ ID NO. 10. In some embodiments, a kit comprising a pair of primers is used to amplify a plurality of amplicons from a DNA sample, wherein a first primer of the primer pair comprises SEQ ID NO: 1 or a sequence at least 80% identical thereto, and a second primer of the primer pair comprises SEQ ID NO: 10, or a sequence at least 80% identical thereto.

In another aspect, the disclosure provides a method of testing for the presence of cancer of a mammal. The method includes: a) amplifying a plurality of chromosomal sequences in a DNA sample with a pair of primers complementary to the chromosomal sequences to form a plurality of amplicons; b) determining at least a portion of the nucleic acid sequence of one or more of the plurality of amplicons; c) mapping the sequenced amplicons to a reference genome; d) dividing the DNA sample into a plurality of genomic intervals; e) quantifying a plurality of features for the amplicons mapped to the genomic intervals; f) comparing the plurality of features of amplicons in a first genomic interval with the plurality of features of amplicons in one or more different genomic intervals; and g) determining the presence of cancer in the mammal when the plurality of features of amplicons in a first genomic interval is different from the plurality of features of amplicons in one or more different genomic intervals. In some embodiments, the method can include at least 100,000 amplicons formed in the step of amplifying. In some embodiments, the cancer can be a Stage I cancer. In some embodiments, the cancer can be a liver cancer, an ovarian cancer, an esophageal cancer, a stomach cancer, a pancreatic cancer, a colorectal cancer, a lung cancer, a breast cancer, or a prostate cancer.

In some embodiments, the method is an in vitro method.

In some embodiments of any of the methods, reaction mixtures or kits disclosed herein, the plurality of amplicons comprise about 1,000,000 amplicons, e.g., about 1,000,000-10,000 amplicons; about 1,000,000-50,000 amplicons; about 1,000,000-100,000 amplicons; about 1,000,000-200,000 amplicons; about 1,000,000-300,000 amplicons; about 1,000,000-400,000 amplicons; about 1,000,000-500,000 amplicons; about 1,000,000-600,000 amplicons; about 1,000,000-700,000 amplicons; about 1,000,000-800,000 amplicons; about 1,000,000-900,000 amplicons; about 900,000-10,000 amplicons; about 800,000-10,000 amplicons; about 700,000-10,000 amplicons; about 600,000-10,000 amplicons; about 500,000-10,000 amplicons; about 400,000-10,000 amplicons; about 300,000-10,000 amplicons; about 200,000-10,000 amplicons; about 100,000-10,000 amplicons or about 50,000-10,000 amplicons.

In some embodiments, the plurality of amplicons comprises about 50,000 amplicons; about 100,000 amplicons; about 150,000 amplicons; about 200,000 amplicons; about 250,000 amplicons; about 300,000 amplicons; about 350,000 amplicons; about 400,000 amplicons; about 450,000 amplicons; about 500,00 amplicons; about 550,000 amplicons; about 600,000 amplicons; about 650,000 amplicons; about 700,000 amplicons; about 750,000 amplicons; about 800,000 amplicons; about 850,000 amplicons; about 900,000 amplicons; about 950,000 amplicons; or about 1,000,000 amplicons.

In some embodiments, the plurality of amplicons comprises about 750,000 amplicons.

In some embodiments, the plurality of amplicons comprises about 350,000 amplicons.

In some embodiments of any of the methods disclosed herein, the number of repetitive elements, e.g., amplicons, amplified by the single primer pair disclosed herein is a function of: the number of repetitive elements present in a sample and/or the length of a repetitive element present in a sample. For example, in some samples, the number of repetitive elements, e.g., amplicons, that can be detected with the single primer pair is about 750,000 amplicons. In some embodiments, in other samples, the number of repetitive elements, e.g., amplicons, that can be detected with the single primer pair is about 350,000 amplicons.

In some embodiments of any of the methods, reaction mixtures or kits disclosed herein, the average length of the amplicons is about 100 basepairs or less. In some embodiments, the average length of the amplicons is less than about 110 bp, e.g., about 10-110 bp, about 10-105 bp, about 10-100 bp, about 10-99 bp, about 10-98 bp, about 10-97 bp, about 10-96 bp, about 10-95 bp, about 10-94 bp, about 10-93 bp, about 10-92 bp, about 10-91 bp, about 10-90 bp, about 10-89 bp, about 10-87 bp, about 10-86 bp, about 10-85 bp, about 10-84 bp, about 10-83 bp, about 10-82 bp, about 10-81 bp, about 10-80 bp, about 10-79 bp, about 10-78 bp, about 10-77 bp, about 10-76 bp, about 10-75 bp, about 10-74 bp, about 10-73 bp, about 10-72 bp, about 10-71 bp, about 10-70 bp, about 10-65 bp, about 10-60 bp, about 10-55 bp, about 10-50 bp, about 10-40 bp, about 10-30 bp, about 10-20 bp, about 15-110 bp, about 20-110 bp, about 25-110 bp, about 30-110 bp, about 35-110 bp, about 40-110 bp, about 45-110 bp, about 50-110 bp, about 55-110 bp about 60-110 bp, about 65-110 bp, about 70-110 bp, about 75-110 bp, about 80-110 bp, about 85-110 bp, about 90-110 bp, about 95-110 bp, about 100-110 bp, or about 105-110 bp.

In some embodiments, the average length of the amplicons is about 10 bp; about 20 bp; about 30 bp; about 40 bp; about 45 bp; about 50 bp; about 60 bp; about 65 bp; about 70 bp; about 75 bp; about 80 bp; about 85 bp; about 90 bp; about 95 bp; about 100 bp; about 105 bp or about 110 bp.

Additional features of any of the methods disclosed herein include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### ENUMERATED EMBODIMENTS

E1. A method of evaluating a subject for the presence of, or the risk of developing, any of a plurality of, e.g., any of at least four, cancers in the subject comprising:

(i) acquiring, e.g., directly acquiring or indirectly acquiring, a value for, e.g., detecting, the presence of one or more genetic biomarkers, e.g., one or more mutations (e.g., one or more driver gene mutations), in each of one or more genes (e.g., one or more driver genes, e.g., in at least four driver genes), and optionally wherein, each gene, e.g., driver gene, is associated with the presence, or risk, of a cancer of the plurality of cancers;

(ii) acquiring, e.g., directly acquiring or indirectly acquiring, a value for, e.g., detecting, the level of each of a plurality of, e.g., at least four, protein biomarkers, and optionally wherein, the level of each protein biomarker of the plurality is associated with the presence, or risk, of a cancer of the plurality of cancers; or

(iii) acquiring, e.g., directly acquiring or indirectly acquiring, a value for, e.g., detecting, aneuploidy, wherein the aneuploidy value is a function of the copy number or length of a genomic sequence disposed between at least two terminal repeated elements of a repeated element family (RE Family), wherein the RE family comprises:

(a) a RE Family other than a long interspersed nucleotide element (LINE);

(b) a RE Family which when amplified with a primer moiety complementary to its repeated terminal elements, provides a plurality of amplicons having an average length of less than X nts, wherein X is 100, 105, or 110,

(c) a RE family which is less than about 700 bp long; or

(d) a RE family which is present in at least 100 copies per genome;

and optionally wherein, the aneuploidy is associated with the presence, or risk, of a cancer of the plurality of cancers;

thereby evaluating the subject for the presence of or risk of developing, any of the plurality of, e.g., any of at least four, cancers.

E2. The method of embodiment E1, wherein:

(a) one of (i), (ii) and (iii) is directly acquired;

(b) (i) and (ii) are directly acquired;

(c) (i) and (iii) are directly acquired;

(d) (ii) and (iii) are directly acquired; or

(e) all of (i), (ii) and (iii) are directly acquired.

E3. The method of embodiment E1, wherein:

(a) one of (i), (ii) and (iii) is indirectly acquired;

(b) (i) and (ii) are indirectly acquired;

(c) (i) and (iii) are indirectly acquired;

(d) (ii) and (iii) are indirectly acquired; or

(e) all of (i), (ii) and (iii) are indirectly acquired.

E4. The method of any one of embodiments E1-E3, comprising:

(1) sequencing one or more subgenomic intervals or amplicons comprising the genetic biomarkers;

(2) analyzing one or more genomic sequences for aneuploidy, and/or

(3) contacting a protein biomarker with a detection reagent.

E5. The method of any one of embodiments E1-E4, wherein the aneuploidy value is a function of:

(a) the copy number of the genomic sequence disposed between at least two terminal repeated elements of a RE Family; and/or

(b) the length of the genomic sequence disposed between at least two terminal repeated elements of a repeated element family (RE Family).

E6. The method of any one of embodiments E1-E5, wherein a biological sample obtained from the subject is evaluated for one, two or all of (i)-(iii).

E7. The method of embodiment E6, wherein the biological sample comprises a liquid sample, e.g., a blood sample.

E8. The method of embodiment E6 or E7, wherein the biological sample comprises a cell-free DNA sample, a plasma sample or a serum sample.

E9. The method of any one of embodiments E6-E8, wherein the biological sample comprises cell-free DNA, e.g., circulating tumor DNA.

E10. The method of any one of embodiment E1-E9, further comprising:

(i) acquiring a sequence for a subgenomic interval from cell-free DNA from a sample;

(ii) acquiring a leukocyte parameter, e.g., sequence of the subgenomic interval, from leukocyte DNA from the sample.

E11. The method of any one of embodiments E1-E10 further comprising:

(i) acquiring a sequence for a subgenomic interval for aneuploidy analysis from cell-free DNA from a sample;

(ii) acquiring a leukocyte parameter, e.g., a sequence for the subgenomic interval for aneuploidy analysis, from leukocyte DNA from the sample.

E12. The method of embodiment E10 or E11 further comprising comparing (i) with (ii) to evaluate a genomic event, e.g., a mutation, found in the cell-free DNA subgenomic interval or cell-free DNA aneuploidy analysis sample.

E13. The method of any one of embodiments E10-E12, further classifying a genomic event, e.g., a mutation, in the subgenomic interval from cell-free DNA or from aneuploidy analysis of cell-free DNA, e.g., assigning the mutation to a first class or a second class.

E14. The method of any one of embodiments E10-E13, further comprising classifying a genomic event, e.g., a mutation, in the subgenomic interval from cell-free DNA or from aneuploidy analysis of cell-free DNA, as growth-deregulating, e.g., cancerous.

E15. The method of any one of embodiments E10-E13, further comprising classifying a genomic event, e.g., a mutation, in the subgenomic interval from cell-free DNA or from aneuploidy analysis of cell-free DNA, as other than growth-deregulating, e.g., as other than cancerous.

E16. The method of any one of embodiments E10-E14, wherein classifying a genomic event, e.g., a mutation, in the subgenomic interval from cell-free DNA or from aneuploidy analysis of cell-free DNA, as cancerous when:

(a) the subgenomic interval is aneuploid in cell-free DNA, and the subgenomic interval is not aneuploid in leukocytes; or

(b) the genomic event is present in the subgenomic interval of cell-free DNA, and the genomic event is not present in the subgenomic interval of leukocytes.

E17. The method of any one of embodiments E10-E13 or E15, wherein classifying a genomic event, e.g., a mutation, in the subgenomic interval from cell-free DNA or form aneuploidy analysis of cell-free DNA, as other than growth-deregulating when:

(a) the subgenomic interval is aneuploid in cell-free DNA, and the subgenomic interval is aneuploid in leukocytes; or

(b) the genomic event is present in the subgenomic interval of cell-free DNA and the genomic event is present in the subgenomic interval of leukocytes.

E18. The method of embodiment E17, wherein the genomic event is associated with clonal expansion of leukocytes, e.g., age-associated clonal hematopoiesis, e.g., clonal hematopoiesis of indeterminate potential (CHIP).

E19. The method of any one of embodiments E1-E18, wherein specificity of detection of the cancer in the plurality of cancers with (i), (ii) and (iii) is substantially the same as, e.g., not substantially lower than, the specificity of detection of the cancer in the plurality of cancers with: (i); (ii); (iii); (i) and (ii); (i) and (iii); or (ii) and (iii).

E20. The method of any one of embodiments E1-E19, wherein sensitivity of detection of the cancer in the plurality of cancers with (i), (ii) and (iii) is higher, e.g., about 1.1, 1.2, 1.3, 1.4, 1.5, 2, 2.5, 3, 3.5, 4, 4.5, 5, 5.5, 6, 6.5, 7, 7.5, 8, 8.5, 9, 9.5, or 10 fold higher, than the sensitivity of detection of the cancer in the plurality of cancers with: (i); (ii); (iii); (i) and (ii); (i) and (iii); or (ii) and (iii).

E21. The method of any one of embodiments E1-E20, wherein (i), (ii) and (iii) result in an increased sensitivity of detection, e.g., about 1.1, 1.2, 1.3, 1.4, 1.5, 2, 2.5, 3, 3.5, 4, 4.5, 5, 5.5, 6, 6.5, 7, 7.5, 8, 8.5, 9, 9.5, or 10 fold increase in sensitivity of detection at a specified specificity, e.g., at a predetermined specificity, e.g., at least about 90%, 91%, 92%, 93%, 94%, 95%, 96%, 97%, 98%, 99% or 100% specificity.

E22. The method of any one of embodiments E20-E21, wherein the increase in sensitivity of detection of the cancer in the plurality of cancers does not affect, e.g., reduce or substantially reduce, the specificity of detection of the cancer in the plurality of cancer.

E23. The method of embodiment E22, wherein the specificity of detection of the cancer in the plurality of cancers is at a plateau.

E24. The method of any one of embodiments E1-E23, wherein the RE family is other than a LINE.

E25. The method of any one of embodiments E1-E24, wherein the RE family comprises a repeated element which when amplified with a primer to its repeated terminal elements, provides a plurality of amplicons having an average length of less than about 110 bp, e.g., about 10-110 bp, about 10-105 bp, about 10-100 bp, about 10-99 bp, about 10-98 bp, about 10-97 bp, about 10-96 bp, about 10-95 bp, about 10-94 bp, about 10-93 bp, about 10-92 bp, about 10-91 bp, about 10-90 bp, about 10-89 bp, about 10-87 bp, about 10-86 bp, about 10-85 bp, about 10-84 bp, about 10-83 bp, about 10-82 bp, about 10-81 bp, about 10-80 bp, about 10-79 bp, about 10-78 bp, about 10-77 bp, about 10-76 bp, about 10-75 bp, about 10-74 bp, about 10-73 bp, about 10-72 bp, about 10-71 bp, about 10-70 bp, about 10-65 bp, about 10-60 bp, about 10-55 bp, about 10-50 bp, about 10-40 bp, about 10-30 bp, about 10-20 bp, about 15-110 bp, about 20-110 bp, about 25-110 bp, about 30-110 bp, about 35-110 bp, about 40-110 bp, about 45-110 bp, about 50-110 bp, about 55-110 bp about 60-110 bp, about 65-110 bp, about 70-110 bp, about 75-110 bp, about 80-110 bp, about 85-110 bp, about 90-110 bp, about 95-110 bp, about 100-110 bp, or about 105-110 bp.

E26. The method of any one of embodiments E1-E25, wherein the RE family comprises one or more repetitive elements shown in Table 1.

E27. The method of any one of embodiments E1-E26, wherein the RE family comprises a SINE or a tandem repeat (e.g., microsatellite DNA, mini-satellite DNA, satellite DNA or DNA of genes with multiple copies (e.g., DNA encoding ribosomal RNA)).

E28. The method of embodiment E27, wherein the RE family is a SINE, e.g., an Alu family, a MIR or a MIR3, or a SINE described in Vassetzky and Kramerov (2013) Nucleic Acids Res. 41: D83-89.

E29. The method of any one of embodiments E1-E28, wherein the value for aneuploidy is further a function of the copy number or length of a genomic sequence disposed between the terminal repeated elements of a LINE repeated element.

E30. The method of any one of embodiments E1-E29, wherein the value for aneuploidy is further a function of the copy number or length of a plurality of genomic sequences disposed between the terminal repeated elements of a repeated element family which when amplified with a primer complementary to its repeated terminal elements, provides amplicons having an average length of more than 100 bp.

E31. The method of any one of embodiments E1-E30, wherein the value for aneuploidy is further a function of:
a) amplifying a plurality of chromosomal sequences in a DNA sample with a pair of primers complementary to the chromosomal sequences to form a plurality of amplicons;
b) determining at least a portion of the nucleic acid sequence of one or more of the plurality of amplicons;
c) mapping the sequenced amplicons to a reference genome;
d) dividing the DNA sample into a plurality of genomic intervals;
e) quantifying a plurality of features for the amplicons mapped to the genomic intervals;
f) comparing the plurality of features of amplicons in a first genomic interval with the plurality of features of amplicons in one or more different genomic intervals; and
g) wherein at least 100,000 amplicons are formed in the step of amplifying.

E32. The method of any one of embodiments E1-E31, comprising providing a value for aneuploidy, wherein the value is a function of the copy number of at least about 5, 10, 20, 30, 50, 100, 200, 500, or 1000 different genomic sequences disposed between the terminal repeated elements of a RE family.

E33. The method of any one of embodiments E1-E32, wherein the copy number is greater than 2 or is less than 2.

E34. The method of any one of embodiments E31-E33, wherein at least about 100,000 amplicons, about 150,000 amplicons, about 200,000 amplicons; about 250,000 amplicons; about 300,000 amplicons; about 350,000 amplicons; about 400,000 amplicons; about 450,000 amplicons; about 500,000 amplicons; about 550,000 amplicons; about 600,000 amplicons; about 650,000 amplicons; about 700,000 amplicons; about 750,000 amplicons; about 800,000 amplicons; about 850,000 amplicons; about 900,000 amplicons; about 950,000 amplicons; or about 1,000,000 amplicons are formed.

E35. The method of any one of embodiments E1-E34, comprising providing a value for aneuploidy, wherein the value is a function of:

(i) the copy number or length of a first genomic sequence disposed between the terminal repeated elements of a RE family, on a first segment of genomic DNA; and

(ii) the copy number or length of a second genomic sequence disposed between the terminal repeated elements of a (e.g., the same or a different) RE family, on a second segment of genomic DNA.

E36. The method of embodiment E35, wherein:

(i) the first segment of genomic DNA and the second segment of genomic DNA are on different arms of the same chromosome, e.g., the first segment is on the q arm and the second segment is on the p arm of the same chromosome; or the first segment is on the p arm and the second segment is on the q arm of the same chromosome;

(ii) the first segment of genomic DNA and the second segment of genomic DNA are on the same arm of the same chromosome, e.g., the first segment and the second segment are both on the p arm, or q arm of a chromosome; and/or

(iii) the first segment of genomic DNA and the second segment of genomic DNA are on different chromosomes, e.g., non-homologous chromosomes.

E37. The method of any one of embodiments E1-E36, comprising providing a value for aneuploidy, wherein the value is a function of:

the copy number or length of a third genomic sequence disposed between the terminal repeated elements of a RE family, on a third chromosome.

E38. The method of any one of embodiments E1-E37, comprising providing a value for aneuploidy, wherein the value is a function of:

the copy number or length of an N^{th} genomic sequence disposed between the terminal repeated elements of a RE family, on an N^{th} chromosome, wherein N is 4, 5, 6, 7, 8, 9, 10, 11, 12, 13, 14, 15, 16, 17, 18, 19, 20, 21, 22, 23.

E39. The method of any one of embodiments E1-E38, comprising contacting subject genomic nucleic acid with a primer moiety which amplifies a sequence comprising a genomic sequence disposed between the terminal repeated elements of a RE family.

E40. The method of embodiment E39, wherein the primer moiety is complementary to a terminal element of the RE family.

E41. The method of embodiment E39 or E40, wherein the primer moiety comprises a pair of primers.

E42. The method of any one of embodiments E39-E41, wherein the primer moiety comprises a single primer, and e.g., is used with isothermal amplification.

E43. The method of any one of embodiments E1-E42, wherein, the number of biomarkers (e.g., number of driver gene mutations) detected is sufficient such that the sensitivity of detection of the cancer in the plurality of cancers with which each gene, e.g., driver gene, is associated with, is not substantially increased by the detection of one or more additional genetic biomarkers.

E44. The method of any one of embodiments E1-E42, wherein detecting the genetic biomarker comprises providing, e.g., by sequencing, the sequence (e.g., nucleotide sequence) of the genetic biomarker.

E45. The method of embodiment E44, wherein the number of genetic biomarker sequences provided is sufficient such that the sensitivity of detection of the cancer in the plurality of cancers with which each gene, e.g., driver gene, is associated with is not substantially increased by the provision of one or more sequences of additional genetic biomarkers.

E46. The method of any one of embodiments E1-E42, wherein detecting the biomarker comprises providing the sequence (e.g., nucleotide sequence) of one or more subgenomic intervals comprising the genetic biomarker.

E47. The method of embodiment E46, wherein, the number of subgenomic interval sequences provided is sufficient such that the sensitivity of detection of the cancer in the plurality of cancers with which each gene, e.g., driver gene, is associated with is not substantially increased by the provision of one or more sequences (e.g., nucleotide sequences) of additional subgenomic intervals.

E48. The method of any one of embodiments E1-E42, wherein detecting the genetic biomarker comprises providing the sequence of an amplicon comprising the genetic biomarker.

E49. The method of embodiment E48, wherein, the number of amplicon sequences provided is sufficient such that the sensitivity of detection of the cancer in the plurality of cancers with which each gene, e.g., driver gene, is associated with is not substantially increased by the provision of one or more sequences of additional amplicons.

E50. The method of embodiment E46, wherein the number of subgenomic interval sequences provided is sufficient such that the specificity of detection of the cancer in the plurality of cancers with which each gene, e.g., driver gene, is associated with is not substantially decreased by the provision of one or more sequences of additional subgenomic intervals.

E51. The method of embodiment E48, wherein the number of amplicons provided is sufficient such that the specificity of detection of the cancer in the plurality of cancers with which each gene, e.g., driver gene, of the plurality is associated with is not substantially decreased by the provision of one or more sequences of additional amplicons.

E52. The method of any of the preceding embodiments, wherein the plurality of cancers comprises 4, 5, 6, 7 or 8 cancers.

E53. The method of any of the preceding embodiments, wherein the plurality of cancers is chosen from solid tumors such as: mesothelioma (e.g., malignant pleural mesothelioma), lung cancer (e.g., non-small cell lung cancer, small cell lung cancer, squamous cell lung cancer, or large cell lung cancer), pancreatic cancer (e.g., pancreatic ductal adenocarcinoma), liver cancer (e.g., hepatocellular carcinoma, or cholangiocarcinoma), esophageal cancer (e.g., esophageal adenocarcinoma or squamous cell carcinoma), head and neck cancer, ovarian cancer, colorectal cancer, bladder cancer, cervical cancer, uterine cancer (endometrial cancer), kidney cancer, breast cancer, prostate cancer, brain cancer (e.g., medulloblastoma, or glioblastoma), or sarcoma (e.g., Ewing sarcoma, osteosarcoma, rhabdomyosarcoma), or a combination thereof.

E54. The method of any of the preceding embodiments, wherein the plurality of cancers is chosen from liver cancer, ovarian cancer, esophageal cancer, stomach cancer, pancreatic cancer, colorectal cancer, lung cancer, breast cancer, or prostate cancer, or a combination thereof.

E55. The method of any of the preceding embodiments, wherein one or more of the plurality of cancers is chosen from liver cancer, ovarian cancer, esophageal cancer, stomach cancer, pancreatic cancer, colorectal cancer, lung cancer, or breast cancer.

E56. The method of any of the preceding embodiments, wherein one or more of the plurality of cancers is a hematological cancer.

E57. The method of any of the preceding embodiments, wherein no more than 60, 100, 150, 200, 300 or 400 subgenomic intervals or amplicons from the one or more genes, e.g., one or more driver genes, e.g., genes listed in Tables 60 and 61 of US2019/0256924A1, e.g., ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CPLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDMSC, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, or SKP2, are sequenced.

E58. The method of any of the preceding embodiments, wherein at least 30, 40, 50 or 60 subgenomic intervals or amplicons from the one or more genes, e.g., one or more driver genes, e.g., genes listed in Tables 60 and 61 of US2019/0256924A1, e.g., ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CYLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDMSC, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, or SKP2, are sequenced.

E59. The method of any of the preceding embodiments, wherein at least 30 and not more than 400, at least 40 and not more than 300, at least 50 and no more than 200, at least 60 and no more than 150, or at least 60 and no more than 100, subgenomic intervals or amplicons from the one or more genes, e.g., one or more driver genes, e.g., one or more genes listed in Tables 60 and 61 of US2019/0256924A1, e.g., ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CYLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDMSC, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, or SKP2, are sequenced.

E60. The method of any of the preceding embodiments, wherein the number of subgenomic intervals or amplicons sequenced for a gene is no greater than 125, 150, 200, or 300% of the lowest number that achieves plateau for sensitivity of detection of the cancer.

E61. The method of any of the preceding embodiments, wherein each subgenomic interval or amplicon of the genetic biomarker comprises 6-800 bp, e.g., 6-750 bp, 6-700 bp, 6-650 bp, 6-600 bp, 6-550 bp, 6-500 bp, 6-450 bp, 6-400 bp, 6-350 bp, 6-300 bp, 6-250 bp, 6-200 bp, 6-150 bp, 6-100 bp, 10-800 bp, 15-800 bp, 20-800 bp, 25-800 bp, 30-800 bp, 35-800 bp, 40-800 bp, 45-800 bp, 50-800 bp, 55-800 bp, 60-800 bp, 65-800 bp, 70-800 bp, 75-800 bp, 80-800 bp, 85-800 bp, 90-800 bp, 95-800 bp, 100-800 bp, 200-800 bp, 300-800 bp, 400-800 bp, 500-800 bp, 600-800 bp, 700-800 bp, 10-700 bp, 20-600 bp, 30-500 bp, 40-400 bp, 50-300 bp, 60-200 bp, 61-150 bp, 62-140 bp, 63-130 bp, 64-120 bp, or 65-100 bp, e.g., 66-80 bp.

E62. The method of any of the preceding embodiments, wherein each subgenomic interval or amplicon of the genetic biomarker comprises about 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 100, or 110 bp.

E63. The method of any of the preceding embodiments, wherein each subgenomic interval or amplicon of the genetic biomarker comprises no more than 50, 55, 60, 65, 70, 75, 80, 85, 90, 95, 100, 200, 300, 400, 500, 600, 700, or 800 bp.

E64. The method of any of the preceding embodiments, wherein each subgenomic interval or amplicon of the genetic biomarker comprises at least 6, 10, 15, 20, 25, 30, 35, 40, 45, or 50 bp.

E65. The method of any of the preceding embodiments, wherein each subgenomic interval or amplicon of the genetic biomarker comprises at least 6 pb and no more than 800 bp, at least 10 bp and no more than 700 bp, at least 15 bp and no more than 600 bp, at least 20 bp and no more than 600 bp, at least 25 bp and no more than 500 bp, at least 30 bp and no more than 400 bp, at least 35 bp and no more than 300 bp, at least 40 bp and no more than 200 bp, at least 45 bp and no more than 100 bp, at least 50 bp and no more than 95 bp, or at least 55 bp and no more than 90 bp.

E66. The method of any of the preceding embodiments, wherein each subgenomic interval or amplicon of the genetic biomarker comprises 66-80 bp.

E67. The method of any of the preceding embodiments, wherein the number of subgenomic intervals or amplicons of the genetic biomarker comprises no more than 2000, 2500, 3000, 3500, 4000, 5000, 6000, 7000, 8000, 9000, 10,000, 15,000, or 20,000 bp.

E68. The method of any of the preceding embodiments, wherein the number of subgenomic intervals or amplicons of the genetic biomarker comprises at least 200, 300, 400, 500, 600, 700, 800, 900, 1000, 1100, 1200, 1300, 1400, 1500, 1600, 1700, 1800, 1900 or 2000 bp.

E69. The method of any of the preceding embodiments, wherein the number of subgenomic intervals or amplicons of the genetic biomarker comprises at least 200 bp and no more than 20,000 bp, at least 300 bp and no more than 15,000 bp, at least 400 bp and no more than 10,000 bp, at least 500 bp and no more than 9000, at least 600 bp and no more than 8000 bp, at least 700 bp and no more than 7000 bp, at least 800 bp and no more than 6000 bp, at least 900 bp and no more than 5000 bp, at least 1000 bp and no more than 4000 bp, at least 1100 bp and no more than 3500 bp, at least 1200 bp and no more than 3000 bp, at least 1300 bp and no more than 2500 bp, or at least 1500 bp and no more than 2000 bp.

E70. The method of any of the preceding embodiments, wherein the number of subgenomic intervals or amplicons of the genetic biomarker comprises 200+15%, 300+15%, 400+15%, 500+15%, 600+15%, 700+15%, 800+15%, 900+15%, 1000+15%, 1100+15%, 1200+15%, 1300+15%, 1400+15%, 1500+15%, 1600+15%, 1700+15%, 1800+15%, 1900+15%, 2000+15%, 2500+15%, 3000+15%, 3500+15%, 4000+15%, 5000+15%, 6000+15%, 7000+15%, 8000+15%, 9000+15%, 10,000+15%, 15,000+15%, or 20,000 bp+15%, e.g., 2000 bp+15%.

E71. The method of any of the preceding embodiments, wherein the number of subgenomic intervals or amplicons of the genetic biomarker comprise 2000 bp.

E72. The method of any of the preceding embodiments, wherein the average depth to which the number of subgenomic intervals or amplicons of the genetic biomarker is sequenced is at least 5× sequencing depth.

E73. The method of any of the preceding embodiments, wherein the average depth to which the number of subgenomic intervals or amplicons of the genetic biomarker is sequenced is no more than 500× sequencing depth.

E74. The method of any of the preceding embodiments, wherein the average depth to which the number of subgenomic intervals or amplicons of the genetic biomarker is sequenced is between 5× to 500× sequencing depth.

E75. The method of any of the preceding embodiments, wherein said detecting step comprises sequencing each subgenomic interval to a depth of at least 50,000 reads per base.

E76. The method of any of the preceding embodiments, wherein said detecting step comprises sequencing each subgenomic interval to a depth of no more than 150,000 reads per base.

E77. The method of any of the preceding embodiments, wherein said detecting step comprises sequencing each subgenomic interval to a depth of from 50,000 reads per base to 150,000 reads per base.

E78. The method of any of the preceding embodiments, wherein said detecting step comprises sequencing each subgenomic interval at a depth sufficient to detect a mutation in said region of interest at a frequency as low as 0.0005%.

E79. The method of any of the preceding embodiments, wherein no more than 20, 21, 22, 23, 24, 25, 26, 27, 28, 29, 30, 31, 32, 33, 34, 35, 40, 45, 50, 55, 60,100, 200 or 300 bp, is sequenced for each biomarker, e.g., each gene, e.g., each driver gene, e.g., each gene disclosed in Table 60 or 61 in US2019/0256924A1 e.g., ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CYLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDMSC, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, or SKP2.

E80. The method of any of the preceding embodiments, wherein at least 6, 7, 8, 9, 10, 11, 12, 13, 14, 15, 16, 17, 18, 19, or 20 bp, is sequenced in each biomarker, e.g., each gene, e.g., each driver gene, e.g., each gene disclosed in Table 60 or 61 in US2019/0256924A1, e.g., ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CYLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDM5C, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, or SKP2.

E81. The method of any of the preceding embodiments, wherein at least 6 and no more than 300 bp, at least 7 and no more than 200 bp, at least 8 bp and no more than 100 bp, at least 9 bp and no more than 60 bp, at least 10 bp and no more than 55 bp, at least 11 bp and no more than 50 bp, at least 12 bp and no more than 45 bp, at least 13 bp and no more than 40 bp, at least 14 bp and no more than 35 bp, at least 15 bp and no more than 34 bp, at least 14 bp and no more than 33 bp, at least 15 bp and no more than 32 bp, at least 16 bp and no more than 31 bp, at least 17 bp and no more than 30 bp, at least 18 bp and no more than 29 bp, at least 19 bp and no more than 28 bp, at least 20 bp and no more than 27 bp, is sequenced in each biomarker, e.g., each gene, e.g., each driver gene, e.g., each gene disclosed in Table 60 or 61 in US2019/0256924A1, e.g., ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CYLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDM5C, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, or SKP2.

E82. The method of any of the preceding embodiments, wherein about 33 bp is sequenced in each biomarker, e.g., each gene, e.g., each driver gene, e.g., each gene disclosed in Table 60 or 61 in US2019/0256924A1, e.g., ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CYLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDM5C, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, or SKP2.

E83. The method of any of the preceding embodiments, wherein detecting the biomarker comprises providing the sequence of the subgenomic interval or amplicon of no more than 20, 21, 22, 23, 24, 25, 26, 27, 28, 29, 30, 31, 32, 33, 34, 35, 40, 45, 50, 55, 60,100, 200 or 300 bp, in length and wherein the subgenomic interval or the amplicon comprises the biomarker, e.g., a driver gene comprising a driver mutation.

E84. The method of any of the preceding embodiments, wherein detecting the biomarker comprises providing the sequence of the subgenomic interval or the amplicon of at least 6, 7, 8, 9, 10, 11, 12, 13, 14, 15, 16, 17, 18, 19, or 20 bp, in length and wherein the subgenomic interval or the amplicon comprises the biomarker, e.g., a driver gene comprising a driver mutation.

E85. The method of any of the preceding embodiments, wherein detecting the biomarker comprises providing the sequence of a subgenomic interval or amplicon of at least 6 and no more than 300 bp, at least 7 and no more than 200 bp, at least 8 bp and no more than 100 bp, at least 9 bp and no more than 60 bp, at least 10 bp and no more than 55 bp, at least 11 bp and no more than 50 bp, at least 12 bp and no more than 45 bp, at least 13 bp and no more than 40 bp, at least 14 bp and no more than 35 bp, at least 15 bp and no more than 34 bp, at least 14 bp and no more than 33 bp, at least 15 bp and no more than 32 bp, at least 16 bp and no more than 31 bp, at least 17 bp and no more than 30 bp, at least 18 bp and no more than 29 bp, at least 19 bp and no more than 28 bp, at least 20 bp and no more than 27 bp, in length and wherein the subgenomic interval or amplicon comprises the biomarker, e.g., driver gene comprising a driver mutation.

E86. The method of any of the preceding embodiments, wherein detecting the biomarker comprises providing the sequence of a subgenomic interval or amplicon of between 6 bp and 300 bp, 7 bp and 200 bp, or 8 and 100 bp, 9 bp and 60 bp, 10 bp and 50 bp, 15 bp and 40 bp, 20 bp and 35 bp in length and wherein the subgenomic interval or amplicon comprises the biomarker, e.g., driver gene comprising a driver mutation.

E87. The method of any of the preceding embodiments, wherein detecting the biomarker comprises providing the sequence of a subgenomic interval or amplicon of about 33 bp in length and wherein the subgenomic interval or amplicon comprises the biomarker, e.g., driver gene comprising a driver mutation.

E88. The method of any of the preceding embodiments, further comprising:

b) detecting the level of each of a plurality of, e.g., at least four, protein biomarkers in a biological sample, wherein the level of each protein biomarker of the plurality is associated with the presence of a cancer of the plurality of cancers;

(optionally) (c) comparing the detected levels of each protein biomarker of the plurality of protein biomarkers to a reference level for the protein biomarker; and d) identifying the presence of a cancer of the plurality of cancers in the subject when the presence of one or more genetic biomarkers and the level of one of the protein biomarkers of the plurality of protein biomarkers is detected.

E89. The method of any of the preceding embodiments, wherein:

(i) the subject has not yet been determined to have a cancer, e.g., a cancer selected from the plurality of cancers,

(ii) the subject has not yet been determined to harbor a cancer cell, e.g., a cancer cell selected from the plurality of cancers, or

(iii) the subject does not exhibit, or has not exhibited a symptom associated with a cancer, e.g., a cancer selected from the plurality of cancers.

E90. The method of any of the preceding embodiments, wherein the subject:

(i) is a pediatric subject or a young adult; e.g., aged 6 months-21 years; or

(ii) is an adult, e.g., aged 18 years or older.

E91. The method of any of the preceding embodiments, wherein the sample comprises a tumor sample, e.g., a biopsy sample (e.g., a liquid biopsy sample (e.g., a circulating tumor DNA sample, or a cell-free DNA sample) or a solid tumor biopsy sample); a blood sample (e.g., a circulating tumor DNA sample, or a cell-free DNA sample), an apheresis sample, a urine sample, a cyst fluid sample (e.g., a pancreatic cyst fluid sample), a Papanicolaou (Pap) sample, or a fixed tumor sample (e.g., a formalin fixed sample or a paraffin embedded sample (FPPE)).

E92. The method of any of the preceding embodiments, wherein the one or more, e.g., plurality of, genes comprises 1, 2, 3, or 4 genes from Tables 60 and 61 of US2019/0256924A1, e.g., ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CPLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDM5C, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, or SKP2.

E93. The method of any of the preceding embodiments, wherein the one or more, e.g., plurality of, genes comprises 5, 6, 7, or 8 genes, chosen from Tables 60 and 61 of US2019/0256924A1, e.g., ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CYLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDM5C, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, or SKP2.

E94. The method of any of the preceding embodiments, wherein the one or more, e.g., plurality of, genes is a gene selected from: NRAS, CTNNB1, PIK3CA, FBXW7, APC, EGFR, BRAF, CDKN2A, PTEN, FGFR2, HRAS, KRAS, AKT1, TP53, PPP2R1A, or GNAS.

E95. The method of any of the preceding embodiments, wherein the one or more, e.g., plurality of, biomarkers (e.g., one or more genes) is chosen from KRAS, PIK3CA, HRAS, CDKN2A, TP53, AKT1, CTNNB1, APC, EGFR, GNAS, PPP2R1A, BRAF, FBXW7, PTEN, or FGFR2, or a combination thereof, and the cancer is chosen from: liver cancer, ovarian cancer, esophageal cancer, stomach cancer, pancreatic cancer, colorectal cancer, lung cancer, breast cancer, or prostate cancer.

E96. The method of any of the preceding embodiments, wherein the one or more, e.g., plurality of, biomarkers (e.g., one or more genes) is chosen from KRAS, PIK3CA, HRAS, CDKN2A, TP53, TERT, ERBB2, FGFR3, MET, MLL, or VHL, or a combination thereof, and the cancer is chosen from a bladder cancer or upper tract urothelial carcinoma (UTUC).

E97. The method of any of the preceding embodiments, wherein the one or more, e.g., plurality of, biomarkers (e.g., one or more genes) is chosen from KRAS, PIK3CA, CDKN2A, TP53, CTNNB1, PPP2R1A, BRAF, PTEN, CSMD3, FAT3, BRCA, or ARID1A, or a combination thereof, and the cancer is an ovarian cancer or an endometrial cancer.

E98. The method of any of the preceding embodiments, wherein the one or more, e.g., plurality of, biomarkers (e.g., one or more genes) is chosen from KRAS, PIK3CA, CDKN2A, TP53, CTNNB1, GNAS, BRAF, NRAS, VHL, RNF43, or SMAD4, or a combination thereof, and the cancer is a pancreatic cancer, e.g., a pancreatic ductal adenocarcinoma (PDAC).

E99. The method of any of the preceding embodiments, wherein the one or more, e.g., plurality of biomarkers, comprises 5, 6, 7, or 8 protein biomarkers.

E100. The method of any of the preceding embodiments, wherein the one or more, e.g., plurality of biomarkers, comprises a protein biomarker selected from: CA19-9, CEA, HGF, OPN, CA125, prolactin (PRL), TIMP-1, CA15-3, AFP or MPO.

E101. The method of any of the preceding embodiments, wherein detecting the presence of one or more genetic biomarkers comprises:

a. assigning a unique identifier (UID) to each of a plurality of template molecules present in the sample;

b. amplifying each uniquely tagged template molecule to create UID-families; and

c. redundantly sequencing the amplification products.

E102. The method of any of the preceding embodiments, further comprising detecting the presence of aneuploidy in the sample, e.g., detecting gain or loss in one or more chromosomes, e.g., using the WALDO method as described in Example 6.

E103. The method of embodiment 102, wherein the method comprises: (i) estimating somatic mutation load; (ii) estimating carcinogen signature, and/or (iii) detecting microsatellite instability (MSI).

E104. The method of embodiment 102 or 103, wherein the method can be used to compare two samples, e.g., two unrelated samples, to evaluate genetic similarities between the samples or to find somatic mutations within the samples, e.g., within the LINE elements in the sample.

E105. The method of embodiment 102 or 103, wherein the method results in an increase in specificity and/or sensitivity of aneuploidy detection.

E106. The method of embodiment 102, wherein the presence of aneuploidy is detected on one or more chromosome arms.

E107. The method of any of the preceding embodiments, further comprising responsive to a value of: a genetic marker, a protein biomarker and/or aneuploidy status, assigning an origin or cancer type to the cancer.

E108. The method of any one of the preceding embodiments, wherein responsive to a value of: a genetic marker, a protein biomarker and/or aneuploidy status, the method comprises identifying the subject as having a cancer, or having a risk of developing a cancer.

E109. The method of embodiment E108, further comprising administering to the subject a therapeutic agent to treat the cancer, or selecting a therapeutic agent for treating the cancer in the subject.

E110. The method of embodiment E109, wherein the subject is administered the therapeutic agent in combination with one or more additional therapeutic agents.

E111. A reaction mixture comprising:

at least 2, 3, 4, 5, 6, 7, 8, 9 or 10 detection reagents, wherein a detection reagent mediates a readout that is a value of the level or presence of:

(i) one or more genetic biomarkers referred to herein;

(ii) one or more protein biomarkers referred to herein; and/or

(iii) the copy number or length, e.g., aneuploidy, of a genomic sequence disposed between at least two terminal repeated elements of a repeated element family (RE Family) referred to herein.

E112. The reaction mixture of embodiment E111, comprising a plurality of detection reagents for (i).

E113. The reaction mixture of any of embodiments E111-E112, comprising a plurality of detection reagents for (ii).

E114. The reaction mixture of any of embodiments E111-E113, comprising a plurality of detection reagents for (iii).

E115. The reaction mixture of any of embodiments E111-E114, comprising a sample from a subject, e.g., a subject sample.

E116. A kit comprising:

(a) at least 2, 3, 4, 5, 6, 7, 8, 9 or 10 detection reagents, wherein a detection reagent mediates a readout that is a value of the level or presence of:

(i) one or more genetic biomarkers referred to herein;

(ii) one or more protein biomarkers referred to herein; and/or

(iii) the copy number or length, e.g., aneuploidy, of a genomic sequence disposed between at least two terminal repeated elements of a repeated element family (RE Family) referred to herein; and

(b) instructions for using said kit.

E117. The reaction mixture of embodiment E116, comprising a plurality of detection reagents for (i).

E118. The reaction mixture of any of embodiments E116-E117, comprising a plurality of detection reagents for (ii).

E119. The reaction mixture of any of embodiments E116 to E118, comprising a plurality of detection reagents for (iii).

E120. The method of any one of embodiments E1-E110, wherein aneuploidy status is evaluated, e.g., determined, using a first primer and a second primer.

E121. The method of embodiment E120, wherein the first primer comprises a sequence that is at least 80%, 85%, 90%, 95%, 96%, 96%, 98%, 99%, or 100% identical to SEQ ID NO: 1

E122. The method of embodiment E121, wherein the first primer comprises the sequence of SEQ ID NO: 1.

E123. The method of embodiment E120, wherein the second primer comprises a sequence that is at least 80%, 85%, 90%, 95%, 96%, 96%, 98%, 99%, or 100% identical to SEQ ID NO: 10.

E124. The method of embodiment E123, wherein the second primer comprises the sequence of SEQ ID NO: 10.

E125. The method of any one of embodiments E1-E110, or E120-E124, further comprising subjecting the subject to a radiologic scan, e.g., a PET-CT scan, of an organ or body region.

E126. The method of embodiment 125, wherein the radiologic scanning of an organ or body region characterizes the cancer.

E127. The method of embodiment 125, wherein the radiologic scanning of an organ or body region identifies the location of the cancer.

E128. The method of any one of embodiments E125-E127, wherein the radiologic scan is a PET-CT scan.

E129. The method of any one of embodiments E125-E128, wherein the radiologic scanning is performed after the subject is evaluated for the presence of each of a plurality of cancers.

E130. The method of any one of embodiments E1-E110, or E120-E129, comprising administering to the subject one or more therapeutic interventions (e.g., surgery, adjuvant chemotherapy, neoadjuvant chemotherapy, radiation therapy, immunotherapy, targeted therapy, and/or an immune checkpoint inhibitor).

E131. The method of any one of embodiments E1-E110, or E120-E130, wherein the evaluation comprises evaluating a sample from the subject at one time point or at different time points.

E132. The method of any one of embodiments E1-E110, or E120-E131, comprising evaluating one or more samples, e.g., multiple samples, obtained from the subject.

E133. The method of E132, wherein the one or more samples, e.g., multiple samples, are obtained yearly, e.g., within 1 year of one another.

E134. The method of any of embodiments E1-E110, or E120-E133, wherein the subject is evaluated simultaneously for the presence or absence of each of a plurality of cancers.

E135. The method of any of embodiments E1-E110, or E120-E134, wherein the subject is co-evaluated for the presence or absence of each of a plurality of cancers.

E136. The method of any of embodiments E1-E110, or E120-E135, comprising evaluating the presence of each of a plurality of cancers in a subject at one or more time points within a predetermined interval, e.g., at the same or substantially the same clinical stage of at least one of the cancers in the subject.

E137. The method of any of embodiments E1-E110, or E120-E136, comprising evaluating a sample, e.g., a single sample or multiple samples, obtained from the subject.

E138. The method of any of embodiments E1-E110, or E120-E137, wherein co-evaluation is performed on a single sample, aliquots of a single sample, or a plurality of samples taken, e.g., within 1, 5, 24 or 48 hours, of one another.

E139. The method of any embodiments E1-E110, or E120-E138, wherein the subject is asymptomatic for cancer.

E140. The method of any of embodiments E1-E110, or E120-E139, wherein the subject is asymptomatic for a cancer of the plurality.

E141. The method of any of embodiments E1-E110, or E120-E140, wherein the subject is not known or determined to harbor a cancer cell.

E142. The method of any of embodiments E1-E110, or E120-E141, wherein the subject has not been determined to have or diagnosed with a cancer.

E143. The method of any of embodiments E1-E110, or E120-E142, wherein the subject has an early stage cancer, e.g., Stage I or Stage II.

E144. The method of any of embodiments E1-E110, or E120-E143, wherein the subject is pre-metastatic.

E145. The method of any of embodiments E1-E110, or E120-E144, wherein the subject has no detectable metastasis.

E146. The method of any of embodiments E1-E110, or E120-E145, wherein the subject has not exhibited a symptom associated with a cancer.

E147. The method of any of embodiments E1-E110, or E120-E146, wherein the subject does not display one, two or more symptoms clinically associated with the cancer.

E148. The method of any of embodiments E1-E110, or E120-E147, wherein when the aneuploidy status is positive, the subject has an early stage cancer, e.g., Stage I or Stage II e.g., as provided in Table 3.

E149. The method of any of embodiments E1-E110, or E120-E147, wherein when the aneuploidy status is negative, the subject has an early stage cancer, e.g., Stage I or Stage II e.g., as provided in Table 3.

E150. A method of detecting aneuploidy in a sample comprising low input DNA.

E151. The method of any of embodiments E1-E110, or E120-E150, wherein the sample comprises about 0.01 picogram (pg) to 500 pg of DNA.

E152. The method of embodiment E151, wherein the sample comprises about 0.01-500 pg, 0.05-400 pg, 0.1-300 pg, 0.5-200 pg, 1-100 pg, 10-90 pg, or 20-50 pg DNA.

E153. The method of embodiment E151, wherein the sample comprises at least 0.01 pg, at least 0.01 pg, at least 0.1 pg, at least 1 pg, at least 2 pg, at least 3 pg, at least 4 pg, at least 5 pg, at least 6 pg, at least 7 pg, at least 8 pg, at least 9 pg at least 10 pg, at least 11 pg, at least 12 pg, at least 13 pg, at least 14 pg, at least 15 pg, at least 16 pg, at least 17 pg, at least 18 pg, at least 19 pg, at least 20 pg, at least 21 pg, at least 22 pg, at least 23 pg, at least 24 pg, at least 25 pg, at least 26 pg, at least 27 pg, at least 28 pg, at least 29 pg, at least 30 pg, at least 31 pg, at least 32 pg, at least 33 pg, at least 34 pg, at least 35 pg, at least 36 pg, at least 37 pg, at least 38 pg, at least 39 pg, at least 40 pg, at least 50 pg, at least 60 pg, at least 70 pg, at least 80 pg, at least 90 pg, at least 100 pg, at least 150 pg, at least 200 pg, at least 300 pg, at least 350 pg, at least 400 pg, at least 450 pg, or at least 500 pg DNA.

E154. A method of identifying or distinguishing a sample, e.g., using any of the methods disclosed herein.

E155. The method of embodiment E154, wherein a sample, e.g., a first sample, from a subject, e.g., a first subject, is distinguished from a second sample from a second subject.

E156. The method of embodiment E154, wherein a sample is identified as being from a subject based on a polymorphism (e.g., a plurality of polymorphisms, e.g., common polymorphisms).

E157. The method of embodiment E156, wherein a polymorphism, e.g., a common polymorphism, is present in a repetitive element, e.g., as described herein.

E158. The method of embodiment E154, wherein a method disclosed in Example 8 is used to identify and/or distinguish the sample.

E159. The method of any of embodiments E1-E110, or E120-E158, wherein the method is an in vitro method.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A shows a distribution of amplicon size when using a single primer pair to amplify repetitive elements (see, e.g., Table 1 for a list of repetitive elements). The amplicon sizes shown in FIG. 1A includes the number of bases in the primers.

FIG. 1B shows a distribution of amplicon size when using a single primer pair to amplify repetitive elements (see, e.g., Table 1 for a list of repetitive elements). The amplicon sizes shown in FIG. 1B do not include the number of bases in the primers.

FIG. 1C shows a distribution of the number of amplicons observed in cell free DNA from 2231 plasma samples.

FIG. 2A. Exemplary overview of an embodiment of a workflow described herein.

FIG. 2B is an exemplary overview of an embodiment of the Repetitive Element AneupLoidy Sequencing System (RealSeqS).

FIG. 3 shows aneuploidy sensitivity vs mutations (@99% specificity) in different cancer types. The percent of aneuploidy detected in each cancer type is depicted on the Y axis.

FIG. 4 shows aneuploidy shows aneuploidy sensitivity compared to other cancer biomarkers. The percent of cancers detected (sensitivity) is depicted on the Y axis.

FIG. 5 shows pseudocode to generate synthetics with multiple arm alterations.

FIG. 6 shows estimation of the relationship between reads and DNA concentration.

FIG. 7A shows a comparison of the sensitivity of cancer detection with different multi-analyte tests. Three different multi-analyte test evaluated sensitivity of detecting the eight indicated cancers. The three tests were: (1) aneuploidy status, somatic mutation analysis and protein biomarker evaluation; (2) aneuploidy status and somatic mutation analysis; and (3) aneuploidy status and protein biomarker evaluation.

FIG. 7B shows the sensitivity of a test incorporating aneuploidy, mutations, and abnormally high levels of 8 proteins compared to a test comparing only aneuploidy+ proteins or only mutations and proteins. All sensitivities were calculated at an aggregate of 99% specificity (i.e., only 1% of the plasma samples was positive for aneuploidy, mutations, or proteins in the test incorporating aneuploidy, mutations, and proteins using 10 iterations of 10 fold cross validation).

FIG. 8 is a graph showing the true positive fraction (sensitivity) on the y-axis and the false positive fraction of cancer detection using the various tests. The tests include: (1) aneuploidy status; somatic mutation; and protein biomarker; (2) aneuploidy status and protein biomarker; (3) somatic mutation and protein biomarker; (4) aneuploidy status and somatic mutation; (5) aneuploidy status; and (6) somatic mutation. The true positive fraction (sensitivity) was calculated using a threshold at 99% specificity.

FIG. 9 shows sensitivity of cancer detection for aneuploidy alone (@98% or 99% specificity) compared to sensitivity with aneuploidy and protein biomarkers (@95% specificity) in different stages of cancer.

FIG. 10 shows aneuploidy (@99% specificity) in different stages of cancer.

FIG. 11 shows aneuploidy (@99% specificity) in different cancer types.

FIG. 12 shows sensitivity when aneuploidy (@99% specificity) is combined with detection of protein biomarkers.

FIG. 13 shows pseudocode to generate in silico trisomy and monosomy samples used for the comparison of whole genome sequencing, FAST-SeqS and Real SeqS.

FIG. 14 shows pseudocode to generate in silico simulated samples with multiple arm alterations that were used in the Genome Wide Aneuploidy SVM training set.

FIGS. 15A-15C show detection of Aneuploidy using Next Generation Sequencing Technologies. Sensitivities were calculated at 99% specificity. Error bars represent 95% confidence intervals. FIG. 15A Comparison of sensitivity for monosomies and trisomies across all 39 non-acrocentric chromosome arms at 5% cell fraction. FIG. 15B Comparison of sensitivity for the 1.5 Mb DiGeorge deletion on 22q at 5% cell fraction. FIG. 15C Comparison of sensitivity for a 20 copy ERBB2 focal amplification at 1% cell fraction.

FIGS. 16A-16B show examples of plasma samples with focal deletions or amplifications. FIG. 16A shows RealSeqS data on a plasma sample from a normal individual with a ˜3 Mb deletion of chromosome 22, characteristic of DiGeorge Syndrome. Note that many patients with microdeletions at this locus have mild signs and symptoms and are clinically undetected. FIG. 16B shows RealSeqS data on a typical plasma sample from a normal individual, showing no deletion at the DiGeorge locus.

FIGS. 17A-17B show examples of plasma samples with focal deletions or amplifications. FIG. 17A shows RealSeqS data on a plasma sample from a patient with cancer showing a 2.5 MB focal amplification that includes the ERBB2 locus on chromosome 17q. FIG. 17B shows RealSeqS data on a typical plasma sample from a normal individual, showing no amplification at the ERBB2 locus.

FIG. 18 shows RealSeqS sensitivity for plasma samples with various amounts of tumor derived DNA. The amount of tumor DNA was estimated by the mutant allele frequency (MAF) of driver mutations present in the plasma sample.

FIGS. 19A-19B show detection of cancer in liquid biopsies from samples with non-metastatic cancers of eight different types. Sensitivities were calculated at 99% specificity during cross validation. Error bars represent 95% confidence intervals. FIG. 19A shows the comparison of aneuploidy status as assessed by RealSeqS to somatic mutations status with respect to tumor type. FIG. 19B shows the comparison of aneuploidy status as calculated by RealSeqS to somatic mutations status with respect to Cancer Stage.

### DETAILED DESCRIPTION

### Definitions

The term “driver gene mutation” or “driver mutation” as used herein, refers to a mutation that (i) occurs in a driver gene; and (ii) provides a growth advantage to the cell in which it occurs. A growth advantage for a cell can include:

a) an increase in the rate of cell division in a cell having a driver gene mutation, e.g., an increase in rate of cell division as compared to a reference cell, e.g., to an otherwise similar cell, e.g., an otherwise similar cell adjacent to the cell, e.g., as compared to a cell of the same type not having the driver gene mutation;

b) an increase in the rate of clonal expansion in a cell having a driver gene mutation, e.g., an increase in rate of clonal expansion as compared to a reference cell, e.g., to an otherwise similar cell, e.g., an otherwise similar cell adjacent to the cell, e.g., as compared to a cell of the same type not having the driver mutation;

c) an increase in the number of cells that are progeny, e.g., a daughter cell, of the cell that has the driver gene mutation, e.g., an increase in number of progeny cells compared to the number of progeny cells expected if the cell did not have the driver gene mutation;

d) an increase in the ability to form tumors or promote tumor growth, e.g., tumor progression, e.g., as compared to a reference cell, e.g., to an otherwise similar cell not having the driver gene mutation; or

e) presence or appearance at a second or subsequent site or location in the subject.

In an embodiment, a driver gene mutation provides a 0.1-5%, e.g., a 0.1-4.5%, 0.1-4%, 0.1-3.5%, 0.1-3%, 0.1-2.5%, 0.1-2%, 0.1-1.5%, 0.1-1%, 0.1-0.5%, 0.5-5%, 1-5%, 1.5-5%, 2-5%, 2.5-5%, 3-5%, 3.5-5%, 4-5%, 4.5-5%, 0.5-4.5%, 1-4%, 1.5-3.5%, or 2-3%, growth advantage, e.g., increase in the difference between cell birth and cell death. In an embodiment, a driver gene mutation provides at least 0.1% 0.2%, 0.3%, 0.4%, 0.5%, 0.6%, 0.7%, 0.8%, 0.9%, 1%, 1.5%, 2%, 2.5%, 3%, 3.5%, 4%, or 4.5%, e.g., about a 0.4%, growth advantage, e.g., increase in the difference between cell birth and cell death. In an embodiment, a driver gene mutation, provides a proliferative capacity to the cell in which it occurs, e.g., allows for cell expansion, e.g., clonal expansion.

In some embodiments, the driver gene mutation can be causally linked to cancer progression.

In an embodiment, the driver gene mutation affects, e.g., alters the regulation, expression or function of, a protein coding gene. In an embodiment, a driver gene mutation affects, e.g., alters the function of, a noncoding region, e.g., non-protein coding region. In an embodiment, a driver gene mutation includes: a translocation, a deletion (e.g., a homozygous deletion), an insertion (e.g., an intragenic insertion), a small insertion and deletion (indels), a single base substitution (e.g., a synonymous mutation, non-synonymous mutation, nonsense mutation or a frameshift mutation), a copy number variation (CNV) (e.g., an amplification), or a single nucleotide variation (SNV) (e.g., a single nucleotide polymorphism (SNP)). Exemplary driver mutations are disclosed in Tables 60 and 61 of US2019/0256924A1.

In some embodiments, the presence of a driver gene mutation in a cell can alter (e.g., increase or decrease) the expression of the gene product in that cell. In some embodiments, the presence of a driver gene mutation in a cell can alter the function of the gene product. In some cases, the presence of a driver gene mutation in a cell can provide that cell with a growth advantage. For example, the presence of a driver gene mutation in a cell can cause an increase the rate of proliferation (e.g., as compared to a reference cell). For example, the presence of a driver gene mutation in a cell can cause an increase in the rate of clonal expansion in a cell having a driver gene mutation (e.g., as compared to a reference cell). For example, the presence of a driver gene mutation in a cell can cause an increase in the number of progeny cells derived from the cell having the driver gene mutation (e.g., as compared to a reference cell). For example, the presence of a driver gene mutation in a cell can cause an increase in the ability of the cell to form a tumor (e.g., as compared to a reference cell). In some cases, a growth advantage can be measures as an increase in the difference between cytogenesis (e.g., the formation of new cells) and cell death. For example, the presence of a driver gene mutation in a cell can provide that cell with a growth advantage of at least about 0.1% (e.g., about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or more). For example, the presence of a driver gene mutation in a cell can provide that cell with a growth advantage of about from 0.1% to about 5% (e.g., from about 0.1 to about 5%, from about 0.1 to about 4.5%, from about 0.1 to about 4%, from about 0.1 to about 3.5%, from about 0.1 to about 3%, from about 0.1 to about 2.5%, from about 0.1 to about 2%, from about 0.1 to about 1.5%, from about 0.1 to about 1%, from about 0.1 to about 0.5%, from about 0.5 to about 5%, from about 1 to about 5%, from about 1.5 to about 5%, from about 2 to about 5%, from about 2.5 to about 5%, from about 3 to about 5%, from about 3.5 to about 5%, from about 4 to about 5%, from about 4.5 to about 5%, from about 0.5 to about 4.5%, from about 1 to about 4%, from about 1.5 to about 3.5%, or from about 2 to about 3%).

In some cases, a driver gene can include more than one (e.g., two, three, four, five, six, seven, eight, nine, ten, or more) driver gene mutations. In some cases, a driver gene including one or more driver gene mutations also can include one or more additional mutations (e.g., passenger gene mutations (somatic mutations which are not a driver mutation)).

The term “driver gene” as used herein, refers to a gene which includes a driver gene mutation. In one embodiment, the driver gene is a gene in which one or more (e.g., one, two, three, four, five, six, seven, eight, nine, ten, or more) acquired mutations, e.g., driver gene mutations, can be causally linked to cancer progression. In an embodiment, a driver gene modulates one or more cellular processes including: cell fate determination, cell survival and genome maintenance. A driver gene can be associated with (e.g., can modulate) one or more signaling pathways. Examples of signaling pathways include, without limitation, a TGF-beta pathway, a MAPK pathway, a STAT pathway, a PI3K pathway, a RAS pathway, a cell cycle pathway, an apoptosis pathway, a NOTCH pathway, a Hedgehog (HH) pathway, an APC pathway, a chromatin modification pathway, a transcriptional regulation pathway, and a DNA damage control pathway. Examples of driver genes include, without limitation, ABL1, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CASP8, CBL, CDC73, CDH1, CDKN2A, CEBPA, CIC, CREBBP, CRLF2, CSF1R, CTNNB1, CYLD, DAXX, DNMT1, DNMT3A, EGFR, EP300, ERBB2, EZH2, FAM123B, FBXW7, FGFR2, FGFR3, FLT3, FOXL2, FUBP1, GATA1, GATA2, GATA3, GNA11, GNAQ, GNAS, H3F3A, HIST1H3B, HNF1A, HRAS, IDH1, IDH2, JAK1, JAK2, JAK3, KDM5C, KDM6A, KIT, KLF4, KRAS, MAP2K1, MAP3K1, MED12, MEN1, MET, MLH1, MLL2, MLL3, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2R1A, PRDM1, PTCH1, PTEN, PTPN11, RB1, RET, RNF43, RUNX1, SETD2, SETBP1, SF3B1, SMAD2, SMAD4, SMARCA4, SMARCB1, SMO, SOCS1, SOX9, SPOP, SRSF2, STAG2, STK11, TET2, TNFAIP3, TRAF7, TP53, TSC1, TSHR, U2AF1, VHL, WT1, CCND1, CDKN2C, IKZF1, LMO1, MAP2K4, MDM2, MDM4, MYC, MYCL1, MYCN, NCOA3, NKX2-1, and SKP2. Exemplary driver genes include oncogenes and tumor suppressors. In an embodiment, a driver gene has one or more driver gene mutations, e.g., as described herein. In an embodiment, a driver gene is a gene listed in Tables 60 or 61 in US2019/0256924A1. In an embodiment, a driver gene is a gene that modulates one or more cellular processes described in Tables 60 or 61 in US2019/0256924A1, e.g., cell fate determination, cell survival and genome maintenance. In an embodiment, a driver gene is a gene that modulates one or more pathways described in Tables 60 or 61 in US2019/0256924A1. In an embodiment, a driver gene is a gene that modulates one or more signaling pathways described in Table 62 of US2019/0256924A1.

In an embodiment, a driver gene includes more than one driver mutation, and the first driver gene mutation, provides a selective growth advantage to the cell in which it occurs. In an embodiment, the subsequent mutation, e.g., second, third, fourth, fifth or later mutation, e.g., driver mutation in the driver gene, provides a proliferative capacity to the cell in which it occurs, e.g., allows for cell expansion, e.g., clonal expansion. In an embodiment, a driver gene has one or more passenger gene mutations, e.g., a somatic mutation that arises in the development of a cancer but which is not a driver mutation. In an embodiment, a driver gene can be present, e.g., expressed, in any cell type, e.g., a cell type derived from any one of the three germ cell layers: ectoderm, endoderm or mesoderm. In an embodiment, a driver gene is present, e.g., expressed, in a somatic cell. In an embodiment, a driver gene is present, e.g., expressed, in a germ cell. In an embodiment, a driver gene can be present in a large number of cancers, e.g., in more than 5% of cancers. In an embodiment, a driver gene can be present in a small number of cancer, e.g., in less than 5% of cancers. In an embodiment, a driver gene has a mutation pattern that is non-random and/or recurrent, i.e., the location at which a driver mutation occurs in the driver gene is the same in different cancer types. Exemplary recurrent driver gene mutations include mutations in the IDH1 gene at the substrate binding site, e.g., at codon 132, and mutations in the PIK3CA gene in the helical domain or the kinase domain, as depicted in Vogelstein et al (2013) Science 339: 1546-1558.

In an embodiment, a driver gene having a driver gene mutation is an oncogene. In an embodiment, an oncogene is a gene with an oncogene score of at least 20%, e.g., at least 25%, 30%, 35%, 40%, 45%, 50%, 55%, 60%, 65%, 70%, 75%, 80%, 85%, 90%, 95%, 99% or 100%. In an embodiment, an oncogene score is defined as the number of mutations, e.g., clustered mutations (e.g., missense mutations at the same amino acid, or identical in-frame insertions or deletions) divided by the total number of mutations. In an embodiment, a driver gene having an amplification, e.g., as described herein, is an oncogene. In an embodiment, a driver gene having a driver gene mutation is a tumor suppressor gene (TSG). In an embodiment, a tumor suppressor gene is a gene with a tumor suppressor gene score of at least 20%, e.g., at least 25%, 30%, 35%, 40%, 45%, 50%, 55%, 60%, 65%, 70%, 75%, 80%, 85%, 90%, 95%, 99% or 100%. In an embodiment, a tumor suppressor gene score is defined as the number of inactivating mutations divided by the total number of mutations. In an embodiment, a driver gene having a deletion, e.g., as described herein, is a tumor suppressor gene.

The phrase “repeated element family” or “RE family” as used herein, refers to a family of repeat DNA elements (also known as repetitive DNA elements or repeating units or DNA repeats) which are present in the genome of an organism. A DNA repeat element can be interspersed throughout the genome of an organism or can be present in select chromosomes. An RE family can include one or more repeat DNA elements. Exemplary RE families in the human genome include: interspersed repeats (e.g., long interspersed nucleotide elements (LINE); short interspersed nucleotide elements (SINE)); and tandem repeats (e.g., microsatellites, mini-satellites, satellite DNA or multiple copy genes (e.g., ribosomal RNA)). In some embodiments, an RE family includes one or more repeat elements listed in Table 1, e.g., SINE.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” as the term is used herein refers to performing a process (e.g., performing a synthetic or analytical method) to obtain the physical entity or value. “Indirectly acquiring” as the term is used herein refers to receiving the physical entity or value from another party or source (e.g., a third party laboratory that directly acquired the physical entity or value). Directly acquiring a physical entity includes performing a process that includes a physical change in a physical substance, e.g., a starting material. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof.

“Biological sample,” “sample,” “patient sample,” or “specimen” as the terms are used herein, each refer to a sample obtained from a subject or a patient. The source of the sample can be a biopsy (e.g., a liquid biopsy), an aspirate; blood or any blood constituents; bodily fluids (e.g., cerebral spinal fluid, amniotic fluid, peritoneal fluid or interstitial fluid). The sample can comprise cells (e.g., any cell from a human body, e.g., normal cells and/or cancer cells) and/or cell-free DNA, e.g., circulating tumor DNA or circulating DNA from a normal cell. In an embodiment, the sample, e.g., the tumor sample, includes tissue or cells from a surgical margin. In another embodiment, the sample, e.g., tumor sample, includes one or more circulating tumor cells (CTC) (e.g., a CTC acquired from a blood sample).

As used herein, the term “sensitivity” refers to the ability of a method to detect or identify the presence of a disease in a subject. For example, when used in reference to any of the variety of methods described herein that can detect the presence of cancer in a subject, a high sensitivity means that the method correctly identifies the presence of cancer in the subject a large percentage of the time. For example, a method described herein that correctly detects the presence of cancer in a subject 95% of the time the method is performed is said to have a sensitivity of 95%. In some embodiments, a method described herein that can detect the presence of cancer in a subject provides a sensitivity of at least 70% (e.g., about 70%, about 72%, about 75%, about 80%, about 85%, about 90%, about 91%, about 92%, about 93%, about 94%, about 95%, about 96%, about 97%, about 98%, about 99%, about 99.5%, or about 100%). In some embodiments, methods provided herein that include detecting the presence of one or more members of two or more classes of biomarkers (e.g., genetic biomarkers and/or protein biomarkers) provide a higher sensitivity than methods that include detecting the presence of one or more members of only one class of biomarkers.

In some embodiments, sensitivity provides a measure of the ability of a method to detect a sequence variant in a heterogeneous population of sequences. A method has a sensitivity of S % for variants of F % if, given a sample in which the sequence variant is present as at least F % of the sequences in the sample, the method can detect the sequence at a confidence of C %, S % of the time. By way of example, a method has a sensitivity of 90% for variants of 5% if, given a sample in which the variant sequence is present as at least 5% of the sequences in the sample, the method can detect the sequence at a confidence of 99%, 9 out of 10 times (F=5%; C=99%; S=90%). Exemplary sensitivities include those of S=90%, 95%, 99%, 99.9% for sequence variants at F=0.5%, 1%, 5%, 10%, 20%, 50%, 100% at confidence levels of C=90%, 95%, 99%, and 99.9%.

As discussed above, in embodiments, sensitivity is the ability of a test method to make an assignment of a first state identity to all first state samples, in other words, to find or identify all first state samples. (Sensitivity does not address the propensity of a method to mis-assign a first state sample as a second state sample). In an embodiment the first state is negativity, and sensitivity is the ability to identify all negative samples. In an embodiment the first state is positivity, and sensitivity is the ability to identify all positive samples.

As used herein, the term “specificity” refers to the ability of a method to detect the presence of a disease in a subject (e.g., the specificity of a method can be described as the ability of the method to identify the true positive over true negative in a subject and/or to distinguish a truly occurring sequence variant from a sequencing artifact or other closely related sequences). For example, when used in reference to any of the variety of methods described herein that can detect the presence of cancer in a subject, a high specificity means that the method correctly identifies the absence of cancer in the subject a large percentage of the time (e.g., the method does not incorrectly identify the presence of cancer in the subject a large percentage of the time). A method has a specificity of X % if, when applied to a sample set of NTotal sequences, in which XTrue sequences are truly variant and XNot true are not truly variant, the method can select at least X % of the not truly variant as not variant. For example, a method has a specificity of 90% if, when applied to a sample set of 1,000 sequences, in which 500 sequences are truly variant and 500 are not truly variant, the method selects 90% of the 500 not truly variant sequences as not variant. For example, a method described herein that correctly detects the absence of cancer in a subject 95% of the time the method is performed is said to have a specificity of 95%. In some embodiments, a method described herein that can detect the absence of cancer in a subject provides a specificity of at least 80% (e.g., at least 80%, 85%, 90%, 91%, 92%, 93%, 94%, 95%, 96%, 97%, 98%, 99%, 99.5%, or higher). A method having high specificity results in minimal or no false positive results (e.g., as compared to other methods). False positive results can arise from any source. For example, in various methods described herein that correctly detect the absence of cancer and include sequencing a nucleic acid, false positives can result from errors introduced into the sequence of interest during sample preparation, sequencing errors, and/or inadvertent sequencing of closely related sequences such as pseudo-genes or members of a gene family. In some embodiments, methods provided herein that include detecting the presence of one or more members of two or more classes of biomarkers (e.g., genetic biomarkers and/or protein biomarkers) provide a higher specificity than methods that include detecting the presence of one or more members of only one class of biomarkers.

As discussed above, in embodiments, specificity is the ability of a test method to make a true assignment of a first state identity to a sample. (Specificity does not address the ability of the method to find all true first state samples, that is sensitivity). In an embodiment the first state is negativity, and specificity is the ability to make true (as opposed to incorrect) assignments of negativity (and not mis-assign second state (e.g., positive) samples as first state (negative) sample). In an embodiment the first state is positivity, and specificity is the ability to make true (as opposed to incorrect) assignments of positivity (and not mis-assign second state (e.g., negative) samples as first state (positive) samples).

As used herein, the phrase “subgenomic interval” refers to a portion of a genomic sequence. A subgenomic interval can be any appropriate size (e.g., can include any appropriate number of nucleotides). In some embodiments, a subgenomic interval can include a single nucleotide (e.g., single nucleotide for which variants thereof are associated (positively or negatively) with a tumor phenotype). In some embodiments, a subgenomic interval can include more than one nucleotide. For example, a subgenomic interval can include at least about 2 (e.g., about 5, about 10, about 50, about 100, about 150, about 250, or about 300) nucleotides. In some cases, a subgenomic interval can include an entire gene. In some cases, a subgenomic interval can include a portion of gene (e.g., a coding region such as an exon, a non-coding region such as an intron, or a regulatory region such as a promoter, enhancer, 5′ untranslated region (5′ UTR), or 3′ untranslated region (3′ UTR)). In some cases, a subgenomic interval can include all or part of a naturally occurring (e.g., genomic) nucleotide sequence. For example, a subgenomic interval can correspond to a fragment of genomic DNA which can be subjected to a sequencing reaction. In some cases, a subgenomic interval can be a continuous nucleotide sequence from a genomic source. In some cases, a subgenomic interval can include nucleotide sequences that are not contiguous within the genome. For example, a subgenomic interval can include a nucleotide sequence that includes an exon-exon junction (e.g., in cDNA reverse transcribed from the subgenomic interval). In some cases, a subgenomic interval can include a mutation (e.g., a SNV, an SNP, a somatic mutation, a germ line mutation, a point mutation, a rearrangement, a deletion mutation (e.g., an in-frame deletion, an intragenic deletion, or a full gene deletion), an insertion mutation (e.g., an intragenic insertion), an inversion mutation (e.g., an intra-chromosomal inversion), an inverted duplication mutation, a tandem duplication (e.g., an intrachromosomal tandem duplication), a translocation (e.g., a chromosomal translocation, or a non-reciprocal translocation), a change in gene copy number, or any combination thereof.

As used herein, the phrase “leukocyte parameter,” refers to the sequence of a leukocyte nucleic acid, e.g., a chromosomal nucleic acid.

As used herein, the phrase “genomic event,” refers to a sequence of a subgenomic interval that differs from the sequence of a reference sequence. A genomic event can be, e.g., a mutation, e.g., a point mutation or a rearrangement, e.g., a translocation.

### Aneuploidy Detection

This document provides methods and materials for identifying one or more chromosomal anomalies (e.g., aneuploidies) in a sample. In some embodiments, methods and materials described herein are used to identify one or more chromosomal anomalies (e.g., aneuploidies) in an embryo. In some embodiments, methods and materials described herein are used to identify one or more chromosomal anomalies (e.g., aneuploidies) in a mammal (e.g., a juvenile mammal or an adult mammal). For example, a mammal (e.g., a sample obtained from a mammal) can be assessed for the presence or absence of one or more chromosomal anomalies. In some cases, this document provides methods and materials for using amplicon-based sequencing data to identify a mammal as having a disease associated with one or more chromosomal anomalies (e.g., cancer). For example, methods and materials described herein can be applied to a sample obtained from a mammal to identify the mammal as having one or more chromosomal anomalies. For example, methods and materials described herein can be applied to a sample obtained from a mammal to identify the mammal as having a disease associated with one or more chromosomal anomalies (e.g., cancer). This document also provides methods and materials for identifying and/or treating a disease or disorder associated with one or more chromosomal anomalies (e.g., one or more chromosomal anomalies identified as described herein). In some cases, one or more chromosomal anomalies can be identified in DNA (e.g., genomic DNA) obtained from a sample obtained from a mammal. For example, a prenatal mammal (e.g., prenatal human) can be identified as having a disease or disorder based, at least in part, on the presence of one or more chromosomal anomalies. In some embodiments, a mammalian embryo identified as having a disease or disease based, at least in part, on one or more chromosomal abnormalities can be assessed for the purposes of in vitro fertilization. In some embodiments, a mammal identified as having cancer based, at least in part, on the presence of one or more chromosomal anomalies can be treated with one or more cancer treatments. In some embodiments, a mammal can be identified as having congenital abnormalities based, at least in part, on the presence of one or more chromosomal abnormalities. In some embodiments, methods and materials provided herein are used to test an embryo (e.g., an embryo generated by in vitro fertilization) for chromosomal abnormalities prior to transfer to the uterus (e.g., a human uterus) for implantation.

Disclosed herein, inter alia, is a method of increasing the sensitivity of detecting one or more cancers, or a plurality of cancers, without altering the specificity of detecting said cancer or a plurality of cancers. In an embodiment, the sensitivity of detection of a cancer by evaluating (i) a genetic biomarker, e.g. a somatic mutation; (ii) a protein biomarker; and (iii) aneuploidy status, is higher, e.g., about 1.1, 1.2, 1.3, 1.4, 1.5, 2, 2.5, 3, 3.5, 4, 4.5, 5, 5.5, 6, 6.5, 7, 7.5, 8, 8.5, 9, 9.5, or 10 fold higher, than the sensitivity of detection of the cancer by evaluating (i) alone; (ii) alone; (iii) alone; (i) and (ii) only; (i) and (iii) only; or (ii) and (iii) only. The increase in sensitivity by a method comprising (i), (ii) and (iii) does not alter, e.g., reduce the specificity of detecting the cancer, or plurality of cancers. Exemplary increase in sensitivity of cancer detection using the method of the disclosure is demonstrated in Example 6 of this disclosure.

Any appropriate mammal can be assessed as described herein. A mammal can be a prenatal mammal (e.g., prenatal human). A mammal can be a mammal suspected of having a disease associated with one or more chromosomal anomalies (e.g., cancer or a congenital abnormality). In some cases, humans or other primates such as monkeys can be assessed for the presence of one or more chromosomal anomalies as described herein. In some cases, dogs, cats, horses, cows, pigs, sheep, mice, and rats can be assessed for the presence of one or more chromosomal anomalies as described herein. For example, a human can be assessed for the presence of one or more chromosomal anomalies as described herein.

Any appropriate sample from a mammal can be assessed as described herein (e.g., assessed for the presence of one or more chromosomal anomalies). A sample can include genomic DNA. In some cases, a sample can include cell-free circulating DNA (e.g., cell-free circulating fetal DNA). In some cases, a sample can include circulating tumor DNA (ctDNA). Examples of samples that can contain DNA (e.g., ctDNA) include, without limitation, blood (e.g., whole blood, serum, or plasma), amnion, tissue, urine, cerebrospinal fluid, saliva, sputum, broncho-alveolar lavage, bile, lymphatic fluid, cyst fluid, stool, ascites, pap smears, cerebral spinal fluid, endo-cervical, endometrial, and fallopian samples. For example, a sample can be a plasma sample. For example, a sample can be a urine sample. For example, a sample can be a saliva sample. For example, a sample can be a cyst fluid sample. For example, a sample can be a sputum sample. In some cases, a sample can include a neoplastic cell fraction (e.g., a low neoplastic cell fraction).

In some embodiments, a sample can be processed to isolate and/or purify DNA from the sample. In some embodiments, DNA isolation and/or purification can include cell lysis (e.g., using detergents and/or surfactants). In some embodiments, further processing of DNA (e.g., an amplification reaction) is performed without purifying DNA from the cell lysis. In such cases, additional reagents are added to facilitate further processing including, without limitation, protease inhibitors. In some embodiments, DNA isolation and/or purification can include removing proteins (e.g., using a protease). In some cases, DNA isolation and/or purification can include removing RNA (e.g., using an RNase). In some embodiments, DNA isolation is performed using commercially available kits (for example, without limitation, Qiagen DNAeasy kit) or buffers known in the art (e.g., detergents in Tris-buffer).

In some embodiments, the amount DNA inputted (“input DNA”) into the isolation and/or purification reaction may vary depending on a variety of factors including, without limitation, average length of DNA fragments, overall DNA quality, and/or type of DNA (e.g., gDNA, mitochondrial DNA, cfDNA). In some embodiments, any suitable amount of input DNA can be used in the methods described herein. In some embodiments, the amount of input DNA can be any amount from 1 picogram (pg) to 500 pg. In some embodiments, the amount of input DNA can be at least 0.01 pg, at least 0.01 pg, at least 0.1 pg or at least 1 pg. In some embodiments, the amount of input DNA can be at least 1 picogram (pg), at least 2 pg, at least 3 pg, at least 4 pg, at least 5 pg, at least 6 pg, at least 7 pg, at least 8 pg, at least 9 pg at least 10 pg, at least 11 pg, at least 12 pg, at least 13 pg, at least 14 pg, at least 15 pg, at least 16 pg, at least 17 pg, at least 18 pg, at least 19 pg, at least 20 pg, at least 21 pg, at least 22 pg, at least 23 pg, at least 24 pg, at least 25 pg, at least 26 pg, at least 27 pg, at least 28 pg, at least 29 pg, at least 30 pg, at least 31 pg, at least 32 pg, at least 33 pg, at least 34 pg, at least 35 pg, at least 36 pg, at least 37 pg, at least 38 pg, at least 39 pg or at least 40 pg. In some embodiments, the amount of input DNA is 3 pg.

In some embodiments, methods and materials for identifying one or more chromosomal anomalies (e.g., aneuploidies) as described herein can include amplification of a plurality of amplicons. In some embodiments, the plurality of amplicons is amplified from a plurality of chromosomal sequences in a DNA sample. In some embodiments, the plurality of amplicons can be amplified from any variety of repetitive elements (see e.g., Table 1 for a list of repetitive elements). In some embodiments, the plurality of amplicons is amplified from a plurality of short interspersed nucleotide elements (SINEs). In some embodiments, the plurality of amplicons is amplified from a plurality of long interspersed nucleotide elements (LINEs). Methods of amplifying a plurality of amplicons include, without limitation, the polymerase chain reaction (PCR) and isothermal amplification methods (e.g., rolling circle amplification or bridge amplification). In some embodiments, a second amplification step is performed. In some embodiments, the amplified DNA from a first amplification reaction is used as a template in a second amplification reaction. In some embodiments, the amplified DNA is purified before the second amplification reaction (e.g., PCR purification using methods known in the art).

In some embodiments, an amplification reaction includes using a single pair of primers comprising a first primer having or including SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9. In some embodiments, an amplification reaction includes using a single pair of primers comprising a first primer having at least 80% (e.g., at least 85%, at least 90%, at least 95%, at least 96%, at least 97%, at least 98%, or at least 99%) sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9. In some embodiments, an amplification reaction includes using a single pair of primers comprising a second primer having or including SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18 or SEQ ID NO: 19. In some embodiments, an amplification reaction includes using a single pair of primers comprising a second primer having at least 80% (e.g., at least 85%, at least 90%, at least 95%, at least 96%, at least 97%, at least 98%, or at least 99%) sequence identity to SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18 or SEQ ID NO: 19.

In some embodiments, the first primer has a sequence that is at least 80% identical (e.g., at least 85%, at least 90%, at least 95% at least 99%, or 100% identical) to CGACGTAAAACGACGGCCAGTNNNNNNNNNNNNNNNNGGTGAAACCCCGTCTC TACA (SEQ ID NO: 1). In some embodiments, the second primer has a sequence that is at least 80% identical (e.g., at least 85%, at least 90%, at least 95% at least 99%, or 100% identical) to CACACAGGAAACAGCTATGACCATGCCTCCTAAGTAGCTGGGACTACAG (SEQ ID NO: 10). In some embodiments, an amplification reaction includes using a single pair of primers comprising a first primer having SEQ ID NO. 1 and a second primer having SEQ ID NO. 10. In some embodiments, an amplification reaction includes using a single pair of primers comprising a first primer having at least 80% (e.g., at least 85%, at least 90%, at least 95%, at least 96%, at least 97%, at least 98%, or at least 99%) sequence identity to SEQ ID NO. 1 and a second primer having at least 80% (e.g., at least 85%, at least 90%, at least 95%, at least 96%, at least 97%, at least 98%, or at least 99%) sequence identity to SEQ ID NO. 10.

In some embodiments, the first primer comprises from the 5′ to 3′ end: a universal primer sequence (UPS), a unique identifier DNA sequence (UID), and an amplification sequence. In some embodiments, the first primer comprises from the 5′ to 3′ end: a UPS sequence and an amplification sequence. In some embodiments, the first primer comprises from the 5′ to 3′ end: an amplification sequence. In such cases in which the first primer comprises at least an amplification sequence, any variety of library generation techniques known in the art can be used to generate a next generation sequencing library from the amplified amplicons.

In some embodiments, the universal primer sequence (UPS) facilitates the generation of a library of amplicons ready for next generation sequencing. For example, an amplicon generated during the amplification reaction using a first primer (SEQ ID NO. 1) and a second primer (SEQ ID NO. 10) is used as a template for a second amplification reaction. In such cases, a second set of primers designed to bind to the UPS includes the 5′ grafting sequences necessary for hybridization to an Illumina flow cell.

In some embodiments, the UID comprises a sequence of 16-20 degenerate bases. In some embodiments, a degenerate sequence is a sequence in which some positions of a nucleotide sequence contain a number of possible bases. In some embodiments of any of the methods described herein, a degenerate sequence can be a degenerate nucleotide sequence comprising about or at least 5, 6, 7, 8, 9, 10, 11, 12, 13, 14, 15, 16, 17, 18, 19, 20, 25, 30, 35, 40, 45, or 50 nucleotides. In some embodiments, a nucleotide sequence contains 1, 2, 3, 4, 5, 6, 7, 8, 9, 0, 10, 15, 20, 25, or more degenerate positions within the nucleotide sequence. In some embodiments, the degenerate sequence is used as a unique identifier DNA sequence (UID). In some embodiments, the degenerate sequence is used to improve the amplification of an amplicon. For example, a degenerate sequence may contain bases complementary to a chromosomal sequence being amplified. In such cases, the increased complementarity may increase a primers affinity for the chromosomal sequence. In some embodiments, the UID (e.g., degenerate bases) is designed to increase a primers affinity to a plurality of chromosomal sequences.

In some embodiments, an amplification reaction includes one or more pairs of primers (e.g., one or more pairs of primers selected from Table 2). In some embodiments, an amplification reaction includes at least 1, at least 2, at least 3, at least 4, at least 5, at least 6, at least 7, at least 8, or at least 9 pairs of primers. In some embodiments, when an amplification reaction includes more than one pair or primers, at least one pair of primers includes a primer having SEQ ID NO: 1 as a first primer and a primer having SEQ ID NO: 10 as a second primer. In some embodiments, when an amplification reaction includes more than one pair of primers, at least one pair of primers includes a first primer with a sequence having at least 80% (e.g., at least 85%, at least 90%, at least 95%, at least 96%, at least 97%, at least 98%, or at least 99%) sequence identity to SEQ ID NO: 1 and a second primer with a sequence having at least 80% (e.g., at least 85%, at least 90%, at least 95%, at least 96%, at least 97%, at least 98%, or at least 99%) sequence identity to SEQ ID NO: 10.

In some embodiments when an amplification reaction includes one or more pairs of primers, any variety of combinations of primers or pairs of primers can be selected from Table 2. For example, an amplification reaction containing 2 pairs of primers (e.g., 4 primers selected from Table 2) can include a first pair of primers (e.g., a first primer pair 1 from Table 2) that includes a first primer (e.g., a first primer having SEQ ID NO: 1) and a second primer (e.g., a second primer having SEQ ID NO: 10) and a second pair of primers (e.g., a second primer pair 2 from Table 2) that includes a third primer (e.g., a third primer having SEQ ID NO: 2) and a fourth primer (e.g., a fourth primer having SEQ ID NO: 11). Combining any of the forward primers listed in Table 2 (e.g., a “FP” having SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9) with any of the reverse primers listed in Table 2 (e.g., a “RP” having SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18 or SEQ ID NO: 19) will generate amplicons from the repetitive elements as described herein (see e.g., Table 1 for a list of exemplary repetitive elements). For example, an amplification reaction containing 2 pairs of primers (e.g., 4 primers selected from Table 2) can include a first pair of primers (e.g., a first primer pair 1 from Table 2) that includes a first primer (e.g., a first primer having SEQ ID NO: 1) and a second primer (e.g., a second primer having SEQ ID NO: 10) and a second pair of primers (e.g., not listed as a primer pair in Table 2) that includes a third primer (e.g., a third primer having SEQ ID NO: 2) and a fourth primer (e.g., a fourth primer having SEQ ID NO: 12). In some embodiments, an amplification reaction includes one or more pairs of primers where a first primer is included in both pairs of primers. For example, an amplification reaction can include a first pair of primers (e.g., a first primer pair 1 from Table 2) that includes a first primer (e.g., a first primer having SEQ ID NO: 1) and a second primer (e.g., a second primer having SEQ ID NO: 10) and a second pair of primers that includes a third primer (e.g., a third primer having SEQ ID NO: 1) and a fourth primer (e.g., a fourth primer having SEQ ID NO: 11).

In some embodiments, a pair of primers are complementary to a plurality of chromosomal sequences. As used herein, the term “complementary” or “complementarity” refers to nucleic acid residues that are capable or participating in Watson-Crick type or analogous base pair interactions that is enough to support amplification. In some embodiments, an amplification sequence of a first primer is designed to amplify one or more chromosomal sequences. In some embodiments, the one or more chromosomal sequence include any of a variety of repetitive elements as described herein (see e.g., Table 1 for a list of exemplary repetitive elements). In some embodiments, the chromosomal sequences are SINEs. In some embodiments, the chromosomal sequences are LINEs. In some embodiments, the chromosomal sequences are a mixture of different types of repetitive elements (e.g., SINEs, LINEs and/or other exemplary repetitive elements list in Table 1). In some embodiments when an amplification reaction includes two or more pairs of primers, each pair of primers amplifies a different type of repetitive element (see, e.g., Table 1 for a list of exemplary repetitive elements). For example, a first pair of primers can amplify SINEs, and a second pair of primers can amplify LINEs. Optionally, a third, fourth, fifth, etc. pair of primers can amplify a third, fourth, fifth, etc. type of repetitive element (see, e.g., Table 1 for a list of additional exemplary repetitive elements). In some embodiments when an amplification reaction includes two or more pairs of primers, each pair of primers generates amplicons from the same type of repetitive element (see, e.g., Table 1 for a list of exemplary repetitive elements). For example, a first pair of primers can amplify SINEs, and a second pair of primers amplify SINEs. Optionally, a third, fourth, fifth, etc. pair of primers can amplify SINEs. In some embodiments when an amplification reaction includes two or more primer pairs, each pair of primers generates amplicons from a mixture of different types of repetitive elements (see e.g., Table 1 for a list of exemplary repetitive elements).

TABLE 1
List of exemplary repetitive elements

| | | | | |
|---|---|---|---|---|
| ACRO1_Satellite | ALR/Alpha_Satellite | (A)n_Simple | A-rich_Low | Arthur1A_DNA |
| Arthur1B_DNA | Arthur1_DNA | AT_rich | BLACKJACK_DNA | (CAAAAA)n_Simple |
| (CAAAA)n_Simple | (CAA)n_Simple | (CAAT)n_Simple | (CA)n_Simple | (CATA)n_Simple |
| (CATATA)n_Simple | (CATTC)n_Satellite | (CCCCAG)n_Simple | Charlie13a_DNA | Charlie15a_DNA |
| Charlie16a_DNA | Charlie17a_DNA | Charlie19a_DNA | Charlie1a_DNA | Charlie1b_DNA |
| Charlie1_DNA | Charlie21a_DNA | Charlie22a_DNA | Charlie23a_DNA | Charlie25_DNA |
| Charlie2b_DNA | Charlie4a_DNA | Charlie4z_DNA | Charlie5_DNA | Charlie6_DNA |
| Charlie7a_DNA | Charlie7_DNA | Charlie8_DNA | Cheshire_DNA | (C)n_Simple |
| C-rich_Low | (CTA)n_Simple | CT-rich_Low | ERV3-16A3_I-int | ERVL-B4-int_LTR |
| ERVL-E-int_LTR | ERVL-int_LTR | Eulor11_DNA | Eulor1_DNA | HERV16-int_LTR |
| HERV30-int_LTR | HERV3-int_LTR | HERV4_I-int | HERVE_a-int | HERVLH19-int_LTR |
| HERVH48-int_LTR | HERVH-int_LTR | HERVI-int_LTR | HERVK14C-int_LTR | HERVK3-int_LTR |
| HERVK-int_LTR | HERVL66-int_LTR | HERVL74-int_LTR | HERVL-int_LTR | HERVP71A-int_LTR |
| HSAT4_Satellite | HSMAR1_DNA | HSMAR2_DNA | HUERS-P1-int_LTR | HUERS-P3-int_LTR |
| Kanga1a_DNA | Kanga1d_DNA | Kanga1_DNA | Looper_DNA | LOR1b_LTR |
| LOR1-int_LTR | LTR10F_LTR | LTR12C_LTR | LTR15_LTR | LTR16A1_LTR |
| LTR16A2_LTR | LTR16A_LTR | LTR16B1_LTR | LTR16B_LTR | LTR16C_LTR |
| LTR16E1_LTR | LTR19B_LTR | LTR1B_LTR | LTR1D_LTR | LTR24_LTR |
| LTR25-int_LTR | LTR25_LTR | LTR27B_LTR | LTR27_LTR | LTR28_LTR |
| LTR32_LTR | LTR33C_LTR | LTR33_LTR | LTR34_LTR | LTR35B_LTR |
| LTR37B_LTR | LTR3B_{—} | LTR40c_LTR | LTR43_LTR | LTR45C_LTR |
| LTR48B_LTR | LTR48_LTR | LTR49-int_LTR | LTR49_LTR | LTR57-int_LTR |
| LTR5B_LTR | LTR5_Hs | LTR64_LTR | LTR66_LTR | LTR67B_LTR |
| LTR6A_LTR | LTR71B_LTR | LTR72_LTR | LTR77_LTR | LTR78B_LTR |
| LTR78_LTR | LTR79_LTR | LTR80B_LTR | LTR81A_LTR | LTR81B_LTR |
| LTR81C_LTR | LTR81_LTR | LTR82A_LTR | LTR84b_LTR | LTR85a_LTR |
| LTR85b_LTR | LTR86A2_LTR | LTR87_LTR | LTR8A_LTR | LTR8_LTR |
| LTR9_LTR | MADE1_DNA | MADE2_DNA | MamGypLTR1b_LTR | MamGypLTR1c_LTR |
| MamRep1161_DNA | MamRep1527_LTR | MamRep38_DNA | MamRep434_DNA | MamRep564_Unknown |
| MARNA_DNA | MER101-int_LTR | MER102a_DNA | MER102b_DNA | MER102c_DNA |
| MER103C_DNA | MER106A_DNA | MER110A_LTR | MER110-int_LTR | MER113B_DNA |
| MER113_DNA | MER115_DNA | MER11D_LTR | MER121_DNA | MER135_DNA |
| MER1A_DNA | MER1B_DNA | MER20B_DNA | MER20_DNA | MER21B_LTR |
| MER21-int_LTR | MER2_DNA | MER30_DNA | MER31A_LTR | MER31B_LTR |
| MER31-int_LTR | MER33_DNA | MER34A1_LTR | MER34A_LTR | MER34B-int_LTR |
| MER34B_LTR | MER34C_{—} | MER34C2_LTR | MER34D_LTR | MER34_LTR |
| MER39_LTR | MER3_DNA | MER41A_LTR | MER41B_LTR | MER41-int_LTR |
| MER44A_DNA | MER44C_DNA | MER45R_DNA | MER46C_DNA | MER49_LTR |
| MER4A1_{—} | MER4A_LTR | MER4B-int_LTR | MER4B_LTR | MER4C_LTR |
| MER4D1_LTR | MER4D_LTR | MER4E1_LTR | MER4-int_LTR | MER50_LTR |
| MER51C_LTR | MER52A_LTR | MER52-int_LTR | MER53_DNA | MER54A_LTR |
| MER57A-int_LTR | MER57B2_LTR | MER57E3_LTR | MER57F_LTR | MER57-int_LTR |
| MER58A_DNA | MER58B_DNA | MER58C_DNA | MER58D_DNA | MER5A1_DNA |
| MER5A_DNA | MER5B_DNA | MER5C_DNA | MER61B_LTR | MER61-int_LTR |
| MER63C_DNA | MER63D_DNA | MER65A_LTR | MER65-int_LTR | MER66B_LTR |
| MER66-int_LTR | MER67B_LTR | MER67C_LTR | MER67D_LTR | MER68-int_LTR |
| MER6_DNA | MER70A_LTR | MER70B_LTR | MER74A_LTR | MER74B_LTR |
| MER74C_LTR | MER81_DNA | MER82_DNA | MER83B-int_LTR | MER87_LTR |
| MER89-int_LTR | MER89_LTR | MER8_DNA | MER92B_LTR | MER94_DNA |
| MER96B_DNA | MLT1A0_LTR | MLT1A1_LTR | MLT1A-int_LTR | MLT1A_LTR |
| MLT1B-int_LTR | MLT1B_LTR | MLT1C_LTR | MLT1D_LTR | MLT1E1A_LTR |
| MLT1E2_LTR | MLT1E3-int_LTR | MLT1E3_LTR | MLT1F2_LTR | MLT1F-int_LTR |
| MLT1F_LTR | MLT1G1-int_LTR | MLT1G1_LTR | MLT1G3_LTR | MLT1G-int_LTR |
| MLT1G_LTR | MLT1G_LTR | MLT1H_LTR | MLT1I_LTR | MLT1J1-int_LTR |
| MLT1J1_LTR | MLT1J2_LTR | MLT1J_LTR | MLT1K_LTR | MLT1L_LTR |
| MLT1M_LTR | MLT1N2_LTR | MLT2B1_LTR | MLT2B4_LTR | MLT2D_LTR |
| MSTA-int_LTR | MSTA_LTR | MSTB1_LTR | MSTB-int_LTR | MSTD-int_LTR |
| ORSL-2b_DNA | PABL_A-int | PABL_B-int | PRIMA41-int_LTR | PRIMA4-int_LTR |
| Ricksha_b | Ricksha_c | Ricksha_DNA | SATR1_Satellite | SVA_B |
| SVA_C | (TAAAA)n_Simple | (TAAA)n_Simple | (TAA)n_Simple | (TAG)n_Simple |
| (TA)n_Simple | (TCCA)n_Simple | (TCTCTG)n_Simple | (TG)n_Simple | THE1A-int_LTR |
| THE1B-int_LTR | THE1B_LTR | THE1C-int_LTR | THE1C_LTR | THE1D_LTR |
| Tigger10_DNA | Tigger12c_DNA | Tigger12_DNA | Tigger13a_DNA | Tigger15a_DNA |
| Tigger16b_DNA | Tigger1a_Art | Tigger1_DNA | Tigger2a_DNA | Tigger2b_Pri |
| Tigger2_DNA | Tigger3a_DNA | Tigger3b_DNA | Tigger3_DNA | Tigger4a_DNA |
| Tigger4b_DNA | Tigger4_DNA | Tigger6a_DNA | Tigger7_DNA | Tigger8_DNA |
| Tigger9b_DNA | UCON23_DNA? | Zaphod2_DNA | Zaphod3_DNA | Zaphod_DNA |

In some embodiments, one or both primers of a primer pair described herein include primer modifications. Examples of primer modifications include, without limitation, a spacer (e.g., C3 spacer, PC spacer, hexanediol, spacer 9, spacer 18, 1′,2′-dideoxyribose (dspacer)), phosphorylation, phosphorothioate bond modifications, modified nucleic acids, attachment chemistry and/or linker modifications. Examples of modified nucleic acids include, without limitation, 2-Aminopurine, 2,6-Diaminopurine (2-Amino-dA), 5-Bromo dU, deoxyUridine, Inverted dT, Inverted Dideoxy-T, Dideoxy-C, 5-Methyl dC, deoxyInosine, Super T®, Super G®, Locked Nucleic Acids (LNA's), 5-Nitroindole, 2′-O-Methyl RNA Bases, Hydroxymethyl dC, Iso-dG, Iso-dC, Fluoro C, Fluoro U, Fluoro A, Fluoro 2-MethoxyEthoxy A, 2-MethoxyEthoxy MeC, 2-MethoxyEthoxy and/or 2-MethoxyEthoxy T. Examples of attachment chemistries and linker modifications include, without limitation, Acrydite™, Adenylation, Azide (NHS Ester), Digoxigenin (NHS Ester), Cholesterol-TEG I-Linker, Amino Modifiers (e.g., amino modifier C6, amino nodifier C12, amino modifier C6 dT, amino modifier, and/or Uni-Link™ amino modifier), Alkynes (e.g., 5′ Hexynyl and/or 5-Octadiynyl dU), Biotinylation (e.g., biotin, biotin (Azide), biotin dT, biotin-TEG dual biotin, pC biotin, and/or desthiobiotin-TEG), and/or Thiol Modifications (e.g., thiol modifier C3 S—S, dithiol, and/or thiol modifier C6 S—S). In some embodiments, any primer as described herein includes synthetic nucleic acids.

In some embodiments, one or both primers of a primer pair described herein include primer modifications that enhance processing of amplified DNA. In some embodiments, any primer as described herein includes primer modifications that facilitate elimination of primers (e.g., elimination of primers following an amplification reaction). In some embodiments, primer modifications are conveyed to a product of an amplification reaction (e.g., an amplification product contains modified bases). In such cases, the amplification product includes the modification and the inherent properties of the modification (e.g., the ability to select the amplification product containing the modification).

In some embodiments, methods for identifying one or more chromosomal anomalies as described herein include using amplicon-based sequencing reads. In some embodiments, a plurality of amplicons (e.g., amplicons obtained from a DNA sample) are sequenced. In some embodiments, each amplicon is sequenced at least 1, 2,3,4, 5, 6, 7, 8, 9, 10, 11, 12, 13, 14, 15, 16, 17, 18, 19, 20, or more times. In some embodiments, each amplicon can be sequenced between about 1 and about 20 (e.g., between about 1 and about 15, between about 1 and about 12, between about 1 and about 10, between about 1 and about 8, between about 1 and about 5, between about 5 and about 20, between about 7 and about 20, between about 10 and about 20, between about 13 and about 20, between about 3 and about 18, between about 5 and about 16, or between about 8 and about 12) times. In some cases, amplicon-based sequencing reads can include continuous sequencing reads. In some cases, amplicons include short interspersed nucleotide elements (SINEs). In some cases, amplicon-based sequencing reads can include from about 100,000 to about 25 million (e.g., from about 100,000 to about 20 million, from about 100,000 to about 15 million, from about 100,000 to about 12 million, from about 100,000 to about 10 million, from about 100,000 to about 5 million, from about 100,000 to about 1 million, from about 100,000 to about 750,000, from about 100,000 to about 500,000, from about 100,000 to about 250,000, from about 250,000 to about 25 million, from about 500,000 to about 25 million, from about 750,000 to about 25 million, from about 1 million to about 25 million, from about 5 million to about 25 million, from about 10 million to about 25 million, from about 15 million to about 25 million, from about 200,000 to about 20 million, from about 250,000 to about 15 million, from about 500,000 to about 10 million, from about 750,000 to about 5 million, or from about 1 million to about 2 million) sequencing reads. For example, sequencing a plurality of amplicons can include assigning a unique identifier (UID) to each template molecule (e.g., to each amplicon), amplifying each uniquely tagged template molecule to create UID-families, and redundantly sequencing the amplification products. For example, sequencing a plurality of amplicons can include calculating a Z-score of a variant on said selected chromosome arm using the equation

$$Z ∼ \frac{{\sum }_{i = 1}^{k} {w}_{i} {z}_{i}}{\sqrt{{\sum }_{i = 1}^{k} {w}_{i}^{z}}} ,$$

where wᵢ is UID depth at a variant i, Zᵢ is the Z-score of variant i, and k is the number of variants observed on the chromosome arm. In some embodiments, methods of sequencing amplicons includes methods known in the art (see, e.g., U.S. Pat. No. 2015/0051085; and Kinde et al. 2012 PloS ONE 7:e41162, which are herein incorporated by reference in their entireties). In some embodiments, amplicons are aligned to a reference genome (e.g., GRC37).

In some embodiments, a plurality of amplicons generated by methods described herein includes from about 10,000 to about 1,000,000 (e.g., from about 15,000 to about 1,000,000, from about 25,000 to about 1,000,000, from about 35,000 to about 1,000,000, from about 50,000 to about 1,000,000, from about 75,000 to about 1,000,000, from about 100,000 to about 1,000,000, from about 125,000 to about 1,000,000, from about 160,000 to about 1,000,000, from about 180,000 to about 1,000,000, from about 200,000 to about 1,000,000, from about 300,000 to about 1,000,000, from about 500,000 to about 1,000,000, from about 750,000 to about 1,000,000, from about 10,000 to about 800,000, from about 10,000 to about 500,000, from about 10,000 to about 250,000, from about 10,000 to about 150,000, from about 10,000 to about 100,000, from about 10,000 to about 75,000, from about 10,000 to about 50,000, from about 10,000 to about 40,000, from about 10,000 to about 30,000, or from about 10,000 to about 20,000) amplicons (e.g., unique amplicons). As one non-limiting example, a plurality of amplicons can include about 745,000 amplicons (e.g., 745,000 unique amplicons). Amplicons in a plurality of amplicons can include from about 50 to about 140 (e.g., from about 60 to about 140, from about 76 to about 140, from about 90 to about 140, from about 100 to about 140, from about 130 to about 140, from about 50 to about 130, from about 50 to about 120, from about 50 to about 110, from about 50 to about 100, from about 50 to about 90, from about 50 to about 80, from about 60 to about 130, from about 70 to about 125, from about 80 to about 120, or from about 90 to about 100) nucleotides. As one non-limiting example, an amplicon can include about 100 nucleotides.

In some embodiments, one or more amplicons in a plurality of amplicons generated by methods described herein can be greater than 1000 basepairs (bp) in length (“long amplicons”). In some embodiments, one or more long amplicons make up at least 4.0% of all amplicons within the total plurality of amplicons. In some embodiments, methods and materials described herein can detect long amplicons when the long amplicons make up at least 4.0% of all the amplicons within the total plurality of amplicons. In some embodiments, methods and materials described herein can detect long amplicons when the long amplicons make up between 0.01% and 3.9% of all amplicons within the total plurality of amplicons.

In some embodiments, one or more amplicons with a length >1000 bp originate from amplification of DNA from cells that do not contain a chromosomal abnormality. In some embodiments, cells that do not contain chromosomal abnormalities are considered contaminating cells. In some embodiments, cells that do not contain chromosomal abnormalities are used as control cells or samples. In some embodiments, contaminating cells can be any variety of cells that might be found in a plasma sample that may dilute amplification of the intended target. In some embodiments, contaminating cells are white blood cells (e.g., leukocyte, granulocyte, eosinophil, basophile, B-cell, T-cell or Natural Killer cell). For example, contaminating cells can be leukocytes.

In some embodiments, methods and materials for identifying one or more chromosomal anomalies as described herein include grouping sequencing reads (e.g., from a plurality of amplicons) into clusters (e.g., unique clusters) of genomic intervals. In some embodiments, a genomic interval is included in one or more clusters. In some embodiments, a genomic interval can belong to from about 100 to about 252 (e.g., from about 125 to about 252, from about 150 to about 252, from about 175 to about 252, from about 200 to about 252, from about 225 to about 252, from about 100 to about 250, from about 100 to about 225, from about 100 to about 200, from about 100 to about 175, from about 100 to about 150, from about 125 to about 225, from about 150 to about 200, or from about 160 to about 180) clusters. As one non-limiting example, a genomic interval can belong to about 176 clusters. In some embodiments, each cluster includes any appropriate number of genomic intervals. In some embodiments, each cluster includes the same number of genomic intervals. In some embodiments, different clusters include varying numbers of genomic clusters. As one non-limiting example, each cluster can include about 200 genomic intervals.

In some embodiments, genomic intervals are identified as having shared amplicon features. As used herein, the term “shared amplicon feature” refers to amplicons with one or more features that are similar. In some embodiments, a plurality of genomic intervals are grouped into a cluster based on one or more shared amplicon features of the sequencing reads mapped to a genomic interval. In some embodiments, the shared amplicon feature is the number amplicons mapped to a genomic interval (e.g., sums of the distributions of the sequencing reads in each genomic interval). In some embodiments, the shared amplicon feature is the average length of the mapped amplicons.

In some embodiments, a cluster of genomic intervals includes from about 5000 to about 6000 (e.g., from about 5100 to about 6000, from about 5200 to about 6000, from about 5300 to about 6000, from about 5400 to about 6000, from about 5500 to about 6000, from about 5600 to about 6000, from about 5700 to about 6000, from about 5800 to about 6000, from about 5900 to about 6000, from about 5000 to about 5900, from about 5000 to about 5800, from about 5000 to about 5700, from about 5000 to about 5600, from about 5000 to about 5500, from about 5000 to about 5400, from about 5000 to about 5300, from about 5000 to about 5200, from about 5000 to about 5100, from about 5100 to about 5800, from about 5100 to about 5700, from about 5100 to about 5600, from about 5100 to about 5500, from about 5100 to about 5400, from about 5100 to about 5300, from about 5100 to about 5200, from about 5200 to about 5600, from about 5200 to about 5500, from about 5200 to about 5400, from about 5200 to about 5300, from about 5300 to about 5500, from about 5300 to about 5400, or from about 5400 to 5500 from about 5200 to about 5700, or from about 5300 to about 5500) genomic intervals. As one non-limiting example, a cluster of genomic intervals can include about 5344 genomic intervals. A genomic interval can be any appropriate length. For example, a genomic interval can be the length of an amplicon sequenced as described herein. For example, a genomic interval can be the length of a chromosome arm. In some cases, a genomic interval can include from about 100 to about 125,000,000 (e.g., from about 250 to about 125,000,000, from about 500 to about 125,000,000, from about 750 to about 125,000,000, from about 1,000 to about 125,000,000, from about 1,500 to about 125,000,000, from about 2,000 to about 125,000,000, from about 5,000 to about 125,000,000, from about 7,500 to about 125,000,000, from about 10,000 to about 125,000,000, from about 25,000 to about 125,000,000, from about 50,000 to about 125,000,000, from about 100,000 to about 125,000,000, from about 250,000 to about 125,000,000, from about 500,000 to about 125,000,000, from about 100 to about 1,000,000, from about 100 to about 750,000, from about 100 to about 500,000, from about 100 to about 250,000, from about 100 to about 100,000, from about 100 to about 50,000, from about 100 to about 25,000, from about 100 to about 10,000, from about 100 to about 5,000, from about 100 to about 2,500, from about 100 to about 1,000, from about 100 to about 750, from about 100 to about 500, from about 100 to about 250, from about 500 to about 1,000,000, from about 5000 to about 900,000, from about 50,000 to about 800,000, or from about 100,000 to about 750,000) nucleotides. As one non-limiting example, a genomic interval can include about 500,000 nucleotides. In some embodiments, clusters of genomic intervals are formed using any appropriate method known in the art. In some embodiments, clusters of genomic intervals are formed based on shared amplicon features of the genomic intervals (see, e.g., Douville et al. PNAS 201 115(8):1871-1876, which is herein incorporated by reference in its entirety).

In some embodiments, methods and materials described herein for identifying one or more chromosomal anomalies include assessing a genome (e.g., a genome of a mammal) for the presence or absence of one or more chromosomal anomalies (e.g., aneuploidies). The presence or absence of one or more chromosomal anomalies in the genome of a mammal can, for example, be determined by sequencing a plurality of amplicons obtained from a sample (e.g., a test sample) obtained from the mammal to obtain sequencing reads, and grouping the sequencing reads into clusters of genomic intervals. In some cases, read counts of genomic intervals can be compared to read counts of other genomic intervals within the same sample. In some cases where read counts of genomic intervals are compared to read counts of other genomic intervals within the same sample, a second (e.g., control or reference) sample is not assayed. In some cases, read counts of genomic intervals can be compared to read counts of genomic intervals in another sample. For example, when using methods and materials described herein to identify genetic relatedness, polymorphisms (e.g., somatic mutations), and/or microsatellite instability, genomic intervals can be compared to read counts of genomic intervals in a reference sample. A reference sample can be a synthetic sample. A reference sample can be from a database. In some cases where methods and materials described herein are used to identify anomalies (e.g., aneuploidies), a reference sample can be a normal sample obtained from the same cancer patient (e.g., a sample from the cancer patient that does not harbor cancer cells) or a normal sample from another source (e.g., a patient that does not have cancer). In some cases where method and materials described herein are used to identify anomalies (e.g., aneuploidies), a reference sample can be a normal sample obtained from the same patient (e.g., a sample from pre-natal human that contains only maternal cells).

In some embodiments, methods and materials described herein are used for detecting aneuploidy in a preimplantation embryo (e.g., an embryo generated via in vitro fertilization). In some embodiments, the presence or absence of one or more chromosomal anomalies in a preimplantation embryo is determined by sequencing a plurality of amplicons obtained from a sample taken from the preimplantation embryo (e.g., a test sample such, as without limitation, one or more cells obtained from a blastocyst) to obtain sequencing reads, and grouping the sequencing reads into clusters of genomic intervals. In some cases, read counts of genomic intervals can be compared to read counts of other genomic intervals within the same sample. In some cases where read counts of genomic intervals are compared to read counts of other genomic intervals within the same sample, a second (e.g., control or reference) sample is not assayed. In some cases, read counts of genomic intervals can be compared to read counts of genomic intervals in another sample (e.g., a reference sample). In some embodiments, a reference sample is a sample obtained from a reference mammal. In some embodiments, a reference sample is obtained from a database (e.g., the reference sample is an in silico sample having a known sequence and/or ploidy at the genomic position of interest). Exemplary aneuploidies that can be detected in preimplantation embryos include trisomies at chromosome 21 (e.g., resulting in Down's Syndrome), trisomies at chromosome 13, trisomies at chromosome 18, Turner Syndrome (e.g., women with only one X chromosome) and Klinefelter Syndrome (e.g., men with two or more X chromosomes). In some embodiments, methods and materials described herein are used for detecting aneuploidy in a genome of mammal. For example, a plurality of amplicons obtained from a sample obtained from a mammal can be sequenced, the sequencing reads can be grouped into clusters of genomic intervals, the sums of the distributions of the sequencing reads in each genomic interval can be calculated, a Z-score of a chromosome arm can be calculated, and the presence or absence of an aneuploidy in the genome of the mammal can be identified.

The distributions of the sequencing reads in each genomic interval can be summed. For example, sums of distributions of the sequencing reads in each genomic interval can be calculated using the equation Σ₁^{I} R˜N(Σ₁^{l} μᵢ, Σ₁^{I} σᵢ²), where Rᵢ is the number of sequencing reads, I is the number of clusters on a chromosome arm, N is a Gaussian distribution with parameters μᵢ and σₗ², and μᵢ is the mean number of sequencing reads in each genomic interval, and σᵢ² is the variance of sequencing reads in each genomic interval. A Z-score of a chromosome arm can be calculated using any appropriate technique. For example, a Z-score of a chromosome arm can be calculated using the quantile function 1-CDF(Σ₁^{I} μᵢ, Σ₁^{I} σᵢ²). The presence of an aneuploidy in the genome of the mammal can be identified in the genome of the mammal when the Z-score is outside a predetermined significance threshold, and the absence of an aneuploidy in the genome of the mammal can be identified in the genome of the mammal when the Z-score is within a predetermined significance threshold. The predetermined threshold can correspond to the confidence in the test and the acceptable number of false positives. For example, a significance threshold can be ±1.96, ±3, or ±5. In some embodiments, methods and materials described herein employ supervised machine learning. In some embodiments, supervised machine learning can detect small changes in one or more chromosome arms. For example, supervised machine learning can detect changes such as chromosome arm gains or losses that are often present in a disease or disorder associated with chromosomal anomalies, such as cancer or congenital anomalies. In some embodiments, supervised machine learning can detect changes such as chromosome arm gains or losses that are present in a preimplantation embryo (e.g., a preimplantation embryo generated by in vitro fertilization methods). In some cases, supervised machine learning can be used to classify samples according to aneuploidy status. For example, supervised machine learning can be employed to make genome-wide aneuploidy calls. In some cases, a support vector machine model can include obtaining an SVM score. An SVM score can be obtained using any appropriate technique. In some cases, an SVM score can be obtained as described elsewhere (see, e.g., Cortes 1995 Machine learning 20:273-297; and Meyer et al. 2015 R package version:1.6-3). At lower read depths, a sample will typically have a higher raw SVM score. Thus, in some cases, raw SVM probabilities can be corrected based on the read depth of a sample using the equation

$$log ( 1 - \frac{1}{r} ) = A x + B ,$$

where r is the ratio of the SVM score at a particular read depth/minimum SVM score of a particular sample given sufficient read depth. A and B can be determined as described in Example 1. For example, A=−7.076*10\hat{ }−7, x=the number of unique template molecules for the given sample, and B=−1.946*10\hat{ }−1.

Also provided herein are new methods of normalization that reduce the amount of variability between samples. In some embodiments, a principal component analysis (PCA) can be used for normalization. In some embodiments, a PCA is performed on sequencing data from the controls. For example, a PCA may reduce the number of 500 kb genomic intervals from n=5,344 to a more manageable number of dimensions. Using the PCA coordinates of the controls, a model can be generated that predicts whether a particular 500 kb interval will be amplified more or less efficiently in future samples based on their PCA coordinates.

Correction Factor for 500 kb Intervalᵢ=βₒᵢ+β₁ᵢ*PCA₁β₂ᵢ*PCA₂+β₃ᵢ*PCA₃+β₄ᵢ*PCA₄+β₅ᵢ*PCA₅

For example, for each test sample, a sample can be projected into PCA space and the correction factor can be calculated for each 500 kb interval as function of its PCA coordinates. After applying the correction factor to each 500 kb genomic interval, the test sample may be matched to one or more control samples based on the closest Euclidean distance of the 500 kb intervals.

In some embodiments, samples are excluded in order to ensure the quality of the data. In some embodiments, samples are excluded before, contemporaneously with, and/or after data analysis. In some embodiments, a list of factors can be applied to the data in order to exclude data that does not meet the criteria set forth in the list of factors. In some embodiments, the list of factors may be any reasonable number of factors. For example, a list of five factors can be used to exclude samples. Any combination of factors can be used to determine that a sample should be excluded. In some embodiments, samples with fewer than 2.5M reads may be excluded. In some embodiments, samples with sufficient evidence of contamination may be excluded. For example, a sample may be considered contaminated if the sample has at least 10 significant allelic imbalanced chromosome arms (z score >=2.5) and fewer than ten significant chromosome arms gains or losses (z>=2.5 or z<=−2.5). In some embodiments, allelic imbalance can be determined from SNPs, while gains or losses can be assessed through WALDO. In some embodiments, when examining the quality of the plasma samples, samples may be excluded in which more than 8.5% of the amplicons were larger than 94 bps (50 base pairs between the forward and reverse primers). Without wishing to be bound by theory, such samples may be contaminated with leukocyte DNA. In some embodiments, samples outside the dynamic range of the assay, as defined by the equation below, may be excluded.

$$QC Dynamic Range Metric = {\sum }_{i}^{2 q , 3 q , 4 q , 5 q , 6 q , 8 q , 13 q} \frac{Reads on {chr}_{i}}{{{\sum }^{39}}_{j = 1} Reads on {chr}_{j}}$$

For example, the distribution of this metric has long tails. The values of >0.2450 and 0.2320 may be selected as a dynamic range that could evaluate cutoffs. In some embodiments, plasma samples with known aneuploidy in the leukocytes of the same patients may be excluded. For example, such patients may have Clonal Hematopoiesis of Indeterminate Potential (CHIP) or congenital disorders.

In some embodiments, provided herein are methods to detect copy number variants (CNVs) of indeterminate length. In some embodiments, provided herein are methods to detect copy number variation of near-fixed length. In some embodiments, detecting copy number variation include calculating the values of one or more variables. In some embodiments, using a log ratio of the observed test sample and WALDO predicted values from every 500 kb interval across each chromosomal arm, a circular binary segmentation algorithm can be applied to determine copy number variants throughout each chromosome arm. For example, copy number variant ≤5 Mb in size can be flagged. In some embodiments, the flagged CNVs can be removed before, contemporaneously with, and/or after the analysis. In some embodiments, small CNVs may be used to assess microdeletions or microamplifications. For example, microdelections or microamplifications occur in DiGeorge Syndrome (chromosome 22q11.2 or in breast cancers (chromosome 17q12).

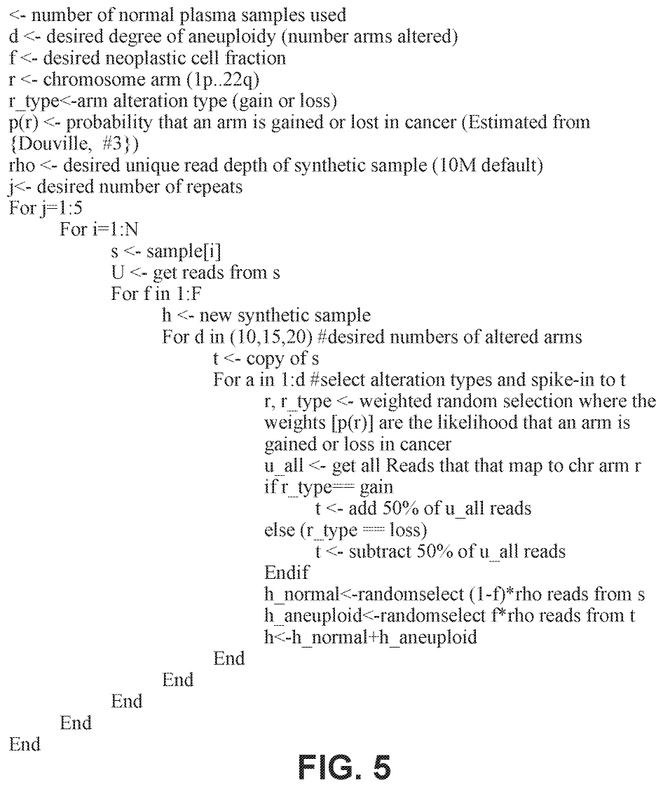

In some embodiments, provided herein are methods of using synthetic aneuploid samples. In some embodiments, synthetic aneuploidy samples can be created by adding (or subtracting) reads from several chromosome arms to the reads from these normal DNA samples. For example, reads can be added or subtracted from 1, 10, 15, or 20 chromosome arms to each sample. The additions and subtractions can be designed to represent neoplastic cell fractions ranging from 0.5% to 1.5% and resulted in synthetic samples containing exactly ten million reads. The reads from each chromosome arm can be added or subtracted uniformly. In some embodiments, provided herein are methods of generating synthetic aneuploid samples using exemplary pseudocode (FIG. 5). In some embodiments, a person of ordinary skill in the art will be able to generate a synthetic sample by applying known coding languages and techniques to the exemplary pseudocode shown in FIG. 5.

Examples of chromosomal anomalies that can be detected using methods and materials described herein include, without limitation, numerical disorders, structural abnormalities, allelic imbalances, and microsatellite instabilities. A chromosomal anomaly can include a numerical disorder. For example, a chromosomal anomaly can include an aneuploidy (e.g., an abnormal number of chromosomes). In some cases, an aneuploidy can include an entire chromosome. In some cases, an aneuploidy can include part of a chromosome (e.g., a chromosome arm gain or a chromosome arm loss). Examples of aneuploidies include, without limitation, monosomy, trisomy, tetrasomy, and pentasomy. A chromosomal anomaly can include a structural abnormality. Examples of structural abnormalities include, without limitation, deletions, duplications, translocations (e.g., reciprocal translocations and Robertsonian translocations), inversions, insertions, rings, and isochromosomes. Chromosomal anomalies can occur on any chromosome pair (e.g., chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, and/or one of the sex chromosomes (e.g., an X chromosome or a Y chromosome). For example, aneuploidy can occur, without limitation, in chromosome 13 (e.g., trisomy 13), chromosome 16 (e.g., trisomy 16), chromosome 18 (e.g., trisomy 18), chromosome 21 (e.g., trisomy 21), and/or the sex chromosomes (e.g., X chromosome monosomy; sex chromosome trisomy such as XXX, XXY, and XYY; sex chromosome tetrasomy such as XXXX and XXYY; and sex chromosome pentasomy such as XXXXX, XXXXY, and XYYYY). For example, structural abnormalities can occur, without limitation, in chromosome 4 (e.g., partial deletion of the short arm of chromosome 4), chromosome 11 (e.g., a terminal 11q deletion), chromosome 13 (e.g., Robertsonian translocation at chromosome 13), chromosome 14 (e.g., Robertsonian translocation at chromosome 14), chromosome 15 (e.g., Robertsonian translocation at chromosome 15), chromosome 17 (e.g., duplication of the gene encoding peripheral myelin protein 22), chromosome 21 (e.g., Robertsonian translocation at chromosome 21), and chromosome 22 (e.g., Robertsonian translocation at chromosome 22).

In some embodiments, methods and materials as described herein are used for identifying and/or treating a disease associated with one or more chromosomal anomalies (e.g., one or more chromosomal anomalies identified as described herein, such as, without limitation, an aneuploidy). In some cases, a DNA sample (e.g., a genomic DNA sample) obtained from a mammal can be assessed for the presence or absence of one or more chromosomal anomalies. For example, a mammal (e.g., a human) can be identified as having a disease based, at least in part, on the presence of one or more chromosomal anomalies can be treated with one or more cancer treatments. In some embodiments, a mammal identified as having cancer based, at least in part, on the presence of one or more chromosomal anomalies is treated with one or more cancer treatments. In some embodiments, a mammal (e.g., a prenatal human) can be identified as having a disease or disorder based, at least in part, on the presence of one or more chromosomal anomalies. In some embodiments, an embryo (e.g., an embryo generated by in vitro fertilization) can be identified as being unsuitable for to transfer to the uterus (e.g., a human uterus) for implantation based, at least in part, on the presence of one or more chromosomal anomalies. In some embodiments, an embryo (e.g., an embryo generated by in vitro fertilization) can be identified as being suitable for to transfer to the uterus (e.g., a human uterus) for implantation based, at least in part, on the absence of one or more chromosomal anomalies.

In some embodiments, a mammal identified as having a disease or disorder associated with one or more chromosomal anomalies as described herein (e.g., based at least in part on the presence of one or more chromosomal anomalies, such as, without limitation, an aneuploidy) can have the disease or disorder diagnosis confirmed using any appropriate method. Examples of methods that can be used to confirm the presence of one or more chromosomal anomalies include, without limitation, karyotyping, fluorescence in situ hybridization (FISH), quantitative PCR of short tandem repeats, quantitative fluorescence PCR (QF-PCR), quantitative PCR dosage analysis, quantitative mass spectrometry of SNPs, comparative genomic hybridization (CGH), whole genome sequencing, and exome sequencing.

### Multi-Analyte Test for Cancer Detection

In some embodiments, detection of aneuploidy is used to identify a mammal as having cancer (e.g., any of the exemplary cancers described herein). In some embodiments, detection of one or more genetic biomarkers is used to confirm or identify a mammal as having cancer (e.g., any of the exemplary cancers described herein). In some embodiments, an elevated level of one or more peptide biomarkers is used to confirm or identify a mammal as having cancer (e.g., any of the exemplary cancers described herein). In some embodiments, a mammal identified as having cancer as described herein (e.g., based on detection of aneuploidy, and/or at least in part on the presence or absence of one or more genetic biomarkers (e.g., mutations) and/or an elevated level of one or more protein biomarkers (e.g., peptides)) can have the cancer diagnosis confirmed using any appropriate method. Examples of methods that can be used to diagnose or confirm diagnosis of a cancer include, without limitation, physical examinations (e.g., pelvic examination), imaging tests (e.g., ultrasound or CT scans), cytology, and tissue tests (e.g., biopsy).

In some embodiments, methods for identifying one or more chromosomal anomalies (e.g., aneuploidy) provided herein are used to identify a mammal as having a distinct stage of cancer. In some embodiments, a cancer can be a Stage I cancer. In some embodiments, a cancer can be a Stage II cancer. In some embodiments, a cancer can be a Stage III cancer. In some embodiments, a cancer can be a Stage IV cancer. In some embodiments, methods for identifying one or more chromosomal anomalies (e.g., aneuploidy) provided herein are used to identify a mammal as having a stage of cancer that conventional methods of detecting cancer cannot reliably detect. For example, methods for identifying one or more chromosomal anomalies (e.g., aneuploidy) provided herein can be used to identify a mammal as having a Stage I cancer that conventional methods of detecting cancer cannot reliably detect. In some embodiments, methods provided herein for identifying: 1) one or more chromosomal anomalies (e.g., aneuploidy), and 2) one or more genetic biomarkers (e.g., any of the genetic biomarkers provided herein) are used to identify a mammal as having a stage of cancer that conventional methods of detecting cancer cannot reliably detect. In some embodiments, methods provided herein for identifying: 1) one or more chromosomal anomalies (e.g., aneuploidy), and 2) one or more protein biomarkers (e.g., any of the protein biomarkers provided herein) are used to identify a mammal as having a stage of cancer that conventional methods of detecting cancer cannot reliably detect. Non-limiting examples of cancers that be identified as described herein (e.g., based on detection of aneuploidy, and/or at least in part on the presence or absence of one or more genetic biomarkers (e.g., mutations) and/or an elevated level of one or more protein biomarkers (e.g., peptides)) include, liver cancer, ovarian cancer, esophageal cancer, stomach cancer, pancreatic cancer, colorectal cancer, lung cancer, breast cancer, and prostate cancer.

In some embodiments, the subject in which the presence of one or more chromosomal anomalies (e.g., aneuploidies) is detected may be selected for further diagnostic testing. In some embodiments, methods provided herein can be used to select a subject for further diagnostic testing at a time period prior to the time period when conventional techniques are capable of diagnosing the subject with an early-stage cancer. For example, methods provided herein for selecting a subject for further diagnostic testing can be used when a subject has not been diagnosed with cancer by conventional methods and/or when a subject is not known to harbor a cancer. In some embodiments, a subject selected for further diagnostic testing can be administered a diagnostic test (e.g., any of the diagnostic tests described herein) at an increased frequency compared to a subject that has not been selected for further diagnostic testing. For example, a subject selected for further diagnostic testing can be administered a diagnostic test at a frequency of twice daily, daily, bi-weekly, weekly, bi-monthly, monthly, quarterly, semi-annually, annually, or any at frequency therein. In some embodiments, a subject selected for further diagnostic testing can be administered one or more additional diagnostic tests compared to a subject that has not been selected for further diagnostic testing. For example, a subject selected for further diagnostic testing can be administered two diagnostic tests or more, whereas a subject that has not been selected for further diagnostic testing is administered only a single diagnostic test (or no diagnostic tests). In some embodiments, the diagnostic testing method can determine the presence of the same type of cancer as the originally detected cancer. Additionally or alternatively, the diagnostic testing method can determine the presence of a different type of cancer from the originally detected cancer.

In some embodiments, the diagnostic testing method is a scan. In some embodiments, the scan is a bone scan, a computed tomography (CT), a CT angiography (CTA), an esophagram (a Barium swallow), a Barium enema, a gallium scan, a magnetic resonance imaging (MRI), a mammography, a monoclonal antibody scan (e.g., ProstaScint® scan for prostate cancer, OncoScint® scan for ovarian cancer, and CEA-Scan® for colon cancer), a multigated acquisition (MUGA) scan, a PET scan, a PET/CT scan, a thyroid scan, an ultrasound (e.g., a breast ultrasound, an endobronchial ultrasound, an endoscopic ultrasound, a transvaginal ultrasound), an X-ray, a DEXA scan.

In some embodiments, the diagnostic testing method is a physical examination, such as, without limitation, an anoscopy, a biopsy, a bronchoscopy (e.g., an autofluorescence bronchoscopy, a white-light bronchoscopy, a navigational bronchoscopy), a digital breast tomosynthesis, a digital rectal exam, an endoscopy, including but not limited to a capsule endoscopy, virtual endoscopy, an arthroscopy, a bronchoscopy, a colonoscopy, a colposcopy, a cystoscopy, an esophagoscopy, a gastroscopy, a laparoscopy, a laryngoscopy, a neuroendoscopy, a proctoscopy, a sigmoidoscopy, a skin cancer exam, a thoracoscopy, an endoscopic retrograde cholangiopancreatography (ERCP), an ensophagogastroduodenoscopy, a pelvic exam.

In some embodiments, the diagnostic testing method is a biopsy (e.g., a bone marrow aspiration, a tissue biopsy). In some embodiments, the biopsy is performed by fine needle aspiration or by surgical excision. In some embodiments, the diagnostic testing method(s) further include obtaining a biological sample (e.g., a tissue sample, a urine sample, a blood sample, a check swab, a saliva sample, a mucosal sample (e.g., sputum, bronchial secretion), a nipple aspirate, a secretion or an excretion). In some embodiments, the diagnostic testing method(s) include determining exosomal proteins (e.g., an exosomal surface protein (e.g., CD24, CD147, PCA-3)) (Soung et al. (2017) Cancers 9(1):pii:E8). In some embodiments, the diagnostic testing method is an oncotype DX® test (Baehner (2016) Ecancermedicalscience 10:675).

In some embodiments, the diagnostic testing method is a test, such as without limitation, an alpha-fetoprotein blood test, a bone marrow test, a fecal occult blood test, a human papillomavirus test, low-dose helical computed tomography, a lumbar puncture, a prostate specific antigen (PSA) test, a pap smear, or a tumor marker test.

In some embodiments, the diagnostic testing method includes determining the level of a known protein biomarker (e.g., CA-125 or prostate specific antigen (PSA)). For example, a high amount of CA-125 can be found in subject's blood, which subject has ovarian cancer, endometrial cancer, fallopian tube cancer, pancreatic cancer, stomach cancer, esophageal cancer, colon cancer, liver cancer, breast cancer, or lung cancer. The term “biomarker” as used herein refers to “a biological molecule found in blood, other bodily fluids, or tissues that is a sign of a normal or abnormal process, or of a condition or disease”, e.g., as defined by the National Cancer Institute. (see, e.g., the URL www.cancer.gov/publications/dictionaries/cancer-terms? CdrID=45 618). A biomarker can include a genetic biomarker such as, without limitation, a nucleic acid (e.g., a DNA molecule, a RNA molecule (e.g., a microRNA, a long non-coding RNA (lncRNA) or other non-coding RNA) A biomarker can include a protein biomarker such as, without limitation, a peptide, a protein, or a fragment thereof.

In some embodiments, the biomarker is FLT3, NPM1, CEBPA, PRAM1, ALK, BRAF, KRAS, EGFR, Kit, NRAS, JAK2, KRAS, HPV virus, ERBB2, BCR-ABL, BRCA1, BRCA2, CEA, AFP, and/or LDH. See e.g., Easton et al. (1995) Am. J. Hum. Genet. 56: 265-271, Hall et al. (1990) Science 250: 1684-1689, Lin et al. (2008) Ann. Intern. Med. 149: 192-199, Allegra et al. (2009) (2009) J. Clin. Oncol. 27: 2091-2096, Paik et al. (2004) N. Engl. J. Med. 351: 2817-2826, Bang et al. (2010) Lancet 376: 687-697, Piccart-Gebhart et al. (2005) N. Engl. J. Med. 353: 1659-1672, Romond et al. (2005) N. Engl. J. Med. 353: 1673-1684, Locker et al. (2006) J. Clin. Oncol. 24: 5313-5327, Giligan et al. (2010) J. Clin. Oncol. 28: 3388-3404, Harris et al. (2007) J. Clin. Oncol. 25: 5287-5312; Henry and Hayes (2012) Mol. Oncol. 6: 140-146. In some embodiments, the biomarker is a biomarker for detection of breast cancer in a subject, such as, without limitation, MUC-1, CEA, p53, urokinase plasminogen activator, BRCA1, BRCA2, and/or HER2 (Gam (2012) World J. Exp. Med. 2(5): 86-91). In some embodiments, the biomarker is a biomarker for detection of lung cancer in a subject, such as, without limitation, KRAS, EGFR, ALK, MET, and/or ROS1 (Mao (2002) Oncogene 21: 6960-6969; Korpanty et al. (2014) Front Oncol. 4: 204). In some embodiments, the biomarker is a biomarker for detection of ovarian cancer in a subject, such as, without limitation, HPV, CA-125, HE4, CEA, VCAM-1, KLK6/7, GST1, PRSS8, FOLR1, ALDH1 (Nolen and Lokshin (2012) Future Oncol. 8(1): 55-71; Sarojini et al. (2012) J. Oncol. 2012:709049). In some embodiments, the biomarker is a biomarker for detection of colorectal cancer in a subject, such as, without limitation, MLH1, MSH2, MSH6, PMS2, KRAS, and BRAF (Gonzalez-Pons and Cruz-Correa (2015) Biomed. Res. Int. 2015: 149014; Alvarez-Chaver et al. (2014) World J. Gastroenterol. 20(14): 3804-3824). In some embodiments, the diagnostic testing method determines the presence and/or expression level of a nucleic acid (e.g., microRNA (Sethi et al. (2011) J. Carcinog. Mutag. S1-005), RNA, a SNP (Hosein et al. (2013) Lab. Invest doi: 10.1038/labinvest.2013.54; Falzoi et al. (2010) Pharmacogenomics 11: 559-571), methylation status (Castelo-Branco et al. (2013) Lancet Oncol 14: 534-542), a hotspot cancer mutation (Yousem et al. (2013) Chest 143: 1679-1684)). Non-limiting examples of methods of detecting a nucleic acid in a sample include: PCR, RT-PCR, sequencing (e.g., next generation sequencing methods, deep sequencing), a DNA microarray, a microRNA microarray, a SNP microarray, fluorescent in situ hybridization (FISH), restriction fragment length polymorphism (RFLP), gel electrophoresis, Northern blot analysis, Southern blot analysis, chromogenic in situ hybridization (CISH), chromatin immunoprecipitation (ChIP), SNP genotyping, and DNA methylation assay. See, e.g., Meldrum et al. (2011) Clin. Biochem. Rev. 32(4): 177-195; Sidranksy (1997) Science 278(5340): 1054-9.

In some embodiments, the diagnostic testing method includes determining the presence of a protein biomarker in a sample (e.g., a plasma biomarker (Mirus et al. (2015) Clin. Cancer Res. 21(7): 1764-1771)). Non-limiting examples of methods of determining the presence of a protein biomarker include: western blot analysis, immunohistochemistry (IHC), immunofluorescence, mass spectrometry (MS) (e.g., matrix assisted laser desorption/ionization (MALDI)-MS, surface enhanced laser desorption/ionization time-of-flight (SELDI-TOF)-MS), enzyme-linked immunosorbent assay (ELISA), flow cytometry, proximity assay (e.g., VeraTag proximity assay (Shi et al. (2009) Diagnostic molecular pathology: the American journal of surgical pathology, part B: 18: 11-21, Huang et al. (2010) AM. J. Clin. Pathol. 134: 303-11)), a protein microarray (e.g., an antibody microarray (Ingvarsson et al. (2008) Proteomics 8: 2211-9, Woodbury et al. (2002) J. Proteome Res. 1: 233-237), an IHC-based microarray (Stromberg et al. (2007) Proteomics 7: 2142-50), a microarray ELISA (Schroder et al. (2010) Mol. Cell. Proteomics 9: 1271-80). In some embodiments, the method of determining the presence of a protein biomarker is a functional assay. In some embodiments, the functional assay is a kinase assay (Ghosh et al. (2010) Biosensors & Bioelectronics 26: 424-31, Mizutani et al. (2010) Clin. Cancer Res. 16: 3964-75, Lee et al. (2012) Biomed. Microdevices 14: 247-57), a protease assay (Lowe et al. (2012) ACS nano. 6: 851-7, Fujiwara et al. (2006) Breast cancer 13: 272-8, Darragh et al. (2010) Cancer Res 70: 1505-12). See, e.g., Powers and Palecek (2015) J. Heathc Eng. 3(4): 503-534, for a review of protein analytical assays for diagnosing cancer patients.

In some embodiments, any appropriate disease or condition associated with one or more chromosomal anomalies as described herein (e.g., based at least in part on the presence of one or more chromosomal anomalies, such as, without limitation, an aneuploidy) is identified as described herein. In some embodiments, the disease is cancer. Examples of cancers that can be associated with one or more chromosomal anomalies include, without limitation, lung cancer (e.g., small cell lung carcinoma or non-small cell lung carcinoma), papillary thyroid cancer, medullary thyroid cancer, differentiated thyroid cancer, recurrent thyroid cancer, refractory differentiated thyroid cancer, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, colorectal cancer (e.g., metastatic colorectal cancer), papillary renal cell carcinoma, ganglioneuromatosis of the gastroenteric mucosa, inflammatory myofibroblastic tumor, or cervical cancer, acute lymphoblastic leukemia (ALL), acute myeloid leukemia (AML), cancer in adolescents, adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, colon cancer, colorectal cancer, craniopharyngioma, cutaneous T-cell lymphoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, non-small cell lung cancer, oral cancer, oral cavity cancer, lip cancer, oropharyngeal cancer, osteosarcoma, ovarian cancer, pancreatic cancer, hepatobiliary cancer, upper urinary tract cancer, papillomatosis, paraganglioma, paranasal sinus and nasal cavity cancer, parathyroid cancer, penile cancer, pharyngeal cancer, pheochromosytoma, pituitary cancer, plasma cell neoplasm, pleuropulmonary blastoma, pregnancy and breast cancer, primary central nervous system lymphoma, primary peritoneal cancer, prostate cancer, rectal cancer, renal cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, Waldenstrom Macroglobulinemia, Wilms' tumor, 1p36 deletion syndrome, 1q21.1 deletion syndrome, 2q37 deletion syndrome, Wolf-Hirschhorn syndrome, Cri du chat, 5q deletion syndrome, Williams syndrome, Monosomy 8p, Monosomy 8q, Alfi's syndrome, Kleefstra syndrome, Monosomy 10p, Monosomy 10q, Jacobsen syndrome, Patau syndrome, Angelman syndrome, Prader-Willi syndrome, Miller-Dieker syndrome, Smith-Magenis syndrome, Edwards syndrome, Down syndrome, DiGeorge syndrome, Phelan-McDermid syndrome, 22q11.2 distal deletion syndrome, Cat eye syndrome, XYY syndrome, Triple X syndrome, Klinefelter syndrome, Wolf-Hirschhorn syndrome, Jacobsen syndrome, Charcot-Marie-Tooth disease type 1A, and Lynch Syndrome.

Once identified as having a disease associated with one or more chromosomal anomalies as described herein (e.g., based at least in part on the presence of one or more chromosomal anomalies, such as, without limitation, an aneuploidy), a mammal (e.g., a human) can be treated accordingly. For example, when a mammal is identified as having a cancer associated with one or more chromosomal anomalies as described herein, the mammal can be treated with one or more cancer treatments. The one or more cancer treatments can include any appropriate cancer treatments. A cancer treatment can include surgery. A cancer treatment can include radiation therapy. A cancer treatment can include administration of a pharmacotherapy such chemotherapy, hormone therapy, targeted therapy, and/or cytotoxic therapy. Examples of cancer treatments include, without limitation, platinum compounds (such as cisplatin or carboplatin), taxanes (such as paclitaxel or docetaxel), albumin bound paclitaxel (nab-paclitaxel), altretamine, capecitabine, cyclophosphamide, etoposide (vp-16), gemcitabine, ifosfamide, irinotecan (cpt-11), liposomal doxorubicin, melphalan, pemetrexed, topotecan, vinorelbine, luteinizing-hormone-releasing hormone (LHRH) agonists (such as goserelin and leuprolide), anti-estrogen therapy (such as tamoxifen), aromatase inhibitors (such as letrozole, anastrozole, and exemestane), angiogenesis inhibitors (such as bevacizumab), poly(ADP)-ribose polymerase (PARP) inhibitors (such as olaparib, rucaparib, and niraparib), external beam radiation therapy, brachytherapy, radioactive phosphorus, and any combinations thereof.

### Multi-Analyte Test to Increase Sensitivity of Detection

In some embodiments, methods provided herein to detect aneuploidy (e.g., using the analysis of chromosomal sequences (see e.g., Table 1 for an exemplary list of repetitive elements that can be analyzed)) increase sensitivity of cancer detection compared to cancer detection using the presence of one or more genetic biomarkers as indicators of cancer. In some embodiments, methods provided herein to detect aneuploidy (e.g., using the analysis of chromosomal sequences (see e.g., Table 1 for an exemplary list of repetitive elements that can be analyzed)) increase sensitivity of cancer detection compared to cancer detection using the presence of one or more protein biomarkers as indicators of cancer.

In some embodiments, methods provided herein to detect aneuploidy (e.g., using the analysis of chromosomal sequences (see e.g., Table 1 for an exemplary list of repetitive elements that can be analyzed)) are combined with one or more methods to detect the presence of one or more genetic biomarkers (e.g., mutations). In some embodiments, the combination of aneuploidy detection with genetic biomarker detection increases the specificity and/or sensitivity of detecting cancer. In some embodiments, methods provided herein to detect aneuploidy (e.g., using the analysis of chromosomal sequences (see e.g., Table 1 for an exemplary list of repetitive elements that can be analyzed)) are combined with one or more methods to detect the presence of one or more members of a panel of protein biomarkers (e.g., peptides). In some embodiments, the combination of aneuploidy detection with protein biomarker detection increases the specificity and/or sensitivity of detecting cancer. In some embodiments, methods provided herein to detect aneuploidy (e.g., using the analysis of chromosomal sequences (see e.g., Table 1 for an exemplary list of repetitive elements that can be analyzed)) are combined with methods to detect the presence of one or more genetic biomarkers (e.g., mutations) and/or methods to detect the presence of one or more members of a panel of protein biomarkers (e.g., peptide). In some embodiments, the combination of aneuploidy detection with genetic and/or protein biomarker detection increases the specificity and/or sensitivity of detecting cancer.

In some embodiments, methods provided herein to detect aneuploidy are combined with methods to detect the presence of one or more genetic biomarkers (e.g., mutations) in one or more genes selected from the group consisting of: NRAS, PTEN, FGFR2, KRAS, POLE, AKT1, TP53, RNF43, PPP2R1A, MAPK1, CTNNB1, PIK3CA, FBXW7, PIK3R1, APC, EGFR, BRAF. In some embodiments, methods provided herein to detect aneuploidy are combined with methods to detect the presence of one or more genetic biomarkers (e.g., mutations) in one or more genes selected from the group consisting of: PTEN, TP53, PIK3CA, PIK3R1, CTNNB1, KRAS, FGFR2, POLE, APC, FBXW7, RNF43, and PPP2R1A. In some embodiments, an assay includes detection of genetic biomarkers (e.g., mutations) in one or more of any of the genes disclosed herein including, without limitation, CDKN2A, FGF2, GNAS, ABL1, EVIL MYC, APC, IL2, TNFAIP3, ABL2, EWSR1, MYCL1, ARHGEF12, JAK2, TP53, AKT1, FEV, MYCN, ATM, MAP2K4, TSC1, AKT2, FGFR1, NCOA4, BCL11B, MDM4, TSC2, ATF1, FGFR1OP, NFKB2, BLM, MEN1, VHL, BCL11A, FGFR2, NRAS, BMPR1A, MLH1, WRN, BCL2, FUS, NTRK1, BRCA1, MSH2, WT1, BCL3, GOLGA5, NUP214, BRCA2, NF1, BCL6, GOPC, PAX8, CARS, NF2, BCR, HMGA1, PDGFB, CBFA2T3, NOTCH1, BRAF, HMGA2, PIK3CA, CDH1, NPM1, CARD11, HRAS, PIM1, CDH11, NR4A3, CBLB, IRF4, PLAG1, CDK6, NUP98, CBLC, JUN, PPARG, SMAD4, PALB2, CCND1, KIT, PTPN11, CEBPA, PML, CCND2, KRAS, RAF1, CHEK2, PTEN, CCND3, LCK, REL, CREB1, RB1, CDX2, LMO2, RET, CREBBP, RUNX1, CTNNB1, MAF, ROS1, CYLD, SDHB, DDB2, MAFB, SMO, DDX5, SDHD, DDIT3, MAML2, SS18, EXT1, SMARCA4, DDX6, MDM2, TCL1A, EXT2, SMARCB1, DEK, MET, TET2, FBXW7, SOCS1, EGFR, MITF, TFG FH, STK11, ELK4, MLL, TLX1, FLT3, SUFU, ERBB2, MPL, TPR, FOXPL SUZ12, ETV4, MYB, USP6, GPC3, SYK, ETV6, IDH1, and/or TCF3. In some embodiments, combining the detection of aneuploidy with the detection of one or more genetic biomarkers (e.g., mutations) increases the specificity and/or sensitivity of detecting cancer.

In some embodiments, detection of a genetic biomarker (e.g., one or more genetic biomarkers) includes any of the variety of methods described in U.S. Pat. No. 7,700,286, which is hereby incorporated by reference in its entirety. Any of the variety of methods of messenger RNA (“mRNA”) isolation known in the art may be used to isolate RNA from a sample (e.g., Qiagen RNeasy Kit). Any of the variety of methods of genomic DNA (“gDNA”) isolation known in the art may be used to isolate gDNA from the sample (e.g., Qiagen DNeasy Kit). In some embodiments, detection of a genetic biomarker includes a cancer detection assay. In some embodiments, the amount of gDNA and/or mRNA in a sample are measured for any of the genetic biomarkers disclosed herein. Changes in the amount of gDNA and/or mRNA may indicate cancer. For example, when measuring gDNA, gene amplification (e.g., increased copy number of chromosomal sequences (e.g., coding regions of genes or non-coding DNA (see e.g., Table 1 for an exemplary list of repetitive elements that can be measured)) may indicate cancer. For example, when measuring mRNA, increases in the amount of RNA (e.g., increased expression of a genetic biomarker) may indicate cancer. In some cases, changes in DNA and RNA may correlate.

In some embodiments, methods provided herein to detect aneuploidy can be combined with methods to detect the presence of one or more protein biomarkers (e.g., peptides) in one or more proteins selected from the group consisting of: AFP, CA19-9, CEA, HGF, OPN, CA-125, CA15-3, MPO, prolactin (PRL) and/or TIMP-1 to determine the presence of cancer (e.g., ovarian or endometrial). In some embodiments, a protein biomarker can be any appropriate peptide biomarker. In some embodiments, a peptide biomarker can be a peptide biomarker associated with cancer. For example, a peptide biomarker can be a peptide having elevated levels in a cancer (e.g., as compared to a reference level of the peptide).

Exemplary and non-limiting threshold levels for certain protein biomarkers include: CA19-9 (>92 U/ml), CEA (>7,507 pg/ml), CA125 (>577 U/ml), AFP (>21,321 pg/ml), Prolactin (>145,345 pg/ml), HGF (>899 pg/ml), OPN (>157,772 pg/ml), TIMP-1 (>176,989 pg/ml), Follistatin (>1,970 pg/ml), and CA15-3 (>98 U/ml). In some embodiments, threshold levels for protein biomarkers can be higher (e.g., about 10%, about 20%, about 30%, about 40%, about 50%, about 60%, about 70%, about 80%, about 90%, about 100%, or higher) than the exemplary threshold levels described herein. In some embodiments, threshold levels for protein biomarkers can be lower (e.g., about 10%, about 20%, about 30%, about 40%, about 50%, or lower) than the exemplary threshold levels described herein.

In some embodiments, a threshold level of CA19-9 can be at least about 92 U/mL (e.g., about 92 U/mL). In some embodiments, a threshold level of CA19-9 can be 92 U/mL. In some embodiments, a threshold level of CEA can be at least about 7,507 pg/ml (e.g., about 7,507 pg/ml). In some embodiments, a threshold level of CEA can be 7.5 ng/mL. In some embodiments, a threshold level of HGF can be at least about 899 pg/ml (e.g., about 899 pg/ml). In some embodiments, a threshold level of HGF can be 0.92 ng/mL. In some embodiments, a threshold level of OPN can be at least about 157,772 pg/ml (e.g., about 157,772 pg/ml). In some embodiments, a threshold level of OPN can be 158 ng/mL. In some embodiments, a threshold level of CA125 can be at least about 577 U/ml (e.g., about 577 U/ml). In some embodiments, a threshold level of CA125 can be 577 U/mL. In some embodiments, a threshold level of AFP can be at least about 21,321 pg/ml (e.g., about 21,321 pg/ml). In some embodiments, a threshold level of AFP can be 21,321 pg/ml. In some embodiments, a threshold level of prolactin can be at least about 145,345 pg/ml (e.g., about 145,345 pg/ml). In some embodiments, a threshold level of prolactin can be 145,345 pg/ml. In some embodiments, a threshold level of TIMP-1 can be at least about 176,989 pg/ml (e.g., about 176,989 pg/ml). In some embodiments, a threshold level of TIMP-1 can be 176,989 pg/ml. In some embodiments, a threshold level of follistatin can be at least about 1,970 pg/ml (e.g., about 1,970 pg/ml). In some embodiments, a threshold level of CA15-3 can be at least about 98 U/ml (e.g., about 98 U/ml). In some embodiments, a threshold level of CA15-3 can be 98 U/ml. In some embodiments, a threshold level of CA19-9, CEA, and/or OPN can be 5%, 10%, 15%, 20%, 25%, 30%, 35%, 40%, 45%, 50%, 55%, 60%, 65%, 70%, 75%, 80%, 85%, 90%, 95%, 100% or more greater than the threshold levels listed above (e.g., greater than a threshold level of 92 U/mL for CA-19-9, 7,507 pg/ml for CEA, 899 pg/ml for HGF, 157,772 pg/ml for OPN, 577 U/ml for CA125, 21,321 pg/ml for AFP, 145,345 pg/ml for prolactin, 176,989 pg/ml for TIMP-1, 1,970 pg/ml for follistatin, and/or 98 U/ml for CA15-3).

In some embodiments, a threshold level of protein biomarker can be greater than the levels that are typically tested for diagnostic or clinical purposes. For example, the threshold level of CA19-9 can be greater than about 37 U/ml (e.g., greater than about 40, 45, 50, 55, 60, 65, 70, 75, 80, 85, 90, 95 or more U/mL). Additionally or alternatively, the threshold level of CEA can be greater than about 2.5 ug/L (e.g., greater than about 3.0, 3.5, 4.0, 4.5, 5.0, 5.5, 6.0, 6.5, 7.0, 7.5 or more ug/L). Additionally or alternatively, the threshold level of CA125 can be greater than about 35 U/mL (e.g., greater than about 40, 45, 50, 55, 60, 65, 70, 75, 80, 85, 90, 95, 100, 150, 200, 250, 300, 350, 400, 450, 500, 550 or more U/mL). Additionally or alternatively, the threshold level of AFP can be greater than about 21 ng/mL (e.g., greater than about 25, 30, 40, 50, 60, 70, 80, 90, 100, 150, 200, 250, 300, 350, 400 or more ng/L). Additionally or alternatively, the threshold level of TIMP-1 can be greater than about 2300 ng/mL (e.g., greater than about 2,500, 3,000, 4,000, 5,000, 6,000, 7,000, 8,000, 9,000, 10,000, 15,000, 20,000, 25,000, 30,000, 35,000, 40,000 or more ng/L). Additionally or alternatively, the threshold level of follistatin can be greater than about 2 ug/mL (e.g., greater than about 2.5, 3.0, 3.5, 4.0, 4.5, 5.0, 5.5, 6.0, 6.5, 7.0, 7.5 or more ug/L). Additionally or alternatively, the threshold level of CA15-3 can be greater than about 30 U/mL (e.g., greater than about 35, 40, 45, 50, 55, 60, 65, 70, 75, 80, 85, 90, 95 or more U/mL). In some embodiments, detecting one or more protein biomarkers at threshold levels that are higher than are typically tested for during traditional diagnostic or clinical assays can improve the sensitivity of cancer detection.

Examples of peptide biomarkers include, without limitation, AFP, Angiopoietin-2, AXL, CA125, CA 15-3, CA19-9, CD44, CEA, CYFRA 21-1, DKK1, Endoglin, FGF2, Follistatin, Galectin-3, G-CSF, GDF15, HE4, HGF, IL-6, IL-8, Kallikrein-6, Leptin, LRG-1, Mesothelin, Midkine, Myeloperoxidase, NSE, OPG OPN, PAR, Prolactin, sEGFR, sFas, SHBG sHER2/sEGFR2/sErbB2, sPECAM-1, TGFa, Thrombospondin-2, TIMP-1, TIMP-2, and Vitronectin. For example, a peptide biomarker can include one or more of OPN, IL-6, CEA, CA125, HGF, Myeloperoxidase, CA19-9, Midkine and/or TIMP-1. In some embodiments, combining the detection of aneuploidy with the detection of one or more protein biomarkers (e.g., peptides) increases the specificity and/or sensitivity of detecting cancer.

In some embodiments, the presence of a genetic and/or protein biomarker may be detected in any of a variety of biological samples isolated or obtained from a subject (e.g., a human subject) including, but not limited to blood, plasma, serum, urine, cerebrospinal fluid, saliva, sputum, broncho-alveolar lavage, bile, lymphatic fluid, cyst fluid, stool, ascites, and combinations thereof. Any protein biomarker known in the art may be detected when a threshold value is obtained above which normal, healthy human subjects do not fall, but human subjects with cancer do fall. Any appropriate method can be used to detect the level of one or more protein biomarkers as described herein. In some embodiments, the level of one or more protein biomarkers is compared to a predetermined threshold. In some embodiments, the predetermined threshold is a general or global threshold. In some embodiments, the predetermined threshold is a threshold that is relevant to a particular protein biomarker. In some embodiments, the level of the one or more protein biomarkers is compared to an absolute amount of a reference protein biomarker. In some embodiments, the level of the one or more protein biomarkers is relative to an amount of a reference protein biomarker. In some embodiments, the level of the one or more protein biomarkers is an elevated level. In some embodiments, the level of the one or more protein biomarkers is above a predetermined threshold. In some embodiments, the level of the one or more protein biomarkers is within a predetermined threshold range. In some embodiments, the level of the one or more protein biomarkers is or approximates a predetermined threshold. In some embodiments, the level of the one or more protein biomarkers is below a predetermined threshold. In some embodiments, the level of the one or more protein biomarkers from a biological sample is lower than a particular threshold. In some embodiments, the level of the one or more protein biomarkers from a biological sample is depressed compared to a predetermined threshold.

In some embodiments, methods and materials described herein can be used for detecting one or more polymorphisms (e.g., somatic mutations) in a genome of a mammal. For example, a plurality of amplicons obtained from a sample obtained from a first mammal (e.g., a test mammal or a mammal suspected of harboring one or more polymorphisms) can be sequenced, a plurality of amplicons obtained from a sample obtained from a second mammal (e.g., a reference mammal) can be sequenced, variant sequencing reads from the sample obtained from the first mammal can be grouped into clusters of genomic intervals, reference sequencing reads from the sample obtained from the second mammal can be grouped into clusters of genomic intervals, a chromosome arm having a sum of the variant sequencing reads and the reference sequencing reads on both alleles that is greater than about 3 (e.g., greater than about 4, greater than about 5, greater than about 6, greater than about 7, greater than about 8, greater than about 9, greater than about 10, greater than about 12, greater than about 15, greater than about 18, greater than about 20, greater than about 22, greater than about 25, or greater than about 30) can be selected, a variant-allele frequency (VAF) of the selected chromosome arm can be determined, and the presence or absence of one or more polymorphisms on the selected chromosome arm can be identified. A VAF of the selected chromosome arm can be determined using any appropriate technique. For example, a VAF of the selected chromosome arm can be the number of variant sequencing reads/total number of sequencing reads. The presence of one or more polymorphisms in the genome of the mammal can be identified in the genome of the mammal when the VAF is between about 0.2 and about 0.8 (e.g., between about 0.3 and about 0.8, between about 0.4 and about 0.8, between about 0.5 and about 0.8, between about 0.6 and about 0.8, between about 0.2 and about 0.7, between about 0.2 and about 0.6, between about 0.2 and about 0.5, or between about 0.2 and about 0.4), and the absence of one or more polymorphisms in the genome of the mammal can be identified in the genome of the mammal when the VAF is within a predetermined significance threshold. For example, without limitation, the presence of one or more polymorphisms in the genome of the mammal can be identified in the genome of the mammal when the VAF is between about 0.4 and 0.6.

In some embodiments, methods and materials described herein can be used for sample identification. The repetitive elements amplified by the methods described herein include common polymorphisms that can be used to establish or refute sample identify among samples (e.g., plasma, tumor, and blood). For example, the genotype at each polymorphic location can be identified and compared across samples. Overall similarities between samples at polymorphic locations can be used to determine sample identity.

In some cases the diseases associated with one or more chromosomal anomalies as described herein (e.g., based at least in part on the presence of one or more chromosomal anomalies, such as, without limitation, an aneuploidy) are also associated with increased mutation rates (e.g., increased mutation rates can be associated with stage of disease) when compared to a control (e.g., non-disease sample). In such cases, the materials and methods described herein can be used to (a) identify the presence of one or more chromosomal anomalies (e.g., aneuploidy) and (b) identify the stage (e.g., cancer stages I, II, III, and IV) of the disease based on a determination of the mutation rate (e.g., number of mutations) compared to a control.

The invention will be further described in the following examples, which do not limit the scope of the invention described in the claims.

### EXAMPLES

### Example 1: Detection of Aneuploidy in Patients with Cancer

This example describes a novel adaptation of amplicon-based aneuploidy detection. An approach called WALDO for Within-Sample-AneupLoidy-DetectiOn, which employs supervised machine learning to detect changes in chromosome arms, improved aneuploidy detection sensitivity compared to previous methods. It is shown here that using WALDO to analyze amplicons of short interspersed nucleotide elements (SINEs) from a DNA sample increases sensitivity of aneuploidy detection. In addition, the 1,000,000 SINE amplicons with an average length of about 100 bp reduce the input requirement for cell free DNA input while also increasing sensitivity of detection.

### Materials and Methods

### Primers

To generate a list of candidate primers, the frequency of all possible 6-mers (4\hat{ }6=4096) within the RepeatMasker track of hg19 were calculated. Next, the frequency of all possible 4-mers (4\hat{ }4=256) within 75 bp upstream or downstream from the 6-mers were calculated. Joining the 6-mers with the 4-mers generated 2,097,152 candidate pairs. These pairs were selected for further assessment based on the number of unique genomic loci expected from their PCR-mediated amplification, the average size between the 6-mer and its corresponding 4-mers, and the distribution of these sizes, aiming for a unimodal distribution. This filtering criteria generated 16 potential k-mer pairs, leading to the design of 16 primer pairs that incorporated these k-mer pairs at their 3-ends. A k-mer is understood in the art to refer to a subsequence of length k which is contained within a sequence.

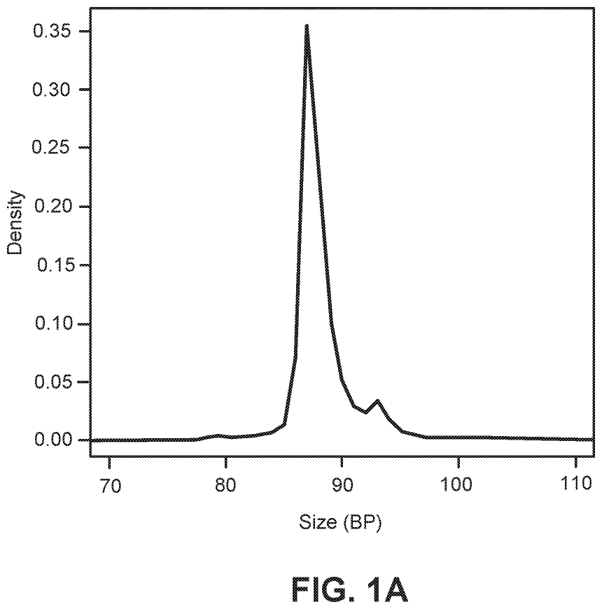

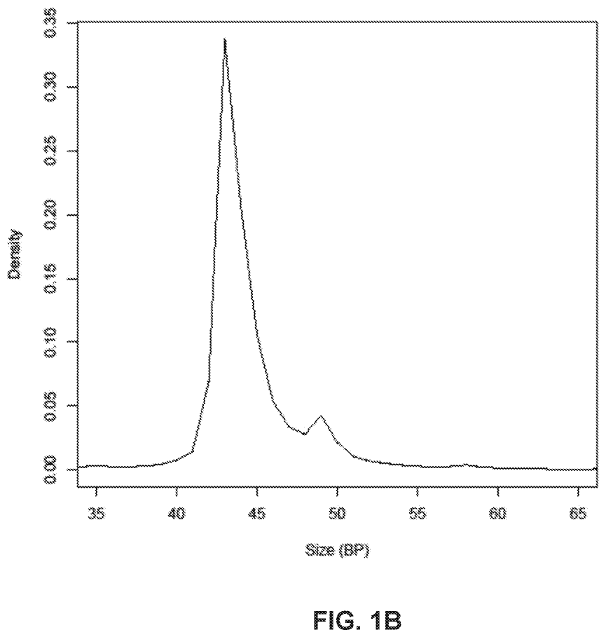

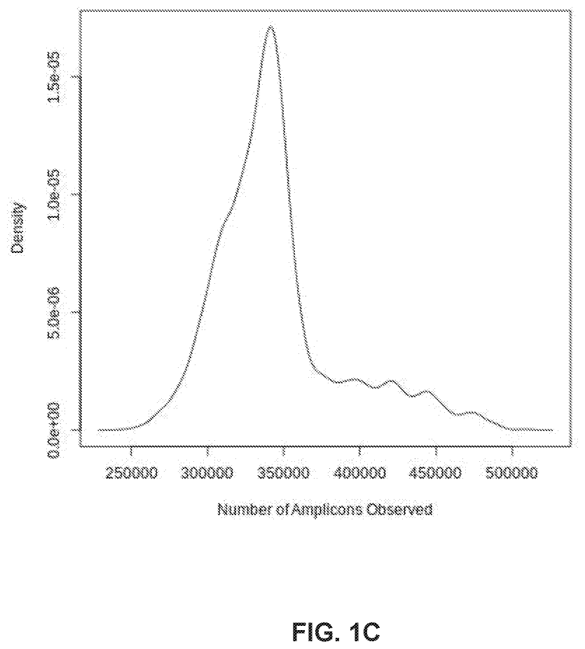

In total, 16 primers were initially designed and tested (Table 2). One primer (SEQ ID NO: 1) consistently had fewer primer dimers and was selected for use in testing a cohort. A primer pair having SEQ ID NO: 1 as one of the primers uniquely amplified 745,184 amplicons, which amplicons had an average amplicon size of ˜88 bp (FIG. 1A). The amplicons sizes shown in FIG. 1A include 45 bp of primers. For example, when not including the primers, the amplicons have an average size of ˜43 base pairs (FIG. 1B).

TABLE 2

| | SEQ | | |
|---|---|---|---|
| Primer | ID | Primer | |
| Pair | NO: | Name | Sequence |
| 1 | 1 | FP1_n16 | cgacgtaaaacgacggccagt |
| | | | NNNNNNNNNNNNNNNNGGTGA |
| | | | AACCCCGTCTCTACA |
| 2 | 2 | FP2_n16 | cgacgtaaaacgacggccagt |
| | | | NNNNNNNNNNNNNNNNGGTGA |
| | | | AACCCCGTCTCTAC |
| 3 | 3 | FP3_n16 | cgacgtaaaacgacggccagt |
| | | | NNNNNNNNNNNNNNNNGGTGA |
| | | | AACCCCGTCTCTACT |
| 4 | 4 | FP4_n16 | cgacgtaaaacgacggccagt |
| | | | NNNNNNNNNNNNNNNNCATGC |
| | | | CTGTAGTCCCAGCTACT |
| 5 | 5 | FP5_n16 | cgacgtaaaacgacggccagt |
| | | | NNNNNNNNNNNNNNNNATAGT |
| | | | GAAACCCCATCTCTACAAAA |
| 6 | 6 | FP6_n16 | cgacgtaaaacgacggccagl |
| | | | NNNNNNNNNNNNNNNNGGTGA |
| | | | AACCCCATCTCTACAA |
| 7 | 7 | FP7_n16 | cgacgtaaaacgacggccagt |
| | | | NNNNNNNNNNNNNNNNATAGT |
| | | | GAAACCCCATCTCTACAAA |
| 8 | 8 | FP8_n16 | cgacglaaaacgacggccagt |
| | | | NNNNNNNNNNNNNNNNGAGGT |
| | | | GGGAGGATTGCTT |
| 9 | 9 | FP9_n16 | cgacgtaaaacgacggccagt |
| | | | NNNNNNNNNNNNNNNNACCAG |
| | | | CCTGGGCAACATA |
| 1 | 10 | RP1_z4 | cacacaggaaacagctatgac |
| | | | catgCCTCCTAAGTAGCTGGG |
| | | | ACTACAG |
| 2 | 11 | RP2_z4 | cacacaggaaacagclatgac |
| | | | catgCCTCCTAAGTAGCTGGG |
| | | | ACTACAG |
| 3 | 12 | RP3_z4 | cacacaggaaacagctatgac |
| | | | catgCCTCCTAAGTAGCTGGG |
| | | | ACTACAG |
| 4 | 14 | RP4_z4 | cacacaggaaacagctatgac |
| | | | catgTGCAGTGGCACGATCAT |
| | | | AGCTCACTGCAGCCTTGA |
| 5 | 15 | RP5_z4 | cacacaggaaacagctatgac |
| | | | catgCTCCCGAGTAGCTGGGA |
| | | | CT |
| 6 | 16 | RP6_z4 | cacacaggaaacagctatgac |
| | | | catgCTCCCGAGTAGCTGGGA |
| | | | CTAC |
| 7 | 17 | RP7_z4 | cacacaggaaacagctatgac |
| | | | catgCCCGAGTAGCTGGGACT |
| | | | ACA |
| 8 | 18 | RP8_z4 | cacacaggaaacagctatgac |
| | | | catgAGGCTGGAGTGCAGTGG |
| 9 | 19 | RP9_z4 | cacacaggaaacagctatgac |
| | | | catgCCACCATGCCTGGCTAA |

### Sequencing Library Preparation

The first primer having SEQ ID NO: 1 included from the 5′ to 3′ end: a universal primer sequence (UPS), a unique identifier DNA sequence (UID), and an amplification sequence. Polymerase chain reaction (PCR) was performed in 25 uL reactions containing 7.25 uL of water, 0.125 uL of each primer, 12.5 uL of NEBNext Ultra II Q5 Master Mix (New England Biolabs cat #M0544S), and 5 uL of DNA. The cycling conditions were: one cycle of 98° C. for 120 s, then 15 cycles of 98° C. for 10 s, 57° C. for 120 s, and 72° C. for 120 s. For experiments with plasma, the amount of DNA in 5 uL was 0.14 ng. A second round of PCR was then performed to add dual indexes (barcodes) to each PCR prior to sequencing. The forward and reverse primers used for the second round of PCR are listed in Table 2. The initial amplification primers were not removed and the amplification product from the first reaction was diluted 1:20. The dilution was used directly for a second round of amplification using primers that annealed to the UPS site introduced by the first round primers and that additionally contained the 5′ grafting sequences necessary for hybridization to the Illumina flow cell.

Flndexes (e.g., sequences used to differentiate between samples) were introduced to each sample using the second reverse primer to later allow multiplexed sequencing. The second round of PCR was performed in 25 uL reactions containing 7.25 uL of water, 0.125 uL of each primer, 12.5 uL of NEBNext Ultra II Q5 Master Mix (New England Biolabs cat #M0544S), and 5 uL of DNA containing 5% of the PCR product from the first round. The cycling conditions were: one cycle of 98° C. for 120 s, then 15 cycles of 98° C. for 10 s, 65° C. for 15 s, and 72° C. for 120 s. Amplification products were run on agarose gels to check for amplification. Amplification products were purified with AMPure XP beads at 1.2× and were quantified by spectrophotometry, real time PCR, an Agilent 2100 Bioanalyzer or an automated electrophoresis using an Aiglent TapeStation. All oligonucleotides were purchased from Integrated DNA Technologies (Coralville, Iowa).

### Sequencing and Sequencing Analysis

Bowtie2 was used to align reads of the amplicons generated with each of the 7 primer pairs to the human reference genome assembly GRC37 (Langmead et al. 2012). With primer pair 1 (the primer having SEQ ID NO: 1 and the primer having SEQ ID NO: 10), an average of 51.1% of the total reads could be uniquely aligned and the average amplicon size was 88 bp (FIG. 1A). The amplicons sizes shown in FIG. 1A include 45 bp of primers. For example, when not including the primers, the amplicons have an average size of ˜43 base pairs (FIG. 1B). Primer pair 1 was theoretically able to amplify up to 745,184 repetitive elements that can be uniquely aligned, but the average sample contained an average of 350,000 repetitive elements, see FIG. 1C. Without wishing to be bound by theory, there were several potential reasons for the discrepancy between the potential number and the actual observed number of amplicons in plasma samples. (1) Polymorphisms within the sequences may have caused misalignment and result in “missing amplicons.” (2) Polymorphisms within the primers may not have amplified. (3) Each amplicon may have had a different PCR efficiency with low efficiency amplicons outcompeted during PCR. (4) Smaller DNA fragments may have been preferentially amplified and long amplicons (>100 bp) may not have been amplified. (5) Long amplicons may have been absent in cell free DNA due to the small sizes of the DNA fragments in cell free DNA. (6) The amount of sequencing used for these samples may not have been high enough to observe every amplicon especially those with low PCR efficiencies. (7) Finally, some repetitive elements may not have been present in every individual. Within the amplicons generated by the primer pair of SEQ ID NO: 1 and SEQ ID NO: 10, 52,762 polymorphisms were identified. The average number of heterozygous sites in the test cohort of 1348 normal plasmas and 883 plasmas from cancer patient individuals was 2,200. These sites could be used to measure allelic imbalance, genetically identify samples, and determine whether samples had been accidentally mixed together. Using the same SNPs, synthetic experiments were used to estimate that sample mixing could be detected when the amount of sample one DNA was >4% of the amount of sample two DNA in a given mixture.

### Statistical Analysis

Read-depth-based analytical methods have been widely applied to whole-genome sequencing (WGS) protocols. Under the assumption that reads are uniformly and independently distributed, regions of normal copy number are expected to follow a Poisson or normal distribution (Zhao et al 2013 and Pirooznia et al 2015). Amplicon-based protocols achieve high coverage depth at relatively low cost, and they are an attractive alternative to WGS, but aligned reads from amplicon sequencing such as those resulting from the above described assay have properties different from those resulting from WGS and WES. Because these reads are limited to a relatively small number of discrete loci, they are discontinuous. The reads are also not randomly distributed, which makes it difficult to use the statistical models of read depth coverage designed for WGS and WES. The Within-Sample AneupLoidy DetectiOn (WALDO), is an algorithm specifically designed for amplicon-based aneuploidy detection (see, e.g., Douville et al. PNAS 201 115(8):1871-1876). WALDO was applied to sequencing reads that mapped to the above described genomic loci (e.g., SINE). The genome-wide aneuploidy score was used to identify whether a sample had the presence of aneuploidy.

### Statistical Principles Underlying WALDO

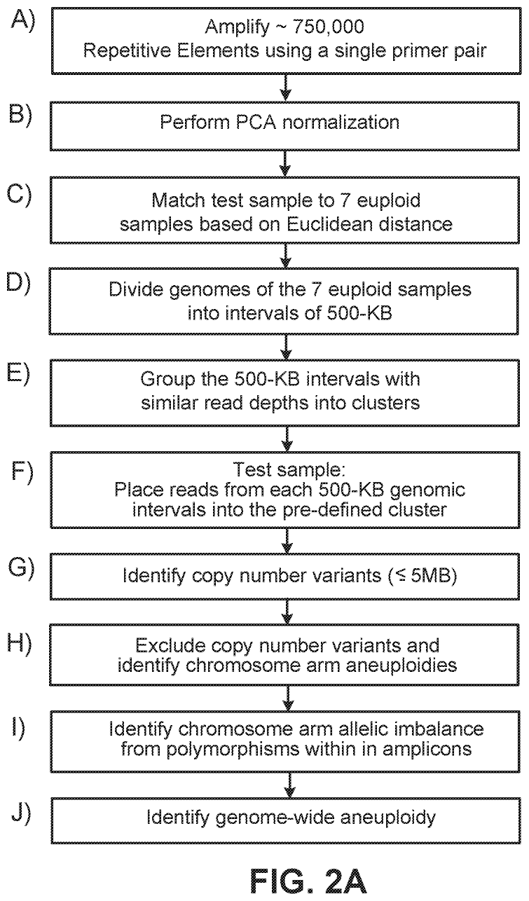

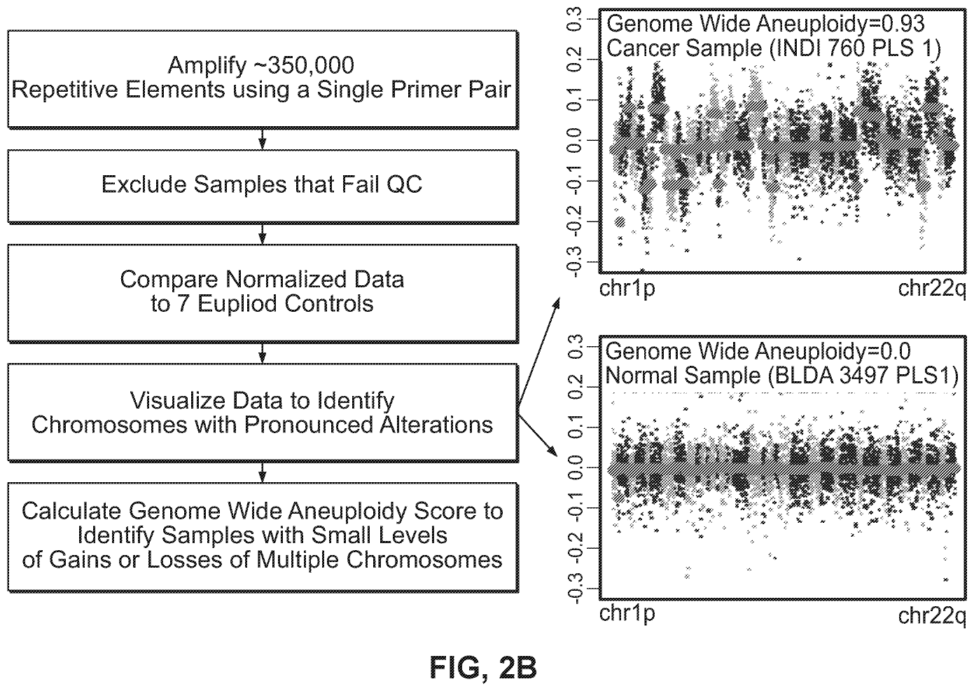

Unlike most conventional approaches for assessing copy number changes, WALDO does not compare normalized read counts from each chromosome arm in a test sample to the fraction of reads in each chromosome arm in other samples. Such conventional comparisons are subject to batch effects and other artifacts associated with variables that are difficult to control. To evaluate whole genome sequencing data, aneuploidy was detected by comparing the read counts within 5344 genomic intervals each containing 500-kb of sequence. The read counts within the 500-kb genomic intervals within a sample were only compared to the read counts of other genomic intervals within the same sample—hence the “Within-Sample” designation in WALDO. The previously described WALDO protocol was tailored in this Example, which resulted in several analytical changes (see FIG. 2). The modifications included a new normalization step, a new way to call small copy number changes of indeterminate length, and an improved way to detect genome-wide aneuploidy, as described below. These analytical improvements coupled with the increased genomic density of amplicons achieved with the SEQ ID NO: 1 and SEQ ID NO: 10 primer pair enabled greater sensitivity as well as the detection of focal amplifications and deletions less than 1 Mb in size.

In euploid samples, the number reads within each 500-kb genomic interval should track with the number of reads in certain other genomic regions. Genomic intervals that track together do so because the amplicons within them amplify to similar extents. Here, such genomic regions that track together are called “clusters”. It is possible identify clusters from sequencing data on euploid samples. In a test sample, it is determined whether the number of reads in each genomic interval in each pre-defined cluster is within the expected bound of the other clusters from that same sample. If the reads within a genomic interval are outside the statistically expected bound, and there are many such outsiders on the same chromosome arm, then that chromosome arm is classified as aneuploid. The statistical basis of this test is described elsewhere (e.g., Douville et al. PNAS 201 115(8):1871-1876). In brief, while the number of reads is not randomly distributed across the genome, the distribution of scaled reads within each cluster is approximately Normal. A convenient property of Normal distributions is that the sum of multiple Normal distributions is also a Normal distribution. It is thus possible to compute the theoretical mean and variance of the summed reads on each chromosome arm simply by summing the means and variances of all the clusters represented on that chromosome arm.

WALDO also employs several other innovations that make it applicable to the analysis of PCR-generated amplicons from clinical samples. One of these innovations is controlling amplification bias stemming from the strong dependence of the data on the size of the initial template. Another is the use of a machine learning algorithm (e.g., a Support Vector Machine (SVM)) to enable the detection of aneuploidy in samples containing low neoplastic fractions.

### Normalization

The improved WALDO methods described in this Example include a new method of normalization that reduced the amount of variability between samples. In this normalization, a principal component analysis (PCA) was first performed on sequencing data from the controls. PCA reduced the number of 500 kb genomic intervals from n=5,344 to a more manageable number of dimensions. Using the PCA coordinates of the controls, a modeled was created to predict whether a particular 500 kb interval will be amplified more or less efficiently in future samples based on their PCA coordinates.

Correction Factor for 500 kb Intervalᵢ=βₒᵢ+β₁ᵢ*PCA₁β₂ᵢ*PCA₂+β₃ᵢ*PCA₃+β₄ᵢ*PCA₄+β₅ᵢ*PCA₅

For each test sample, the sample was projected into PCA space and the correction factor was calculated for each 500 kb interval as function of its PCA coordinates. After applying the correction factor to each 500 kb genomic interval, the test sample was matched to 7 control samples based on the closest Euclidean distance of the 500 kb intervals.

### Generation of Synthetic Aneuploidy Samples.

Data was selected from 84 presumably euploid plasma samples, each containing at least 10 million reads, and each derived from the DNA of normal WBCs. Synthetic aneuploid samples were created by adding (or subtracting) reads from several chromosome arms to the reads from these normal DNA samples. The reads were added or subtracted from 1, 10, 15, or 20 chromosome arms to each sample. The additions and subtractions were designed to represent neoplastic cell fractions ranging from 0.5% to 1.5% and resulted in synthetic samples containing exactly ten million reads. The reads from each chromosome arm were added or subtracted uniformly. For example, when modeling five chromosome arms that were lost, each was lost to the identical degree and we did not incorporate tumor heterogeneity into the model. Furthermore, synthetic samples were not created containing more than three of any chromosome arm; e.g. 4 copies of chromosome 3p. This simplified approach did not comprehensively cover all biologically plausible aneuploidy events. However, limiting the possible combinations of altered arms made sample generation computationally tractable, and the resulting support vector machine worked well in practice. The synthetically generated samples in which reads from only a single chromosome arm were added or subtracted enabled us to estimate the performance of WALDO when only a single chromosome arm of interest was gained or lost. The pseudocode to generate synthetic samples is shown in FIG. 5.

### Determination of Genome Wide Aneuploidy

A two-class support vector machine (SVM) was trained to discriminate between euploid samples and aneuploid samples. The training set contained a negative class of 1348 presumably euploid plasma samples from normal individuals containing at least 2.5M reads and 635 aneuploid samples. The aneuploid class contained a mixture of synthetic and actual aneuploid samples. SVM training was done with the e1071 package in R, using radial basis kernel and default parameters. Each sample had 39 Z-score features, representing chromosome arm gains and losses. During training, the positive class was randomly sampled so that the positive class was 10% the size of the negative class. The positive class was randomly sampled at a ratio of two real samples to one synthetic sample. Ten iterations of this procedure were performed. The final genome wide aneuploidy score was the average of the raw svm score across the 10 iterations.

### Results

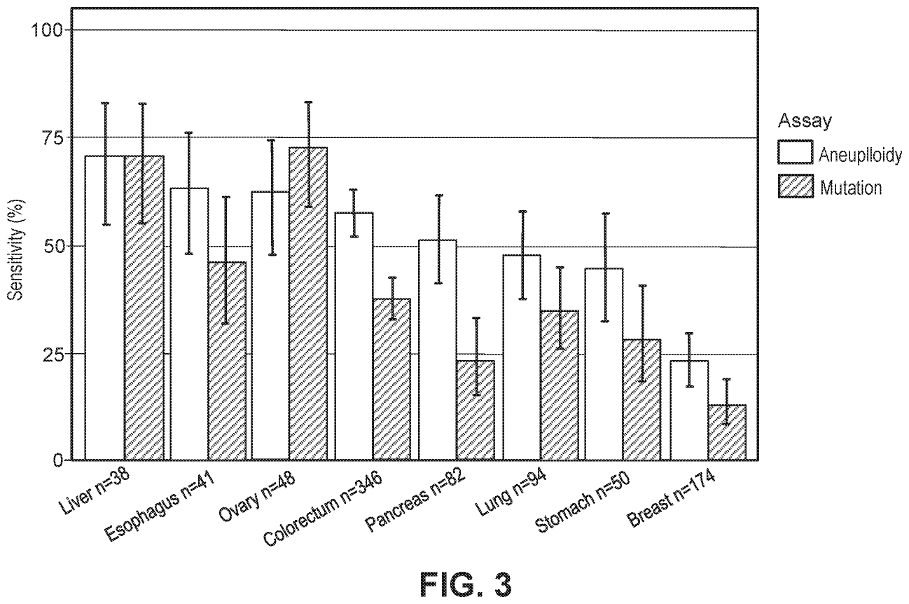

The performance of this assay was assessed on a cohort of 1348 euploid plasma samples and 883 plasma samples from cancer patients (Table 3). The samples from cancer patients included Breast, Colorectum, Esophagus, Liver, Lung, Ovary, Pancreas, and Stomach cancers (FIG. 3). Using a cutoff of that resulted in 99% specificity defined in our cohort of 1348 euploid samples, it was found that 49% plasmas from cancer samples had aneuploidy.

### Sample Exclusion Criteria

To ensure that all samples included in the results section of paper were of high quality, several exclusion criteria were developed. First, samples with fewer than 2.5M reads were excluded. Second, samples with sufficient evidence of contamination were excluded. To be labeled as contaminated, the sample had to have at least 10 significant allelic imbalanced chromosome arms (z score>=2.5) and fewer than ten significant chromosome arms gains or losses (z>=2.5 or z<=−2.5). Allelic imbalance is determined from SNPs, while gains or losses were assessed through WALDO. As determined through mixing experiments, a relatively large number of allelic imbalanced chromosome arms in the absence of a large number of gains or losses indicated contamination of the sample with DNA from another individual. Third, in plasma analyses, samples in which more than 8.5% of the amplicons were larger than 94 bps (50 base pairs between the forward and reverse primers) were excluded. Such samples were likely to be contaminated with leukocyte DNA. Fourth, samples outside the dynamic range of the assay, as defined by the equation below, were excluded.

$$QC Dynamic Range Metric = {\sum }_{i}^{2 q , 3 q , 4 q , 5 q , 6 q , 8 q , 13 q} \frac{Reads on {chr}_{i}}{{{\sum }_{j = 1}}^{39} Reads on {chr}_{j}}$$

The distribution of this metric has long tails. The values of >0.2450 and 0.2320 were selected as a dynamic range that we could evaluate cutoffs. Fifth, plasma samples with known aneuploidy in the leukocytes of the same patients; such patients were assumed to have Clonal Hematopoiesis of Indeterminate Potential (CHIP) or congenital disorders.

### Detection of Cancer Using a Multi-Analyte Test

Whether aneuploidy could be integrated as an additional biomarker into the published framework, as well as the predictive ability of a logistic regression model with aneuploidy and protein markers against the original logistic regression model that uses somatic mutations and protein markers, was compared.

Here, 1348 plasma samples from healthy people and 883 cancer patients were analyzed. Of the 1348 healthy samples, only 248 overlapped with the original study. All 883 cancer samples were included in the original study. The sample demographic information was provided in Table 3.

Using the original 812 healthy samples (Cohen et al.) and the 883 cancer samples, a logistic regression model was trained and then used to assess performance using ten rounds of tenfold cross validation. A full list of samples and their biomarker values was provided in Table 3. Because 564 of the original healthy samples were not analyzed for aneuploidy, the list of scores from the 1348 normal samples was randomly sampled and assigned each missing sample an aneuploidy value. Ten rounds of analysis were performed and each new round, the collection of 1348 normal scores again randomly sampled to assign the 564 samples a new score.

To account for variations in the lower limits of detection across different experiments, the 90^{th} percentile feature value was used in the healthy training samples. Any feature value below this threshold and set all values to the 90^{th} percentile threshold. This transformation was done for all training and testing samples. This procedure was done for aneuploidy scores, somatic mutation scores, and protein concentrations. The 90^{th} percentile thresholds and final feature coefficients from the logistic regression model were listed in Table 4.

TABLE 4
Logistic regression coefficients and thresholds.

| | 90th Percentile Values | Coefficients |
|---|---|---|
| Intercept | | −11.8552 |
| Aneuploidy | 0.116389196 | 8.014704 |
| Omega | 1.145773698 | 2.343129 |
| AFP | 2866.68 | 9.26E−06 |
| CA.125 | 6.9024 | 0.085206 |
| CA19.9 | 22.6652 | 0.019665 |
| CEA | 2063.1449 | 0.00037 |
| HGF | 264.6446 | 0.004534 |
| OPN | 53651.1756 | 2.19E−05 |
| Prolactin | 21304.0703 | 4.84E−05 |
| TIMP.1 | 85363.9233 | 1.18E−05 |

Comparison of Aneuploidy Sensitivity Detection with Other Cancer Biomarkers

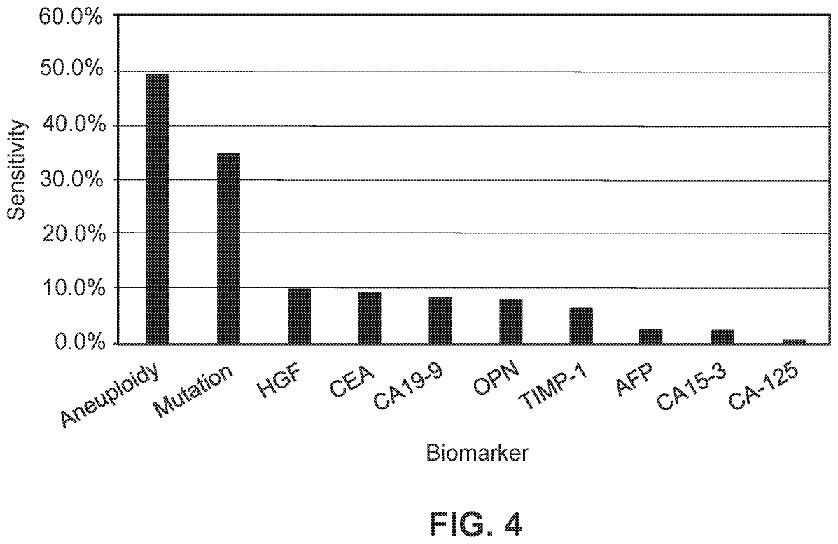

The aneuploidy results were benchmarked against a driver gene mutation panel and collection of 7 proteins markers (AFP, CA-125, CA15-3, CA19-9, CEA, HGF, OPN, TIMP1) that were recently published as key biomarkers for cancer detection in plasma samples (FIG. 4) (Cohen et. al 2018, Science 359(6378): 926-930). Aneuploidy outperformed all protein markers. Aneuploidy was also able to detect 42% of the samples that were missed by mutations and 34% of the samples that were missed by the mutation panel as well as the proteins. Due to the high specificity of this aneuploidy assay and the utility of each additional cancer biomarker, it will be understood that these components can be combined into a multi-analyte test for cancer detection.

### Example 2: Detection of Aneuploidy with Low Input DNA from Trisomy 21 Samples

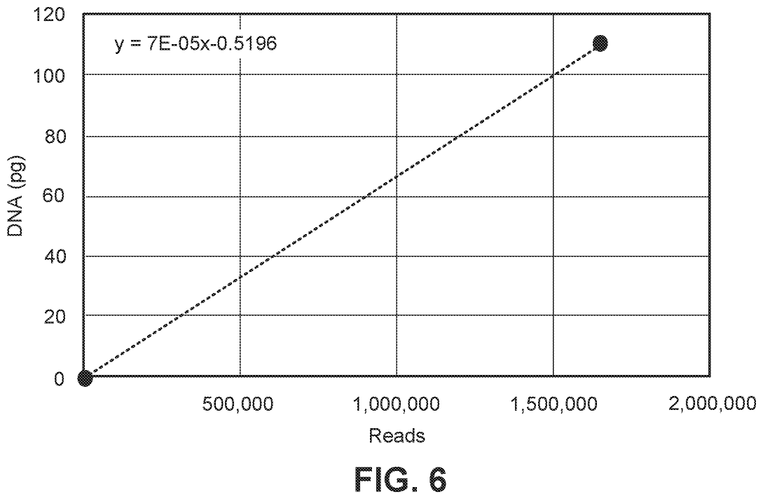

Reliably detecting aneuploidy in only a few picograms (pg) of DNA is necessary for preimplantation diagnostics as well as forensic applications. In preimplantation diagnosis, a few cells picked from a blastocyst are used to assess copy number variations. For example, preimplantation diagnosis includes identifying a mammals as having aneuploidy related to Down Syndrome. To test the limit of detection with respect to input DNA for the methods featured in this disclosure, samples with aneuploidy associated with trisomy 21 were analyzed at input DNA concentrations ranging from 3-225 pg. The relationship of reads to DNA was based on negative controls (water wells with no DNA) and the known concentration of the euploid control (FIG. 6). Trisomy 21 aneuploidy was detected in every sample tested, even those with 3 pg of input DNA, representing half of a diploid cell. No chromosome arms other than chromosome 21 were found to be aneuploid in the Trisomy 21 samples. No chromosome arms, including chromosome 21, were found to be aneuploid in the euploid controls used in these experiments.

### Example 3: Detection of Aneuploidy with Low Input DNA from Biobank Samples

Samples from biobanks with low input DNA were assessed for either aneuploidy or identification purposes. The methods as described herein were applied to 793 plasma DNA samples, which had been stored in PCR plates for as long as 10 years. For each of the wells in the PCR plates, all of the DNA volume had been used for other experiments. Five microliters of water was added to the dried (empty) wells and then subjected to the methods as described herein. In 728 samples, more than 2.5 million aligned reads were sequenced, which is a number sufficient to reliably assess aneuploidy. In 768 of these samples, more than 1 million aligned reads were sequenced, a number sufficient to confirm the identity of the plasma DNA to other samples from the same donor.

### Example 4: Detection of Leukocyte DNA Contamination in Plasma Sample

Plasma cfDNA is often contaminated with DNA that has leaked out of leukocytes, either through phlebotomy or preparation of plasma. This contaminating leukocyte DNA can reduce the sensitivity of aneuploidy testing from plasma samples because leukocytes are not derived from either fetal cells (in NIPT) or cancer cells (in liquid biopsies). Leukocyte genomic DNA (gDNA) has an average fragment size of >1000 bp while cell-free plasma DNA has an average size of <160 bp. Given that small fragments are amplified more efficiently during a PCR reaction, detection of contaminating leukocyte gDNA is difficult because the shorter cfDNA is preferentially amplified. Application of the methods described herein enabled the detection of contaminating leukocyte gDNA by virtue of the amplicons generated with primers SEQ ID NO: 1 and SEQ ID NO: 10. Using these methods, 1241 amplicons were identified that are typically present in gDNA but not cfDNA. Sequencing reads of these amplicons thereby indicated leukocyte contamination in plasma samples. Through mixing of leukocyte DNA with cell-free plasma DNA and using the methods described herein, samples containing >4% of leukocyte DNA could be detected, as shown in Table 5.

TABLE 5
Prediction of gDNA contamination in plasma.

| | | Reads | Fraction of | Ratio of |
|---|---|---|---|---|
| | | that map | Reads that | Fraction of |
| | Total | to 1241 | map to 1241 | reads to |
| | Reads | amplicons | amplicons | cfDNA |
| gDNA control | 1420121 | 1302 | 0.000916823 | 13.6900667 |
| euploid cell free | 9810368 | 657 | 6.697E−05 | 1 |
| DNA | | | | |
| 54% gDNA | 16666542 | 31138 | 0.001868294 | 27.8974908 |
| 37% gDNA | 13990980 | 19264 | 0.001376887 | 20.5597705 |
| 26% gDNA | 10760112 | 10907 | 0.001013651 | 15.135907 |
| 19% gDNA | 10976478 | 8769 | 0.00079889 | 11.929081 |
| 10% gDNA | 9408703 | 3415 | 0.000362962 | 5.41977026 |
| 5.5% gDNA | 9904155 | 2058 | 0.000207792 | 3.10275776 |
| 5.0% gDNA | 9083013 | 1987 | 0.00021876 | 3.2665391 |
| 4.5% gDNA | 8470920 | 1790 | 0.000211311 | 3.15531251 |
| 4% gDNA | 8852813 | 2336 | 0.000263871 | 3.9401384 |

### Example 5: Copy Number Analysis of Indeterminate Length

Copy number variants of indeterminate length were detected. First, the log ratio of the observed test sample and WALDO predicted values from every 500 kb interval across each chromosomal arm were calculated. Using the log ratio, a circular binary segmentation algorithm was applied to find copy number variants throughout each chromosome arm. Any copy number variant ≤5 Mb in size was flagged. Before calculating the statistical significance across each chromosome arm, these flagged CNVs were removed. In general, small CNVs can be used to assess microdeletions or microamplifications, such as those occurring in DiGeorge Syndrome (chromosome 22q11.2 or in breast cancers (chromosome 17q12).

### Example 6: Sensitivity of Cancer Detection with Multi-Analyte Tests

This Example describes the sensitivity of cancer detection with different multi-analyte tests.

Three different multi-analyte tests were used to evaluate the sensitivity of detecting eight cancers: breast, ovary, liver, lung, pancreas, esophagus, stomach, and colorectum, in the plasma sample from patients. The three tests were: (1) a three component test using aneuploidy status, somatic mutation analysis and protein biomarker evaluation; (2) a two component test using aneuploidy status and somatic mutation analysis; and (3) a two component test using aneuploidy status and protein biomarker evaluation. The eight protein biomarkers tested and somatic mutations tested were as described in Cohen et al., Science 359, pp. 926-930, the entire contents of which are hereby incorporated by reference.

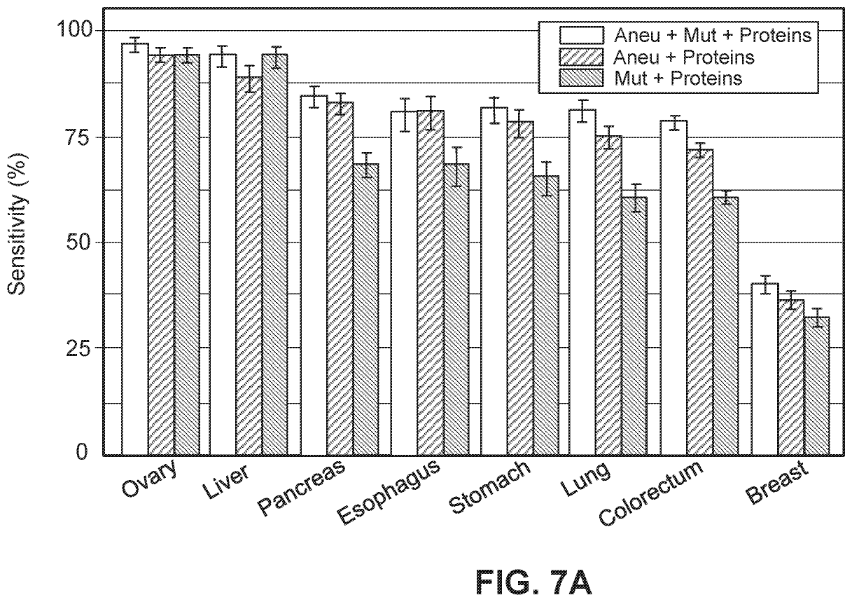

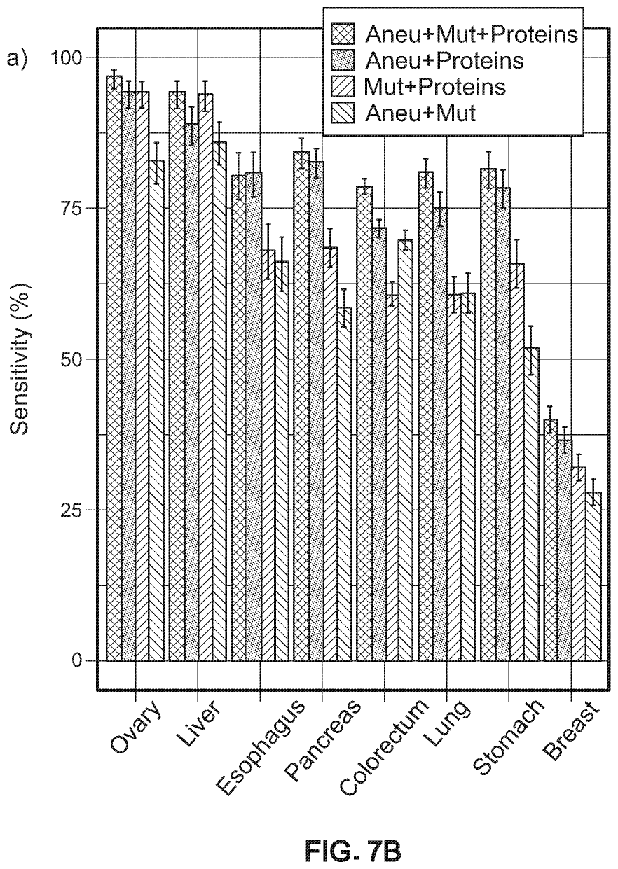

As shown in FIGS. 7A-7B, the median sensitivity of detection of ovary, liver, lung, pancreas, esophagus, stomach, and colorectum cancer with the three component multi-analyte test was 80%, with a range of sensitivity of detection of 77% to 97%. The sensitivity of detection of breast cancer with the three component multi-analyte test was 38%. The sensitivities were calculated using a threshold at 99% specificity.

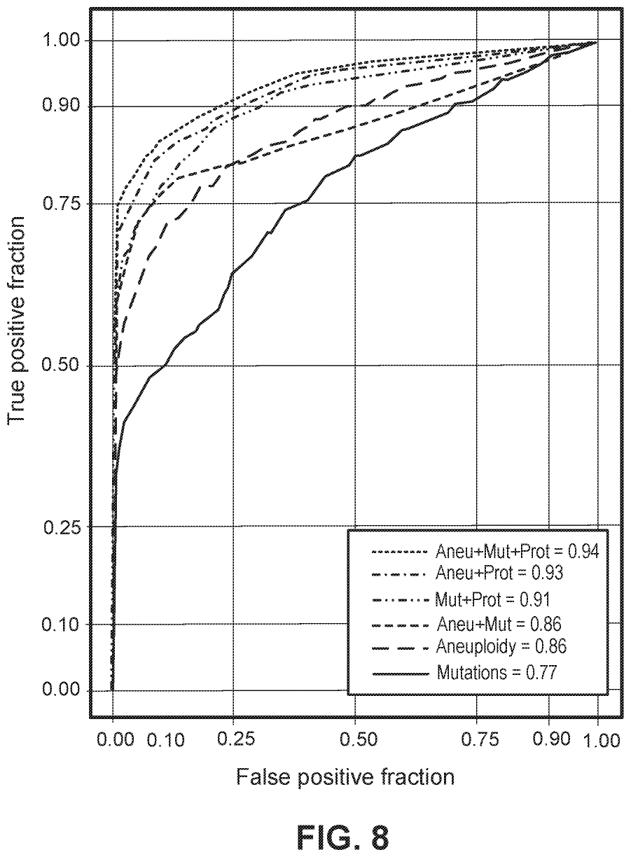

FIG. 8 further demonstrates true positive fraction (measure of sensitivity) of cancer detection using the following tests: (1) aneuploidy status; somatic mutation; and protein biomarker; (2) aneuploidy status and protein biomarker; (3) somatic mutation and protein biomarker; (4) aneuploidy status and somatic mutation; (5) aneuploidy status; and (6) somatic mutation. The specificity of detection was maintained at 99%.

As shown in FIG. 8, the three component multi-analyte test (aneuploidy status, somatic mutation analysis and protein biomarker evaluation) detected cancer at a sensitivity of 73% and with a specificity of 99%. The true positive fraction (a measure of sensitivity) was highest with the three component multi-analyte test as compared to the other tests.

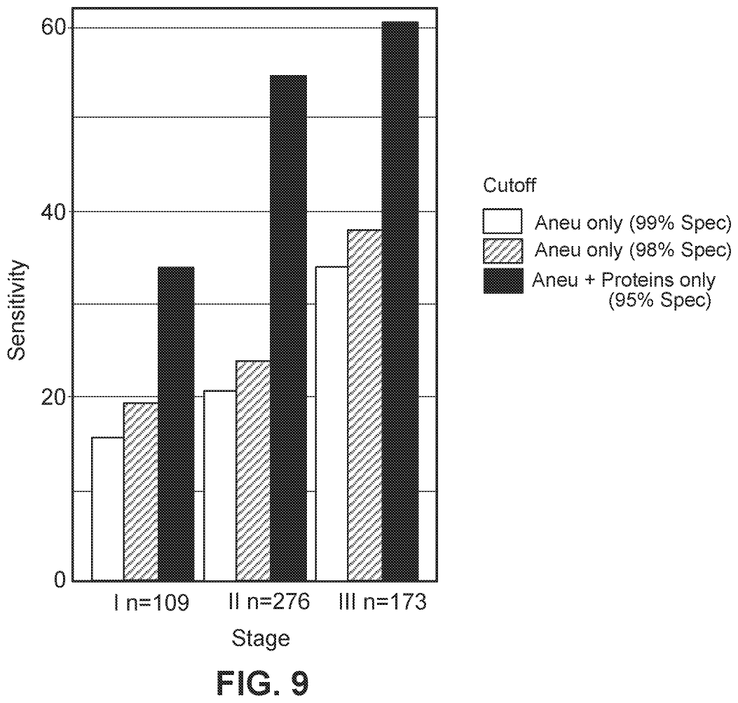

As shown in FIG. 9, a multi-analyte test (aneuploidy status and protein biomarker evaluation) detected cancer at a greater sensitivity than aneuploidy alone when looking at samples based cancer stage.

Thus, the data disclosed in this Example shows that the three component multi-analyte test with aneuploidy status, somatic mutation analysis and protein biomarker evaluation can increase the sensitivity of detecting cancer while maintaining a high specificity of cancer detection.

### Example 7: Determining Somatic/Germline Status

The materials and methods described herein can be used to identify somatic mutations within the sequences of repetitive elements amplified from a sample (e.g., a tumor sample or a non-tumor sample (i.e., a normal sample)). For example, when two samples, a non-tumor sample and a tumor sample, are available from the same patient, mutations that are in one sample but not the other can be discerned. For each sample, the number of somatic mutations can be counted and the spectrum of single base substitutions (SBS) (e.g., A->T, A->C, etc.) determined. When the samples are also analyzed by exomic sequencing, a correlation between the number of SBSs in the repetitive elements amplified herein and the number of SBS in the exomes can be determined. Thus, the materials and methods as described herein can be used identify somatic mutations within a sample.

### Example 8: Sample Identification

The materials and methods described herein can be used to identify and/or distinguish samples (e.g., distinguish between a sample from one subject from a sample from a second subject). In such cases, samples are identified based on the common polymorphisms present in the repetitive elements amplified by materials and methods described herein. Samples are then distinguished from other samples by comparing the sequence at common polymorphisms between samples. Determining the genotype of each polymorphism for each of the amplicons assigns a genotype to the sample. Genotypes can be compared across samples in order to identify samples (e.g., distinguish tumor sample from non-tumor sample or a sample from one subject from a sample from a different subject). Samples can be considered to be from different samples if concordance (e.g., percent similarity between the genoytpes) was <0.99 and at least 5,000 amplicons had adequate coverage.

### Example 9: Detecting Aneuploidy in Different Stages and Different Types of Cancer

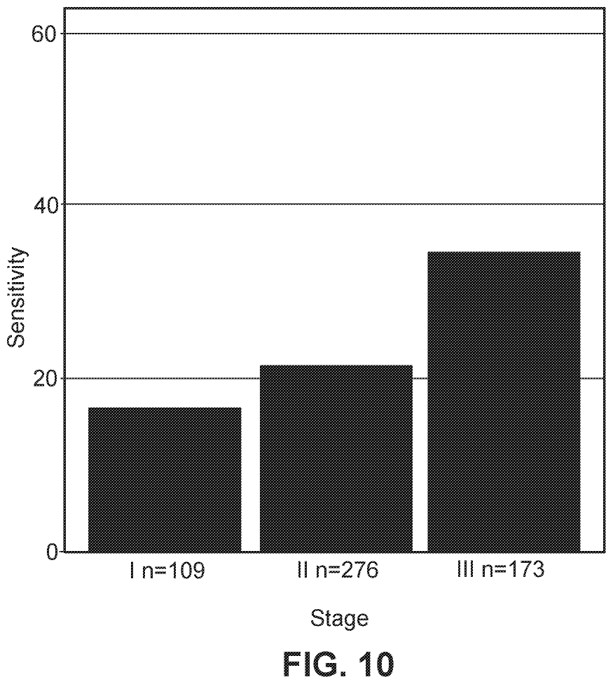

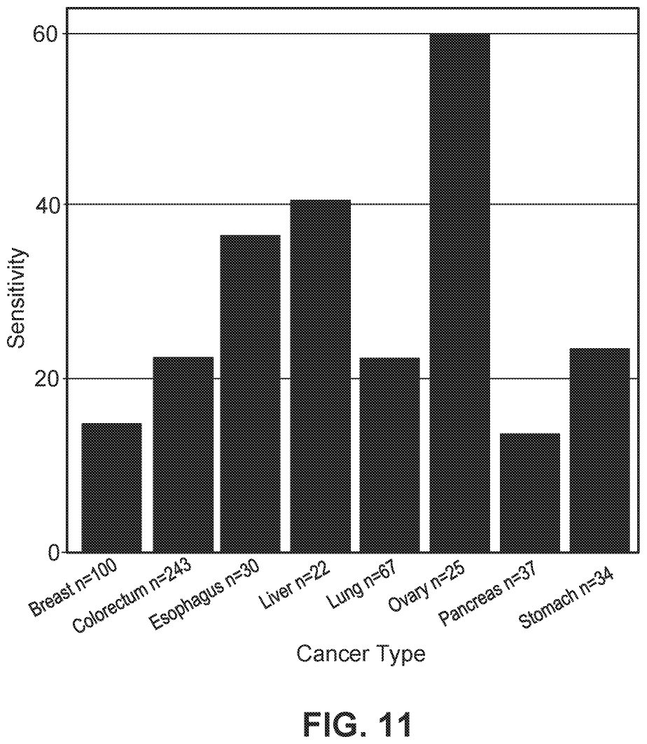

A set of experiments was performed to assess detection of aneuploidy in different stages and different types of cancer. In these experiments, plasma from subjects having different stages of breast, colorectum, esophagus, liver, lung, ovary, pancreas and stomach cancers were isolated according to the methods described herein. FIG. 10 shows aneuploidy (at 99% specificity) for Stage I (n=109), Stage II (n=276), and Stage III (173). FIG. 11 shows aneuploidy (at 99% specificity) for the same cancers in FIG. 7 displayed by cancer type (FIG. 11) rather than cancer stage (FIG. 10).

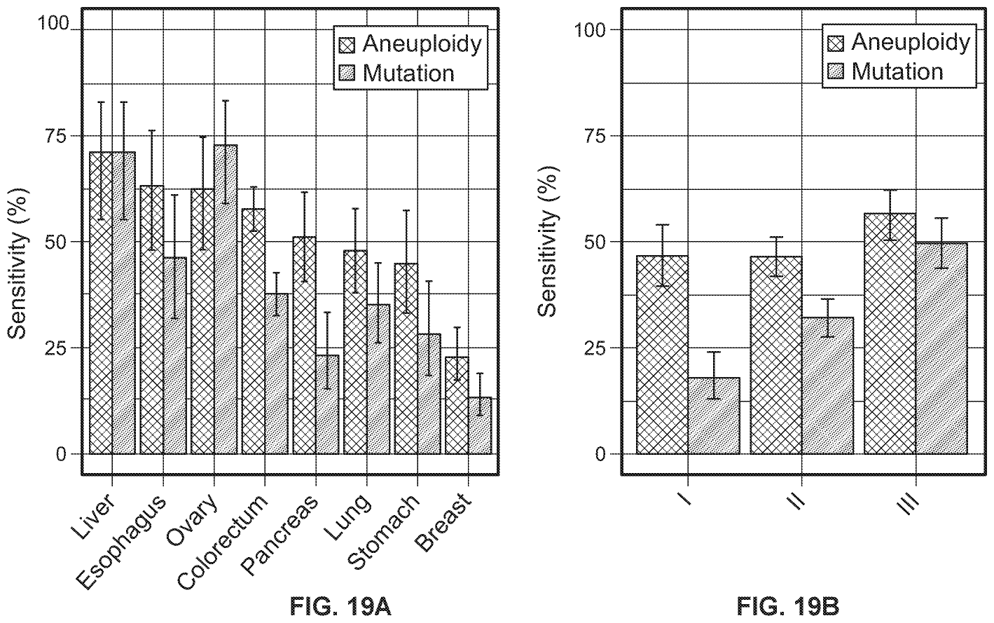

Using the Real Seq method, aneuploidy was detected more commonly than mutations in plasma samples from cancer patients. Aneuploidy was detected more commonly than mutations in plasma samples from cancer patients (49% and 34% of 883 samples, respectively; P<10-20, one sided binomial test, FIG. 19A). With respect to tissue type, aneuploidy was detected more commonly than mutations in samples from patients with cancers of the esophagus, colorectum, pancreas, lung, stomach, and breast, (all P-values<0.01), less commonly in ovary (P=0.048), and equally commonly in liver cancer (FIG. 19A). With respect to stage, aneuploidy was detected more commonly than mutations in all stages especially stages I and II (FIG. 19B, P-values<10-9).

### Example 10: Detecting Cancer in Samples Using Aneuploidy and Protein Biomarkers

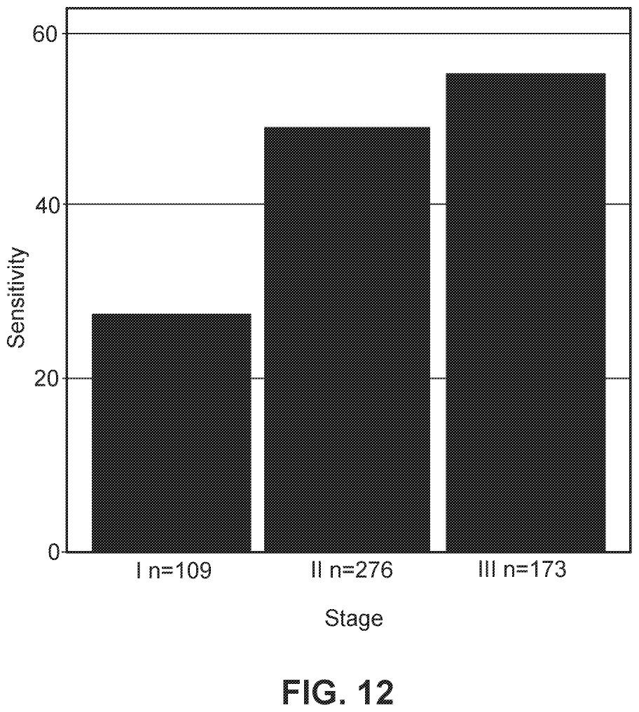

A set of experiments was performed to assess sensitivity of cancer detection when combining aneuploidy detection with protein biomarker detection as described herein. In these experiments, plasma from the same cohort as in example 8 (e.g., different stages of breast, colorectum, esophagus, liver, lung, ovary, pancreas and stomach cancer) were assayed for aneuploidy and protein biomarkers. FIG. 12 shows sensitivity of detection in the different stages of cancer (Stage I (n=109), Stage II (n=276), and Stage III (n=173).

### Example 11: Comparison of Real Seq to Other Next Generation Sequencing Technologies

A set of experiments was performed to assess performance of Real Seq compared to other next generation sequencing technologies.

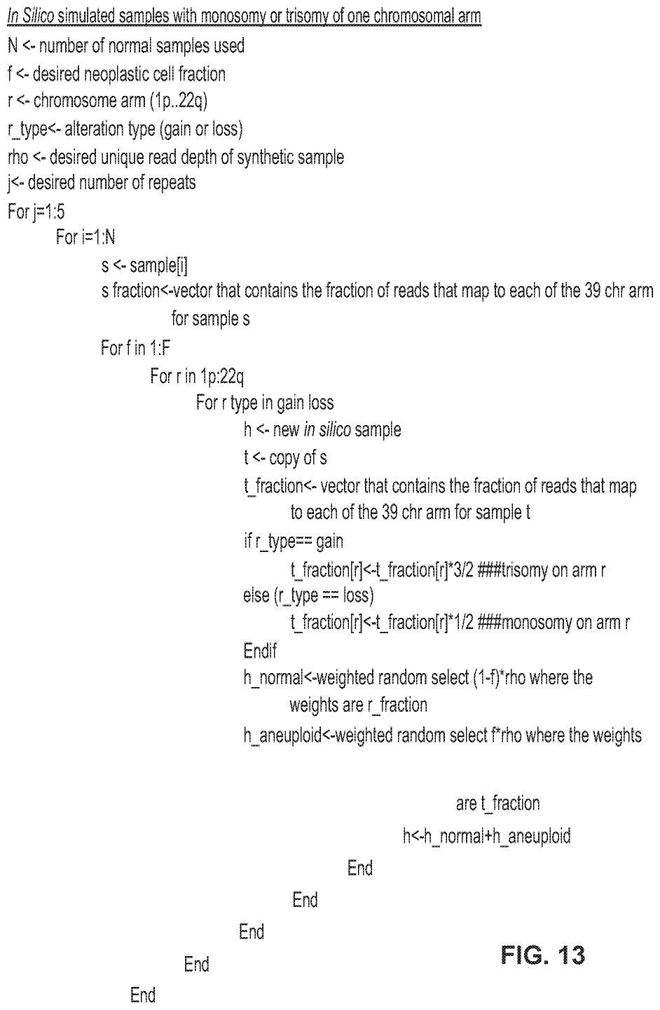

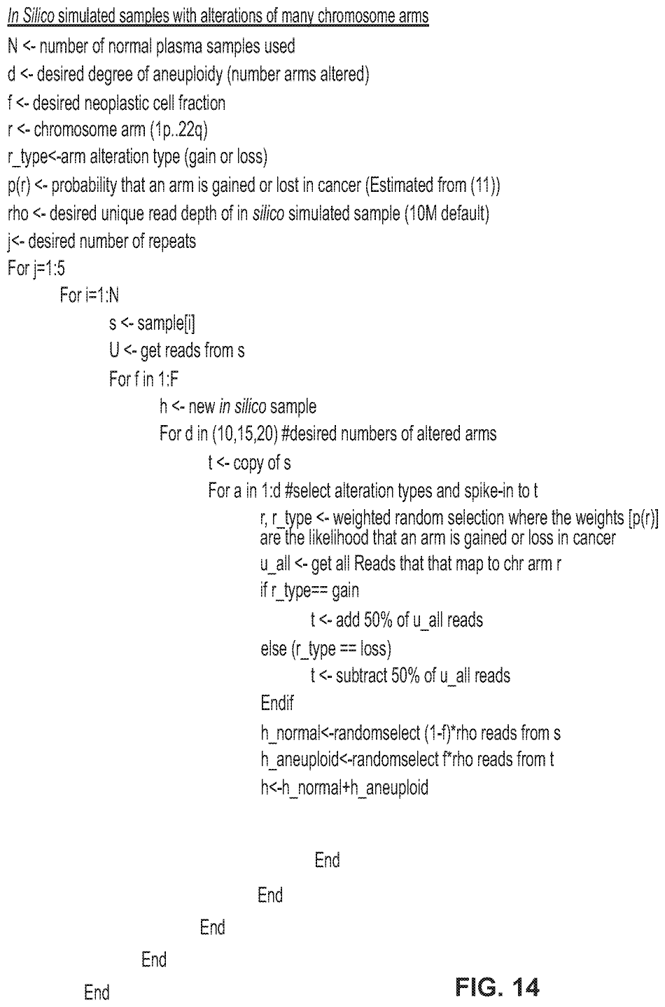

In the most common form of NIPT, detection of a gain or loss of a chromosome (e.g., chromosome 21 in Down Syndrome) is the goal. Whole genome sequencing (WGS), FAST-SeqS, and RealSeqS were used to assess performance on samples for DNA admixtures typically encountered in non-invasive prenatal testing (NIPT), i.e., when the fraction of fetal DNA was 5%. For this purpose, actual data obtained with the three methods was used, but then a defined number of reads from various chromosome regions from the same samples were added to simulate what would happen if there was aneuploidy in these regions. The pseudocode used to generate these in silico simulated samples is described FIG. 13 and FIG. 14. The performance was calculated using a frequently used z-score that compares the observed fraction of reads on a particular chromosome arm to the average fraction of reads from a normal panel divided by the standard deviation in the normal panel. The results in total reads needed for all three approaches is reported, assuming single-end 100 bp reads and accounting for differences in alignment rates and filtering criteria typically used.

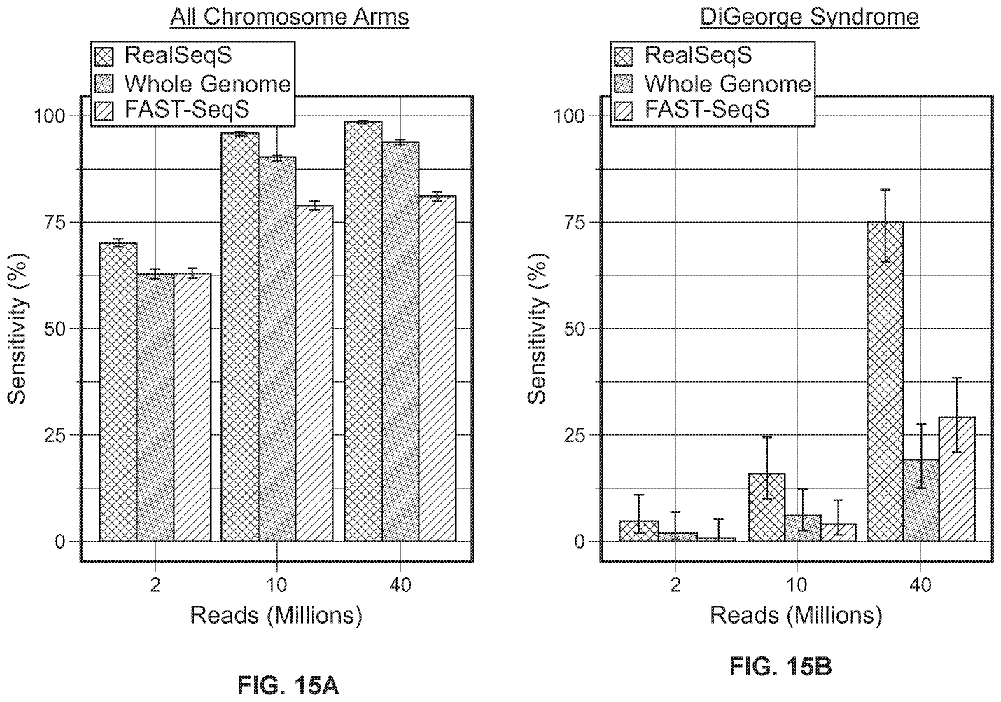

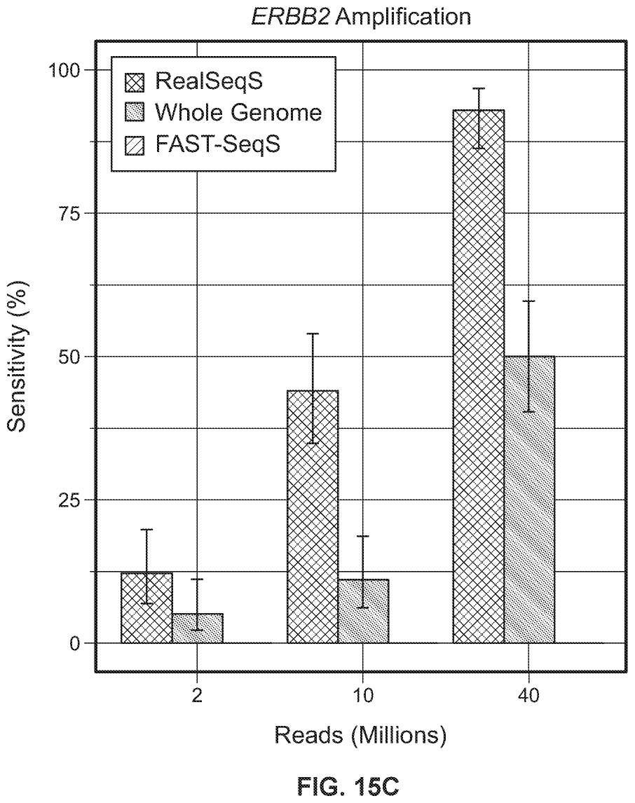

As shown in FIG. 15A, RealSeqS consistently achieved higher sensitivity at lower amounts of sequencing. For example, RealSeqS had 99% sensitivity (at 99% specificity) for monosomies and trisomies at a 5% cell fraction, while WGS and FAST-SeqS had 94% and 81% sensitivity, respectively (FIG. 15A).

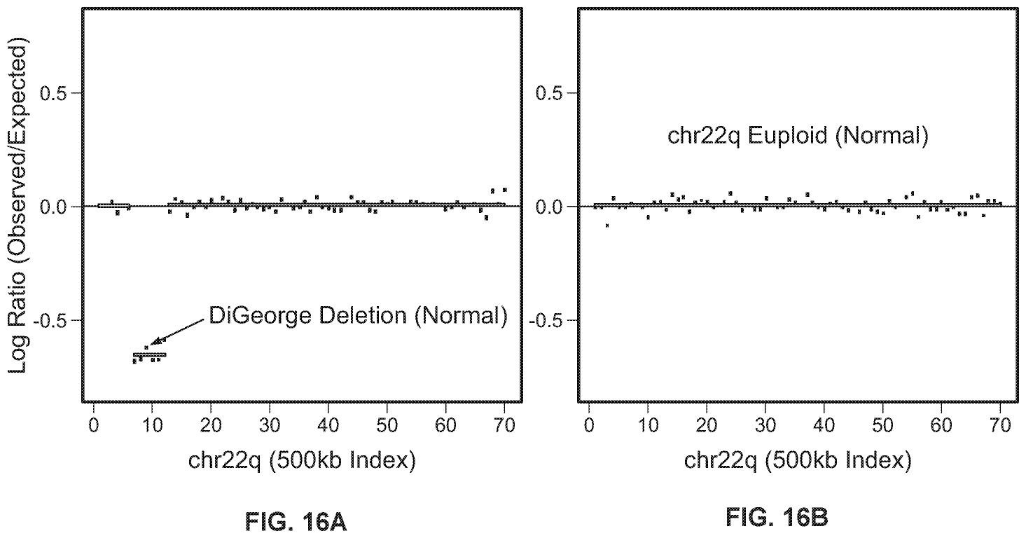

Another important aspect of assays for copy number variation is the detection of relatively small regions which are deleted or amplified. For example, the DiGeorge Syndrome deletions are often as small as 1.5 Mb. For data simulating a 5% deletion-containing cell fraction, RealSeqS had 75.0% sensitivity for the 1.5 Mb DiGeorge deletion (at 99% Specificity) while WGS and FAST-SeqS had 19.0% and 29.0% sensitivity, respectively (FIG. 15B; and FIGS. 16A-16B).

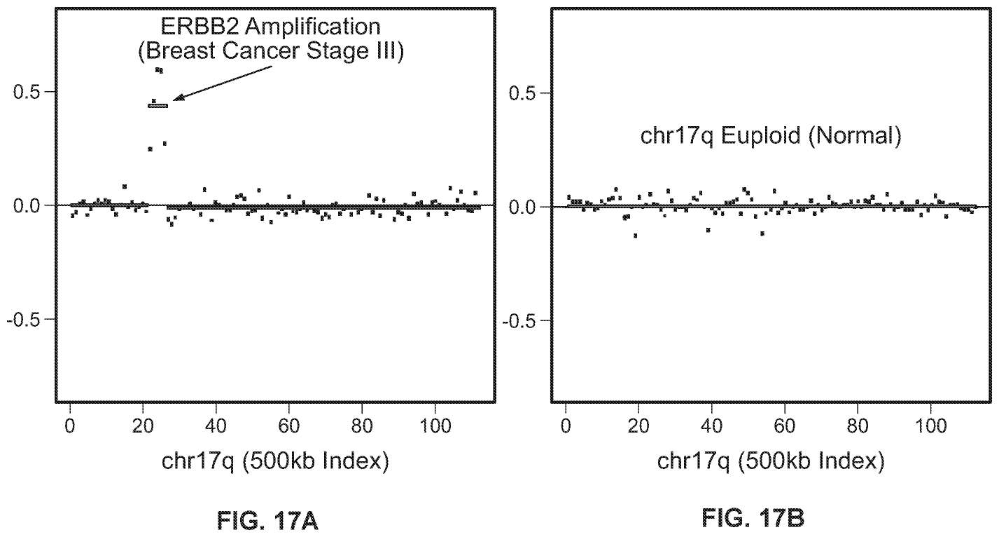

The detection of amplifications, such as those on ERBB2 in breast cancer, are important for deciding whether patients should be treated with trastuzumab or other targeted therapies. Following the same protocol as described above in this Example, in silico simulated samples with focal amplifications of the ˜42 Kb ERBB2 gene (20 copies) were generated for WGS, FAST-SeqS, and RealSeqS. RealSeqS detected amplifications in the in silico simulated samples with significantly less sequencing compared to WGS or Fast-SeqS. For a 1% cell fraction, RealSeqS had a 91.0% sensitivity while WGS had 50.0% (FIG. 15C; and FIGS. 17A-17B).

This data shows that the Real SEQ technique can detect small regions that are amplified or deleted and the method has a higher sensitivity at lower amounts of sequencing.

### Example 12: Detection of Aneuploidy in Samples with Small Concentration of Tumor-Derived DNA

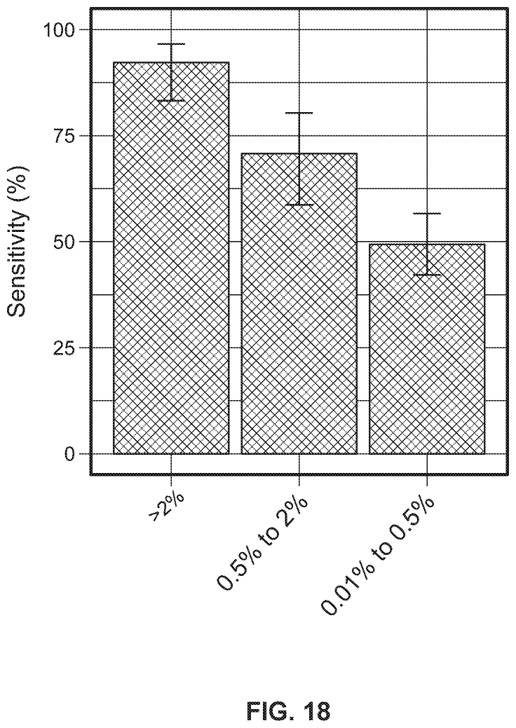

A set of experiments was performed to assess detection of aneuploidy using the Real SEQ method in samples with varying concentrations of tumor-derived DNA. In assessing 302 samples in which the mutant allele fraction had been determined by the analysis of mutations that were present in the plasma (Cohen et al., Science 359; 926-930), aneuploidy was detected in 92% of 65 samples that had mutant allele fraction ≥2%, 71% of 65 samples with mutant allele fractions of 0.5% to 2%, and in 49% of 172 samples with mutant allele frequencies ranging from 0.01% to 0.5% (FIG. 18). The differences in aneuploidy among these three classes of samples was significant (P<10-3, one sided binomial test).

The data shows that the Real Seq method can detect aneuploidy, e.g., even at low concentrations of tumor DNA. Therefore, the sensitivity of detecting aneuploidy is related to the concentration of circulating tumor DNA in the sample.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

TABLE 3

| | | | | | | | | AFP + | CA-125 |
|---|---|---|---|---|---|---|---|---|---|
| Unique Name | Repeats | Subject ID | Sample Na [symbol] | Cancer Typ [symbol] | Stage | Coverage | Aneuploidy | (>21321) | (577 U/ml) |
| 7666.9_faster1 | | 110962 | INDI 250 PL [symbol] | Colorectum | II | 1001795 | Negative | Negative | Negative |
| 7561.9_faster1 | | 10772 | INDI 260 PL [symbol] | Stomach | I | 1004248 | Negative | Negative | Negative |
| 7649.7_faster1 | | 110913 | INDI 548 PL [symbol] | Breast | II | 1053642 | Negative | Negative | Negative |
| 7678.11_faster1 | | 111047 | INDI 927 PL [symbol] | Breast | II | 1091466 | Negative | Negative | Negative |
| 7560.11_faster1 | | 108985 | INDI 534 PL [symbol] | Stomach | III | 1112490 | Positive | Negative | Negative |
| 7537.3_faster1 | | 110697 | PAPA 1350 [symbol] | Ovary | II | 1118254 | Negative | Negative | Negative |
| 7662.10_faster1 | | 110925 | INDI 354 PL [symbol] | Colorectum | III | 1196533 | Negative | Negative | Negative |
| 7613.11_faster1 | | 109125 | INDI 854 PL [symbol] | Colorectum | II | 1213691 | Negative | Negative | Negative |
| 7563.8_faster1 | | 109084 | INDI 762 PL [symbol] | Esophagus | II | 1217677 | Negative | Negative | Negative |
| 7537.1_faster1 | | 110621 | PANC 675 P [symbol] | Pancreas | II | 1228946 | Positive | Negative | Negative |
| 7591.11_faster1 | | 109110 | INDI 818 PL [symbol] | Ovary | III | 1320220 | Positive | Negative | Negative |
| 7586.5_faster1 | | 109014 | INDI 622 PL [symbol] | Liver | III | 1336412 | Negative | Negative | Negative |
| 7561.4_faster1 | | 109075 | INDI 743 PL [symbol] | Stomach | II | 1388535 | Negative | Negative | Negative |
| 7589.8_faster1 | | 109143 | INDI 896 PL [symbol] | Lung | I | 1412655 | Positive | Negative | Negative |
| 7586.6_faster1 | | 109020 | INDI 636 PL [symbol] | Liver | III | 1470803 | Positive | Negative | Negative |
| 7671.10_faster1 | | 110996 | INDI 767 PL [symbol] | Colorectum | II | 1492197 | Negative | Positive | Negative |
| 7560.10_faster1 | | 108981 | INDI 526 PL [symbol] | Esophagus | III | 1514882 | Negative | Negative | Negative |
| 7740.10_faster1 | | 111066 | CRC 478 PL [symbol] | Colorectum | III | 1559066 | Negative | Negative | Negative |
| 7671.12_faster1 | | 110998 | INDI 769 PL [symbol] | Colorectum | II | 1562172 | Positive | Negative | Negative |
| 7013.8_faster1 | | 109015 | INDI 623 PL [symbol] | Breast | III | 1597394 | Positive | Negative | Negative |
| 7645.6_faster1 | | 110872 | INDI 593 PL [symbol] | Breast | II | 1598322 | Negative | Negative | Negative |
| 7014.12_faster1 | | 110796 | INDI 244 PL [symbol] | Colorectum | III | 1615175 | Negative | Negative | Negative |
| 7009.10_faster1 | | 10888 | INDI 445 PL [symbol] | Breast | II | 1629340 | Positive | Negative | Negative |
| 7541.9_faster1 | | 110631 | PANCA 100 [symbol] | Pancreas | II | 1666957 | Negative | Negative | Negative |
| 7665.5_faster1 | | 110951 | INDI 679 PL [symbol] | Colorectum | II | 1693864 | Negative | Negative | Negative |
| 7541.11_faster1 | | 110633 | PANCA 100 [symbol] | Pancreas | II | 1733623 | Positive | Negative | Negative |
| 7567.8_faster1 | | 108991 | INDI 545 PL [symbol] | Lung | I | 1734591 | Negative | Negative | Negative |
| 7542.10_faster1 | | 110642 | PANCA 102 [symbol] | Pancreas | II | 1773560 | Negative | Negative | Negative |
| 7678.10_faster1 | | 111046 | INDI 926 PL [symbol] | Breast | II | 1778136 | Negative | Negative | Negative |
| 7673.12_faster1 | | 111018 | INDI 816 PL [symbol] | Colorectum | II | 1780759 | Negative | Negative | Negative |
| 7666.4_faster1 | | 110968 | INDI 903 PL [symbol] | Breast | I | 1795451 | Negative | Negative | Negative |
| 7562.10_faster1 | | 109108 | INDI 814 PL [symbol] | Stomach | I | 1843118 | Negative | Negative | Negative |
| 7537.9_faster1 | | 110619 | PANC 673 P [symbol] | Pancreas | II | 1851442 | Negative | Negative | Negative |
| 7594.3_faster1 | | 10859 | INDI 389 PL [symbol] | Lung | III | 1900877 | Negative | Positive | Negative |
| 6837.6_faster1 | | 109118 | INDI 844 PL [symbol] | Stomach | I | 1953864 | Negative | Negative | Negative |
| 7613.10_faster1 | | 109122 | INDI 849 PL [symbol] | Colorectum | II | 1998995 | Negative | Negative | Negative |
| 7589.12_faster1 | | 10856 | INDI 384 PL [symbol] | Pancreas | II | 2067166 | Negative | Negative | Negative |
| 7537.12_faster1 | | 110622 | PANC 676 [symbol] | Pancreas | II | 2084372 | Negative | Negative | Negative |
| 7591.12_faster1 | | 109129 | INDI 862 PL [symbol] | Pancreas | II | 2093931 | Negative | Positive | Negative |
| 7012.7_faster1 | Yes | 109090 | INDI 778 PL [symbol] | Liver | II | 2117457 | Positive | Negative | Negative |
| 7667.9_faster1 | | 110975 | INDI 286 PL [symbol] | Colorectum | I | 2160308 | Negative | Negative | Negative |
| 7548.9_faster1 | | 10811 | INDI 324 PL [symbol] | Lung | I | 2184419 | Negative | Negative | Negative |
| 7665.12_faster1 | | 110957 | INDI 898 PL [symbol] | Breast | I | 2253404 | Negative | Negative | Negative |
| 7671.3_faster1 | | 110991 | INDI 750 PL [symbol] | Colorectum | III | 2288848 | Negative | Negative | Negative |
| 7536.3_faster1 | | 110687 | PAPA 1335 [symbol] | Ovary | III | 2325428 | Positive | Negative | Negative |
| 6857.4_faster1 | | 109067 | INDI 729 PL [symbol] | Colorectum | II | 2350770 | Negative | Negative | Negative |
| 7676.8_faster1 | | 111019 | INDI 793 PL [symbol] | Breast | II | 2366123 | Negative | Negative | Negative |
| 7561.7_faster1 | | 109072 | INDI 737 PL [symbol] | Stomach | I | 2456741 | Negative | Negative | Negative |
| 7590.9_faster1 | | 109070 | INDI 735 PL [symbol] | Pancreas | II | 2464857 | Negative | Negative | Negative |
| 7591.8_faster1 | | 109068 | INDI 731 PL [symbol] | Pancreas | II | 2481059 | Negative | Negative | Negative |
| 7643.9_faster1 | | 110852 | INDI 610 PL [symbol] | Colorectum | II | 2503195 | Negative | Negative | Negative |
| 7613.6_faster1 | | 10758 | INDI 230 PL [symbol] | Colorectum | I | 2539061 | Negative | Negative | Negative |
| 7666.5_faster1 | | 110966 | INDI 897 PL [symbol] | Breast | III | 2577755 | Positive | Negative | Negative |
| 7666.8_faster1 | | 110961 | INDI 248 PL [symbol] | Colorectum | II | 2641351 | Negative | Negative | Negative |
| 7560.12_faster1 | | 109141 | INDI 892 PL [symbol] | Stomach | I | 2671691 | Negative | Negative | Negative |
| 7590.4_faster1 | | 108984 | INDI 533 PL [symbol] | Pancreas | II | 2692393 | Negative | Negative | Negative |
| 7639.4_faster1 | | 110820 | INDI 431 PL [symbol] | Colorectum | II | 2783016 | Negative | Negative | Negative |
| 7637.3_faster1 | | 110799 | INDI 295 PL [symbol] | Colorectum | II | 2793496 | Negative | Negative | Negative |
| 7611.9 _faster1 | | 10724 | INDI 192 PL [symbol] | Colorectum | II | 2812098 | Negative | Negative | Negative |
| 7612.3 _faster1 | | 10728 | INDI 197 PL [symbol] | Colorectum | II | 2816342 | Negative | Negative | Negative |
| 7645.12_faster1 | | 110878 | INDI 606 PL [symbol] | Breast | II | 2818021 | Positive | Positive | Negative |
| 7646.5_faster1 | | 110881 | INDI 627 PL [symbol] | Breast | II | 2833357 | Negative | Negative | Negative |
| 7546.10_faster1 | | 10572 | INDI 022 PL [symbol] | Lung | III | 2834538 | Positive | Negative | Negative |
| 7613.7_faster1 | | 10760 | INDI 233 PL [symbol] | Colorectum | I | 2871111 | Positive | Negative | Negative |
| 7543.11_faster1 | | 110655 | PANCA 105 [symbol] | Pancreas | II | 2959475 | Negative | Negative | Negative |
| 7676.11_faster1 | | 111022 | INDI 819 PL [symbol] | Breast | III | 2969142 | Positive | Negative | Negative |
| 7563.6_faster1 | | 109079 | INDI 753 PL [symbol] | Esophagus | III | 2987451 | Positive | Negative | Negative |
| 6851.10_faster1 | | 109077 | INDI 751 PL [symbol] | Colorectum | IIII | 2991204 | Negative | Negative | Negative |
| 7613.5_faster1 | | 10757 | INDI 229 PL [symbol] | Colorectum | II | 3055625 | Positive | Negative | Negative |
| 7672.10_faster1 | | 111001 | INDI 725 PL [symbol] | Colorectum | II | 3073272 | Negative | Negative | Negative |
| 7673.10_faster1 | | 111016 | INDI 809 PL [symbol] | Colorectum | IIII | 3105813 | Negative | Negative | Negative |
| 7544.3_faster1 | | 110657 | PANCA 105 [symbol] | Pancreas | II | 3128340 | Negative | Negative | Negative |
| 7646.6_faster1 | | 110882 | INDI 629 PL [symbol] | Breast | I | 3214436 | Negative | Negative | Negative |
| 7041.3_faster1 | | 109053 | INDI 701 PL [symbol] | Colorectum | II | 3276672 | Negative | Negative | Negative |
| 6851.12_faster1 | | 109092 | INDI 781 PL [symbol] | Colorectum | II | 3295690 | Negative | Negative | Negative |
| 6837.11_faster1 | | 10739 | INDI 210 PL [symbol] | Stomach | IIII | 3317316 | Positive | Negative | Negative |
| 7590.8_faster1 | | 109069 | INDI 734 PL [symbol] | Pancreas | II | 3318380 | Negative | Negative | Negative |
| 7638.11_faster1 | | 110817 | INDI 427 PL [symbol] | Colorectum | II | 3323134 | Negative | Negative | Negative |
| 7561.5_faster1 | | 109106 | INDI 803 PL [symbol] | Esophagus | II | 3348473 | Negative | Negative | Negative |
| 7567.10_faster1 | | 108994 | INDI 549 PL [symbol] | Lung | III | 3353566 | Negative | Negative | Negative |
| 7678.7_faster1 | | 111043 | INDI 922 PL [symbol] | Breast | II | 3355196 | Negative | Negative | Negative |
| 7740.7_faster1 | | 111063 | CRC 475 PL [symbol] | Colorectum | I | 3360401 | Negative | Negative | Negative |
| 7590.3_faster1 | | 10858 | INDI 388 PL [symbol] | Pancreas | II | 3361282 | Negative | Negative | Negative |
| 7589.3_faster1 | | 109045 | INDI 691 PL [symbol] | Lung | I | 3394538 | Negative | Negative | Negative |
| 7542.12_faster1 | | 110646 | PANCA 103 [symbol] | Pancreas | II | 3397925 | Negative | Negative | Negative |
| 7678.4_faster1 | | 111040 | INDI 919 PL [symbol] | Breast | II | 3424684 | Negative | Negative | Negative |
| 7534.12_faster1 | | 110667 | PAP 944 PL [symbol] | Ovary | III | 3428688 | Negative | Negative | Negative |
| 7642.12_faster1 | | 110848 | INDI 599 PL [symbol] | Colorectum | II | 3480043 | Negative | Negative | Negative |
| 7611.6_faster1 | | 10711 | INDI 178 PL [symbol] | Colorectum | III | 3487293 | Negative | Negative | Negative |
| 7541.12_faster1 | | 110634 | PANCA 101 [symbol] | Pancreas | I | 3488313 | Negative | Negative | Negative |
| 7585.5_faster1 | | 10722 | INDI 190 PL [symbol] | Liver | III | 3507555 | Negative | Negative | Negative |
| 7639.6_faster1 | | 110822 | INDI 434 PL [symbol] | Colorectum | II | 3557770 | Negative | Negative | Negative |
| 7592_combined.7_fast [symbol] | Yes | 10818 | INDI 335 PL [symbol] | Lung | I | 3560831 | Positive | Negative | Negative |
| 7677.4_faster1 | | 111030 | INDI 830 PL [symbol] | Breast | II | 3584227 | Negative | Negative | Negative |
| 7566.8_faster1 | | 10730 | INDI 199 PL [symbol] | Stomach | II | 3594027 | Negative | Negative | Negative |
| 7739.8_faster1 | | 111054 | CRC 462 PL [symbol] | Colorectum | I | 3597935 | Negative | Negative | Negative |
| 7584.8_faster1 | | 10701 | INDI 167 PL [symbol] | Stomach | II | 3615650 | Negative | Negative | Negative |
| 7670.10_faster1 | | 110986 | INDI 658 PL [symbol] | Colorectum | I | 3655451 | Negative | Negative | Negative |
| 7549.6_faster1 | | 108964 | INDI 495 PL [symbol] | Lung | I | 3683648 | Negative | Negative | Negative |
| 7535.5_faster1 | | 110677 | PAP 957 PL [symbol] | Ovary | III | 3701875 | Negative | Negative | Negative |
| 7740.6_faster1 | | 111062 | CRC 474 PL [symbol] | Colorectum | III | 3704739 | Negative | Negative | Negative |
| 7542.9_faster1 | | 110641 | PANCA 102 [symbol] | Pancreas | II | 3743768 | Positive | Negative | Negative |
| 7589.10_faster1 | | 10786 | INDI 278 PL [symbol] | Ovary | III | 3747939 | Positive | Negative | Positive |
| 7640.7_faster1 | | 110833 | INDI 477 PL [symbol] | Colorectum | III | 3762894 | Negative | Positive | Negative |
| 6857.9_faster1 | Yes | 10855 | INDI 383 PL [symbol] | Lung | III | 3790321 | Negative | Negative | Negative |
| 7667.10_faster1 | | 110976 | INDI 290 PL [symbol] | Colorectum | I | 3795297 | Negative | Negative | Negative |
| 7649.4_faster1 | | 110910 | INDI 512 PL [symbol] | Breast | II | 3801892 | Negative | Negative | Negative |
| 7640.8_faster1 | | 110834 | INDI 483 PL [symbol] | Colorectum | II | 3821378 | Negative | Negative | Negative |
| 7643.6_faster1 | | 110853 | INDI 611 PL [symbol] | Colorectum | II | 3824328 | Negative | Negative | Negative |
| 7543.4_faster1 | | 110648 | PANCA 103 [symbol] | Pancreas | II | 3835324 | Negative | Negative | Negative |
| 7589.6_faster1 | | 10624 | INDI 075 PL [symbol] | Ovary | III | 3846965 | Positive | Negative | Negative |
| 7638.12_faster1 | | 110818 | INDI 428 PL [symbol] | Colorectum | III | 3869695 | Negative | Negative | Negative |
| 7563.11_faster1 | | 10803 | INDI 313 PL [symbol] | Esophagus | II | 3887013 | Negative | Negative | Negative |
| 6839_redo.8_faster1 | | 109001 | INDI 574 PL [symbol] | Colorectum | III | 3890070 | Negative | Negative | Negative |
| 7643.10_faster1 | | 110856 | INDI 617 PL [symbol] | Colorectum | III | 3901698 | Negative | Negative | Negative |
| 7547.6_faster1 | | 10759 | INDI 231 PL [symbol] | Lung | III | 3915987 | Positive | Negative | Negative |
| 7664.12_faster1 | | 110948 | INDI 551 PL [symbol] | Colorectum | II | 3925074 | Negative | Negative | Negative |
| 7677.10_faster1 | | 111036 | INDI 842 PL [symbol] | Breast | I | 3973438 | Negative | Positive | Negative |
| 7591.4_faster1 | | 109051 | INDI 699 PL [symbol] | Pancreas | II | 3993186 | Positive | Negative | Negative |
| 7671.11_faster1 | | 110997 | INDI 768 PL [symbol] | Colorectum | II | 4013502 | Negative | Negative | Negative |
| 7591.5_faster1 | | 109055 | INDI 703 PL [symbol] | Lung | I | 4019055 | Negative | Negative | Negative |
| 7672.12_faster1 | | 111008 | INDI 782 PL [symbol] | Colorectum | III | 4055831 | Positive | Positive | Negative |
| 7586.7_faster1 | | 109024 | INDI 645 PL [symbol] | Liver | III | 4073694 | Positive | Negative | Negative |
| 7562.4_faster1 | | 10761 | INDI 236 PL [symbol] | Stomach | II | 4149785 | Negative | Negative | Negative |
| 7566.9_faster1 | | 10733 | INDI 203 PL [symbol] | Stomach | II | 4178571 | Negative | Positive | Negative |
| 7664.10_faster1 | | 110946 | INDI 528 PL [symbol] | Colorectum | II | 4210687 | Positive | Negative | Negative |
| 7676.4_faster1 | | 111025 | INDI 825 PL [symbol] | Colorectum | III | 4269747 | Positive | Negative | Negative |
| 7547.11_faster1 | | 10775 | INDI 264 PL [symbol] | Lung | III | 4275481 | Negative | Negative | Negative |
| 7638.5_faster1 | | 110811 | INDI 314 PL [symbol] | Colorectum | I | 4287508 | Negative | Negative | Negative |
| 7664.9_faster1 | | 110945 | INDI 527 PL [symbol] | Colorectum | III | 4309014 | Positive | Negative | Negative |
| 7603.12_faster1 | | 10652 | INDI 108 PL [symbol] | Colorectum | II | 4311628 | Positive | Negative | Negative |
| 6835.3_faster1 | | 110675 | PAP 955 PL [symbol] | Ovary | III | 4325563 | Negative | Negative | Negative |
| 7677.3_faster1 | | 111029 | INDI 823 PL [symbol] | Breast | II | 4334923 | Negative | Negative | Negative |
| 7013.6_faster1 | | 109011 | INDI 609 PL [symbol] | Colorectum | II | 4345601 | Negative | Negative | Negative |
| 7305.10_faster1 | | 109137 | INDI 880 PL [symbol] | Breast | II | 4389478 | Negative | Negative | Negative |
| 7678.12_faster1 | | 111048 | INDI 929 PL [symbol] | Stomach | II | 4398098 | Negative | Negative | Negative |
| 7011.7_faster1 | | 10717 | INDI 185 PL [symbol] | Stomach | II | 4409233 | Positive | Negative | Negative |
| 7610.10_faster1 | | 10698 | INDI 163 PL [symbol] | Colorectum | III | 4431295 | Negative | Negative | Negative |
| 7637.7_faster1 | | 110803 | INDI 302 PL [symbol] | Colorectum | I | 4442068 | Negative | Negative | Negative |
| 7589.7_faster1 | | 109140 | INDI 888 PL [symbol] | Pancreas | II | 4482143 | Negative | Negative | Negative |
| 7013.12_faster1 | | 10825 | INDI 343 PL [symbol] | Colorectum | III | 4512396 | Negative | Negative | Negative |
| 7536.6_faster1 | | 110690 | PAPA 1342 [symbol] | Ovary | III | 4547749 | Positive | Negative | Negative |
| 7560.7_faster1 | | 10807 | INDI 318 PL [symbol] | Stomach | III | 4556278 | Positive | Negative | Negative |
| 7585.10_faster1 | | 10889 | INDI 446 PL [symbol] | Esophagus | II | 4580159 | Negative | Negative | Negative |
| 7640.12_faster1 | | 110838 | INDI 564 PL [symbol] | Colorectum | I | 4604216 | Negative | Negative | Negative |
| 7543.3_faster1 | | 110647 | PANCA 103- [symbol] | Pancreas | II | 4606920 | Negative | Negative | Negative |
| 7042.3_faster1 | | 109113 | INDI 827 PL [symbol] | Colorectum | III | 4629206 | Negative | Negative | Negative |
| 7645.8_faster1 | | 110874 | INDI 597 PL [symbol] | Breast | II | 4629350 | Negative | Negative | Negative |
| 6858.5_faster1 | | 10543 | CRC 494 PL [symbol] | Colorectum | II | 4631558 | Positive | Negative | Negative |
| 7663.11_faster1 | | 110937 | INDI 484 PL [symbol] | Colorectum | II | 4635397 | Negative | Negative | Negative |
| 7610.12_faster1 | | 10700 | INDI 165 PL [symbol] | Colorectum | I | 4671536 | Negative | Negative | Negative |
| 7672.5_faster1 | | 111005 | INDI 774 PL [symbol] | Colorectum | I | 4676699 | Negative | Positive | Negative |
| 7549.11_faster1 | | 108973 | INDI 513 PL [symbol] | Lung | I | 4706497 | Negative | Negative | Negative |
| 7662.12_faster1 | | 110927 | INDI 374 PL [symbol] | Colorectum | III | 4752071 | Negative | Negative | Negative |
| 7666.11_faster1 | | 110964 | INDI 253 PL [symbol] | Colorectum | I | 4756889 | Negative | Negative | Negative |
| 7542.4_faster1 | | 110636 | PANCA 101 [symbol] | Pancreas | II | 4841695 | Negative | Negative | Negative |
| 7677.7_faster1 | | 111033 | INDI 833 PL [symbol] | Breast | I | 4842387 | Negative | Negative | Negative |
| 7677.8_faster1 | | 111034 | INDI 839 PL [symbol] | Breast | II | 4846046 | Negative | Negative | Negative |
| 7672.3_faster1 | | 111003 | INDI 772 PL [symbol] | Colorectum | II | 4846546 | Negative | Negative | Negative |
| 7593.8_faster1 | | 10847 | INDI 371 PL [symbol] | Lung | III | 4871802 | Positive | Negative | Negative |
| 7560.8_faster1 | | 10808 | INDI 319 PL [symbol] | Stomach | I | 4915897 | Negative | Negative | Negative |
| 7535.8_faster1 | | 110678 | PAP 959 PL [symbol] | Ovary | III | 4940585 | Positive | Negative | Negative |
| 7562.6_faster1 | | 10765 | INDI 241 PL [symbol] | Stomach | III | 4948571 | Negative | Negative | Negative |
| 7662.3_faster1 | | 110928 | INDI 889 PL [symbol] | Colorectum | II | 4974578 | Negative | Negative | Negative |
| 7640.6_faster1 | | 110832 | INDI 472 PL [symbol] | Colorectum | II | 4977178 | Negative | Negative | Negative |
| 7546.6_faster1 | | 10566 | INDI 013 PL [symbol] | Lung | III | 4982928 | Negative | Negative | Negative |
| 7010.8_faster1 | | 10567 | INDI 014 PL [symbol] | Lung | II | 4993575 | Positive | Negative | Negative |
| 7041.11_faster1 | | 109085 | INDI 764 PL [symbol] | Colorectum | II | 5040751 | Positive | Negative | Negative |
| 7542.7_faster1 | | 110639 | PANCA 101 [symbol] | Pancreas | III | 5064729 | Negative | Negative | Negative |
| 7676.10_faster1 | | 111021 | INDI 795 PL [symbol] | Breast | II | 5082422 | Negative | Positive | Negative |
| 7010.11_faster1 | | 108980 | INDI 524 PL [symbol] | Lung | III | 5095014 | Positive | Negative | Negative |
| 7593.10_faster1 | | 10849 | INDI 373 PL [symbol] | Lung | I | 5110714 | Negative | Negative | Negative |
| 7601.12_faster1 | | 10585 | INDI 036 PL [symbol] | Colorectum | I | 5136788 | Negative | Negative | Negative |
| 7611.7_faster1 | | 10712 | INDI 179 PL [symbol] | Colorectum | III | 5145980 | Negative | Negative | Negative |
| 7603.5_faster1 | | 10641 | INDI 094 PL [symbol] | Colorectum | I | 5157994 | Negative | Negative | Negative |
| 7672.7_faster1 | | 111007 | INDI 779 PL [symbol] | Colorectum | II | 5168461 | Negative | Negative | Negative |
| 7563.10_faster1 | | 109089 | INDI 776 PL [symbol] | Esophagus | II | 5205936 | Negative | Negative | Negative |
| 7536.10_faster1 | | 110694 | PAPA 1347 [symbol] | Ovary | I | 5206646 | Negative | Negative | Negative |
| 7664.11_faster1 | | 110947 | INDI 544 PL [symbol] | Colorectum | III | 5295602 | Negative | Negative | Negative |
| 7534.8_faster1 | | 110673 | PAP 951 PL [symbol] | Ovary | III | 5341189 | Negative | Negative | Negative |
| 6836.4_faster1 | | 10579 | INDI 029 PL [symbol] | Colorectum | II | 5358468 | Negative | Negative | Negative |
| 7603.10_faster1 | | 10648 | INDI 102 PL [symbol] | Colorectum | III | 5389348 | Negative | Negative | Negative |
| 7542.8_faster1 | | 110640 | PANCA 101 [symbol] | Pancreas | I | 5459643 | Negative | Negative | Negative |
| 7676.3_faster1 | | 111023 | INDI 821 PL [symbol] | Colorectum | III | 5507106 | Positive | Negative | Negative |
| 7609.5_faster1 | | 10662 | INDI 122 PL [symbol] | Colorectum | II | 5524030 | Negative | Negative | Negative |
| 6838_redo.4_faster1 | | 109120 | INDI 847 PL [symbol] | Liver | III | 5531516 | Negative | Negative | Negative |
| 7584.9_faster1 | | 10702 | INDI 168 PL [symbol] | Stomach | II | 5532836 | Negative | Negative | Negative |
| 7612.5_faster1 | | 10736 | INDI 207 PL [symbol] | Colorectum | II | 5541362 | Negative | Negative | Negative |
| 7541.4_faster1 | | 110624 | PANC 679 P [symbol] | Pancreas | II | 5689544 | Negative | Negative | Negative |
| 7613.9_faster1 | | 109121 | INDI 848 PL [symbol] | Colorectum | II | 5696303 | Negative | Negative | Negative |
| 7606.6_faster1 | Yes | 10634 | INDI 085 PL [symbol] | Stomach | I | 5707150 | Negative | Negative | Negative |
| 7602.3_faster1 | | 10586 | INDI 037 PL [symbol] | Colorectum | I | 5727492 | Negative | Negative | Negative |
| 7042.7_faster1 | Yes | 10827 | INDI 346 PL [symbol] | Lung | I | 5738285 | Negative | Negative | Negative |
| 7041.12_faster1 | | 109091 | INDI 780 PL [symbol] | Colorectum | II | 5750070 | Negative | Negative | Negative |
| 7642.5_faster1 | | 110841 | INDI 573 PL [symbol] | Colorectum | I | 5761449 | Negative | Negative | Negative |
| 7672.4_faster1 | | 111004 | INDI 773 PL [symbol] | Colorectum | II | 5842790 | Negative | Negative | Negative |
| 7535.6_faster1 | | 110669 | PAP 946 PL [symbol] | Ovary | III | 5847253 | Negative | Negative | Positive |
| 7662.6_faster1 | | 110921 | INDI 325 PL [symbol] | Colorectum | III | 5847693 | Negative | Negative | Negative |
| 7673.8_faster1 | | 111009 | INDI 708 PL [symbol] | Colorectum | III | 5876494 | Positive | Negative | Negative |
| 7534.3_faster1 | | 110663 | PAP 938 PL [symbol] | Ovary | III | 5879145 | Positive | Positive | Negative |
| 7613.8_faster1 | | 109119 | INDI 846 PL [symbol] | Colorectum | II | 5880716 | Negative | Negative | Negative |
| 7759.5_faster1 | Yes | 10657 | INDI 113 PL [symbol] | Colorectum | II | 5880730 | Negative | Negative | Negative |
| 7739.6_faster1 | | 111052 | CRC 459 PL [symbol] | Colorectum | II | 5886699 | Negative | Negative | Negative |
| 7637.5_faster1 | | 110801 | INDI 298 PL [symbol] | Colorectum | I | 5905840 | Negative | Negative | Negative |
| 7603.6_faster1 | | 10643 | INDI 096 PL [symbol] | Colorectum | I | 5947450 | Negative | Negative | Negative |
| 7537.5_faster1 | | 110700 | PAPA 1356 [symbol] | Ovary | II | 5951081 | Negative | Negative | Positive |
| 6857.5_faster1 | | 109112 | INDI 826 PL [symbol] | Colorectum | II | 5985530 | Negative | Negative | Negative |
| 7678.9_faster1 | | 111045 | INDI 924 PL [symbol] | Breast | III | 6013647 | Negative | Negative | Negative |
| 7541.7_faster1 | | 110629 | PANCA 100 [symbol] | Pancreas | II | 6099173 | Negative | Negative | Negative |
| 7667.4_faster1 | | 110970 | INDI 259 PL [symbol] | Colorectum | II | 6123288 | Positive | Negative | Negative |
| 7672.8_faster1 | | 110999 | INDI 718 PL [symbol] | Colorectum | II | 6127303 | Negative | Negative | Negative |
| 7535.12_faster1 | | 110686 | PAPA 1334 [symbol] | Ovary | III | 6128628 | Positive | Negative | Negative |
| 7665.9_faster1 | | 110956 | INDI 732 PL [symbol] | Colorectum | III | 6144208 | Negative | Negative | Negative |
| 7306.11_faster1 | Yes | 10810 | INDI 323 PL [symbol] | Breast | I | 6160359 | Negative | Negative | Negative |
| 7536.4_faster1 | | 110688 | PAPA 1336 [symbol] | Ovary | I | 6166344 | Negative | Negative | Negative |
| 7742.8_faster1 | | 111084 | CRC 508 PL [symbol] | Colorectum | I | 6174674 | Negative | Negative | Negative |
| 7546.5_faster1 | | 10564 | INDI 011 PL [symbol] | Lung | I | 6198380 | Negative | Negative | Negative |
| 7663.7_faster1 | | 110933 | INDI 386 PL [symbol] | Colorectum | III | 6229120 | Negative | Negative | Negative |
| 7671.5_faster1 | | 110990 | INDI 747 PL [symbol] | Colorectum | III | 6268955 | Negative | Negative | Negative |
| 7662.11_faster1 | | 110926 | INDI 3S6 PL [symbol] | Colorectum | I | 6313478 | Negative | Negative | Negative |
| 7566.6_faster1 | | 10679 | INDI 140 PL [symbol] | Stomach | III | 6315974 | Negative | Negative | Negative |
| 7013.5_faster1 | | 109010 | INDI 602 PL [symbol] | Colorectum | II | 6319245 | Positive | Negative | Negative |
| 7010.9_faster1 | | 10790 | INDI 285 PL [symbol] | Lung | II | 6332234 | Positive | Negative | Negative |
| 7642.8_faster1 | | 110844 | INDI 584 PL [symbol] | Colorectum | II | 6358208 | Negative | Negative | Negative |
| 7759.4_faster1 | Yes | 10655 | INDI 111 PL [symbol] | Stomach | II | 6406233 | Negative | Negative | Negative |
| 7544.7_faster1 | | 110661 | PANCA 106 [symbol] | Pancreas | II | 6418433 | Negative | Negative | Negative |
| 7546.7_faster1 | | 10568 | INDI 015 PL [symbol] | Lung | I | 6421654 | Positive | Negative | Negative |
| 6836.5_faster1 | | 10584 | INDI 035 PL [symbol] | Colorectum | II | 6441354 | Negative | Negative | Negative |
| 7673.7_faster1 | | 111014 | INDI 806 PL [symbol] | Colorectum | II | 6483291 | Negative | Negative | Negative |
| 7642.9_faster1 | | 110845 | INDI 587 PL [symbol] | Colorectum | II | 6510398 | Negative | Negative | Negative |
| 7562.7_faster1 | | 109102 | INDI 799 PL [symbol] | Stomach | II | 6515486 | Negative | Negative | Negative |
| 7612.11_faster1 | | 10749 | INDI 221 PL [symbol] | Colorectum | III | 6591134 | Negative | Negative | Negative |
| 7670.12_faster1 | | 110988 | INDI 749 PL [symbol] | Colorectum | III | 6600357 | Negative | Negative | Negative |
| 6835.9_faster1 | | 110643 | PANCA 102 [symbol] | Pancreas | III | 6600637 | Negative | Negative | Negative |
| 7590.10_faster1 | | 109071 | INDI 736 PL [symbol] | Lung | III | 6627333 | Negative | Negative | Negative |
| 7548.4_faster1 | | 10781 | INDI 273 PL [symbol] | Lung | I | 6643421 | Negative | Negative | Negative |
| 7742.9_faster1 | | 111085 | CRC 509 PL [symbol] | Colorectum | I | 6656386 | Negative | Negative | Negative |
| 7009.5_faster1 | | 10613 | INDI 064 PL [symbol] | Breast | II | 6670500 | Negative | Negative | Negative |
| 7673.11_faster1 | | 111017 | INDI 815 PL [symbol] | Colorectum | II | 6700399 | Negative | Negative | Negative |
| 7611.5_faster1 | | 10708 | INDI 175 PL [symbol] | Colorectum | III | 6720895 | Negative | Negative | Negative |
| 6838_redo.11_faster1 | | 10823 | INDI 341 PL [symbol] | Liver | II | 6731601 | Positive | Negative | Negative |
| 7565.10_faster1 | | 10672 | INDI 132 PL [symbol] | Esophagus | III | 6748979 | Negative | Negative | Negative |
| 7593.3_faster1 | | 10834 | INDI 357 PL [symbol] | Lung | I | 6771463 | Negative | Negative | Negative |
| 7673.6_faster1 | | 111013 | INDI 805 PL [symbol] | Colorectum | II | 6780095 | Negative | Negative | Negative |
| 7011.9_faster1 | | 10729 | INDI 198 PL [symbol] | Stomach | III | 6793614 | Positive | Negative | Negative |
| 7603.7_faster1 | | 10645 | INDI 098 PL [symbol] | Colorectum | II | 6797379 | Negative | Negative | Negative |
| 7642.10_faster1 | | 110846 | INDI 594 PL [symbol] | Colorectum | III | 6797686 | Negative | Negative | Negative |
| 6851.3_faster1 | | 10766 | INDI 245 PL [symbol] | Colorectum | III | 6799700 | Positive | Negative | Negative |
| 7534.6_faster1 | | 110681 | PAP 974 PL [symbol] | Ovary | I | 6896369 | Negative | Negative | Negative |
| 7586.4_faster1 | | 109006 | INDI 586 PL [symbol] | Liver | III | 6916059 | Negative | Negative | Negative |
| 7663.3_faster1 | | 110929 | INDI 376 PL [symbol] | Colorectum | I | 6954053 | Negative | Negative | Negative |
| 7544.5_faster1 | | 110659 | PANCA 105 [symbol] | Pancreas | II | 7045546 | Negative | Negative | Negative |
| 6851.4_faster1 | | 10770 | INDI 255 PL [symbol] | Colorectum | II | 7093072 | Negative | Negative | Negative |
| 7307.12_faster1 | | 109035 | INDI 672 PL [symbol] | Breast | II | 7145901 | Negative | Negative | Negative |
| 7667.3_faster1 | | 110969 | INDI 256 PL [symbol] | Colorectum | III | 7157328 | Positive | Negative | Negative |
| 7757.10_faster1 | Yes | 10608 | INDI 059 PL [symbol] | Breast | II | 7168762 | Negative | Negative | Negative |
| 7536.12_faster1 | | 110696 | PAPA 1349 [symbol] | Ovary | III | 7185446 | Positive | Negative | Positive |
| 6836.11_faster1 | | 10573 | INDI 023 PL [symbol] | Lung | II | 7249605 | Positive | Negative | Negative |
| 7535.7_faster1 | | 110670 | PAP 947 PL [symbol] | Ovary | III | 7250255 | Negative | Negative | Negative |
| 7640.3_faster1 | | 110829 | INDI 463 PL [symbol] | Colorectum | II | 7284271 | Positive | Negative | Negative |
| 7541.5_faster1 | | 110625 | PANC 680 PL [symbol] | Pancreas | II | 7288598 | Negative | Negative | Negative |
| 7609.12_faster1 | | 10686 | INDI 148 PL [symbol] | Colorectum | II | 7304513 | Positive | Negative | Negative |
| 7640.5_faster1 | | 110831 | INDI 468 PL [symbol] | Colorectum | II | 7330421 | Negative | Negative | Negative |
| 7014.3_faster1 | | 10826 | INDI 344 PL [symbol] | Colorectum | II | 7374671 | Negative | Negative | Negative |
| 7543.8_faster1 | | 110652 | PANCA 104 [symbol] | Pancreas | II | 7374965 | Positive | Negative | Negative |
| 7639.11_faster1 | | 110827 | INDI 452 PL [symbol] | Colorectum | II | 7402986 | Positive | Negative | Negative |
| 6837.4_faster1 | | 10878 | INDI 415 PL [symbol] | Lung | II | 7421121 | Positive | Negative | Negative |
| 7665.8_faster1 | | 110954 | INDI 685 PL [symbol] | Colorectum | III | 7485730 | Negative | Negative | Negative |
| 7759.7_faster1 | Yes | 10660 | INDI 120 PL [symbol] | Stomach | III | 7527786 | Negative | Negative | Negative |
| 7535.11_faster1 | | 110685 | PAPA 1333 [symbol] | Ovary | III | 7562225 | Positive | Negative | Negative |
| 7639.12_faster1 | | 110828 | INDI 456 PL [symbol] | Colorectum | II | 7585556 | Negative | Negative | Negative |
| 7671.6_faster1 | | 110992 | INDI 758 PL [symbol] | Colorectum | II | 7592504 | Negative | Negative | Negative |
| 7664.6_faster1 | | 110942 | INDI 521 PL [symbol] | Colorectum | II | 7619123 | Negative | Negative | Negative |
| 7739.10_faster1 | | 111056 | CRC 464 PL [symbol] | Colorectum | III | 7661499 | Negative | Negative | Negative |
| 7308.4_faster1 | | 109061 | INDI 715 PL [symbol] | Breast | III | 7674698 | Positive | Negative | Negative |
| 7645.11_faster1 | | 110877 | INDI 604 PL [symbol] | Breast | II | 7683175 | Negative | Negative | Negative |
| 7541.8_faster1 | | 110630 | PANCA 100 [symbol] | Pancreas | II | 7733461 | Negative | Negative | Negative |
| 7758.5_faster1 | Yes | 10631 | INDI 082 PL [symbol] | Stomach | II | 7735285 | Negative | Negative | Negative |
| 7541.6_faster1 | | 110626 | PANC 757 P [symbol] | Pancreas | II | 7765953 | Negative | Negative | Negative |
| 6835.7_faster1 | | 110698 | PAPA 1354 [symbol] | Ovary | I | 7771957 | Positive | Negative | Negative |
| 7593.6_faster1 | | 10841 | INDI 364 PL [symbol] | Lung | I | 7821491 | Negative | Negative | Negative |
| 7676.12_faster1 | | 111024 | INDI 822 PL [symbol] | Colorectum | II | 7842252 | Negative | Negative | Negative |
| 7667.6_faster1 | | 110972 | INDI 271 PL [symbol] | Colorectum | III | 7853461 | Negative | Positive | Negative |
| 7643.8_faster1 | | 110855 | INDI 615 PL [symbol] | Colorectum | II | 7866361 | Negative | Negative | Negative |
| 7544.8_faster1 | | 10556 | INDI 001 PL [symbol] | Lung | II | 7887098 | Negative | Negative | Negative |
| 7548.8_faster1 | | 10795 | INDI 292 PL [symbol] | Lung | III | 7923986 | Negative | Negative | Negative |
| 7670.6_faster1 | | 110982 | INDI 650 PL [symbol] | Colorectum | III | 7954809 | Negative | Negative | Negative |
| 7601.7_faster1 | | 108954 | INDI 480 PL [symbol] | Breast | III | 7955468 | Negative | Negative | Negative |
| 7606.8_faster1 | Yes | 10764 | INDI 239 PL [symbol] | Lung | III | 7977349 | Negative | Negative | Negative |
| 7014.11_faster1 | | 10623 | INDI 074 PL [symbol] | Pancreas | II | 7990226 | Negative | Negative | Negative |
| 7670.9_faster1 | | 110985 | INDI 655 PL [symbol] | Colorectum | II | 8062805 | Negative | Negative | Negative |
| 7664.4_faster1 | | 110940 | INDI 508 PL [symbol] | Colorectum | III | 8148475 | Negative | Negative | Negative |
| 7759.3_faster1 | Yes | 10654 | INDI 110 PL [symbol] | Stomach | I | 8193900 | Negative | Negative | Negative |
| 6836.8_faster1 | | 10562 | INDI 009 PL [symbol] | Lung | II | 8217852 | Positive | Negative | Negative |
| 7564.4_faster1 | | 10718 | INDI 186 PL [symbol] | Stomach | II | 8224625 | Positive | Negative | Negative |
| 6850.10_faster1 | | 108996 | INDI 555 PL [symbol] | Colorectum | II | 8231841 | Negative | Negative | Negative |
| 7307.9_faster1 | | 108962 | INDI 493 PL [symbol] | Breast | II | 8263363 | Negative | Negative | Negative |
| 7014.4_faster1 | | 10842 | INDI 365 PL [symbol] | Colorectum | II | 8272863 | Positive | Negative | Negative |
| 7676.5_faster1 | | 111026 | INDI 829 PL [symbol] | Colorectum | III | 8309300 | Positive | Negative | Negative |
| 7673.3_faster1 | | 111010 | INDI 783 PL [symbol] | Colorectum | III | 8375574 | Negative | Negative | Negative |
| 7639.9_faster1 | | 110825 | INDI 438 PL [symbol] | Colorectum | II | 8414491 | Negative | Negative | Negative |
| 7308.8_faster1 | | 109095 | INDI 787 PL [symbol] | Breast | II | 8446437 | Negative | Negative | Negative |
| 6851.5_faster1 | | 10796 | INDI 293 PL [symbol] | Colorectum | II | 8469925 | Negative | Negative | Negative |
| 7756.3_faster1 | Yes | 10536 | CRC 470 PL [symbol] | Colorectum | II | 8479440 | Negative | Negative | Negative |
| 7637.8_faster1 | | 110804 | INDI 303 PL [symbol] | Colorectum | I | 8479921 | Negative | Negative | Negative |
| 7643.7_faster1 | | 110854 | INDI 612 PL [symbol] | Colorectum | II | 8511230 | Positive | Negative | Negative |
| 6835.11_faster1 | | 10619 | INDI 070 PL [symbol] | Breast | III | 8512193 | Positive | Negative | Negative |
| 7304.12_faster1 | Yes | 109027 | INDI 659 PL [symbol] | Breast | II | 8533292 | Negative | Negative | Negative |
| 7308.7_faster1 | | 109065 | INDI 722 PL [symbol] | Breast | I | 8569420 | Negative | Negative | Negative |
| 7542.11_faster1 | | 110644 | PANCA 102 [symbol] | Pancreas | III | 8585600 | Negative | Negative | Negative |
| 6836.12_faster1 | | 10748 | INDI 220 PL [symbol] | Lung | I | 8603684 | Positive | Negative | Negative |
| 7547.12_faster1 | | 10777 | INDI 266 PL [symbol] | Lung | I | 8648492 | Negative | Negative | Negative |
| 7590.11_faster1 | | 109050 | INDI 698 PL [symbol] | Ovary | III | 8685561 | Negative | Negative | Negative |
| 7612.9_faster1 | | 10745 | INDI 216 PL [symbol] | Colorectum | III | 8704765 | Negative | Negative | Negative |
| 7663.6_faster1 | | 110932 | INDI 385 PL [symbol] | Colorectum | I | 8710642 | Negative | Negative | Negative |
| 7546.4_faster1 | | 10563 | INDI 010 PL [symbol] | Lung | II | 8717278 | Negative | Negative | Negative |
| 7607.8_faster1 | Yes | 108934 | INDI 454 PL [symbol] | Breast | III | 8718537 | Negative | Negative | Negative |
| 7308.10_faster1 | | 109097 | INDI 790 PL [symbol] | Breast | II | 8724316 | Negative | Negative | Negative |
| 7547.5_faster1 | | 10755 | INDI 227 PL [symbol] | Lung | II | 8748209 | Negative | Negative | Negative |
| 7677.12_faster1 | | 111037 | INDI 915 PL [symbol] | Breast | II | 8772028 | Negative | Negative | Negative |
| 7534.9_faster1 | | 110665 | PAP 940 PL [symbol] | Ovary | III | 8792693 | Positive | Negative | Negative |
| 7561.6_faster1 | | 108974 | INDI 514 PL [symbol] | Stomach | I | 8794282 | Negative | Negative | Negative |
| 7012.8_faster1 | | 10630 | INDI 081 PL [symbol] | Colorectum | II | 8817504 | Positive | Negative | Negative |
| 7306.10_faster1 | Yes | 10756 | INDI 228 PL [symbol] | Breast | II | 8829767 | Positive | Negative | Negative |
| 7662.7_faster1 | | 110922 | INDI 328 PL [symbol] | Colorectum | I | 8840984 | Negative | Negative | Negative |
| 7610.4_faster1 | | 10689 | INDI 152 PL [symbol] | Colorectum | I | 8956285 | Negative | Negative | Negative |
| 7548.12_faster1 | | 10877 | INDI 414 PL [symbol] | Lung | II | 8958632 | Negative | Negative | Negative |
| 7592_combined.6_fast [symbol] | Yes | 10870 | INDI 405 PL [symbol] | Lung | I | 8959025 | Negative | Negative | Negative |
| 7638.6_faster1 | | 110812 | INDI 418 PL [symbol] | Colorectum | I | 8981237 | Negative | Negative | Negative |
| 7594.10_faster1 | | 109147 | INDI 909 PL [symbol] | Esophagus | II | 9046563 | Negative | Negative | Negative |
| 7041.10_faster1 | | 109078 | INDI 752 PL [symbol] | Colorectum | III | 9074211 | Negative | Negative | Negative |
| 7609.11_faster1 | | 10684 | INDI 146 PL [symbol] | Colorectum | II | 9111916 | Negative | Negative | Negative |
| 6857.8_faster1 | | 10789 | INDI 284 PL [symbol] | Colorectum | II | 9113264 | Positive | Negative | Negative |
| 7543.9_faster1 | | 110653 | PANCA 104 [symbol] | Pancreas | II | 9121697 | Negative | Negative | Negative |
| 6838_redo.10_faster1 | | 10885 | INDI 441 PL [symbol] | Esophagus | II | 9134834 | Negative | Negative | Negative |
| 7739.3_faster1 | | 111049 | CRC 455 PL [symbol] | Colorectum | I | 9143292 | Negative | Negative | Negative |
| 6858.8_faster1 | | 10546 | CRC 502 PL [symbol] | Colorectum | I | 9149440 | Negative | Negative | Negative |
| 7303.4_faster1 | Yes | 10616 | INDI 067 PL [symbol] | Breast | III | 9228387 | Negative | Negative | Negative |
| 7043.10_faster1 | | 10548 | CRC 506 PL [symbol] | Colorectum | III | 9248101 | Negative | Negative | Negative |
| 7548.6_faster1 | | 10792 | INDI 288 PL [symbol] | Lung | III | 9282231 | Positive | Negative | Negative |
| 7305.11_faster1 | | 109138 | INDI 882 PL [symbol] | Breast | II | 9338220 | Positive | Negative | Negative |
| 7308.3_faster1 | | 109060 | INDI 713 PL [symbol] | Breast | II | 9357095 | Negative | Negative | Negative |
| 7757.7_faster1 | Yes | 10591 | INDI 043 PL [symbol] | Colorectum | II | 9366800 | Negative | Negative | Negative |
| 7611.10_faster1 | | 10725 | INDI 194 PL [symbol] | Colorectum | II | 9392094 | Negative | Positive | Negative |
| 7608.4_faster1 | Yes | 109040 | INDI 684 PL [symbol] | Liver | III | 9409297 | Negative | Negative | Negative |
| 7757.12_faster1 | Yes | 10614 | INDI 065 PL [symbol] | Breast | II | 9414696 | Negative | Negative | Negative |
| 7606.9_faster1 | Yes | 10767 | INDI 246 PL [symbol] | Lung | II | 9434379 | Negative | Negative | Negative |
| 7603.8_faster1 | | 10646 | INDI 100 PL [symbol] | Colorectum | III | 9451781 | Negative | Positive | Negative |
| 7608.10_faster1 | Yes | 109116 | INDI 841 PL [symbol] | Colorectum | III | 9466736 | Positive | Negative | Negative |
| 7041.4_faster1 | | 109057 | INDI 709 PL [symbol] | Colorectum | II | 9517584 | Positive | Negative | Negative |
| 7592_combined.12_fas [symbol] | Yes | 10833 | INDI 355 PL [symbol] | Lung | I | 9526010 | Negative | Negative | Negative |
| 7640.10_faster1 | | 110836 | INDI 560 PL [symbol] | Colorectum | I | 9530486 | Negative | Positive | Negative |
| 7601.6_faster1 | | 108953 | INDI 479 PL [symbol] | Breast | III | 9541909 | Negative | Negative | Negative |
| 7606.10_faster1 | Yes | 10774 | INDI 262 PL [symbol] | Breast | III | 9555061 | Negative | Negative | Negative |
| 7305.3_faster1 | Yes | 109030 | INDI 664 PL [symbol] | Breast | I | 9557747 | Negative | Negative | Negative |
| 7303.10_faster1 | | 10887 | INDI 443 PL [symbol] | Breast | II | 9563313 | Negative | Negative | Negative |
| 7042.9_faster1 | Yes | 10865 | INDI 395 PL [symbol] | Lung | I | 9572740 | Positive | Negative | Negative |
| 7592_combined.5_fast [symbol] | Yes | 10816 | INDI 333 PL [symbol] | Lung | II | 9598766 | Positive | Negative | Negative |
| 7536.8_faster1 | | 110692 | PAPA 1345 [symbol] | Ovary | III | 9607992 | Positive | Negative | Negative |
| 7543.7_faster1 | | 110651 | PANCA 104 [symbol] | Pancreas | II | 9631283 | Negative | Negative | Negative |
| 7671.8_faster1 | | 110994 | INDI 763 PL [symbol] | Colorectum | I | 9670355 | Negative | Negative | Negative |
| 7646.10_faster1 | | 110886 | INDI 641 PL [symbol] | Breast | III | 9693253 | Negative | Negative | Negative |
| 7589.5_faster1 | | 109047 | INDI 694 PL [symbol] | Lung | I | 9721620 | Negative | Negative | Negative |
| 7642.4_faster1 | | 110840 | INDI 572 PL [symbol] | Colorectum | II | 9722277 | Positive | Negative | Negative |
| 7306.8_faster1 | | 10604 | INDI 056 PL [symbol] | Breast | II | 9734458 | Negative | Negative | Negative |
| 7563.12_faster1 | | 10715 | INDI 183 PL [symbol] | Stomach | I | 9735365 | Positive | Negative | Negative |
| 7672.11_faster1 | | 111002 | INDI 726 PL [symbol] | Colorectum | I | 9752953 | Negative | Negative | Negative |
| 7760.10_faster1 | Yes | 10695 | INDI 160 PL [symbol] | Colorectum | II | 9782990 | Negative | Negative | Negative |
| 7663.8_faster1 | | 110934 | INDI 396 PL [symbol] | Colorectum | I | 9785678 | Negative | Negative | Negative |
| 7011.3_faster1 | | 109009 | INDI 595 PL [symbol] | Esophagus | II | 9794909 | Positive | Negative | Negative |
| 7303.3_faster1 | Yes | 10612 | INDI 063 PL [symbol] | Breast | III | 9856414 | Negative | Negative | Negative |
| 7637.11_faster1 | | 110807 | INDI 306 PL [symbol] | Colorectum | III | 9948291 | Positive | Negative | Negative |
| 7665.7_faster1 | | 110953 | INDI 683 PL [symbol] | Colorectum | II | 9971674 | Negative | Negative | Negative |
| 7606.11_faster1 | Yes | 10788 | INDI 283 PL [symbol] | Lung | I | 9981617 | Negative | Negative | Negative |
| 7676.6_faster1 | | 111027 | INDI 835 PL [symbol] | Colorectum | III | 10028345 | Negative | Negative | Negative |
| 6857.10_faster1 | Yes | 10864 | INDI 394 PL [symbol] | Lung | II | 10043000 | Negative | Negative | Negative |
| 7646.12_faster1 | | 110888 | INDI 656 PL [symbol] | Breast | III | 10052211 | Negative | Negative | Negative |
| 7644.4_faster1 | | 110865 | INDI 628 PL [symbol] | Colorectum | II | 10122473 | Negative | Negative | Negative |
| 7012.6_faster1 | | 109021 | INDI 639 PL [symbol] | Liver | II | 10166996 | Negative | Negative | Negative |
| 7759.12_faster1 | Yes | 10671 | INDI 131 PL [symbol] | Stomach | II | 10187523 | Negative | Negative | Negative |
| 7758.8_faster1 | Yes | 10636 | INDI 087 PL [symbol] | Stomach | I | 10207839 | Negative | Negative | Negative |
| 7642.3_faster1 | | 110839 | INDI 570 PL [symbol] | Colorectum | II | 10224108 | Negative | Negative | Negative |
| 7757.8_faster1 | Yes | 10595 | INDI 047 PL [symbol] | Colorectum | II | 10273757 | Negative | Negative | Negative |
| 7637.4_faster1 | | 110800 | INDI 296 PL [symbol] | Colorectum | II | 10302905 | Negative | Negative | Negative |
| 7607.7_faster1 | Yes | 10854 | INDI 382 PL [symbol] | Breast | II | 10327454 | Negative | Negative | Negative |
| 6837.12_faster1 | | 10650 | INDI 104 PL [symbol] | Stomach | III | 10345980 | Negative | Negative | Negative |
| 6851.6_faster1 | | 108975 | INDI 516 PL [symbol] | Pancreas | II | 10382498 | Negative | Negative | Negative |
| 7041.8_faster1 | | 109016 | INDI 624 PL [symbol] | Colorectum | II | 10523846 | Positive | Negative | Negative |
| 6838_redo.8_faster1 | | 10744 | INDI 215 PL [symbol] | Liver | II | 10635260 | Negative | Negative | Negative |
| 7666.12_faster1 | | 110965 | INDI 258 PL [symbol] | Colorectum | I | 10661884 | Negative | Negative | Negative |
| 7590.5_faster1 | | 108987 | INDI 537 PL [symbol] | Ovary | I | 10688024 | Negative | Negative | Negative |
| 7755.12_faster1 | Yes | 10577 | INDI 027 PL [symbol] | Colorectum | II | 10705925 | Positive | Negative | Negative |
| 7608.9_faster1 | Yes | 109066 | INDI 723 PL [symbol] | Ovary | III | 10787127 | Negative | Negative | Negative |
| 7670.3_faster1 | | 110979 | INDI 644 PL [symbol] | Colorectum | I | 10792862 | Negative | Negative | Negative |
| 7644.3_faster1 | | 110864 | INDI 621 PL [symbol] | Colorectum | II | 10808736 | Negative | Negative | Negative |
| 7667.11_faster1 | | 110978 | INDI 742 PL [symbol] | Breast | II | 10866829 | Positive | Negative | Negative |
| 7544.10_faster1 | | 10558 | INDI 003 PL [symbol] | Lung | III | 10894563 | Negative | Negative | Negative |
| 7663.12_faster1 | | 110938 | INDI 501 PL [symbol] | Colorectum | III | 10895229 | Negative | Negative | Negative |
| 7593.4_faster1 | | 10835 | INDI 358 PL [symbol] | Lung | II | 10897648 | Negative | Negative | Negative |
| 7304.10_faster1 | Yes | 109003 | INDI 579 PL [symbol] | Breast | I | 10914894 | Positive | Negative | Negative |
| 7638.9_faster1 | | 110815 | INDI 422 PL [symbol] | Colorectum | II | 10943979 | Negative | Negative | Negative |
| 7740.5_faster1 | | 111061 | CRC 473 PL [symbol] | Colorectum | II | 10946083 | Positive | Negative | Negative |
| 7608.7_faster1 | Yes | 109052 | INDI 700 PL [symbol] | Lung | III | 10952423 | Negative | Negative | Negative |
| 6857.6_faster1 | | 109115 | INDI 838 PL [symbol] | Colorectum | III | 10963339 | Negative | Negative | Negative |
| 7307.11_faster1 | | 108990 | INDI 540 PL [symbol] | Breast | II | 10967901 | Negative | Negative | Negative |
| 7758.9_faster1 | Yes | 10637 | INDI 089 PL [symbol] | Colorectum | II | 10985306 | Negative | Negative | Negative |
| 7759.9_faster1 | Yes | 10668 | INDI 128 PL [symbol] | Colorectum | III | 11005383 | Negative | Negative | Negative |
| 7537.10_faster1 | | 110620 | PANC 674 [symbol] | Pancreas | II | 11007356 | Negative | Negative | Negative |
| 7739.9_faster1 | | 111055 | CRC 463 PL [symbol] | Colorectum | I | 11037586 | Negative | Negative | Negative |
| 7646.9_faster1 | | 110885 | INDI 638 PL [symbol] | Breast | I | 11134045 | Negative | Negative | Negative |
| 7609.10_faster1 | | 10683 | INDI 145 PL [symbol] | Colorectum | II | 11146634 | Negative | Negative | Negative |
| 7756.11_faster1 | Yes | 10576 | INDI 026 PL [symbol] | Lung | I | 11148157 | Negative | Negative | Negative |
| 7607.12_faster1 | Yes | 108971 | INDI 507 PL [symbol] | Lung | II | 11155220 | Negative | Negative | Negative |
| 7758.4_faster1 | Yes | 10625 | INDI 076 PL [symbol] | Esophagus | II | 11178927 | Negative | Negative | Negative |
| 6850.8_faster1 | | 108982 | INDI 529 PL [symbol] | Colorectum | I | 11205319 | Negative | Negative | Negative |
| 7756.5_faster1 | Yes | 10554 | CRC 531 PL [symbol] | Colorectum | III | 11205988 | Negative | Negative | Negative |
| 7759.8_faster1 | Yes | 10667 | INDI 127 PL [symbol] | Colorectum | II | 11226932 | Negative | Negative | Negative |
| 7547.10_faster1 | | 10771 | INDI 257 PL [symbol] | Lung | III | 11239146 | Positive | Negative | Negative |
| 7758.6_faster1 | Yes | 10633 | INDI 084 PL [symbol] | Stomach | II | 11265793 | Negative | Negative | Negative |
| 6858.10_faster1 | | 10551 | CRC 521 PL [symbol] | Colorectum | III | 11273801 | Negative | Negative | Negative |
| 7541.10_faster1 | | 110632 | PANCA 100 [symbol] | Pancreas | II | 11278288 | Negative | Negative | Negative |
| 7662.4_faster1 | | 110919 | INDI 321 PL [symbol] | Colorectum | III | 11281006 | Negative | Negative | Negative |
| 7561.3_faster1 | | 108993 | INDI 547 PL [symbol] | Stomach | III | 11284513 | Negative | Negative | Negative |
| 7607.10_faster1 | Yes | 108959 | INDI 490 PL [symbol] | Breast | III | 11318226 | Negative | Negative | Negative |
| 7306.12_faster1 | Yes | 10815 | INDI 332 PL [symbol] | Breast | I | 11332438 | Negative | Negative | Negative |
| 7304.11_faster1 | Yes | 109013 | INDI 619 PL [symbol] | Breast | II | 11334967 | Negative | Negative | Negative |
| 6850.12_faster1 | | 109038 | INDI 678 PL [symbol] | Colorectum | III | 11356102 | Positive | Negative | Negative |
| 7590.6_faster1 | | 109041 | INDI 686 PL [symbol] | Pancreas | II | 11430231 | Negative | Negative | Negative |
| 7306.6_faster1 | | 10607 | INDI 058 PL [symbol] | Breast | I | 11455317 | Negative | Negative | Negative |
| 7643.12_faster1 | | 110858 | INDI 620 PL [symbol] | Colorectum | II | 11474235 | Negative | Negative | Negative |
| 7665.10_faster1 | | 110955 | INDI 692 PL [symbol] | Colorectum | III | 11477099 | Negative | Negative | Negative |
| 7760.3_faster1 | Yes | 10673 | INDI 133 PL [symbol] | Stomach | I | 11499086 | Negative | Positive | Negative |
| 7756.9_faster1 | Yes | 10574 | INDI 024 PL [symbol] | Lung | II | 11518949 | Negative | Negative | Negative |
| 7304.5_faster1 | Yes | 108949 | INDI 474 PL [symbol] | Breast | II | 11532501 | Negative | Negative | Negative |
| 7534.7_faster1 | | 110664 | PAP 939 PL [symbol] | Ovary | III | 11546417 | Positive | Negative | Negative |
| 6837.8_faster1 | | 10632 | INDI 083 PL [symbol] | Stomach | II | 11547580 | Negative | Negative | Negative |
| 7304.3_faster1 | Yes | 108939 | INDI 460 PL [symbol] | Breast | III | 11621552 | Negative | Negative | Negative |
| 7014.10_faster1 | | 109043 | INDI 688 PL [symbol] | Colorectum | II | 11688516 | Negative | Negative | Negative |
| 7643.5_faster1 | | 110851 | INDI 608 PL [symbol] | Colorectum | II | 11689087 | Positive | Negative | Negative |
| 7607.4_faster1 | Yes | 10688 | INDI 151 PL [symbol] | Stomach | II | 11728749 | Positive | Negative | Negative |
| 7593.7_faster1 | | 10846 | INDI 369 PL [symbol] | Lung | I | 11728834 | Negative | Negative | Negative |
| 7303.8_faster1 | Yes | 10798 | INDI 300 PL [symbol] | Breast | III | 11740790 | Negative | Negative | Negative |
| 7756.6_faster1 | Yes | 10557 | INDI 002 PL [symbol] | Lung | II | 11741917 | Negative | Negative | Negative |
| 7757.3_faster1 | Yes | 10580 | INDI 030 PL [symbol] | Colorectum | II | 11752353 | Negative | Negative | Negative |
| 7546.3_faster1 | | 10561 | INDI 007 PL [symbol] | Lung | II | 11877295 | Negative | Negative | Negative |
| 7610.6_faster1 | | 10692 | INDI 156 PL [symbol] | Colorectum | III | 11879034 | Positive | Negative | Negative |
| 7670.5_faster1 | | 110981 | INDI 649 PL [symbol] | Colorectum | III | 11893568 | Negative | Negative | Negative |
| 7646.3_faster1 | | 110880 | INDI 613 PL [symbol] | Breast | II | 11896835 | Negative | Negative | Negative |
| 7677.11_faster1 | | 111038 | PANCA 115 [symbol] | Pancreas | I | 11899685 | Negative | Negative | Negative |
| 7760.8_faster1 | Yes | 10681 | INDI 143 PL [symbol] | Colorectum | II | 11955147 | Negative | Negative | Negative |
| 6839_redo.7_faster1 | | 109000 | INDI 567 PL [symbol] | Colorectum | II | 11976877 | Positive | Negative | Negative |
| 7756.4_faster1 | Yes | 10541 | CRC 486 PL [symbol] | Colorectum | I | 12013803 | Positive | Negative | Negative |
| 7307.7_faster1 | Yes | 108957 | INDI 487 PL [symbol] | Breast | II | 12026717 | Negative | Negative | Negative |
| 7303.9_faster1 | Yes | 10806 | INDI 317 PL [symbol] | Breast | II | 12074956 | Negative | Negative | Negative |
| 7543.5_faster1 | | 110649 | PANCA 103 [symbol] | Pancreas | II | 12078995 | Negative | Negative | Negative |
| 7549.7_faster1 | | 108965 | INDI 497 PL [symbol] | Lung | II | 12080651 | Negative | Negative | Negative |
| 7012.11_faster1 | | 108940 | INDI 461 PL [symbol] | Colorectum | III | 12087006 | Positive | Negative | Negative |
| 7760.6_faster1 | Yes | 10677 | INDI 137 PL [symbol] | Stomach | II | 12102716 | Negative | Negative | Negative |
| 7304.7_faster1 | Yes | 108997 | INDI 559 PL [symbol] | Breast | III | 12106034 | Negative | Negative | Negative |
| 7758.10_faster1 | Yes | 10639 | INDI 092 PL [symbol] | Stomach | II | 12202185 | Negative | Negative | Negative |
| 7673.4_faster1 | | 111011 | INDI 785 PL [symbol] | Colorectum | I | 12202785 | Negative | Negative | Negative |
| 7756.10_faster1 | Yes | 10575 | INDI 025 PL [symbol] | Lung | I | 12225917 | Negative | Negative | Negative |
| 7758.12_faster1 | Yes | 10651 | INDI 107 PL [symbol] | Colorectum | II | 12270627 | Negative | Negative | Negative |
| 7760.4_faster1 | Yes | 10674 | INDI 134 PL [symbol] | Stomach | III | 12288756 | Positive | Negative | Negative |
| 7758.3_faster1 | Yes | 10615 | INDI 066 PL [symbol] | Breast | III | 12290640 | Negative | Negative | Negative |
| 6850.4_faster1 | | 109059 | INDI 711 PL [symbol] | Breast | III | 12330713 | Negative | Negative | Negative |
| 7607.5_faster1 | Yes | 10812 | INDI 326 PL [symbol] | Lung | I | 12357550 | Positive | Negative | Negative |
| 7759.11_faster1 | Yes | 10670 | INDI 130 PL [symbol] | Stomach | II | 12357763 | Negative | Negative | Negative |
| 6836.9_faster1 | | 10565 | INDI 012 PL [symbol] | Lung | I | 12388482 | Negative | Negative | Negative |
| 7307.5_faster1 | Yes | 10838 | INDI 361 PL [symbol] | Breast | III | 12406295 | Negative | Negative | Negative |
| 7011.4_faster1 | | 109127 | INDI 859 PL [symbol] | Esophagus | II | 12414216 | Positive | Positive | Negative |
| 7042.11_faster1 | | 10934 | CRC 465 PL [symbol] | Colorectum | II | 12423748 | Negative | Negative | Negative |
| 7536.7_faster1 | | 110691 | PAPA 1343 [symbol] | Ovary | III | 12457913 | Positive | Negative | Negative |
| 7758.11_faster1 | Yes | 10647 | INDI 101 PL [symbol] | Colorectum | III | 12471222 | Negative | Negative | Negative |
| 7043.8_faster1 | | 10542 | CRC 489 PL [symbol] | Colorectum | II | 12501319 | Negative | Negative | Negative |
| 7305.12_faster1 | | 109139 | INDI 887 PL [symbol] | Breast | III | 12506601 | Negative | Negative | Negative |
| 7649.10_faster1 | | 110916 | INDI 704 PL [symbol] | Breast | II | 12508209 | Negative | Negative | Negative |
| 7596.8_faster1 | | 10609 | INDI 060 PL [symbol] | Breast | II | 12527347 | Negative | Negative | Negative |
| 7742.4_faster1 | | 111080 | CRC 501 PL [symbol] | Colorectum | I | 12534877 | Negative | Negative | Negative |
| 7591.9_faster1 | | 109101 | INDI 798 PL [symbol] | Ovary | II | 12542225 | Positive | Negative | Negative |
| 7304.9_faster1 | Yes | 108999 | INDI 565 PL [symbol] | Breast | III | 12568286 | Positive | Negative | Negative |
| 7760.9_faster1 | Yes | 10690 | INDI 153 PL [symbol] | Stomach | II | 12571286 | Negative | Negative | Negative |
| 6839_redo.4_faster1 | | 109103 | INDI 800 PL [symbol] | Liver | II | 12571619 | Negative | Negative | Negative |
| 7305.4_faster1 | Yes | 109032 | INDI 668 PL [symbol] | Breast | III | 12597944 | Negative | Negative | Negative |
| 7760.7_faster1 | Yes | 10680 | INDI 141 PL [symbol] | Stomach | III | 12602463 | Negative | Negative | Negative |
| 7596.9_faster1 | | 10610 | INDI 061 PL [symbol] | Breast | II | 12603504 | Negative | Negative | Negative |
| 7043.12_faster1 | | 10552 | CRC 524 PL [symbol] | Colorectum | II | 12612680 | Negative | Negative | Negative |
| 7644.7_faster1 | | 110868 | INDI 633 PL [symbol] | Colorectum | II | 12637868 | Negative | Negative | Negative |
| 7757.9_faster1 | Yes | 10598 | INDI 050 PL [symbol] | Breast | II | 12708371 | Negative | Negative | Negative |
| 7042.5_faster1 | | 109105 | INDI 802 PL [symbol] | Pancreas | II | 12736111 | Negative | Negative | Negative |
| 7543.12_faster1 | | 110656 | PANCA 105 [symbol] | Pancreas | II | 12750761 | Negative | Negative | Negative |
| 6839_redo.3_faster1 | | 109094 | INDI 786 PL [symbol] | Liver | I | 12774784 | Positive | Negative | Negative |
| 7547.9_faster1 | | 10768 | INDI 247 PL [symbol] | Lung | I | 12774797 | Positive | Negative | Negative |
| 7606.5_faster1 | Yes | 10587 | INDI 038 PL [symbol] | Colorectum | II | 12779723 | Negative | Negative | Negative |
| 7665.4_faster1 | | 110950 | INDI 677 PL [symbol] | Colorectum | II | 12822270 | Negative | Negative | Negative |
| 7663.4_faster1 | | 110930 | INDI 378 PL [symbol] | Colorectum | I | 12844324 | Negative | Negative | Negative |
| 7306.5_faster1 | | 10600 | INDI 052 PL [symbol] | Breast | II | 12876008 | Negative | Negative | Negative |
| 7742.3_faster1 | | 111079 | CRC 500 PL [symbol] | Colorectum | I | 12878766 | Negative | Negative | Negative |
| 7307.3_faster1 | Yes | 10829 | INDI 348 PL [symbol] | Breast | II | 12894091 | Negative | Negative | Negative |
| 7011.8_faster1 | | 10666 | INDI 126 PL [symbol] | Stomach | III | 12932905 | Positive | Negative | Negative |
| 7757.5_faster1 | Yes | 10582 | INDI 032 PL [symbol] | Colorectum | III | 12935921 | Negative | Negative | Negative |
| 7739.5_faster1 | | 111051 | CRC 457 PL [symbol] I | Colorectum | II | 12949574 | Negative | Negative | Negative |
| 7307.8_faster1 | | 108958 | INDI 489 PL [symbol] | Breast | II | 13000860 | Negative | Negative | Negative |
| 7308.6_faster1 | | 109064 | INDI 721 PL [symbol] | Breast | II | 13019464 | Negative | Negative | Negative |
| 7563.7_faster1 | | 109080 | INDI 755 PL [symbol] | Esophagus | III | 13023659 | Negative | Negative | Negative |
| 6839_redo.10_faster1 | | 109012 | INDI 614 PL [symbol] | Colorectum | II | 13025861 | Negative | Negative | Negative |
| 7649.8_faster1 | | 110914 | INDI 673 PL [symbol] | Breast | II | 13086229 | Negative | Negative | Negative |
| 7638.8_faster1 | | 110814 | INDI 421 PL [symbol] | Colorectum | II | 13095095 | Negative | Negative | Negative |
| 7306.7_faster1 | | 10603 | INDI 055 PL [symbol] | Breast | II | 13118272 | Negative | Negative | Negative |
| 6835.10_faster1 | | 110645 | PANCA 103 [symbol] | Pancreas | II | 13135905 | Negative | Negative | Negative |
| 7549.4_faster1 | | 108960 | INDI 491 PL [symbol] | Lung | I | 13143161 | Negative | Negative | Negative |
| 7756.8_faster1 | Yes | 10571 | INDI 018 PL [symbol] | Lung | III | 13162781 | Negative | Negative | Negative |
| 7663.9_faster1 | | 110935 | INDI 398 PL [symbol] | Colorectum | II | 13172563 | Negative | Negative | Negative |
| 7670.7_faster1 | | 110983 | INDI 651 PL [symbol] | Colorectum | III | 13201680 | Negative | Negative | Negative |
| 7306.9_faster1 | | 10605 | INDI 057 PL [symbol] | Breast | III | 13242175 | Negative | Negative | Negative |
| 7304.6_faster1 | Yes | 109002 | INDI 577 PL [symbol] | Breast | I | 13313274 | Negative | Negative | Negative |
| 7042.8_faster1 | Yes | 10832 | INDI 353 PL [symbol] | Lung | II | 13337284 | Positive | Negative | Negative |
| 7011.11_faster1 | | 108943 | INDI 465 PL [symbol] | Esophagus | II | 13379186 | Negative | Negative | Negative |
| 6836.6_faster1 | | 10664 | INDI 124 PL [symbol] | Colorectum | II | 13390707 | Negative | Negative | Negative |
| 7305.8_faster1 | | 109135 | INDI 876 PL [symbol] | Breast | II | 13403320 | Negative | Negative | Negative |
| 7304.8_faster1 | Yes | 108998 | INDI 561 PL [symbol] | Breast | III | 13464530 | Negative | Negative | Negative |
| 7666.6_faster1 | | 110959 | INDI 234 PL [symbol] | Colorectum | I | 13511305 | Negative | Negative | Negative |
| 7308.9_faster1 | | 109096 | INDI 789 PL [symbol] | Breast | II | 13516563 | Negative | Negative | Negative |
| 7666.10_faster1 | | 110963 | INDI 251 PL [symbol] | Colorectum | II | 13571140 | Negative | Negative | Negative |
| 7666.7_faster1 | | 110960 | INDI 243 PL [symbol] | Colorectum | I | 13578246 | Positive | Negative | Negative |
| 7043.4_faster1 | | 10537 | CRC 471 PL [symbol] | Colorectum | III | 13587546 | Positive | Negative | Negative |
| 6839_redo.9_faster1 | | 109008 | INDI 591 PL [symbol] | Colorectum | II | 13660283 | Negative | Negative | Negative |
| 7670.11_faster1 | | 110987 | INDI 660 PL [symbol] | Colorectum | II | 13703592 | Negative | Negative | Negative |
| 7607.6_faster1 | Yes | 10844 | INDI 367 PL [symbol] | Liver | I | 13774303 | Positive | Negative | Negative |
| 6850.5_faster1 | | 10813 | INDI 327 PL [symbol] | Colorectum | II | 13817537 | Negative | Negative | Negative |
| 7594.11_faster1 | | 109148 | INDI 911 PL [symbol] | Esophagus | II | 13867646 | Negative | Negative | Negative |
| 7305.9_faster1 | | 109136 | INDI 877 PL [symbol] | Breast | I | 13951918 | Negative | Negative | Negative |
| 7667.8_faster1 | | 110974 | INDI 281 PL [symbol] | Colorectum | I | 13953579 | Negative | Negative | Negative |
| 7303.6_faster1 | | 10620 | INDI 071 PL [symbol] | Breast | II | 13959147 | Negative | Negative | Negative |
| 7610.8_faster1 | | 10694 | INDI 159 PL [symbol] | Colorectum | II | 13985715 | Negative | Negative | Negative |
| 7740.12_faster1 | | 111068 | CRC 483 PL [symbol] | Colorectum | I | 14034018 | Negative | Negative | Negative |
| 7757.4_faster1 | Yes | 10581 | INDI 031 PL [symbol] | Colorectum | II | 14048988 | Negative | Negative | Negative |
| 7596.3_faster1 | | 10597 | INDI 049 PL [symbol] | Breast | III | 14117560 | Negative | Negative | Negative |
| 7757.11_faster1 | Yes | 10611 | INDI 062 PL [symbol] | Breast | II | 14121104 | Negative | Negative | Negative |
| 7760.5_faster1 | Yes | 10676 | INDI 136 PL [symbol] | Colorectum | II | 14149268 | Negative | Negative | Negative |
| 7537.7_faster1 | | 109153 | LCR 814 PL [symbol] | Pancreas | II | 14181633 | Negative | Negative | Negative |
| 7667.5_faster1 | | 110971 | INDI 268 PL [symbol] | Colorectum | III | 14199962 | Positive | Negative | Negative |
| 7610.7_faster1 | | 10693 | INDI 158 PL [symbol] | Colorectum | I | 14204452 | Negative | Negative | Negative |
| 7638.7_faster1 | | 110813 | INDI 419 PL [symbol] | Colorectum | I | 14241999 | Negative | Negative | Negative |
| 7757.6_faster1 | Yes | 10590 | INDI 042 PL [symbol] | Colorectum | I | 14247573 | Negative | Negative | Negative |
| 7612.8_faster1 | | 10742 | INDI 213 PL [symbol] | Colorectum | III | 14302511 | Negative | Negative | Negative |
| 7740.3_faster1 | | 111059 | CRC 469 PL [symbol] | Colorectum | II | 14346493 | Negative | Negative | Negative |
| 7643.3_faster1 | | 110849 | INDI 600 PL [symbol] | Colorectum | II | 14392099 | Negative | Negative | Negative |
| 7565.5_faster1 | | 10659 | INDI 119 PL [symbol] | Esophagus | I | 14393602 | Positive | Negative | Negative |
| 7305.7_faster1 | Yes | 109134 | INDI 875 PL [symbol] | Breast | II | 14409097 | Negative | Negative | Negative |
| 7667.7_faster1 | | 110973 | INDI 279 PL [symbol] | Colorectum | II | 14426072 | Positive | Negative | Negative |
| 7592_combined.4_fast [symbol] | Yes | 10814 | INDI 331 PL [symbol] | Lung | III | 14455257 | Positive | Negative | Negative |
| 7307.6_faster1 | Yes | 10860 | INDI 390 PL [symbol] | Breast | II | 14465100 | Negative | Negative | Negative |
| 7534.10_faster1 | | 110666 | PAP 941 PL [symbol] | Ovary | III | 14471184 | Negative | Negative | Negative |
| 7606.4_faster1 | Yes | 10569 | INDI 016 PL [symbol] | Lung | III | 14478527 | Negative | Negative | Negative |
| 7585.3_faster1 | | 10710 | INDI 177 PL [symbol] | Liver | III | 14516564 | Negative | Negative | Negative |
| 7543.10_faster1 | | 110654 | PANCA 105 [symbol] | Pancreas | II | 14534837 | Negative | Negative | Negative |
| 7303.5_faster1 | | 10618 | INDI 069 PL [symbol] | Breast | II | 14572791 | Negative | Negative | Negative |
| 6835.6_faster1 | | 110682 | PAPA 1330 [symbol] | Ovary | III | 14623813 | Positive | Negative | Negative |
| 7759.6_faster1 | Yes | 10658 | INDI 116 PL [symbol] | Stomach | I | 14651561 | Negative | Positive | Negative |
| 7638.10_faster1 | | 110816 | INDI 423 PL [symbol] | Colorectum | II | 14681242 | Negative | Negative | Negative |
| 7610.5_faster1 | | 10691 | INDI 155 PL [symbol] | Colorectum | II | 14715914 | Negative | Negative | Negative |
| 6851.11_faster1 | | 109086 | INDI 770 PL [symbol] | Colorectum | II | 14739471 | Negative | Negative | Negative |
| 7549.5_faster1 | | 108961 | INDI 492 PL [symbol] | Lung | I | 14747011 | Negative | Negative | Negative |
| 6839_redo.6_faster1 | | 10883 | INDI 439 PL [symbol] | Colorectum | III | 14782970 | Positive | Negative | Negative |
| 7602.4_faster1 | | 10588 | INDI 039 PL [symbol] | Colorectum | III | 14879768 | Negative | Negative | Negative |
| 7608.12_faster1 | Yes | 109133 | INDI 872 PL [symbol] | Breast | III | 14923030 | Negative | Negative | Negative |
| 7678.5_faster1 | | 111041 | INDI 920 PL [symbol] | Ovary | III | 14942044 | Negative | Negative | Negative |
| 7305.5_faster1 | Yes | 109081 | INDI 756 PL [symbol] | Breast | II | 14975145 | Negative | Positive | Negative |
| 7535.9_faster1 | | 110683 | PAPA 1331 [symbol] | Ovary | III | 15069331 | Positive | Negative | Negative |
| 7742.5_faster1 | | 111081 | CRC 503 PL [symbol] | Colorectum | I | 15091681 | Positive | Negative | Negative |
| 7639.3_faster1 | | 110819 | INDI 429 PL [symbol] | Colorectum | II | 15107456 | Negative | Negative | Negative |
| 7606.12_faster1 | Yes | 10791 | INDI 287 PL [symbol] | Breast | III | 15110509 | Positive | Negative | Negative |
| 7606.3_faster1 | Yes | 109150 | INDI 928 PL [symbol] | Stomach | II | 15199986 | Negative | Negative | Negative |
| 7565.3_faster1 | | 10656 | INDI 112 PL [symbol] | Esophagus | II | 15242109 | Negative | Negative | Negative |
| 7645.4_faster1 | | 110870 | INDI 588 PL [symbol] | Breast | III | 15244435 | Negative | Negative | Negative |
| 7043.5_faster1 | | 10538 | CRC 479 PL [symbol] | Colorectum | II | 15245644 | Negative | Negative | Negative |
| 7014.9 _faster1 | | 108977 | INDI 518 PL [symbol] | Colorectum | III | 15270476 | Positive | Negative | Negative |
| 7041.7_faster1 | | 109023 | INDI 643 PL [symbol] | Colorectum | II | 15328844 | Negative | Negative | Negative |
| 7560.6_faster1 | | 108988 | INDI 538 PL [symbol] | Lung | III | 15413435 | Negative | Negative | Negative |
| 7308.12_faster1 | | 109114 | INDI 828 PL [symbol] | Breast | II | 15523762 | Positive | Negative | Negative |
| 7586.10_faster1 | | 109083 | INDI 760 PL [symbol] | Liver | III | 15527805 | Positive | Negative | Negative |
| 7307.10_faster1 | | 108976 | INDI 517 PL [symbol] | Breast | III | 15624023 | Negative | Negative | Negative |
| 7607.9_faster1 | Yes | 108956 | INDI 485 PL [symbol] | Esophagus | III | 15637371 | Negative | Negative | Negative |
| 7664.5_faster1 | | 110941 | INDI 515 PL [symbol] | Colorectum | III | 15688198 | Negative | Negative | Negative |
| 7534.5_faster1 | | 110680 | PAP 962 PL [symbol] | Ovary | III | 15695598 | Negative | Negative | Negative |
| 7611.8_faster1 | | 10713 | INDI 180 PL [symbol] | Colorectum | III | 15784294 | Negative | Negative | Negative |
| 6858.3_faster1 | | 10533 | CRC 460 PL [symbol] | Colorectum | II | 15786364 | Negative | Negative | Negative |
| 7566.10_faster1 | | 10735 | INDI 206 PL [symbol] | Stomach | III | 15861333 | Positive | Negative | Negative |
| 7642.7_faster1 | | 110843 | INDI 583 PL [symbol] | Colorectum | II | 15884110 | Negative | Negative | Negative |
| 7665.6_faster1 | | 110952 | INDI 682 PL [symbol] | Colorectum | III | 15898942 | Negative | Negative | Negative |
| 7544.4_faster1 | | 110658 | PANCA 105 [symbol] | Pancreas | II | 15938986 | Negative | Negative | Negative |
| 6858.7_faster1 | | 10545 | CRC 499 PL [symbol] | Colorectum | I | 15947500 | Negative | Negative | Negative |
| 7601.5_faster1 | | 108952 | INDI 478 PL [symbol] | Breast | II | 15952190 | Negative | Negative | Negative |
| 7603.4_faster1 | | 10640 | INDI 093 PL [symbol] | Colorectum | I | 15978437 | Negative | Negative | Negative |
| 7677.9_faster1 | | 111035 | INDI 840 PL [symbol] | Breast | II | 16140068 | Negative | Negative | Negative |
| 7562.3_faster1 | | 109099 | INDI 792 PL [symbol] | Stomach | I | 16179994 | Negative | Negative | Negative |
| 7639.7_faster1 | | 110823 | INDI 436 PL [symbol] | Colorectum | III | 16208714 | Negative | Negative | Negative |
| 7544.11_faster1 | | 10559 | INDI 004 PL [symbol] | Lung | I | 16262105 | Negative | Negative | Negative |
| 6838_redo.3_faster1 | | 109005 | INDI 585 PL [symbol] | Esophagus | III | 16300134 | Positive | Negative | Negative |
| 7561.12_faster1 | | 109117 | INDI 843 PL [symbol] | Esophagus | I | 16335896 | Negative | Negative | Negative |
| 7670.8_faster1 | | 110984 | INDI 654 PL [symbol] | Colorectum | II | 16345406 | Negative | Negative | Negative |
| 7664.3_faster1 | | 110939 | INDI 506 PL [symbol] | Colorectum | I | 16419133 | Negative | Negative | Negative |
| 7009.11_faster1 | | 10578 | INDI 028 PL [symbol] | Colorectum | II | 16523538 | Positive | Negative | Negative |
| 7638.4_faster1 | | 110810 | INDI 311 PL [symbol] | Colorectum | III | 16541062 | Negative | Negative | Negative |
| 7304.4_faster1 | Yes | 108948 | INDI 473 PL [symbol] | Breast | III | 16558913 | Positive | Negative | Negative |
| 7305.6_faster1 | Yes | 109082 | INDI 757 PL [symbol] | Breast | II | 16672564 | Negative | Negative | Negative |
| 7548.7_faster1 | | 10794 | INDI 291 PL [symbol] | Lung | I | 16675122 | Negative | Negative | Negative |
| 7549.9_faster1 | | 108969 | INDI 503 PL [symbol] | Lung | I | 16752817 | Negative | Negative | Negative |
| 7544.6_faster1 | | 110660 | PANCA 106 [symbol] | Pancreas | II | 16838030 | Positive | Negative | Negative |
| 7535.3_faster1 | | 110676 | PAP 956 PL [symbol] | Ovary | III | 16844133 | Positive | Positive | Negative |
| 7643.4_faster1 | | 110850 | INDI 605 PL [symbol] | Colorectum | II | 16865887 | Negative | Negative | Negative |
| 7672.9_faster1 | | 111000 | INDI 724 PL [symbol] | Colorectum | II | 17102657 | Negative | Negative | Negative |
| 7756.7_faster1 | Yes | 10570 | INDI 017 PL [symbol] | Lung | I | 17102879 | Negative | Negative | Negative |
| 7642.11_faster1 | | 110847 | INDI 598 PL [symbol] | Colorectum | II | 17127403 | Negative | Negative | Negative |
| 7307.4_faster1 | Yes | 10831 | INDI 352 PL [symbol] | Breast | III | 17185278 | Negative | Negative | Negative |
| 7758.7_faster1 | Yes | 10635 | INDI 086 PL [symbol] | Stomach | I | 17283778 | Negative | Negative | Negative |
| 6858.9_faster1 | | 10550 | CRC 520 PL [symbol] | Colorectum | III | 17374052 | Negative | Negative | Negative |
| 7607.3_faster1 | | 10793 | INDI 289 PL [symbol] | Breast | II | 17378202 | Positive | Negative | Negative |
| 7663.5_faster1 | | 110931 | INDI 379 PL [symbol] | Colorectum | III | 17447719 | Negative | Negative | Negative |
| 7584.4 _faster1 | | 10627 | INDI 078 PL [symbol] | Liver | II | 17497723 | Negative | Negative | Negative |
| 6858.4_faster1 | | 10539 | CRC 480 PL [symbol] | Colorectum | III | 17512125 | Negative | Negative | Negative |
| 7306.3_faster1 | | 10596 | INDI 048 PL [symbol] | Breast | II | 17518393 | Negative | Negative | Negative |
| 7041.5_faster1 | | 109019 | INDI 635 PL [symbol] | Colorectum | II | 17523673 | Negative | Positive | Negative |
| 7608.5_faster1 | Yes | 109049 | INDI 696 PL [symbol] | Lung | II | 17541286 | Negative | Negative | Negative |
| 7740.9_faster1 | | 111065 | CRC 477 PL [symbol] | Colorectum | II | 17644267 | Negative | Negative | Negative |
| 7043.6_faster1 | | 10540 | CRC 481 PL [symbol] | Colorectum | II | 17728894 | Negative | Negative | Negative |
| 7608.3_faster1 | Yes | 108972 | INDI 511 PL [symbol] | Colorectum | III | 17764622 | Negative | Negative | Negative |
| 7602.5_faster1 | | 10589 | INDI 040 PL [symbol] | Colorectum | I | 17912621 | Negative | Negative | Negative |
| 7560.4_faster1 | | 108983 | INDI 532 PL [symbol] | Lung | I | 17926546 | Negative | Negative | Negative |
| 7637.12_faster1 | | 110808 | INDI 307 PL [symbol] | Colorectum | III | 18031641 | Negative | Negative | Negative |
| 6850.9_faster1 | | 108989 | INDI 539 PL [symbol] | Colorectum | II | 18211342 | Negative | Negative | Negative |
| 6858.11_faster1 | | 10553 | CRC 526 PL [symbol] | Colorectum | III | 18336045 | Negative | Negative | Negative |
| 7613.12_faster1 | | 109126 | INDI 855 PL [symbol] | Colorectum | II | 18343702 | Negative | Negative | Negative |
| 7542.3_faster1 | | 110635 | PANCA 101 [symbol] | Pancreas | II | 18351665 | Negative | Negative | Negative |
| 7011.6_faster1 | | 109031 | INDI 665 PL [symbol] | Esophagus | III | 18412737 | Positive | Positive | Negative |
| 7536.11_faster1 | | 110695 | PAPA 1348 [symbol] | Ovary | III | 18417631 | Positive | Negative | Negative |
| 7543.6_faster1 | | 110650 | PANCA 104 [symbol] | Pancreas | II | 18443523 | Negative | Negative | Negative |
| 7303.7_faster1 | Yes | 109130 | INDI 865 PL [symbol] | Breast | III | 18466940 | Negative | Negative | Negative |
| 7610.11_faster1 | | 10699 | INDI 164 PL [symbol] | Colorectum | I | 18473786 | Negative | Negative | Negative |
| 7590.7_faster1 | | 109048 | INDI 695 PL [symbol] | Pancreas | II | 18525122 | Positive | Negative | Negative |
| 7742.6_faster1 | | 111082 | CRC 50 [symbol] PL [symbol] | Colorectum | III | 18541814 | Negative | Negative | Negative |
| 7594.8_faster1 | | 109145 | INDI 907 PL [symbol] | Esophagus | II | 18542868 | Negative | Negative | Negative |
| 7586.9_faster1 | | 109033 | INDI 669 PL [symbol] | Liver | III | 18602855 | Negative | Negative | Negative |
| 7585.7_faster1 | | 10737 | INDI 208 PL [symbol] | Liver | II | 18613650 | Positive | Negative | Negative |
| 7010.4_faster1 | | 10644 | INDI 097 PL [symbol] | Colorectum | III | 18645760 | Negative | Negative | Negative |
| 7563.3_faster1 | | 109124 | INDI 853 PL [symbol] | Esophagus | II | 18770983 | Negative | Negative | Negative |
| 7591.7_faster1 | | 109076 | INDI 745 PL [symbol] | Pancreas | II | 18782675 | Negative | Negative | Negative |
| 7739.12_faster1 | | 111058 | CRC 467 PL [symbol] | Colorectum | III | 18793851 | Positive | Negative | Negative |
| 7639.8_faster1 | | 110824 | INDI 437 PL [symbol] | Colorectum | I | 18869016 | Negative | Negative | Negative |
| 7564.5_faster1 | | 10719 | INDI 187 PL [symbol] | Stomach | I | 18873266 | Negative | Negative | Negative |
| 7308.5_faster1 | | 109062 | INDI 717 PL [symbol] | Breast | I | 18970952 | Negative | Negative | Negative |
| 7664.7_faster1 | | 110943 | INDI 523 PL [symbol] | Colorectum | I | 18978926 | Negative | Negative | Negative |
| 6850.6_faster1 | | 108966 | INDI 498 PL [symbol] | Colorectum | II | 18997615 | Positive | Negative | Negative |
| 7534.4_faster1 | | 110672 | PAP 950 PL [symbol] | Ovary | III | 19030714 | Positive | Negative | Positive |
| 7637.6_faster1 | | 110802 | INDI 299 PL [symbol] | Colorectum | I | 19165467 | Negative | Negative | Negative |
| 7591.6_faster1 | | 109056 | INDI 706 PL [symbol] | Lung | I | 19176306 | Negative | Negative | Negative |
| 7613.3_faster1 | | 10751 | INDI 223 PL [symbol] | Colorectum | I | 19229885 | Negative | Negative | Negative |
| 7742.11_faster1 | | 111087 | CRC 511 PL [symbol] | Colorectum | II | 19303564 | Negative | Negative | Negative |
| 7611.4_faster1 | | 10704 | INDI 170 PL [symbol] | Colorectum | I | 19410722 | Negative | Negative | Negative |
| 7306.4_faster1 | | 10599 | INDI 051 PL [symbol] | Breast | III | 19496002 | Negative | Negative | Negative |
| 7561.11_faster1 | | 10778 | INDI 267 PL [symbol] | Esophagus | II | 19771902 | Negative | Negative | Negative |
| 7536.5_faster1 | | 110689 | PAPA 1339 [symbol] | Ovary | I | 19787879 | Positive | Negative | Positive |
| 7640.9_faster1 | | 110835 | INDI 558 PL [symbol] | Colorectum | I | 19824684 | Negative | Negative | Negative |
| 7739.7_faster1 | | 111053 | CRC 461 PL [symbol] | Colorectum | I | 19956732 | Negative | Negative | Negative |
| 7665.11_faster1 | | 110958 | INDI 899 PL [symbol] | Breast | I | 19981298 | Negative | Negative | Negative |
| 6851.8_faster1 | | 109058 | INDI 710 PL [symbol] | Colorectum | III | 20001862 | Negative | Negative | Negative |
| 7592_combined.9_fast [symbol] | Yes | 10824 | INDI 342 PL [symbol] | Lung | III | 20199922 | Negative | Negative | Negative |
| 6850.11_faster1 | | 109037 | INDI 675 PL [symbol] | Colorectum | III | 20230333 | Positive | Negative | Negative |
| 7646.7_faster1 | | 110883 | INDI 630 PL [symbol] | Breast | I | 20320861 | Negative | Negative | Negative |
| 7596.5_faster1 | | 10601 | INDI 053 PL [symbol] | Breast | II | 20810618 | Positive | Negative | Negative |
| 7611.11_faster1 | | 10726 | INDI 195 PL [symbol] | Colorectum | III | 20853590 | Negative | Negative | Negative |
| 6850.7_faster1 | | 108968 | INDI 502 PL [symbol] | Colorectum | III | 20932649 | Negative | Negative | Negative |
| 7013.4_faster1 | | 109004 | INDI 581 PL [symbol] | Colorectum | II | 20977458 | Positive | Negative | Negative |
| 7639.5_faster1 | | 110821 | INDI 433 PL [symbol] | Colorectum | II | 21461355 | Negative | Negative | Negative |
| 7585.3_faster1 | | 108955 | INDI 482 PL [symbol] | Liver | II | 21477232 | Negative | Negative | Negative |
| 6839_redo.5_faster1 | | 109109 | INDI 817 PL [symbol] | Liver | III | 21545705 | Negative | Negative | Negative |
| 7602.9_faster1 | | 10593 | INDI 045 PL [symbol] | Colorectum | III | 21580012 | Negative | Negative | Negative |
| 7676.9_faster1 | | 111020 | INDI 794 PL [symbol] | Breast | II | 21787265 | Negative | Negative | Negative |
| 7671.7_faster1 | | 110993 | INDI 761 PL [symbol] | Colorectum | II | 21899955 | Negative | Negative | Negative |
| 7649.9_faster1 | | 110915 | INDI 697 PL [symbol] | Breast | II | 21937982 | Negative | Negative | Negative |
| 7662.9_faster1 | | 110924 | INDI 338 PL [symbol] | Colorectum | II | 21995745 | Negative | Negative | Negative |
| 7012.12_faster1 | | 108944 | INDI 466 PL [symbol] | Colorectum | II | 22048167 | Positive | Negative | Negative |
| 7549.3_faster1 | | 10879 | INDI 417 PL [symbol] | Lung | I | 22199612 | Negative | Negative | Negative |
| 7561.10_faster1 | | 109111 | INDI 824 PL [symbol] | Esophagus | II | 22217017 | Negative | Negative | Negative |
| 7671.4_faster1 | | 110989 | INDI 746 PL [symbol] | Colorectum | III | 22277590 | Negative | Negative | Negative |
| 7549.8_faster1 | | 108967 | INDI 499 PL [symbol] | Lung | I | 22290340 | Negative | Negative | Negative |
| 7043.9_faster1 | | 10547 | CRC 504 PL [symbol] | Colorectum | III | 22408133 | Positive | Negative | Negative |
| 7670.4_faster1 | | 110980 | INDI 648 PL [symbol] | Colorectum | III | 22510880 | Positive | Negative | Negative |
| 7043.11_faster1 | | 10549 | CRC 512 PL [symbol] | Colorectum | II | 22667843 | Negative | Negative | Negative |
| 6835.5_faster1 | | 110679 | PAP 961 PL [symbol] | Ovary | III | 22676074 | Negative | Negative | Negative |
| 7649.12_faster1 | | 110918 | INDI 714 PL [symbol] | Breast | III | 22684049 | Positive | Negative | Negative |
| 7662.5_faster1 | | 110920 | INDI 322 PL [symbol] | Colorectum | III | 22726250 | Positive | Negative | Negative |
| 6858.6_faster1 | | 10544 | CRC 497 PL [symbol] | Colorectum | III | 22727295 | Negative | Negative | Negative |
| 7014.6_faster1 | | 10852 | INDI 380 PL [symbol] | Colorectum | II | 23010226 | Negative | Negative | Negative |
| 7010.7_faster1 | | 10740 | INDI 211 PL [symbol] | Colorectum | II | 23031581 | Negative | Negative | Negative |
| 7009.3_faster1 | | 110627 | PANC 762 PL [symbol] | Pancreas | II | 23155883 | Negative | Negative | Negative |
| 7012.5_faster1 | | 108942 | INDI 464 PL [symbol] | Liver | III | 23250231 | Positive | Negative | Positive |
| 7013.11_faster1 | | 10822 | INDI 340 PL [symbol] | Colorectum | III | 23297382 | Negative | Negative | Negative |
| 7637.9_faster1 | | 110805 | INDI 304 PL [symbol] | Colorectum | I | 23389735 | Negative | Positive | Negative |
| 7594.12_faster1 | | 109149 | INDI 913 PL [symbol] | Esophagus | III | 23543415 | Negative | Negative | Negative |
| 7612.7_faster1 | | 10741 | INDI 212 PL [symbol] | Colorectum | III | 23614346 | Positive | Negative | Negative |
| 7010.10_faster1 | | 108970 | INDI 505 PL [symbol] | Lung | II | 23634307 | Negative | Negative | Negative |
| 7606.7_faster1 | Yes | 10628 | INDI 079 PL [symbol] | Liver | II | 23851041 | Negative | Negative | Negative |
| 7013.7_faster1 | | 109017 | INDI 625 PL [symbol] | Colorectum | II | 23884864 | Negative | Negative | Negative |
| 6837.5_faster1 | | 108963 | INDI 494 PL [symbol] | Lung | III | 23965761 | Negative | Negative | Negative |
| 7544.12_faster1 | | 10560 | INDI 005 PL [symbol] | Lung | I | 23999311 | Positive | Negative | Negative |
| 7542.6_faster1 | | 110638 | PANCA 101 [symbol] | Pancreas | II | 24010534 | Negative | Negative | Negative |
| 7594.7_faster1 | | 109144 | INDI 905 PL [symbol] | Esophagus | II | 24032028 | Negative | Negative | Negative |
| 7562.8_faster1 | | 109104 | INDI 801 PL [symbol] | Stomach | I | 24096871 | Negative | Negative | Negative |
| 7645.9_faster1 | | 110875 | INDI 601 PL [symbol] | Breast | III | 24221059 | Positive | Negative | Negative |
| 7009.12_faster1 | | 10583 | INDI 034 PL [symbol] | Colorectum | III | 24367937 | Negative | Negative | Negative |
| 7014.7_faster1 | | 10866 | INDI 397 PL [symbol] | Colorectum | III | 24491709 | Negative | Negative | Negative |
| 7639.10_faster1 | | 110826 | INDI 444 PL [symbol] | Colorectum | II | 24690075 | Negative | Negative | Negative |
| 7589.4_faster1 | | 109046 | INDI 693 PL [symbol] | Lung | III | 24743114 | Positive | Negative | Negative |
| 7602.8_faster1 | | 10592 | INDI 044 PL [symbol] | Colorectum | II | 25416842 | Negative | Negative | Negative |
| 7662.8_faster1 | | 110923 | INDI 329 PL [symbol] | Colorectum | IIII | 25554567 | Positive | Negative | Negative |
| 7637.10_faster1 | | 110806 | INDI 305 PL [symbol] | Colorectum | II | 25620843 | Positive | Negative | Negative |
| 7013.10_faster1 | | 10809 | INDI 320 PL [symbol] | Colorectum | II | 25848426 | Negative | Negative | Negative |
| 6835.4_faster1 | | 110671 | PAP 949 PL [symbol] | Ovary | IIII | 26009990 | Negative | Negative | Negative |
| 7610.3_faster1 | | 10687 | INDI 150 PL [symbol] | Colorectum | II | 26190880 | Positive | Negative | Negative |
| 7663.10_faster1 | | 110936 | INDI 400 PL [symbol] | Colorectum | II | 26205126 | Positive | Negative | Negative |
| 7013.3_faster1 | | 108946 | INDI 470 PL [symbol] | Colorectum | III | 26290647 | Positive | Negative | Negative |
| 7678.3_faster1 | | 111039 | INDI 917 PL [symbol] | Liver | III | 26418631 | Positive | Negative | Negative |
| 7740.8_faster1 | | 111064 | CRC 476 PL [symbol] | Colorectum | II | 26527000 | Positive | Negative | Negative |
| 7665.3_faster1 | | 110949 | INDI 670 PL [symbol] | Colorectum | III | 26759855 | Positive | Negative | Negative |
| 6857.11_faster1 | | 110662 | PANCA 115 [symbol] | Pancreas | II | 26760223 | Negative | Negative | Negative |
| 6839_redo.11_faster1 | | 109018 | INDI 626 PL [symbol] | Colorectum | II | 26767307 | Negative | Negative | Negative |
| 7673.5_faster1 | | 111012 | INDI 788 PL [symbol] | Colorectum | II | 27075277 | Negative | Negative | Negative |
| 6837.10_faster1 | | 10665 | INDI 125 PL [symbol] | Esophagus | II | 27099980 | Positive | Positive | Negative |
| 7535.10_faster1 | | 110684 | PAPA 1332 [symbol] | Ovary | I | 27521236 | Negative | Negative | Negative |
| 7011.10_faster1 | | 108936 | INDI 457 PL [symbol] | Esophagus | II | 27665822 | Positive | Negative | Negative |
| 7677.5_faster1 | | 111031 | INDI 831 PL [symbol] | Breast | I | 27831412 | Negative | Negative | Negative |
| 7537.4_faster1 | | 110699 | PAPA 135 [symbol] | Ovary | III | 27866616 | Positive | Negative | Negative |
| 7638.3_faster1 | | 110809 | INDI 309 PL [symbol] | Colorectum | II | 27979944 | Negative | Negative | Negative |
| 6838_redo.9_faster1 | | 10747 | INDI 219 PL [symbol] | Liver | III | 28101753 | Positive | Negative | Negative |
| 7566.3_faster1 | | 10675 | INDI 135 PL [symbol] | Stomach | III | 28195882 | Negative | Negative | Negative |
| 7640.4_faster1 | | 110830 | INDI 467 PL [symbol] | Colorectum | II | 28285870 | Positive | Negative | Negative |
| 7561.8_faster1 | | 109093 | INDI 784 PL [symbol] | Stomach | I | 28355787 | Negative | Negative | Negative |
| 7602.10_faster1 | | 10994 | INDI 046 PL [symbol] | Colorectum | II | 28577142 | Negative | Negative | Negative |
| 7041.6_faster1 | | 109022 | INDI 640 PL [symbol] | Colorectum | II | 28800336 | Positive | Positive | Negative |
| 7567.5_faster1 | | 10884 | INDI 440 PL [symbol] | Esophagus | II | 29087757 | Negative | Negative | Negative |
| 6851.9_faster1 | | 109025 | INDI 647 PL [symbol] | Colorectum | III | 29099826 | Positive | Negative | Negative |
| 7644.5_faster1 | | 110866 | INDI 631 PL [symbol] | Colorectum | II | 29183100 | Negative | Negative | Negative |
| 7042.10_faster1 | | 10532 | CRC 458 PL [symbol] | Colorectum | II | 29493266 | Negative | Negative | Negative |
| 7609.6_faster1 | | 10663 | INDI 123 PL [symbol] | Colorectum | II | 29592186 | Negative | Negative | Negative |
| 7593.5_faster1 | | 10837 | INDI 360 PL [symbol] | Lung | II | 29802114 | Negative | Negative | Negative |
| 7011.5_faster1 | | 109029 | INDI 663 PL [symbol] | Esophagus | II | 30466055 | Positive | Negative | Negative |
| 7677.6_faster1 | | 111032 | INDI 832 PL [symbol] | Breast | II | 31310565 | Negative | Negative | Negative |
| 7041.9_faster1 | | 109026 | INDI 657 PL [symbol] | Colorectum | II | 31653547 | Positive | Negative | Negative |
| 6857.3_faster1 | | 109063 | INDI 720 PL [symbol] | Colorectum | III | 31915897 | Negative | Negative | Negative |
| 7010.6_faster1 | | 10723 | INDI 191 PL [symbol] | Colorectum | I | 32107486 | Negative | Negative | Negative |
| 7542.5_faster1 | | 110637 | PANCA 101 [symbol] | Pancreas | III | 32393583 | Positive | Negative | Negative |
| 7592_combined.8_fast [symbol] | Yes | 10819 | INDI 336 PL [symbol] | Lung | III | 32529369 | Negative | Negative | Negative |
| 7586.8_faster1 | | 109028 | INDI 661 PL [symbol] | Liver | III | 32614452 | Negative | Negative | Negative |
| 7560.3_faster1 | | 108979 | INDI 522 PL [symbol] | Lung | I | 32694027 | Negative | Negative | Negative |
| 7667.12_faster1 | | 110977 | INDI 716 PL [symbol] | Colorectum | III | 32862529 | Negative | Negative | Negative |
| 6851.7_faster1 | | 109054 | INDI 702 PL [symbol] | Lung | II | 33124065 | Negative | Negative | Negative |
| 7673.9_faster1 | | 111015 | INDI 808 PL [symbol] | Colorectum | III | 33215174 | Negative | Negative | Negative |
| 7640.11_faster1 | | 110837 | INDI 562 PL [symbol] | Colorectum | I | 33273561 | Positive | Negative | Negative |
| 7566.12_faster1 | | 10642 | INDI 095 PL [symbol] | Stomach | II | 33345327 | Positive | Negative | Negative |
| 6838_redo.7_faster1 | | 10714 | INDI 182 PL [symbol] | Liver | III | 33731393 | Negative | Negative | Negative |
| 7563.9_faster1 | | 109131 | INDI 867 PL [symbol] | Esophagus | II | 33771332 | Positive | Negative | Negative |
| 7664.8_faster1 | | 110944 | INDI 525 PL [symbol] | Colorectum | I | 33945588 | Negative | Negative | Negative |
| 7589.11_faster1 | | 10843 | INDI 366 PL [symbol] | Pancreas | II | 33972894 | Negative | Negative | Negative |
| 7759.10_faster1 | Yes | 10669 | INDI 129 PL [symbol] | Liver | II | 34138426 | Negative | Negative | Negative |
| 7564.3_faster1 | | 10716 | INDI 184 PL [symbol] | Esophagus | II | 34571164 | Positive | Negative | Negative |
| 7560.5_faster1 | | 108986 | INDI 535 PL [symbol] | Lung | I | 35034507 | Negative | Negative | Negative |
| 7646.4_faster1 | | 110879 | INDI 607 PL [symbol] | Breast | II | 35885421 | Negative | Negative | Negative |
| 7592_combined.3_fast [symbol] | Yes | 10868 | INDI 403 PL [symbol] | Lung | III | 35927206 | Negative | Negative | Negative |
| 7742.12_faster1 | | 111088 | CRC 513 PL [symbol] | Colorectum | II | 36090588 | Negative | Negative | Negative |
| 7594.9_faster1 | | 109146 | INDI 908 PL [symbol] | Esophagus | II | 36616898 | Negative | Negative | Negative |
| 7548.3_faster1 | | 10779 | INDI 269 PL [symbol] | Lung | III | 36802049 | Negative | Negative | Negative |
| 7649.11_faster1 | | 110917 | INDI 712 PL [symbol] | Breast | I | 37318457 | Negative | Negative | Negative |
| 7585.6_faster1 | | 10734 | INDI 205 PL [symbol] | Liver | III | 37490440 | Positive | Positive | Negative |
| 7042.12_faster1 | | 10535 | CRC 468 PL [symbol] | Colorectum | III | 38370073 | Negative | Negative | Negative |
| 7014.5_faster1 | | 10851 | INDI 377 PL [symbol] | Colorectum | II | 38574612 | Negative | Negative | Negative |
| 7566.5_faster1 | | 10678 | INDI 139 PL [symbol] | Stomach | III | 38605637 | Positive | Negative | Negative |
| 7567.3_faster1 | | 10649 | INDI 103 PL [symbol] | Stomach | II | 39168812 | Negative | Negative | Negative |
| 7562.12_faster1 | | 109123 | INDI 850 PL [symbol] | Esophagus | II | 39489197 | Positive | Negative | Negative |
| 7740.4_faster1 | | 111060 | CRC 472 PL [symbol] | Colorectum | II | 39783916 | Negative | Negative | Negative |
| 7676.7_faster1 | | 111028 | INDI 837 PL [symbol] | Colorectum | II | 41486671 | Negative | Negative | Negative |
| 7563.5_faster1 | | 10697 | INDI 162 PL [symbol] | Stomach | III | 41846036 | Negative | Negative | Negative |
| 6837.3_faster1 | | 10769 | INDI 252 PL [symbol] | Lung | II | 41868738 | Negative | Negative | Negative |
| 7589.9_faster1 | | 109142 | INDI 893 PL [symbol] | Pancreas | II | 42034627 | Negative | Negative | Negative |
| 7596.6_faster1 | | 10602 | INDI 054 PL [symbol] | Breast | II | 43241792 | Negative | Negative | Negative |
| 7584.3_faster1 | | 10626 | INDI 077 PL [symbol] | Liver | II | 43439264 | Positive | Negative | Negative |
| 7612.12_faster1 | | 10750 | INDI 222 PL [symbol] | Colorectum | II | 44049554 | Positive | Negative | Negative |
| 7541.3_faster1 | | 110623 | PANC 677 [symbol] | Pancreas | II | 44382768 | Negative | Negative | Negative |
| 7672.6_faster1 | | 111006 | INDI 777 PL [symbol] | Colorectum | II | 44818555 | Negative | Negative | Negative |
| 7671.9_faster1 | | 110995 | INDI 765 PL [symbol] | Colorectum | I | 45084409 | Negative | Negative | Negative |
| 7012.10_faster1 | | 10801 | INDI 310 PL [symbol] | Colorectum | II | 45238888 | Negative | Negative | Negative |
| 7642.6_faster1 | | 110842 | INDI 582 PL [symbol] | Colorectum | II | 45318338 | Negative | Negative | Negative |
| 7739.4_faster1 | | 111050 | CRC 456 PL [symbol] | Colorectum | I | 45555883 | Negative | Negative | Negative |
| 7645.3_faster1 | | 110869 | INDI 578 PL [symbol] | Breast | I | 46297286 | Negative | Negative | Negative |
| 7643.11_faster1 | | 110857 | INDI 618 PL [symbol] | Colorectum | III | 46621165 | Negative | Positive | Negative |
| 7593.9_faster1 | | 10848 | INDI 372 PL [symbol] | Lung | II | 47063234 | Negative | Negative | Negative |
| 7678.S_faster1 | | 111044 | INDI 923 PL [symbol] | Esophagus | I | 48092734 | Negative | Positive | Negative |
| 7609.3_faster1 | | 10653 | INDI 109 PL [symbol] | Colorectum | III | 48226837 | Positive | Negative | Negative |
| 7537.6_faster1 | Yes | 109152 | LCR 812 PL [symbol] | Pancreas | II | 51896117 | Positive | Negative | Negative |
| 7547.4_faster1 | | 10752 | INDI 224 PL [symbol] | Lung | III | 51931089 | Positive | Negative | Negative |
| 7603.3_faster1 | Yes | 10638 | INDI 090 PL [symbol] | Colorectum | II | 52478584 | Positive | Negative | Negative |
| 7567.4_faster1 | | 108951 | INDI 476 PL [symbol] | Esophagus | II | 52566550 | Negative | Negative | Negative |
| 7590.12_faster1 | | 109073 | INDI 740 PL [symbol] | Pancreas | II | 53237733 | Negative | Negative | Negative |
| 7644.6_faster1 | | 110867 | INDI 632 PL [symbol] | Colorectum | II | 55764474 | Negative | Negative | Negative |
| 7009.4_faster1 | | 110628 | PANC 765 [symbol] | Pancreas | II | 56998190 | Negative | Negative | Negative |
| 7591.3_faster1 | | 109074 | INDI 741 PL [symbol] | Pancreas | III | 57886079 | Negative | Negative | Negative |
| 7666.3_faster1 | | 110967 | INDI 902 PL [symbol] | Breast | III | 59817502 | Positive | Negative | Negative |
| 7613.4_faster1 | | 10753 | INDI 225 PL [symbol] | Colorectum | I | 68078206 | Positive | Negative | Negative |

| | | CA19-9 | CEA | HGF | OPN | TIMP-1 | |
|---|---|---|---|---|---|---|---|
| | CA 15-3 + | (U/ml) + | (pg/ml) | (pg/ml) + | (pg/ml) + | (pg/ml) + | |
| Unique Name | (>98 U/ml) | >92 | >7507 | >899 | >157772 | >176989 | Mut+ |
| 7666.9_faster1 | Negative | Negative | Negative | Negative | Negative | Negative | Positive |
| 7561.9_faster1 | Negative | Negative | Negative | Negative | Negative | Negative | Negative |
| 7649.7_faster1 | Negative | Negative | Negative | Negative | Negative | Negative | Negative |
| 7678.11_faster1 | Negative | Negative | Negative | Negative | Negative | Negative | Negative |
| 7560.11_faster1 | Negative | Negative | Negative | Negative | Negative | Negative | Negative |
| 7537.3_faster1 | Negative | Negative | Negative | Negative | Negative | Negative | Negative |
| 7662.10_faster1 | Negative | Negative | Negative | Negative | Negative | Negative | Negative |
| 7613.11_faster1 | Negative | Negative | Negative | Negative | Negative | Negative | Negative |
| 7563.8_faster1 | Negative | Negative | Negative | Negative | Negative | Negative | Negative |
| 7537.1_faster1 | Negative | Positive | Positive | Negative | Negative | Negative | Positive |
| 7591.11_faster1 | Positive | Negative | Negative | Negative | Negative | Negative | Positive |
| 7586.5_faster1 | Negative | Negative | Positive | Positive | Positive | Positive | Positive |
| 7561.4_faster1 | Negative | Negative | Negative | Negative | Negative | Negative | Negative |
| 7589.8_faster1 | Negative | Negative | Negative | Negative | Negative | Negative | Positive |
| 7586.6_faster1 | Negative | Negative | Negative | Negative | Negative | Negative | Positive |
| 7671.10_faster1 | Negative | Negative | Positive | Negative | Negative | Negative | Negative |
| 7560.10_faster1 | Negative | Negative | Positive | Negative | Negative | Negative | Negative |
| 7740.10_faster1 | Negative | Negative | Negative | Negative | Negative | Negative | Negative |
| 7671.12_faster1 | Negative | Negative | Negative | Negative | Negative | Negative | Positive |
| 7013.8_faster1 | Negative | Negative | Negative | Negative | Negative | Negative | Positive |
| 7645.6_faster1 | Negative | Negative | Negative | Negative | Negative | Negative | Negative |
| 7014.12_faster1 | Negative | Negative | Negative | Negative | Negative | Negative | Positive |
| 7009.10_faster1 | Negative | Negative | Negative | Negative | Negative | Negative | Positive |
| 7541.9_faster1 | Negative | Negative | Negative | Negative | Negative | Negative | Negative |
| 7665.5_faster1 | Negative | Negative | Negative | Negative | Negative | Negative | Negative |
| 7541.11_faster1 | Negative | Positive | Positive | Negative | Negative | Negative | Positive |
| 7567.8_faster1 | Negative | Negative | Negative | Negative | Negative | Negative | Negative |
| 7542.10_faster1 | Negative | Negative | Negative | Negative | Negative | Negative | Negative |
| 7678.10_faster1 | Negative | Negative | Negative | Negative | Negative | Negative | Negative |
| 7673.12_faster1 | Negative | Negative | Negative | Negative | Negative | Negative | Negative |
| 7666.4_faster1 | Negative | Negative | Negative | Negative | Negative | Negative | Negative |
| 7562.10_faster1 | Negative | Positive | Negative | Negative | Negative | Negative | Negative |
| 7537.9_faster1 | Negative | Negative | Negative | Negative | Negative | Negative | Negative |
| 7594.3_faster1 | Negative | Negative | Negative | Negative | Negative | Negative | Positive |
| 6837.6_faster1 | Negative | Negative | Negative | Negative | Negative | Negative | Positive |
| 7613.10_faster1 | Negative | Negative | Negative | Negative | Negative | Negative | Negative |
| 7589.12_faster1 | Negative | Negative | Negative | Negative | Negative | Negative | Negative |
| 7537.12_faster1 | Negative | Positive | Negative | Negative | Positive | Negative | Negative |
| 7591.12_faster1 | Negative | Negative | Negative | Positive | Negative | Negative | Negative |
| 7012.7_faster1 | Negative | Negative | Negative | Negative | Negative | Negative | Positive |
| 7667.9_faster1 | Negative | Negative | Negative | Negative | Negative | Negative | Negative |
| 7548.9_faster1 | Negative | Negative | Negative | Negative | Negative | Negative | Negative |
| 7665.12_faster1 | Negative | Negative | Negative | Negative | Negative | Negative | Negative |
| 7671.3_faster1 | Negative | Negative | Negative | Negative | Negative | Negative | Negative |
| 7536.3_faster1 | Negative | Negative | Negative | Negative | Negative | Negative | Positive |
| 6857.4_faster1 | Negative | Negative | Negative | Negative | Negative | Negative | Positive |
| 7676.8_faster1 | Negative | Negative | Negative | Negative | Negative | Negative | Negative |
| 7561.7_faster1 | Negative | Negative | Negative | Negative | Negative | Negative | Negative |
| 7590.9_faster1 | Negative | Negative | Negative | Negative | Negative | Negative | Positive |
| 7591.8_faster1 | Negative | Positive | Negative | Negative | Negative | Negative | Negative |
| 7643.9_faster1 | Negative | Negative | Negative | Negative | Negative | Negative | Negative |
| 7613.6_faster1 | Negative | Negative | Negative | Negative | Negative | Negative | Negative |
| 7666.5_faster1 | Negative | Negative | Negative | Negative | Negative | Negative | Positive |
| 7666.8_faster1 | Negative | Negative | Negative | Negative | Negative | Negative | Negative |
| 7560.12_faster1 | Negative | Negative | Negative | Negative | Negative | Negative | Negative |
| 7590.4_faster1 | Negative | Negative | Negative | Negative | Negative | Negative | Negative |
| 7639.4_faster1 | Negative | Negative | Negative | Negative | Negative | Negative | Negative |
| 7637.3_faster1 | Negative | Negative | Positive | Negative | Negative | Negative | Negative |
| 7611.9 _faster1 | Negative | Negative | Negative | Positive | Negative | Negative | Positive |
| 7612.3 _faster1 | Negative | Negative | Negative | Positive | Negative | Negative | Negative |
| 7645.12_faster1 | Negative | Negative | Negative | Negative | Negative | Negative | Negative |
| 7646.5_faster1 | Negative | Negative | Negative | Negative | Negative | Negative | Negative |
| 7546.10_faster1 | Negative | Negative | Negative | Negative | Negative | Negative | Positive |
| 7613.7_faster1 | Negative | Negative | Negative | Negative | Negative | Negative | Positive |
| 7543.11_faster1 | Negative | Negative | Negative | Negative | Negative | Negative | Negative |
| 7676.11_faster1 | Negative | Negative | Negative | Negative | Negative | Negative | Positive |
| 7563.6_faster1 | Negative | Negative | Negative | Positive | Negative | Negative | Positive |
| 6851.10_faster1 | Negative | Negative | Negative | Negative | Negative | Negative | Positive |
| 7613.5_faster1 | Negative | Negative | Negative | Negative | Negative | Negative | Negative |
| 7672.10_faster1 | Negative | Negative | Negative | Negative | Negative | Negative | Negative |
| 7673.10_faster1 | Negative | Negative | Negative | Negative | Negative | Negative | Negative |
| 7544.3_faster1 | Negative | Positive | Negative | Negative | Negative | Negative | Negative |
| 7646.6_faster1 | Negative | Negative | Negative | Negative | Negative | Negative | Negative |
| 7041.3_faster1 | Negative | Negative | Negative | Negative | Negative | Negative | Positive |
| 6851.12_faster1 | Negative | Negative | Negative | Negative | Negative | Negative | Positive |
| 6837.11_faster1 | Negative | Negative | Negative | Negative | Negative | Negative | Positive |
| 7590.8_faster1 | Negative | Negative | Negative | Negative | Negative | Negative | Positive |
| 7638.11_faster1 | Negative | Negative | Positive | Negative | Negative | Positive | Negative |
| 7561.5_faster1 | Negative | Negative | Negative | Negative | Positive | Negative | Negative |
| 7567.10_faster1 | Negative | Negative | Negative | Negative | Negative | Negative | Negative |
| 7678.7_faster1 | Negative | Negative | Negative | Negative | Negative | Negative | Negative |
| 7740.7_faster1 | Negative | Negative | Negative | Negative | Negative | Negative | Negative |
| 7590.3_faster1 | Negative | Negative | Negative | Negative | Negative | Negative | Negative |
| 7589.3_faster1 | Negative | Negative | Negative | Negative | Negative | Negative | Negative |
| 7542.12_faster1 | Negative | Positive | Negative | Negative | Negative | Negative | Negative |
| 7678.4_faster1 | Negative | Negative | Negative | Negative | Negative | Negative | Positive |
| 7534.12_faster1 | Negative | Positive | Negative | Negative | Negative | Negative | Positive |
| 7642.12_faster1 | Negative | Negative | Positive | Negative | Negative | Negative | Negative |
| 7611.6_faster1 | Negative | Negative | Negative | Positive | Negative | Negative | Negative |
| 7541.12_faster1 | Negative | Negative | Negative | Negative | Negative | Negative | Negative |
| 7585.5_faster1 | Negative | Negative | Negative | Positive | Negative | Positive | Negative |
| 7639.6_faster1 | Negative | Negative | Negative | Negative | Negative | Negative | Negative |
| 7592_combined.7_fast [symbol] | Negative | Negative | Negative | Negative | Negative | Negative | Negative |
| 7677.4_faster1 | Negative | Negative | Negative | Negative | Negative | Negative | Negative |
| 7566.8_faster1 | Negative | Negative | Negative | Negative | Negative | Negative | Negative |
| 7739.8_faster1 | Negative | Negative | Negative | Negative | Negative | Negative | Negative |
| 7584.8_faster1 | Negative | Negative | Negative | Positive | Negative | Negative | Negative |
| 7670.10_faster1 | Negative | Negative | Negative | Negative | Negative | Negative | Negative |
| 7549.6_faster1 | Negative | Negative | Negative | Negative | Negative | Negative | Negative |
| 7535.5_faster1 | Negative | Negative | Negative | Negative | Negative | Negative | Positive |
| 7740.6_faster1 | Negative | Negative | Negative | Negative | Negative | Negative | Negative |
| 7542.9_faster1 | Negative | Negative | Negative | Negative | Negative | Positive | Negative |
| 7589.10_faster1 | Positive | Negative | Negative | Negative | Negative | Negative | Positive |
| 7640.7_faster1 | Negative | Negative | Negative | Negative | Negative | Negative | Negative |
| 6857.9_faster1 | Negative | Negative | Negative | Negative | Negative | Negative | Positive |
| 7667.10_faster1 | Negative | Negative | Negative | Negative | Negative | Negative | Negative |
| 7649.4_faster1 | Negative | Negative | Negative | Negative | Negative | Negative | Negative |
| 7640.8_faster1 | Negative | Negative | Negative | Negative | Negative | Negative | Negative |
| 7643.6_faster1 | Negative | Negative | Negative | Negative | Negative | Negative | Negative |
| 7543.4_faster1 | Negative | Negative | Negative | Negative | Negative | Negative | Negative |
| 7589.6_faster1 | Negative | Negative | Negative | Negative | Negative | Negative | Positive |
| 7638.12_faster1 | Negative | Negative | Positive | Negative | Negative | Positive | Negative |
| 7563.11_faster1 | Negative | Negative | Negative | Positive | Negative | Negative | Negative |
| 6839_redo.8_faster1 | Negative | Negative | Negative | Negative | Negative | Negative | Positive |
| 7643.10_faster1 | Negative | Negative | Negative | Negative | Negative | Negative | Negative |
| 7547.6_faster1 | Negative | Negative | Positive | Negative | Negative | Negative | Positive |
| 7664.12_faster1 | Negative | Negative | Negative | Negative | Negative | Negative | Negative |
| 7677.10_faster1 | Negative | Negative | Negative | Negative | Negative | Negative | Negative |
| 7591.4_faster1 | Negative | Negative | Negative | Negative | Negative | Negative | Positive |
| 7671.11_faster1 | Negative | Negative | Negative | Negative | Negative | Negative | Negative |
| 7591.5_faster1 | Negative | Negative | Negative | Negative | Negative | Negative | Negative |
| 7672.12_faster1 | Negative | Negative | Positive | Negative | Negative | Negative | Positive |
| 7586.7_faster1 | Negative | Negative | Negative | Negative | Positive | Negative | Positive |
| 7562.4_faster1 | Negative | Negative | Negative | Negative | Negative | Negative | Negative |
| 7566.9_faster1 | Negative | Positive | Negative | Negative | Negative | Negative | Positive |
| 7664.10_faster1 | Negative | Negative | Negative | Negative | Negative | Negative | Positive |
| 7676.4_faster1 | Negative | Negative | Negative | Negative | Negative | Negative | Negative |
| 7547.11_faster1 | Positive | Negative | Negative | Negative | Negative | Negative | Negative |
| 7638.5_faster1 | Negative | Negative | Negative | Negative | Negative | Negative | Negative |
| 7664.9_faster1 | Negative | Negative | Negative | Negative | Negative | Negative | Negative |
| 7603.12_faster1 | Negative | Negative | Negative | Negative | Negative | Negative | Positive |
| 6835.3_faster1 | Negative | Negative | Negative | Negative | Negative | Negative | Positive |
| 7677.3_faster1 | Negative | Negative | Negative | Negative | Negative | Negative | Negative |
| 7013.6_faster1 | Negative | Negative | Negative | Negative | Negative | Negative | Positive |
| 7305.10_faster1 | Negative | Negative | Negative | Positive | Negative | Negative | Negative |
| 7678.12_faster1 | Negative | Negative | Negative | Negative | Negative | Negative | Negative |
| 7011.7_faster1 | Negative | Negative | Positive | Positive | Negative | Negative | Positive |
| 7610.10_faster1 | Negative | Negative | Negative | Negative | Negative | Negative | Negative |
| 7637.7_faster1 | Negative | Negative | Negative | Negative | Negative | Negative | Negative |
| 7589.7_faster1 | Negative | Positive | Negative | Negative | Negative | Negative | Negative |
| 7013.12_faster1 | Negative | Negative | Negative | Negative | Negative | Negative | Positive |
| 7536.6_faster1 | Negative | Negative | Negative | Negative | Negative | Negative | Positive |
| 7560.7_faster1 | Negative | Positive | Positive | Negative | Negative | Negative | Positive |
| 7585.10_faster1 | Negative | Negative | Negative | Positive | Positive | Negative | Positive |
| 7640.12_faster1 | Negative | Negative | Negative | Negative | Negative | Negative | Negative |
| 7543.3_faster1 | Negative | Negative | Negative | Negative | Negative | Negative | Negative |
| 7042.3_faster1 | Negative | Negative | Negative | Negative | Negative | Negative | Positive |
| 7645.8_faster1 | Negative | Negative | Negative | Negative | Negative | Negative | Negative |
| 6858.5_faster1 | Negative | Negative | Negative | Negative | Negative | Negative | Positive |
| 7663.11_faster1 | Negative | Negative | Negative | Negative | Negative | Negative | Negative |
| 7610.12_faster1 | Negative | Negative | Negative | Negative | Negative | Negative | Negative |
| 7672.5_faster1 | Negative | Negative | Negative | Negative | Negative | Negative | Negative |
| 7549.11_faster1 | Negative | Negative | Negative | Negative | Negative | Negative | Negative |
| 7662.12_faster1 | Negative | Negative | Positive | Negative | Negative | Negative | Negative |
| 7666.11_faster1 | Negative | Negative | Negative | Negative | Negative | Negative | Negative |
| 7542.4_faster1 | Negative | Positive | Negative | Negative | Negative | Negative | Negative |
| 7677.7_faster1 | Negative | Negative | Negative | Negative | Negative | Negative | Negative |
| 7677.8_faster1 | Negative | Negative | Negative | Negative | Negative | Negative | Negative |
| 7672.3_faster1 | Negative | Negative | Negative | Negative | Negative | Negative | Negative |
| 7593.8_faster1 | Negative | Negative | Negative | Negative | Negative | Negative | Positive |
| 7560.8_faster1 | Negative | Negative | Negative | Negative | Negative | Negative | Negative |
| 7535.8_faster1 | Negative | Negative | Negative | Negative | Negative | Negative | Positive |
| 7562.6_faster1 | Negative | Negative | Negative | Negative | Negative | Negative | Negative |
| 7662.3_faster1 | Negative | Negative | Negative | Negative | Negative | Negative | Positive |
| 7640.6_faster1 | Negative | Negative | Positive | Negative | Negative | Negative | Negative |
| 7546.6_faster1 | Negative | Negative | Negative | Negative | Negative | Negative | Negative |
| 7010.8_faster1 | Negative | Negative | Negative | Negative | Negative | Negative | Positive |
| 7041.11_faster1 | Negative | Negative | Negative | Negative | Positive | Negative | Positive |
| 7542.7_faster1 | Negative | Positive | Negative | Negative | Negative | Negative | Negative |
| 7676.10_faster1 | Negative | Negative | Negative | Negative | Negative | Negative | Negative |
| 7010.11_faster1 | Negative | Negative | Negative | Negative | Negative | Negative | Negative |
| 7593.10_faster1 | Negative | Negative | Negative | Negative | Negative | Negative | Negative |
| 7601.12_faster1 | Negative | Negative | Negative | Negative | Negative | Negative | Negative |
| 7611.7_faster1 | Negative | Negative | Negative | Positive | Positive | Negative | Negative |
| 7603.5_faster1 | Negative | Negative | Negative | Negative | Positive | Positive | Negative |
| 7672.7_faster1 | Negative | Negative | Negative | Negative | Negative | Negative | Positive |
| 7563.10_faster1 | Negative | Negative | Negative | Negative | Negative | Negative | Negative |
| 7536.10_faster1 | Negative | Positive | Negative | Negative | Negative | Negative | Positive |
| 7664.11_faster1 | Negative | Negative | Negative | Negative | Negative | Negative | Positive |
| 7534.8_faster1 | Negative | Negative | Negative | Negative | Negative | Negative | Negative |
| 6836.4_faster1 | Negative | Negative | Negative | Negative | Negative | Negative | Negative |
| 7603.10_faster1 | Negative | Negative | Negative | Negative | Negative | Negative | Negative |
| 7542.8_faster1 | Negative | Negative | Negative | Negative | Negative | Negative | Negative |
| 7676.3_faster1 | Negative | Negative | Negative | Negative | Negative | Negative | Negative |
| 7609.5_faster1 | Negative | Negative | Negative | Positive | Negative | Negative | Negative |
| 6838_redo.4_faster1 | Negative | Negative | Negative | Negative | Negative | Negative | Negative |
| 7584.9_faster1 | Negative | Positive | Negative | Positive | Negative | Positive | Negative |
| 7612.5_faster1 | Negative | Negative | Positive | Negative | Negative | Negative | Negative |
| 7541.4_faster1 | Negative | Negative | Negative | Negative | Negative | Negative | Negative |
| 7613.9_faster1 | Negative | Negative | Negative | Negative | Negative | Negative | Negative |
| 7606.6_faster1 | Negative | Negative | Negative | Negative | Negative | Negative | Negative |
| 7602.3_faster1 | Negative | Negative | Negative | Negative | Negative | Negative | Negative |
| 7042.7_faster1 | Negative | Negative | Negative | Negative | Negative | Negative | Positive |
| 7041.12_faster1 | Negative | Negative | Negative | Negative | Negative | Negative | Positive |
| 7642.5_faster1 | Negative | Negative | Negative | Negative | Negative | Negative | Negative |
| 7672.4_faster1 | Negative | Negative | Negative | Negative | Negative | Negative | Negative |
| 7535.6_faster1 | Negative | Positive | Negative | Negative | Negative | Negative | Positive |
| 7662.6_faster1 | Negative | Negative | Negative | Negative | Negative | Negative | Negative |
| 7673.8_faster1 | Negative | Negative | Positive | Negative | Negative | Negative | Positive |
| 7534.3_faster1 | Negative | Negative | Negative | Negative | Negative | Negative | Positive |
| 7613.8_faster1 | Negative | Negative | Positive | Negative | Negative | Negative | Negative |
| 7759.5_faster1 | Negative | Positive | Positive | Negative | Negative | Negative | Negative |
| 7739.6_faster1 | Negative | Negative | Negative | Negative | Negative | Negative | Negative |
| 7637.5_faster1 | Negative | Negative | Negative | Negative | Negative | Negative | Negative |
| 7603.6_faster1 | Negative | Negative | Negative | Negative | Negative | Negative | Negative |
| 7537.5_faster1 | Negative | Negative | Negative | Positive | Negative | Negative | Negative |
| 6857.5_faster1 | Negative | Negative | Negative | Negative | Negative | Negative | Positive |
| 7678.9_faster1 | Negative | Negative | Negative | Negative | Negative | Negative | Positive |
| 7541.7_faster1 | Negative | Positive | Negative | Negative | Negative | Negative | Negative |
| 7667.4_faster1 | Negative | Negative | Negative | Negative | Negative | Negative | Positive |
| 7672.8_faster1 | Negative | Negative | Negative | Negative | Negative | Negative | Negative |
| 7535.12_faster1 | Negative | Negative | Negative | Negative | Negative | Positive | Positive |
| 7665.9_faster1 | Negative | Negative | Negative | Negative | Negative | Negative | Negative |
| 7306.11_faster1 | Negative | Negative | Negative | Negative | Negative | Negative | Negative |
| 7536.4_faster1 | Negative | Negative | Negative | Negative | Negative | Positive | Positive |
| 7742.8_faster1 | Negative | Negative | Negative | Negative | Negative | Negative | Negative |
| 7546.5_faster1 | Negative | Negative | Negative | Negative | Negative | Negative | Negative |
| 7663.7_faster1 | Negative | Negative | Negative | Negative | Negative | Negative | Negative |
| 7671.5_faster1 | Negative | Negative | Negative | Negative | Negative | Positive | Negative |
| 7662.11_faster1 | Negative | Negative | Negative | Negative | Negative | Negative | Negative |
| 7566.6_faster1 | Negative | Negative | Negative | Positive | Positive | Negative | Negative |
| 7013.5_faster1 | Negative | Negative | Negative | Negative | Negative | Negative | Positive |
| 7010.9_faster1 | Negative | Negative | Positive | Negative | Negative | Negative | Positive |
| 7642.8_faster1 | Negative | Negative | Negative | Negative | Positive | Negative | Negative |
| 7759.4_faster1 | Negative | Negative | Negative | Negative | Negative | Negative | Negative |
| 7544.7_faster1 | Negative | Positive | Negative | Negative | Negative | Negative | Negative |
| 7546.7_faster1 | Negative | Negative | Negative | Negative | Negative | Negative | Negative |
| 6836.5_faster1 | Negative | Negative | Negative | Negative | Negative | Negative | Positive |
| 7673.7_faster1 | Negative | Negative | Negative | Negative | Negative | Negative | Negative |
| 7642.9_faster1 | Negative | Negative | Negative | Negative | Positive | Negative | Negative |
| 7562.7_faster1 | Negative | Negative | Negative | Negative | Negative | Negative | Negative |
| 7612.11_faster1 | Negative | Negative | Positive | Negative | Negative | Negative | Positive |
| 7670.12_faster1 | Negative | Negative | Negative | Negative | Negative | Negative | Negative |
| 6835.9_faster1 | Negative | Positive | Negative | Negative | Negative | Negative | Positive |
| 7590.10_faster1 | Negative | Negative | Negative | Negative | Negative | Negative | Positive |
| 7548.4_faster1 | Negative | Negative | Negative | Negative | Negative | Negative | Positive |
| 7742.9_faster1 | Negative | Negative | Negative | Negative | Negative | Negative | Negative |
| 7009.5_faster1 | Negative | Negative | Negative | Negative | Negative | Negative | Positive |
| 7673.11_faster1 | Negative | Negative | Positive | Negative | Negative | Negative | Positive |
| 7611.5_faster1 | Negative | Negative | Negative | Positive | Positive | Negative | Positive |
| 6838_redo.11_faster1 | Negative | Positive | Negative | Negative | Positive | Negative | Positive |
| 7565.10_faster1 | Negative | Negative | Negative | Negative | Negative | Negative | Negative |
| 7593.3_faster1 | Negative | Negative | Negative | Negative | Negative | Negative | Negative |
| 7673.6_faster1 | Negative | Negative | Negative | Negative | Negative | Negative | Negative |
| 7011.9_faster1 | Negative | Negative | Positive | Negative | Negative | Negative | Positive |
| 7603.7_faster1 | Negative | Negative | Negative | Negative | Negative | Negative | Negative |
| 7642.10_faster1 | Negative | Negative | Negative | Negative | Negative | Negative | Positive |
| 6851.3_faster1 | Negative | Negative | Negative | Negative | Negative | Negative | Positive |
| 7534.6_faster1 | Negative | Negative | Negative | Negative | Negative | Negative | Positive |
| 7586.4_faster1 | Negative | Negative | Negative | Positive | Positive | Negative | Negative |
| 7663.3_faster1 | Negative | Negative | Negative | Negative | Negative | Negative | Negative |
| 7544.5_faster1 | Negative | Positive | Negative | Negative | Negative | Negative | Negative |
| 6851.4_faster1 | Negative | Negative | Negative | Negative | Negative | Negative | Positive |
| 7307.12_faster1 | Negative | Negative | Negative | Negative | Negative | Negative | Negative |
| 7667.3_faster1 | Negative | Negative | Negative | Negative | Negative | Negative | Positive |
| 7757.10_faster1 | Negative | Negative | Negative | Negative | Negative | Negative | Negative |
| 7536.12_faster1 | Positive | Negative | Negative | Negative | Negative | Positive | Positive |
| 6836.11_faster1 | Negative | Negative | Negative | Negative | Negative | Negative | Positive |
| 7535.7_faster1 | Negative | Negative | Negative | Negative | Negative | Negative | Positive |
| 7640.3_faster1 | Negative | Negative | Positive | Negative | Negative | Negative | Positive |
| 7541.5_faster1 | Negative | Negative | Negative | Negative | Negative | Negative | Negative |
| 7609.12_faster1 | Negative | Negative | Negative | Positive | Positive | Positive | Negative |
| 7640.5_faster1 | Negative | Negative | Positive | Negative | Negative | Negative | Negative |
| 7014.3_faster1 | Negative | Negative | Positive | Negative | Negative | Negative | Positive |
| 7543.8_faster1 | Negative | Negative | Negative | Negative | Negative | Negative | Positive |
| 7639.11_faster1 | Negative | Negative | Negative | Positive | Negative | Negative | Positive |
| 6837.4_faster1 | Negative | Negative | Negative | Negative | Negative | Negative | Positive |
| 7665.8_faster1 | Negative | Negative | Negative | Negative | Negative | Negative | Negative |
| 7759.7_faster1 | Negative | Negative | Negative | Positive | Negative | Negative | Negative |
| 7535.11_faster1 | Positive | Positive | Negative | Negative | Negative | Negative | Negative |
| 7639.12_faster1 | Negative | Positive | Negative | Negative | Positive | Negative | Negative |
| 7671.6_faster1 | Negative | Negative | Negative | Negative | Negative | Positive | Negative |
| 7664.6_faster1 | Negative | Negative | Negative | Negative | Negative | Negative | Negative |
| 7739.10_faster1 | Negative | Negative | Negative | Negative | Negative | Negative | Negative |
| 7308.4_faster1 | Negative | Negative | Negative | Negative | Positive | Negative | Positive |
| 7645.11_faster1 | Negative | Negative | Negative | Negative | Negative | Negative | Negative |
| 7541.8_faster1 | Negative | Positive | Negative | Negative | Negative | Negative | Positive |
| 7758.5_faster1 | Negative | Negative | Negative | Negative | Negative | Negative | Negative |
| 7541.6_faster1 | Negative | Positive | Negative | Negative | Negative | Negative | Negative |
| 6835.7_faster1 | Negative | Negative | Negative | Negative | Negative | Negative | Positive |
| 7593.6_faster1 | Negative | Negative | Negative | Negative | Negative | Negative | Negative |
| 7676.12_faster1 | Negative | Negative | Negative | Negative | Negative | Negative | Negative |
| 7667.6_faster1 | Negative | Negative | Negative | Negative | Negative | Negative | Negative |
| 7643.8_faster1 | Negative | Negative | Negative | Negative | Negative | Negative | Negative |
| 7544.8_faster1 | Negative | Negative | Negative | Negative | Negative | Negative | Negative |
| 7548.8_faster1 | Negative | Negative | Negative | Negative | Negative | Negative | Negative |
| 7670.6_faster1 | Negative | Negative | Negative | Negative | Negative | Negative | Negative |
| 7601.7_faster1 | Negative | Negative | Negative | Negative | Negative | Negative | Negative |
| 7606.8_faster1 | Negative | Negative | Negative | Negative | Negative | Negative | Negative |
| 7014.11_faster1 | Negative | Positive | Negative | Negative | Negative | Negative | Positive |
| 7670.9_faster1 | Negative | Negative | Negative | Negative | Negative | Negative | Negative |
| 7664.4_faster1 | Negative | Negative | Negative | Negative | Negative | Negative | Negative |
| 7759.3_faster1 | Negative | Negative | Negative | Negative | Negative | Negative | Negative |
| 6836.8_faster1 | Negative | Negative | Negative | Negative | Negative | Negative | Positive |
| 7564.4_faster1 | Negative | Negative | Positive | Positive | Positive | Negative | Positive |
| 6850.10_faster1 | Negative | Negative | Negative | Negative | Negative | Negative | Positive |
| 7307.9_faster1 | Negative | Negative | Negative | Negative | Negative | Negative | Negative |
| 7014.4_faster1 | Negative | Negative | Negative | Negative | Negative | Negative | Positive |
| 7676.5_faster1 | Negative | Negative | Negative | Negative | Negative | Negative | Negative |
| 7673.3_faster1 | Negative | Negative | Negative | Negative | Negative | Negative | Negative |
| 7639.9_faster1 | Negative | Negative | Negative | Negative | Negative | Negative | Negative |
| 7308.8_faster1 | Negative | Negative | Negative | Negative | Negative | Negative | Negative |
| 6851.5_faster1 | Negative | Negative | Positive | Negative | Negative | Negative | Positive |
| 7756.3_faster1 | Negative | Negative | Negative | Negative | Negative | Negative | Positive |
| 7637.8_faster1 | Negative | Negative | Negative | Negative | Negative | Negative | Negative |
| 7643.7_faster1 | Negative | Negative | Negative | Negative | Negative | Negative | Negative |
| 6835.11_faster1 | Negative | Negative | Negative | Negative | Negative | Negative | Positive |
| 7304.12_faster1 | Negative | Negative | Negative | Negative | Negative | Negative | Negative |
| 7308.7_faster1 | Negative | Negative | Negative | Negative | Negative | Negative | Negative |
| 7542.11_faster1 | Negative | Negative | Negative | Negative | Negative | Negative | Negative |
| 6836.12_faster1 | Negative | Negative | Negative | Negative | Negative | Negative | Positive |
| 7547.12_faster1 | Negative | Negative | Negative | Negative | Negative | Negative | Negative |
| 7590.11_faster1 | Negative | Negative | Negative | Negative | Negative | Negative | Negative |
| 7612.9_faster1 | Negative | Negative | Negative | Negative | Negative | Negative | Negative |
| 7663.6_faster1 | Negative | Negative | Negative | Negative | Negative | Negative | Negative |
| 7546.4_faster1 | Negative | Positive | Negative | Negative | Negative | Negative | Negative |
| 7607.8_faster1 | Negative | Negative | Negative | Negative | Negative | Negative | Positive |
| 7308.10_faster1 | Negative | Negative | Negative | Negative | Negative | Negative | Negative |
| 7547.5_faster1 | Negative | Negative | Negative | Negative | Negative | Negative | Negative |
| 7677.12_faster1 | Negative | Negative | Negative | Negative | Negative | Negative | Negative |
| 7534.9_faster1 | Positive | Negative | Negative | Negative | Negative | Negative | Positive |
| 7561.6_faster1 | Negative | Negative | Negative | Negative | Negative | Negative | Positive |
| 7012.8_faster1 | Negative | Negative | Negative | Negative | Negative | Negative | Positive |
| 7306.10_faster1 | Negative | Negative | Negative | Negative | Negative | Negative | Negative |
| 7662.7_faster1 | Negative | Negative | Negative | Negative | Negative | Negative | Negative |
| 7610.4_faster1 | Negative | Negative | Negative | Negative | Negative | Negative | Negative |
| 7548.12_faster1 | Negative | Negative | Negative | Negative | Negative | Negative | Negative |
| 7592_combined.6_fast [symbol] | Negative | Negative | Negative | Negative | Negative | Negative | Negative |
| 7638.6_faster1 | Negative | Negative | Negative | Negative | Negative | Negative | Negative |
| 7594.10_faster1 | Negative | Negative | Negative | Negative | Negative | Negative | Negative |
| 7041.10_faster1 | Negative | Negative | Negative | Negative | Negative | Negative | Positive |
| 7609.11_faster1 | Negative | Negative | Negative | Negative | Negative | Negative | Negative |
| 6857.8_faster1 | Negative | Negative | Positive | Negative | Negative | Negative | Positive |
| 7543.9_faster1 | Negative | Negative | Negative | Negative | Negative | Negative | Negative |
| 6838_redo.10_faster1 | Negative | Negative | Negative | Positive | Negative | Positive | Negative |
| 7739.3_faster1 | Negative | Negative | Negative | Negative | Negative | Negative | Positive |
| 6858.8_faster1 | Negative | Negative | Negative | Negative | Negative | Negative | Negative |
| 7303.4_faster1 | Negative | Negative | Negative | Positive | Negative | Negative | Negative |
| 7043.10_faster1 | Negative | Negative | Negative | Negative | Negative | Negative | Positive |
| 7548.6_faster1 | Positive | Negative | Negative | Negative | Negative | Negative | Positive |
| 7305.11_faster1 | Negative | Negative | Negative | Positive | Negative | Negative | Negative |
| 7308.3_faster1 | Negative | Negative | Negative | Negative | Negative | Negative | Negative |
| 7757.7_faster1 | Negative | Negative | Negative | Negative | Negative | Negative | Negative |
| 7611.10_faster1 | Negative | Negative | Negative | Positive | Negative | Negative | Negative |
| 7608.4_faster1 | Negative | Negative | Negative | Negative | Negative | Negative | Positive |
| 7757.12_faster1 | Negative | Negative | Negative | Negative | Negative | Negative | Negative |
| 7606.9_faster1 | Negative | Negative | Negative | Negative | Negative | Negative | Negative |
| 7603.8_faster1 | Negative | Negative | Negative | Negative | Negative | Negative | Negative |
| 7608.10_faster1 | Negative | Negative | Positive | Negative | Negative | Negative | Positive |
| 7041.4_faster1 | Negative | Negative | Negative | Negative | Negative | Negative | Positive |
| 7592_combined.12_fas [symbol] | Negative | Negative | Negative | Negative | Negative | Negative | Positive |
| 7640.10_faster1 | Negative | Negative | Negative | Negative | Negative | Negative | Negative |
| 7601.6_faster1 | Negative | Negative | Negative | Negative | Negative | Negative | Negative |
| 7606.10_faster1 | Negative | Negative | Negative | Negative | Negative | Negative | Positive |
| 7305.3_faster1 | Negative | Negative | Negative | Negative | Negative | Negative | Negative |
| 7303.10_faster1 | Negative | Negative | Negative | Negative | Positive | Negative | Negative |
| 7042.9_faster1 | Negative | Negative | Negative | Negative | Negative | Negative | Positive |
| 7592_combined.5_fast [symbol] | Negative | Negative | Negative | Negative | Negative | Negative | Positive |
| 7536.8_faster1 | Negative | Negative | Negative | Negative | Negative | Negative | Positive |
| 7543.7_faster1 | Negative | Positive | Negative | Negative | Negative | Negative | Negative |
| 7671.8_faster1 | Negative | Negative | Negative | Negative | Negative | Negative | Negative |
| 7646.10_faster1 | Negative | Negative | Negative | Negative | Negative | Negative | Negative |
| 7589.5_faster1 | Negative | Negative | Negative | Negative | Negative | Negative | Negative |
| 7642.4_faster1 | Negative | Negative | Negative | Negative | Negative | Negative | Negative |
| 7306.8_faster1 | Negative | Negative | Negative | Negative | Negative | Negative | Negative |
| 7563.12_faster1 | Negative | Negative | Negative | Positive | Negative | Negative | Negative |
| 7672.11_faster1 | Negative | Negative | Negative | Negative | Negative | Negative | Negative |
| 7760.10_faster1 | Negative | Negative | Negative | Negative | Negative | Negative | Negative |
| 7663.8_faster1 | Negative | Negative | Negative | Negative | Negative | Negative | Negative |
| 7011.3_faster1 | Negative | Negative | Negative | Negative | Negative | Positive | Positive |
| 7303.3_faster1 | Negative | Negative | Negative | Negative | Negative | Negative | Negative |
| 7637.11_faster1 | Negative | Negative | Positive | Negative | Negative | Positive | Positive |
| 7665.7_faster1 | Negative | Negative | Negative | Negative | Negative | Negative | Negative |
| 7606.11_faster1 | Negative | Negative | Negative | Negative | Negative | Negative | Negative |
| 7676.6_faster1 | Negative | Negative | Negative | Negative | Negative | Negative | Negative |
| 6857.10_faster1 | Negative | Negative | Negative | Negative | Negative | Negative | Positive |
| 7646.12_faster1 | Negative | Negative | Negative | Negative | Negative | Negative | Negative |
| 7644.4_faster1 | Negative | Negative | Negative | Negative | Negative | Negative | Negative |
| 7012.6_faster1 | Negative | Positive | Negative | Positive | Negative | Negative | Positive |
| 7759.12_faster1 | Negative | Negative | Negative | Negative | Negative | Negative | Negative |
| 7758.8_faster1 | Negative | Negative | Negative | Negative | Negative | Negative | Negative |
| 7642.3_faster1 | Negative | Negative | Negative | Negative | Negative | Negative | Negative |
| 7757.8_faster1 | Negative | Negative | Negative | Negative | Negative | Negative | Negative |
| 7637.4_faster1 | Negative | Negative | Negative | Negative | Negative | Negative | Negative |
| 7607.7_faster1 | Negative | Negative | Negative | Negative | Negative | Negative | Positive |
| 6837.12_faster1 | Negative | Negative | Positive | Negative | Negative | Negative | Positive |
| 6851.6_faster1 | Negative | Positive | Negative | Negative | Negative | Negative | Positive |
| 7041.8_faster1 | Negative | Negative | Negative | Negative | Negative | Negative | Positive |
| 6838_redo.8_faster1 | Negative | Negative | Negative | Negative | Negative | Negative | Positive |
| 7666.12_faster1 | Negative | Negative | Negative | Negative | Negative | Negative | Negative |
| 7590.5_faster1 | Negative | Negative | Negative | Negative | Negative | Negative | Negative |
| 7755.12_faster1 | Negative | Negative | Negative | Negative | Negative | Negative | Positive |
| 7608.9_faster1 | Positive | Negative | Negative | Negative | Negative | Negative | Positive |
| 7670.3_faster1 | Negative | Negative | Negative | Negative | Negative | Negative | Negative |
| 7644.3_faster1 | Negative | Negative | Negative | Negative | Negative | Negative | Negative |
| 7667.11_faster1 | Negative | Negative | Negative | Negative | Negative | Negative | Positive |
| 7544.10_faster1 | Negative | Positive | Positive | Negative | Negative | Negative | Positive |
| 7663.12_faster1 | Negative | Negative | Negative | Negative | Negative | Negative | Negative |
| 7593.4_faster1 | Negative | Negative | Negative | Negative | Negative | Negative | Negative |
| 7304.10_faster1 | Negative | Negative | Negative | Negative | Negative | Negative | Negative |
| 7638.9_faster1 | Negative | Negative | Negative | Negative | Negative | Positive | Negative |
| 7740.5_faster1 | Negative | Negative | Negative | Negative | Negative | Negative | Positive |
| 7608.7_faster1 | Negative | Negative | Positive | Negative | Negative | Negative | Positive |
| 6857.6_faster1 | Negative | Negative | Negative | Negative | Negative | Negative | Positive |
| 7307.11_faster1 | Negative | Negative | Negative | Negative | Negative | Negative | Negative |
| 7758.9_faster1 | Negative | Negative | Negative | Negative | Negative | Negative | Negative |
| 7759.9_faster1 | Negative | Positive | Positive | Negative | Negative | Negative | Positive |
| 7537.10_faster1 | Negative | Positive | Positive | Negative | Negative | Negative | Negative |
| 7739.9_faster1 | Negative | Negative | Negative | Negative | Negative | Negative | Negative |
| 7646.9_faster1 | Negative | Negative | Negative | Negative | Negative | Negative | Negative |
| 7609.10_faster1 | Negative | Negative | Negative | Negative | Negative | Negative | Negative |
| 7756.11_faster1 | Negative | Negative | Negative | Negative | Negative | Negative | Negative |
| 7607.12_faster1 | Negative | Negative | Negative | Negative | Negative | Negative | Positive |
| 7758.4_faster1 | Negative | Negative | Negative | Negative | Negative | Negative | Negative |
| 6850.8_faster1 | Negative | Negative | Negative | Negative | Negative | Negative | Positive |
| 7756.5_faster1 | Negative | Negative | Negative | Negative | Negative | Negative | Positive |
| 7759.8_faster1 | Negative | Negative | Negative | Positive | Negative | Negative | Negative |
| 7547.10_faster1 | Negative | Negative | Positive | Negative | Negative | Negative | Negative |
| 7758.6_faster1 | Negative | Negative | Negative | Negative | Negative | Negative | Negative |
| 6858.10_faster1 | Negative | Negative | Negative | Negative | Negative | Negative | Positive |
| 7541.10_faster1 | Negative | Negative | Negative | Negative | Negative | Negative | Negative |
| 7662.4_faster1 | Negative | Negative | Negative | Negative | Negative | Negative | Negative |
| 7561.3_faster1 | Negative | Negative | Negative | Negative | Negative | Negative | Negative |
| 7607.10_faster1 | Negative | Negative | Negative | Negative | Negative | Negative | Positive |
| 7306.12_faster1 | Negative | Negative | Negative | Negative | Negative | Negative | Negative |
| 7304.11_faster1 | Negative | Negative | Negative | Negative | Negative | Negative | Negative |
| 6850.12_faster1 | Negative | Negative | Positive | Negative | Negative | Negative | Positive |
| 7590.6_faster1 | Negative | Positive | Negative | Negative | Negative | Negative | Negative |
| 7306.6_faster1 | Negative | Negative | Negative | Negative | Negative | Negative | Negative |
| 7643.12_faster1 | Negative | Negative | Negative | Negative | Positive | Positive | Negative |
| 7665.10_faster1 | Negative | Negative | Positive | Negative | Negative | Negative | Negative |
| 7760.3_faster1 | Negative | Negative | Negative | Positive | Negative | Negative | Negative |
| 7756.9_faster1 | Negative | Negative | Negative | Negative | Negative | Negative | Negative |
| 7304.5_faster1 | Negative | Negative | Negative | Negative | Positive | Negative | Negative |
| 7534.7_faster1 | Negative | Negative | Negative | Negative | Negative | Positive | Positive |
| 6837.8_faster1 | Negative | Negative | Negative | Negative | Positive | Negative | Negative |
| 7304.3_faster1 | Negative | Negative | Negative | Negative | Negative | Negative | Negative |
| 7014.10_faster1 | Negative | Negative | Negative | Negative | Negative | Negative | Positive |
| 7643.5_faster1 | Negative | Negative | Negative | Negative | Negative | Negative | Negative |
| 7607.4_faster1 | Negative | Negative | Positive | Positive | Negative | Negative | Positive |
| 7593.7_faster1 | Negative | Negative | Negative | Negative | Negative | Negative | Negative |
| 7303.8_faster1 | Negative | Negative | Negative | Positive | Negative | Negative | Negative |
| 7756.6_faster1 | Negative | Negative | Negative | Negative | Negative | Negative | Negative |
| 7757.3_faster1 | Negative | Negative | Negative | Negative | Negative | Negative | Negative |
| 7546.3_faster1 | Negative | Negative | Positive | Negative | Negative | Negative | Negative |
| 7610.6_faster1 | Negative | Positive | Positive | Positive | Negative | Negative | Positive |
| 7670.5_faster1 | Negative | Negative | Positive | Negative | Negative | Negative | Negative |
| 7646.3_faster1 | Negative | Negative | Negative | Negative | Negative | Negative | Negative |
| 7677.11_faster1 | Negative | Negative | Negative | Negative | Negative | Negative | Negative |
| 7760.8_faster1 | Negative | Negative | Negative | Negative | Negative | Negative | Negative |
| 6839_redo.7_faster1 | Negative | Negative | Positive | Negative | Positive | Positive | Positive |
| 7756.4_faster1 | Negative | Negative | Negative | Negative | Negative | Negative | Positive |
| 7307.7_faster1 | Negative | Negative | Negative | Negative | Negative | Negative | Negative |
| 7303.9_faster1 | Negative | Negative | Negative | Negative | Negative | Negative | Negative |
| 7543.5_faster1 | Negative | Positive | Negative | Negative | Negative | Negative | Negative |
| 7549.7_faster1 | Negative | Negative | Negative | Negative | Negative | Negative | Negative |
| 7012.11_faster1 | Negative | Negative | Negative | Negative | Negative | Negative | Positive |
| 7760.6_faster1 | Negative | Negative | Positive | Positive | Positive | Negative | Negative |
| 7304.7_faster1 | Negative | Negative | Negative | Negative | Negative | Negative | Positive |
| 7758.10_faster1 | Negative | Negative | Negative | Negative | Negative | Negative | Negative |
| 7673.4_faster1 | Negative | Negative | Negative | Negative | Negative | Negative | Negative |
| 7756.10_faster1 | Negative | Negative | Negative | Negative | Negative | Negative | Negative |
| 7758.12_faster1 | Negative | Negative | Negative | Negative | Negative | Negative | Negative |
| 7760.4_faster1 | Negative | Negative | Negative | Negative | Negative | Negative | Negative |
| 7758.3_faster1 | Negative | Negative | Negative | Negative | Negative | Negative | Negative |
| 6850.4_faster1 | Negative | Negative | Positive | Negative | Negative | Negative | Positive |
| 7607.5_faster1 | Negative | Negative | Positive | Negative | Negative | Negative | Negative |
| 7759.11_faster1 | Negative | Negative | Negative | Negative | Positive | Negative | Negative |
| 6836.9_faster1 | Negative | Negative | Negative | Negative | Negative | Negative | Positive |
| 7307.5_faster1 | Negative | Negative | Negative | Negative | Negative | Negative | Negative |
| 7011.4_faster1 | Negative | Negative | Negative | Negative | Negative | Negative | Positive |
| 7042.11_faster1 | Negative | Negative | Negative | Negative | Negative | Negative | Positive |
| 7536.7_faster1 | Positive | Negative | Negative | Negative | Negative | Negative | Positive |
| 7758.11_faster1 | Negative | Negative | Negative | Negative | Negative | Negative | Negative |
| 7043.8_faster1 | Negative | Negative | Positive | Negative | Negative | Negative | Positive |
| 7305.12_faster1 | Negative | Positive | Negative | Negative | Positive | Negative | Negative |
| 7649.10_faster1 | Negative | Negative | Negative | Negative | Negative | Negative | Positive |
| 7596.8_faster1 | Negative | Negative | Negative | Positive | Negative | Negative | Negative |
| 7742.4_faster1 | Negative | Negative | Negative | Negative | Negative | Negative | Negative |
| 7591.9_faster1 | Negative | Negative | Negative | Negative | Negative | Negative | Positive |
| 7304.9_faster1 | Negative | Negative | Negative | Positive | Negative | Negative | Negative |
| 7760.9_faster1 | Negative | Negative | Negative | Positive | Negative | Negative | Negative |
| 6839_redo.4_faster1 | Negative | Negative | Negative | Negative | Negative | Negative | Positive |
| 7305.4_faster1 | Negative | Negative | Positive | Negative | Negative | Negative | Negative |
| 7760.7_faster1 | Negative | Negative | Negative | Negative | Negative | Negative | Negative |
| 7596.9_faster1 | Negative | Negative | Negative | Negative | Negative | Negative | Negative |
| 7043.12_faster1 | Negative | Negative | Negative | Negative | Negative | Negative | Positive |
| 7644.7_faster1 | Negative | Negative | Negative | Negative | Negative | Negative | Negative |
| 7757.9_faster1 | Negative | Negative | Negative | Negative | Negative | Negative | Negative |
| 7042.5_faster1 | Negative | Positive | Negative | Negative | Negative | Negative | Positive |
| 7543.12_faster1 | Negative | Negative | Negative | Negative | Negative | Negative | Negative |
| 6839_redo.3_faster1 | Negative | Negative | Negative | Negative | Negative | Negative | Positive |
| 7547.9_faster1 | Negative | Negative | Negative | Negative | Negative | Negative | Positive |
| 7606.5_faster1 | Negative | Negative | Negative | Negative | Negative | Negative | Positive |
| 7665.4_faster1 | Negative | Negative | Negative | Negative | Negative | Negative | Negative |
| 7663.4_faster1 | Negative | Negative | Negative | Negative | Negative | Negative | Negative |
| 7306.5_faster1 | Negative | Negative | Negative | Negative | Negative | Negative | Negative |
| 7742.3_faster1 | Negative | Negative | Negative | Negative | Negative | Negative | Negative |
| 7307.3_faster1 | Negative | Negative | Negative | Negative | Negative | Negative | Negative |
| 7011.8_faster1 | Negative | Negative | Negative | Positive | Negative | Negative | Positive |
| 7757.5_faster1 | Negative | Negative | Negative | Negative | Negative | Negative | Negative |
| 7739.5_faster1 | Negative | Negative | Negative | Negative | Negative | Negative | Negative |
| 7307.8_faster1 | Negative | Negative | Negative | Negative | Negative | Negative | Negative |
| 7308.6_faster1 | Negative | Negative | Negative | Negative | Negative | Negative | Negative |
| 7563.7_faster1 | Negative | Negative | Negative | Negative | Negative | Negative | Negative |
| 6839_redo.10_faster1 | Negative | Negative | Negative | Negative | Negative | Negative | Positive |
| 7649.8_faster1 | Negative | Negative | Negative | Negative | Negative | Negative | Negative |
| 7638.8_faster1 | Negative | Negative | Negative | Negative | Positive | Negative | Negative |
| 7306.7_faster1 | Negative | Negative | Negative | Negative | Negative | Negative | Negative |
| 6835.10_faster1 | Negative | Negative | Negative | Negative | Negative | Positive | Positive |
| 7549.4_faster1 | Negative | Negative | Negative | Negative | Negative | Negative | Negative |
| 7756.8_faster1 | Negative | Negative | Positive | Negative | Negative | Negative | Negative |
| 7663.9_faster1 | Negative | Negative | Negative | Negative | Negative | Negative | Negative |
| 7670.7_faster1 | Negative | Negative | Negative | Negative | Negative | Negative | Negative |
| 7306.9_faster1 | Negative | Negative | Negative | Negative | Negative | Negative | Negative |
| 7304.6_faster1 | Negative | Negative | Negative | Negative | Negative | Negative | Negative |
| 7042.8_faster1 | Negative | Negative | Negative | Negative | Negative | Negative | Positive |
| 7011.11_faster1 | Negative | Negative | Negative | Negative | Negative | Negative | Positive |
| 6836.6_faster1 | Negative | Negative | Negative | Positive | Positive | Positive | Positive |
| 7305.8_faster1 | Negative | Negative | Negative | Negative | Negative | Negative | Negative |
| 7304.8_faster1 | Negative | Negative | Negative | Negative | Negative | Negative | Positive |
| 7666.6_faster1 | Negative | Negative | Negative | Negative | Negative | Negative | Negative |
| 7308.9_faster1 | Negative | Negative | Negative | Negative | Negative | Negative | Negative |
| 7666.10_faster1 | Negative | Negative | Positive | Negative | Negative | Negative | Positive |
| 7666.7_faster1 | Negative | Negative | Negative | Negative | Negative | Negative | Positive |
| 7043.4_faster1 | Negative | Negative | Negative | Negative | Negative | Negative | Positive |
| 6839_redo.9_faster1 | Negative | Negative | Negative | Positive | Negative | Positive | Positive |
| 7670.11_faster1 | Negative | Negative | Negative | Negative | Negative | Negative | Negative |
| 7607.6_faster1 | Negative | Negative | Negative | Negative | Negative | Negative | Positive |
| 6850.5_faster1 | Negative | Negative | Negative | Negative | Negative | Negative | Positive |
| 7594.11_faster1 | Negative | Negative | Negative | Negative | Negative | Positive | Negative |
| 7305.9_faster1 | Negative | Negative | Negative | Negative | Negative | Negative | Negative |
| 7667.8_faster1 | Negative | Negative | Negative | Negative | Negative | Negative | Negative |
| 7303.6_faster1 | Negative | Negative | Negative | Negative | Negative | Negative | Negative |
| 7610.8_faster1 | Negative | Negative | Negative | Negative | Negative | Negative | Negative |
| 7740.12_faster1 | Negative | Negative | Negative | Negative | Negative | Negative | Negative |
| 7757.4_faster1 | Negative | Negative | Negative | Negative | Negative | Negative | Negative |
| 7596.3_faster1 | Negative | Negative | Negative | Negative | Negative | Negative | Negative |
| 7757.11_faster1 | Negative | Negative | Negative | Negative | Negative | Negative | Negative |
| 7760.5_faster1 | Negative | Negative | Negative | Positive | Negative | Negative | Negative |
| 7537.7_faster1 | Negative | Negative | Negative | Negative | Negative | Negative | Negative |
| 7667.5_faster1 | Negative | Negative | Negative | Negative | Negative | Negative | Positive |
| 7610.7_faster1 | Negative | Negative | Negative | Negative | Negative | Negative | Negative |
| 7638.7_faster1 | Negative | Negative | Negative | Negative | Positive | Positive | Negative |
| 7757.6_faster1 | Negative | Negative | Negative | Negative | Negative | Negative | Negative |
| 7612.8_faster1 | Negative | Negative | Negative | Negative | Negative | Negative | Negative |
| 7740.3_faster1 | Negative | Negative | Negative | Negative | Negative | Negative | Negative |
| 7643.3_faster1 | Negative | Negative | Negative | Negative | Negative | Negative | Negative |
| 7565.5_faster1 | Positive | Positive | Negative | Positive | Positive | Negative | Positive |
| 7305.7_faster1 | Negative | Negative | Negative | Negative | Negative | Negative | Negative |
| 7667.7_faster1 | Negative | Negative | Negative | Negative | Negative | Negative | Negative |
| 7592_combined.4_fast [symbol] | Negative | Negative | Negative | Negative | Negative | Negative | Positive |
| 7307.6_faster1 | Negative | Negative | Negative | Negative | Negative | Negative | Negative |
| 7534.10_faster1 | Negative | Negative | Negative | Negative | Negative | Negative | Positive |
| 7606.4_faster1 | Negative | Negative | Positive | Negative | Negative | Negative | Positive |
| 7585.3_faster1 | Negative | Negative | Negative | Positive | Negative | Negative | Positive |
| 7543.10_faster1 | Negative | Negative | Negative | Negative | Negative | Negative | Negative |
| 7303.5_faster1 | Negative | Negative | Negative | Negative | Positive | Negative | Negative |
| 6835.6_faster1 | Positive | Negative | Negative | Negative | Negative | Negative | Positive |
| 7759.6_faster1 | Negative | Negative | Negative | Negative | Negative | Negative | Positive |
| 7638.10_faster1 | Negative | Negative | Negative | Negative | Positive | Negative | Negative |
| 7610.5_faster1 | Negative | Negative | Negative | Negative | Negative | Negative | Negative |
| 6851.11_faster1 | Negative | Negative | Negative | Negative | Negative | Negative | Positive |
| 7549.5_faster1 | Negative | Negative | Negative | Negative | Negative | Negative | Negative |
| 6839_redo.6_faster1 | Negative | Negative | Negative | Negative | Positive | Negative | Positive |
| 7602.4_faster1 | Negative | Negative | Negative | Negative | Negative | Negative | Positive |
| 7608.12_faster1 | Negative | Negative | Negative | Negative | Negative | Negative | Positive |
| 7678.5_faster1 | Negative | Negative | Negative | Negative | Negative | Negative | Negative |
| 7305.5_faster1 | Negative | Negative | Negative | Negative | Negative | Negative | Negative |
| 7535.9_faster1 | Positive | Negative | Negative | Negative | Negative | Negative | Positive |
| 7742.5_faster1 | Negative | Negative | Negative | Negative | Negative | Negative | Negative |
| 7639.3_faster1 | Negative | Negative | Negative | Negative | Negative | Negative | Negative |
| 7606.12_faster1 | Negative | Negative | Positive | Negative | Negative | Negative | Positive |
| 7606.3_faster1 | Negative | Negative | Negative | Negative | Positive | Negative | Positive |
| 7565.3_faster1 | Negative | Negative | Negative | Negative | Positive | Positive | Negative |
| 7645.4_faster1 | Negative | Negative | Negative | Negative | Negative | Negative | Negative |
| 7043.5_faster1 | Negative | Negative | Negative | Negative | Negative | Negative | Positive |
| 7014.9 _faster1 | Negative | Negative | Negative | Negative | Negative | Negative | Positive |
| 7041.7_faster1 | Negative | Negative | Negative | Negative | Negative | Negative | Positive |
| 7560.6_faster1 | Negative | Negative | Negative | Negative | Negative | Negative | Negative |
| 7308.12_faster1 | Negative | Negative | Negative | Negative | Negative | Negative | Negative |
| 7586.10_faster1 | Negative | Negative | Negative | Positive | Positive | Negative | Negative |
| 7307.10_faster1 | Negative | Negative | Negative | Negative | Negative | Negative | Negative |
| 7607.9_faster1 | Negative | Negative | Negative | Negative | Negative | Negative | Positive |
| 7664.5_faster1 | Negative | Negative | Negative | Negative | Negative | Negative | Positive |
| 7534.5_faster1 | Negative | Negative | Negative | Negative | Negative | Negative | Positive |
| 7611.8_faster1 | Negative | Negative | Negative | Negative | Negative | Negative | Positive |
| 6858.3_faster1 | Negative | Negative | Negative | Negative | Negative | Negative | Positive |
| 7566.10_faster1 | Negative | Negative | Negative | Positive | Negative | Negative | Negative |
| 7642.7_faster1 | Negative | Negative | Negative | Positive | Negative | Negative | Negative |
| 7665.6_faster1 | Negative | Negative | Negative | Negative | Negative | Negative | Positive |
| 7544.4_faster1 | Negative | Negative | Negative | Negative | Negative | Negative | Negative |
| 6858.7_faster1 | Negative | Negative | Negative | Negative | Negative | Negative | Positive |
| 7601.5_faster1 | Negative | Negative | Negative | Negative | Negative | Negative | Negative |
| 7603.4_faster1 | Negative | Negative | Negative | Negative | Negative | Negative | Negative |
| 7677.9_faster1 | Negative | Negative | Negative | Negative | Negative | Negative | Negative |
| 7562.3_faster1 | Positive | Negative | Negative | Negative | Negative | Negative | Negative |
| 7639.7_faster1 | Negative | Negative | Negative | Negative | Positive | Negative | Negative |
| 7544.11_faster1 | Negative | Positive | Negative | Negative | Negative | Negative | Negative |
| 6838_redo.3_faster1 | Negative | Negative | Negative | Positive | Negative | Negative | Positive |
| 7561.12_faster1 | Negative | Negative | Negative | Negative | Negative | Negative | Negative |
| 7670.8_faster1 | Negative | Negative | Negative | Negative | Negative | Negative | Negative |
| 7664.3_faster1 | Negative | Negative | Negative | Negative | Negative | Negative | Negative |
| 7009.11_faster1 | Negative | Negative | Negative | Negative | Negative | Negative | Positive |
| 7638.4_faster1 | Negative | Negative | Positive | Positive | Negative | Negative | Negative |
| 7304.4_faster1 | Negative | Negative | Negative | Negative | Negative | Negative | Negative |
| 7305.6_faster1 | Negative | Negative | Negative | Negative | Negative | Negative | Negative |
| 7548.7_faster1 | Negative | Negative | Negative | Negative | Negative | Negative | Negative |
| 7549.9_faster1 | Negative | Negative | Negative | Negative | Negative | Negative | Negative |
| 7544.6_faster1 | Negative | Positive | Negative | Negative | Negative | Positive | Positive |
| 7535.3_faster1 | Negative | Negative | Negative | Negative | Negative | Negative | Positive |
| 7643.4_faster1 | Negative | Negative | Negative | Positive | Negative | Negative | Negative |
| 7672.9_faster1 | Negative | Negative | Negative | Negative | Negative | Negative | Negative |
| 7756.7_faster1 | Negative | Negative | Negative | Negative | Negative | Negative | Negative |
| 7642.11_faster1 | Negative | Negative | Negative | Negative | Positive | Negative | Negative |
| 7307.4_faster1 | Negative | Negative | Negative | Negative | Negative | Negative | Negative |
| 7758.7_faster1 | Negative | Negative | Negative | Positive | Positive | Negative | Negative |
| 6858.9_faster1 | Negative | Negative | Negative | Negative | Negative | Negative | Positive |
| 7607.3_faster1 | Negative | Negative | Negative | Negative | Negative | Negative | Positive |
| 7663.5_faster1 | Negative | Negative | Negative | Negative | Negative | Negative | Negative |
| 7584.4 _faster1 | Negative | Negative | Negative | Negative | Negative | Negative | Positive |
| 6858.4_faster1 | Negative | Negative | Negative | Negative | Negative | Negative | Positive |
| 7306.3_faster1 | Negative | Negative | Positive | Negative | Negative | Negative | Negative |
| 7041.5_faster1 | Negative | Negative | Negative | Negative | Negative | Negative | Positive |
| 7608.5_faster1 | Negative | Negative | Negative | Negative | Negative | Negative | Positive |
| 7740.9_faster1 | Negative | Negative | Negative | Negative | Negative | Negative | Negative |
| 7043.6_faster1 | Negative | Positive | Negative | Negative | Negative | Negative | Positive |
| 7608.3_faster1 | Negative | Negative | Negative | Negative | Negative | Negative | Positive |
| 7602.5_faster1 | Negative | Negative | Negative | Negative | Negative | Negative | Negative |
| 7560.4_faster1 | Negative | Negative | Negative | Negative | Negative | Negative | Negative |
| 7637.12_faster1 | Negative | Negative | Negative | Negative | Negative | Positive | Negative |
| 6850.9_faster1 | Negative | Negative | Negative | Negative | Negative | Negative | Positive |
| 6858.11_faster1 | Negative | Negative | Negative | Negative | Negative | Negative | Positive |
| 7613.12_faster1 | Negative | Negative | Negative | Positive | Positive | Negative | Negative |
| 7542.3_faster1 | Negative | Positive | Negative | Negative | Negative | Positive | Positive |
| 7011.6_faster1 | Negative | Positive | Positive | Positive | Negative | Negative | Positive |
| 7536.11_faster1 | Positive | Negative | Negative | Negative | Negative | Negative | Positive |
| 7543.6_faster1 | Negative | Negative | Negative | Negative | Negative | Negative | Positive |
| 7303.7_faster1 | Negative | Negative | Negative | Negative | Negative | Negative | Negative |
| 7610.11_faster1 | Negative | Negative | Negative | Negative | Negative | Negative | Negative |
| 7590.7_faster1 | Positive | Negative | Negative | Negative | Negative | Negative | Negative |
| 7742.6_faster1 | Negative | Negative | Negative | Negative | Negative | Negative | Negative |
| 7594.8_faster1 | Negative | Negative | Negative | Negative | Negative | Negative | Negative |
| 7586.9_faster1 | Negative | Negative | Negative | Negative | Negative | Negative | Positive |
| 7585.7_faster1 | Negative | Negative | Negative | Positive | Positive | Negative | Positive |
| 7010.4_faster1 | Negative | Negative | Negative | Negative | Negative | Negative | Positive |
| 7563.3_faster1 | Negative | Negative | Negative | Negative | Negative | Positive | Positive |
| 7591.7_faster1 | Negative | Positive | Negative | Negative | Negative | Negative | Negative |
| 7739.12_faster1 | Negative | Negative | Positive | Negative | Negative | Negative | Negative |
| 7639.8_faster1 | Negative | Negative | Negative | Negative | Negative | Negative | Negative |
| 7564.5_faster1 | Negative | Negative | Negative | Negative | Negative | Negative | Negative |
| 7308.5_faster1 | Negative | Negative | Negative | Negative | Negative | Negative | Negative |
| 7664.7_faster1 | Negative | Negative | Negative | Negative | Negative | Negative | Negative |
| 6850.6_faster1 | Negative | Negative | Negative | Negative | Negative | Negative | Positive |
| 7534.4_faster1 | Positive | Negative | Negative | Negative | Positive | Negative | Positive |
| 7637.6_faster1 | Negative | Negative | Negative | Negative | Negative | Negative | Negative |
| 7591.6_faster1 | Negative | Negative | Negative | Negative | Negative | Negative | Negative |
| 7613.3_faster1 | Negative | Negative | Negative | Negative | Negative | Negative | Negative |
| 7742.11_faster1 | Negative | Negative | Negative | Negative | Negative | Negative | Negative |
| 7611.4_faster1 | Negative | Negative | Negative | Positive | Positive | Negative | Negative |
| 7306.4_faster1 | Negative | Negative | Negative | Negative | Negative | Negative | Negative |
| 7561.11_faster1 | Negative | Negative | Negative | Negative | Negative | Negative | Positive |
| 7536.5_faster1 | Negative | Positive | Negative | Negative | Negative | Negative | Positive |
| 7640.9_faster1 | Negative | Negative | Negative | Negative | Negative | Negative | Positive |
| 7739.7_faster1 | Negative | Negative | Negative | Negative | Negative | Negative | Negative |
| 7665.11_faster1 | Negative | Negative | Negative | Negative | Negative | Negative | Negative |
| 6851.8_faster1 | Negative | Negative | Negative | Negative | Negative | Negative | Positive |
| 7592_combined.9_fast [symbol] | Positive | Negative | Negative | Negative | Negative | Negative | Negative |
| 6850.11_faster1 | Negative | Negative | Negative | Negative | Negative | Negative | Positive |
| 7646.7_faster1 | Negative | Negative | Negative | Negative | Negative | Negative | Negative |
| 7596.5_faster1 | Negative | Negative | Negative | Negative | Negative | Negative | Negative |
| 7611.11_faster1 | Negative | Negative | Negative | Positive | Negative | Negative | Negative |
| 6850.7_faster1 | Negative | Negative | Negative | Negative | Negative | Negative | Positive |
| 7013.4_faster1 | Negative | Negative | Negative | Negative | Negative | Positive | Positive |
| 7639.5_faster1 | Negative | Negative | Negative | Negative | Negative | Positive | Positive |
| 7585.3_faster1 | Negative | Negative | Negative | Negative | Negative | Negative | Positive |
| 6839_redo.5_faster1 | Negative | Negative | Negative | Negative | Positive | Negative | Positive |
| 7602.9_faster1 | Negative | Negative | Positive | Negative | Positive | Negative | Negative |
| 7676.9_faster1 | Negative | Negative | Negative | Negative | Negative | Negative | Negative |
| 7671.7_faster1 | Negative | Negative | Negative | Negative | Positive | Positive | Negative |
| 7649.9_faster1 | Negative | Negative | Negative | Negative | Negative | Negative | Negative |
| 7662.9_faster1 | Negative | Negative | Negative | Negative | Negative | Negative | Negative |
| 7012.12_faster1 | Negative | Negative | Negative | Negative | Negative | Positive | Positive |
| 7549.3_faster1 | Negative | Negative | Negative | Negative | Negative | Negative | Positive |
| 7561.10_faster1 | Negative | Negative | Negative | Negative | Negative | Negative | Positive |
| 7671.4_faster1 | Negative | Negative | Negative | Negative | Negative | Negative | Negative |
| 7549.8_faster1 | Negative | Negative | Negative | Negative | Negative | Negative | Negative |
| 7043.9_faster1 | Negative | Negative | Positive | Negative | Negative | Negative | Positive |
| 7670.4_faster1 | Negative | Positive | Positive | Negative | Negative | Positive | Positive |
| 7043.11_faster1 | Negative | Negative | Positive | Negative | Negative | Negative | Positive |
| 6835.5_faster1 | Negative | Positive | Negative | Negative | Positive | Negative | Positive |
| 7649.12_faster1 | Negative | Negative | Negative | Negative | Negative | Negative | Positive |
| 7662.5_faster1 | Negative | Negative | Negative | Negative | Negative | Negative | Negative |
| 6858.6_faster1 | Negative | Negative | Positive | Negative | Negative | Negative | Positive |
| 7014.6_faster1 | Negative | Negative | Negative | Negative | Positive | Negative | Positive |
| 7010.7_faster1 | Negative | Negative | Negative | Negative | Negative | Negative | Positive |
| 7009.3_faster1 | Negative | Positive | Positive | Negative | Negative | Negative | Positive |
| 7012.5_faster1 | Positive | Positive | Positive | Positive | Positive | Positive | Positive |
| 7013.11_faster1 | Negative | Negative | Negative | Negative | Negative | Negative | Positive |
| 7637.9_faster1 | Negative | Negative | Negative | Negative | Negative | Positive | Negative |
| 7594.12_faster1 | Negative | Negative | Negative | Negative | Negative | Negative | Negative |
| 7612.7_faster1 | Negative | Negative | Negative | Negative | Negative | Negative | Positive |
| 7010.10_faster1 | Negative | Negative | Negative | Negative | Negative | Negative | Positive |
| 7606.7_faster1 | Negative | Negative | Negative | Positive | Positive | Negative | Positive |
| 7013.7_faster1 | Negative | Negative | Negative | Negative | Negative | Negative | Positive |
| 6837.5_faster1 | Negative | Negative | Positive | Negative | Negative | Negative | Positive |
| 7544.12_faster1 | Negative | Negative | Negative | Negative | Negative | Negative | Negative |
| 7542.6_faster1 | Negative | Negative | Positive | Negative | Negative | Positive | Positive |
| 7594.7_faster1 | Negative | Negative | Negative | Negative | Negative | Positive | Positive |
| 7562.8_faster1 | Negative | Negative | Negative | Negative | Negative | Negative | Negative |
| 7645.9_faster1 | Positive | Positive | Negative | Negative | Negative | Negative | Positive |
| 7009.12_faster1 | Negative | Negative | Negative | Negative | Negative | Positive | Positive |
| 7014.7_faster1 | Negative | Negative | Positive | Negative | Negative | Negative | Positive |
| 7639.10_faster1 | Negative | Negative | Positive | Negative | Negative | Negative | Negative |
| 7589.4_faster1 | Negative | Negative | Negative | Negative | Negative | Negative | Positive |
| 7602.8_faster1 | Negative | Negative | Negative | Negative | Negative | Negative | Negative |
| 7662.8_faster1 | Negative | Negative | Negative | Positive | Negative | Negative | Positive |
| 7637.10_faster1 | Negative | Negative | Positive | Positive | Negative | Negative | Positive |
| 7013.10_faster1 | Negative | Negative | Positive | Negative | Negative | Negative | Positive |
| 6835.4_faster1 | Negative | Negative | Negative | Positive | Negative | Negative | Positive |
| 7610.3 _faster1 | Negative | Positive | Positive | Positive | Negative | Negative | Positive |
| 7663.10_faster1 | Negative | Negative | Negative | Negative | Negative | Negative | Positive |
| 7013.3_faster1 | Negative | Negative | Negative | Negative | Negative | Positive | Positive |
| 7678.3_faster1 | Negative | Negative | Negative | Negative | Negative | Negative | Positive |
| 7740.8_faster1 | Negative | Negative | Negative | Negative | Negative | Negative | Positive |
| 7665.3_faster1 | Negative | Positive | Positive | Negative | Negative | Negative | Positive |
| 6857.11_faster1 | Negative | Negative | Positive | Positive | Positive | Positive | Positive |
| 6839_redo.11_faster1 | Negative | Positive | Positive | Negative | Negative | Negative | Positive |
| 7673.5_faster1 | Negative | Negative | Positive | Negative | Negative | Negative | Negative |
| 6837.10_faster1 | Negative | Negative | Negative | Positive | Negative | Negative | Positive |
| 7535.10_faster1 | Negative | Negative | Negative | Negative | Negative | Negative | Negative |
| 7011.10_faster1 | Negative | Negative | Negative | Positive | Positive | Positive | Positive |
| 7677.5_faster1 | Negative | Negative | Negative | Negative | Negative | Negative | Negative |
| 7537.4_faster1 | Negative | Negative | Negative | Negative | Negative | Negative | Positive |
| 7638.3_faster1 | Negative | Negative | Negative | Negative | Negative | Negative | Negative |
| 6838_redo.9_faster1 | Negative | Negative | Negative | Negative | Negative | Negative | Positive |
| 7566.3_faster1 | Positive | Negative | Negative | Positive | Negative | Positive | Positive |
| 7640.4_faster1 | Negative | Negative | Negative | Negative | Negative | Negative | Negative |
| 7561.8_faster1 | Negative | Negative | Negative | Negative | Negative | Negative | Positive |
| 7602.10_faster1 | Negative | Negative | Negative | Positive | Positive | Negative | Negative |
| 7041.6_faster1 | Negative | Negative | Negative | Negative | Negative | Negative | Positive |
| 7567.5_faster1 | Negative | Negative | Negative | Positive | Negative | Negative | Negative |
| 6851.9_faster1 | Negative | Negative | Positive | Negative | Negative | Negative | Negative |
| 7644.5_faster1 | Negative | Negative | Positive | Negative | Negative | Negative | Negative |
| 7042.10_faster1 | Negative | Negative | Negative | Negative | Negative | Negative | Positive |
| 7609.6_faster1 | Negative | Negative | Negative | Negative | Negative | Negative | Negative |
| 7593.5_faster1 | Negative | Negative | Negative | Negative | Negative | Negative | Positive |
| 7011.5_faster1 | Negative | Negative | Negative | Positive | Positive | Negative | Positive |
| 7677.6_faster1 | Negative | Negative | Negative | Negative | Negative | Negative | Negative |
| 7041.9_faster1 | Negative | Positive | Positive | Negative | Negative | Negative | Positive |
| 6857.3_faster1 | Negative | Negative | Negative | Negative | Negative | Negative | Positive |
| 7010.6_faster1 | Negative | Negative | Negative | Positive | Negative | Negative | Negative |
| 7542.5_faster1 | Negative | Positive | Negative | Negative | Negative | Positive | Negative |
| 7592_combined.8_fast [symbol] | Negative | Negative | Negative | Negative | Negative | Negative | Positive |
| 7586.8_faster1 | Negative | Negative | Negative | Negative | Negative | Negative | Negative |
| 7560.3_faster1 | Negative | Negative | Negative | Negative | Negative | Negative | Negative |
| 7667.12_faster1 | Negative | Negative | Negative | Negative | Negative | Negative | Negative |
| 6851.7_faster1 | Negative | Negative | Negative | Negative | Negative | Negative | Positive |
| 7673.9_faster1 | Negative | Negative | Negative | Negative | Negative | Negative | Negative |
| 7640.11_faster1 | Negative | Negative | Negative | Positive | Positive | Positive | Negative |
| 7566.12_faster1 | Negative | Negative | Negative | Negative | Negative | Negative | Negative |
| 6838_redo.7_faster1 | Negative | Negative | Negative | Positive | Positive | Negative | Positive |
| 7563.9_faster1 | Negative | Negative | Negative | Positive | Positive | Positive | Negative |
| 7664.8_faster1 | Negative | Negative | Negative | Negative | Negative | Negative | Positive |
| 7589.11_faster1 | Negative | Positive | Negative | Negative | Negative | Negative | Negative |
| 7759.10_faster1 | Negative | Negative | Negative | Negative | Negative | Negative | Negative |
| 7564.3_faster1 | Negative | Negative | Negative | Positive | Negative | Negative | Positive |
| 7560.5_faster1 | Negative | Negative | Positive | Negative | Negative | Negative | Negative |
| 7646.4_faster1 | Negative | Negative | Negative | Negative | Negative | Negative | Negative |
| 7592_combined.3_fast [symbol] | Negative | Negative | Negative | Negative | Negative | Negative | Negative |
| 7742.12_faster1 | Negative | Negative | Negative | Negative | Negative | Negative | Positive |
| 7594.9_faster1 | Negative | Negative | Negative | Positive | Positive | Positive | Positive |
| 7548.3_faster1 | Negative | Negative | Negative | Negative | Negative | Negative | Negative |
| 7649.11_faster1 | Negative | Negative | Negative | Negative | Negative | Negative | Negative |
| 7585.6_faster1 | Negative | Negative | Negative | Positive | Positive | Positive | Positive |
| 7042.12_faster1 | Negative | Negative | Positive | Negative | Negative | Negative | Positive |
| 7014.5_faster1 | Negative | Negative | Negative | Negative | Negative | Negative | Positive |
| 7566.5_faster1 | Negative | Negative | Positive | Positive | Positive | Positive | Positive |
| 7567.3_faster1 | Negative | Negative | Negative | Negative | Negative | Negative | Negative |
| 7562.12_faster1 | Positive | Negative | Negative | Negative | Negative | Negative | Positive |
| 7740.4_faster1 | Negative | Positive | Positive | Negative | Negative | Negative | Negative |
| 7676.7_faster1 | Negative | Negative | Negative | Negative | Negative | Negative | Negative |
| 7563.5_faster1 | Negative | Negative | Negative | Negative | Positive | Positive | Negative |
| 6837.3_faster1 | Negative | Negative | Negative | Negative | Negative | Negative | Positive |
| 7589.9_faster1 | Negative | Negative | Negative | Negative | Positive | Negative | Negative |
| 7596.6_faster1 | Negative | Negative | Negative | Negative | Negative | Negative | Negative |
| 7584.3_faster1 | Negative | Negative | Negative | Positive | Positive | Negative | Positive |
| 7612.12_faster1 | Negative | Negative | Positive | Negative | Negative | Negative | Positive |
| 7541.3_faster1 | Negative | Positive | Negative | Negative | Negative | Negative | Positive |
| 7672.6_faster1 | Negative | Negative | Negative | Negative | Negative | Negative | Positive |
| 7671.9_faster1 | Negative | Negative | Negative | Positive | Negative | Positive | Positive |
| 7012.10_faster1 | Negative | Negative | Negative | Positive | Negative | Positive | Positive |
| 7642.6_faster1 | Negative | Negative | Positive | Negative | Negative | Negative | Negative |
| 7739.4_faster1 | Negative | Negative | Negative | Negative | Negative | Negative | Positive |
| 7645.3_faster1 | Negative | Negative | Negative | Negative | Negative | Negative | Negative |
| 7643.11_faster1 | Negative | Positive | Negative | Negative | Negative | Negative | Positive |
| 7593.9_faster1 | Negative | Negative | Negative | Negative | Negative | Negative | Positive |
| 7678.S_faster1 | Negative | Negative | Negative | Negative | Negative | Negative | Negative |
| 7609.3_faster1 | Negative | Negative | Negative | Negative | Negative | Negative | Positive |
| 7537.6_faster1 | Negative | Positive | Negative | Negative | Negative | Positive | Positive |
| 7547.4_faster1 | Negative | Negative | Negative | Positive | Positive | Negative | Positive |
| 7603.3_faster1 | Negative | Negative | Negative | Negative | Negative | Negative | Negative |
| 7567.4_faster1 | Negative | Negative | Negative | Negative | Positive | Positive | Negative |
| 7590.12_faster1 | Negative | Negative | Negative | Negative | Negative | Negative | Negative |
| 7644.6_faster1 | Negative | Negative | Negative | Negative | Negative | Negative | Negative |
| 7009.4_faster1 | Negative | Positive | Positive | Positive | Positive | Negative | Positive |
| 7591.3_faster1 | Negative | Positive | Negative | Negative | Positive | Negative | Positive |
| 7666.3_faster1 | Negative | Negative | Negative | Negative | Negative | Negative | Positive |
| 7613.4_faster1 | Negative | Positive | Negative | Negative | Negative | Negative | Positive |

[symbol]

indicates data missing or illegible when filed

What is claimed is:

1. A method of testing for the presence of aneuploidy in a genome of a mammal comprising:
a) amplifying a plurality of chromosomal sequences in a DNA sample with a pair of primers complementary to the chromosomal sequences to form a plurality of amplicons, wherein the primer pair amplifies a sufficient number of sequences to allow aneuploidy detection;
b) determining at least a portion of the nucleic acid sequence of one or more of the plurality of amplicons;
c) mapping the sequenced amplicons to a reference genome;
d) dividing the DNA sample into a plurality of genomic intervals;
e) quantifying a plurality of features for the amplicons mapped to the genomic intervals;
f) comparing the plurality of features of amplicons in a first genomic interval with the plurality of features of amplicons in one or more different genomic intervals; and
g) wherein a number of amplicons sufficient to detect aneuploidy are formed in the step of amplifying,
thereby testing for the presence of aneuploidy in the genome of the mammal.

2. The method of claim 1, wherein the DNA sample comprises a plurality of euploid DNA samples.

3. The method of claim 1, wherein the DNA sample comprises a plurality of test DNA samples.

4. The method of claim 3, wherein the test DNA comprises DNA of unknown ploidy.

5. The method of claim 1, wherein the DNA sample is from plasma.

6. The method of claim 1, wherein the DNA sample is from serum.

7. The method of claim 1, wherein the DNA sample comprises cell free DNA.

8. The method of any one of claims 1 to 7, wherein the DNA sample comprises at least 3 picograms of DNA.

9. The method of any one of claims 1 to 8, wherein the mammal is a human.

10. The method of any one of claims 1 to 9, wherein the pair of primers comprises a first primer and a second primer chosen from Table 1, e.g., a first primer comprising SEQ ID NO: 1 and a second primer comprising SEQ ID NO: 10, or a first primer with at least 80%, 85%, 90%, 95%, 96%, 97%, 98%, or 99% identity to SEQ ID NO: 1 and a second primer with at least 80%, 85%, 90%, 95%, 96%, 97%, 98%, or 99% identity to SEQ ID NO: 10.

11. The method of any one of claims 1 to 10, wherein one or more additional pairs of primers amplifies one or more additional pluralities of chromosomal sequences in the DNA sample in step (a).

12. The method of any one of claims 1 to 11, wherein said amplicons comprise one or more repetitive elements shown in Table 1.

13. The method of claim 12, wherein said amplicons comprise unique short interspersed nucleotide elements (SINEs).

14. The method of any one of claims 1 to 13, wherein the average length of the amplicons is 100 basepairs or less.

15. The method of any one of claims 1 to 14, wherein said amplicons comprise one or more long amplicons where the average length of the long amplicons is 1000 basepairs or greater.

16. The method of claim 15, wherein said long amplicons comprise DNA from a contaminating cell.

17. The method of claim 16, wherein the contaminating cell is a leukocyte.

18. The method of any one of claims 1 to 17, wherein the plurality of amplicons comprises sequences on a plurality of, e.g., 2 or more, different chromosomes.

19. The method of any one of claims 1 to 18, wherein the genomic intervals comprise from about 100 nucleotides to about 125,000,000 nucleotides.

20. The method of any one of claims 1 to 19, wherein quantifying amplicons mapped to genomic intervals comprises identifying a plurality of genomic intervals with one or more shared amplicon features.

21. The method of claim 20, wherein the shared amplicon feature is the number of the mapped amplicons.

22. The method of claim 20, wherein the shared amplicon feature is the average length of the mapped amplicons.

23. The method as in any one of claims 20 to 22, wherein the plurality of genomic intervals with shared amplicon features are grouped into one or more clusters.

24. The method of claim 23, wherein each cluster comprises about two hundred genomic intervals.

25. The method of claim 23, wherein the clusters comprise predefined clusters.

26. The method of any one of claims 1 to 25, wherein the comparison of the genomic intervals further comprises matching one or more genomic intervals from test samples to predefined clusters.

27. The method of claim 26, wherein matching genomic intervals from test samples to predefined clusters further comprises identifying one or more genomic intervals with shared amplicon features outside a predetermined significance threshold for a predefined cluster.

28. The method of any one of claims 1 to 27, wherein testing for the presence of aneuploidy comprises supervised machine learning.

29. The method of claim 28, wherein the supervised machine learning employs a support vector machine model.

30. A pair of primers for the amplification of a plurality of amplicons from a DNA sample comprising a first primer comprising a sequence that is at least 80% identical to SEQ ID NO: 1 and a second primer comprising a sequence that is at least 80% identical to SEQ ID NO: 10.

31. The pair of primers of claim 30, wherein the sequence of the first primer is at least 90% identical to SEQ ID NO. 1

32. The pair of primers of claim 30, wherein the sequence of the first primer is at least 95% identical to SEQ ID NO. 1.

33. The pair of primers of claim 30, wherein the sequence of the first primer is or comprises a sequence that is 100% identical to SEQ ID NO. 1 and/or the sequence of the second primer is or comprises a sequence that is 100% identical to SEQ ID NO. 2.

34. The pair of primers of any one of claims 30 to 32, wherein the sequence of the second primer is at least 90% identical to SEQ ID NO. 10.

35. The pair of primers of any one of claims 30 to 32, wherein the sequence of the second primer is at least 95% identical to SEQ ID NO. 10.

36. The pair of primers of any one of claims 30 to 32, wherein the sequence of the second primer is or comprises a sequence that is 100% identical to SEQ ID NO. 10.

37. A kit for the amplification of a plurality of amplicons from a DNA sample comprising a pair of primers, wherein a first primer of the primer pair comprises SEQ ID NO: 1 and a second primer of the primer pair comprises SEQ ID NO: 10.

38. The method of any one of claims 1 to 29, wherein at least 10,000 amplicons are formed in the step of amplifying.

39. The method of any one of claims 1 to 37, wherein at least 20,000 amplicons are formed in the step of amplifying.

40. The method of any one of claims 1 to 37, wherein at least 50,000 amplicons are formed in the step of amplifying.

41. The method of any one of claims 1 to 37, wherein at least 100,000 amplicons are formed in the step of amplifying.

42. A method of evaluating a subject for the presence of, or the risk of developing, each of a plurality of cancers in the subject comprising:
(i) acquiring a value for the presence of one or more mutations in each of one or more driver genes, wherein each driver gene is associated with the presence, or risk, of a cancer of the plurality of cancers;
(ii) acquiring, a value for the level of each of a plurality of protein biomarkers, wherein the level of each protein biomarker of the plurality is associated with the presence, or risk, of a cancer of the plurality of cancers;
(iii) acquiring a value for aneuploidy, wherein the aneuploidy value is a function of the copy number or length of a genomic sequence disposed between at least two terminal repeated elements of a repeated element family (RE Family), wherein the RE family comprises:
(a) a RE Family other than a long interspersed nucleotide element (LINE);
(b) a RE Family which when amplified with a primer moiety complementary to its repeated terminal elements, provides amplicons having an average length of less than X nts, wherein X is 100, 105, or 110,
(c) a RE family which is less than about 700 bp long; or
(d) a RE family which is present in at least 100 copies per genome;
wherein the aneuploidy is associated with the presence, or risk, of a cancer of the plurality of cancers;
thereby evaluating the subject for the presence of or risk of developing, any of the plurality of cancers.

43. The method of claim 42, wherein one of (i), (ii) and (iii) is directly acquired.

44. The method of claim 42, wherein (i) and (ii) are directly acquired.

45. The method of claim 42, wherein (i) and (iii) are directly acquired.

46. The method of claim 42, wherein (i) and (ii) are directly acquired.

47. The method of claim 42, wherein all of (i), (ii) and (iii) are directly acquired.

48. The method of claim 42, wherein one of (i), (ii) and (iii) is indirectly acquired.

49. The method of claim 42, wherein (i) and (ii) are indirectly acquired.

50. The method of claim 42, wherein (i) and (iii) are indirectly acquired.

51. The method of claim 42, wherein (i) and (ii) are indirectly acquired.

52. The method of claim 42, wherein all of (i), (ii) and (iii) are indirectly acquired.

53. The method of any one of claims 42 to 52, comprising:
(1) sequencing one or more subgenomic intervals or amplicons comprising the genetic biomarkers;
(2) analyzing one or more genomic sequences for aneuploidy, and/or
(3) contacting a protein biomarker with a detection reagent.

54. The method of any one of claims 42 to 53, wherein the aneuploidy value is a function of the copy number of the genomic sequence disposed between at least two terminal repeated elements of a RE Family.

55. The method of any one of claims 42 to 54, wherein the aneuploidy value is a function of the length of the genomic sequence disposed between at least two terminal repeated elements of a repeated element family (RE Family).

56. The method of any one of claims 42 to 55, further comprising:
(i) acquiring a sequence for a subgenomic interval from cell-free DNA from a sample; and
(ii) acquiring a leukocyte parameter from leukocyte DNA from the sample.

57. The method of claim 55 or 56, wherein the leukocyte parameter comprises a sequence of the subgenomic interval.

58. The method of claim 55 or 56, further comprising comparing (i) with (ii) to evaluate a genomic event found in the cell-free DNA subgenomic interval or cell-free DNA aneuploidy analysis sample.

59. The method of claim 58, wherein the genomic event comprises a mutation.

60. The method of any one of claims 42 to 59, wherein specificity of detection of the cancer in the plurality of cancers with (i), (ii) and (iii) is substantially the same as the specificity of detection of the cancer in the plurality of cancers with: (i); (ii); (iii); (i) and (ii); (i) and (iii); or (ii) and (iii).

61. The method of any one of claims 42 to 59, wherein specificity of detection of the cancer in the plurality of cancers with (i), (ii) and (iii) is not substantially lower than the specificity of detection of the cancer in the plurality of cancers with: (i); (ii); (iii); (i) and (ii); (i) and (iii); or (ii) and (iii).

62. The method of any one of claims 42 to 61, wherein sensitivity of detection of the cancer in the plurality of cancers with (i), (ii) and (iii) is higher than the sensitivity of detection of the cancer in the plurality of cancers with: (i); (ii); (iii); (i) and (ii); (i) and (iii); or (ii) and (iii).

63. The method of claim 62, wherein sensitivity of detection of the cancer in the plurality of cancers with (i), (ii) and (iii) is about 1.1, 1.2, 1.3, 1.4, 1.5, 2, 2.5, 3, 3.5, 4, 4.5, 5, 5.5, 6, 6.5, 7, 7.5, 8, 8.5, 9, 9.5, or 10 fold higher, than the sensitivity of detection of the cancer in the plurality of cancers with: (i); (ii); (iii); (i) and (ii); (i) and (iii); or (ii) and (iii).

64. The method of any one of claims 42 to 63, wherein (i), (ii) and (iii) result in an increased sensitivity of detection at a specified specificity.

65. The method of claim 64, wherein the increased sensitivity of detection is increased by about 1.1, 1.2, 1.3, 1.4, 1.5, 2, 2.5, 3, 3.5, 4, 4.5, 5, 5.5, 6, 6.5, 7, 7.5, 8, 8.5, 9, 9.5, or 10 fold at the specified specificity.

66. The method of claim 64 or 65, wherein the specificity is a predetermined specificity.

67. The method of claim 66, wherein the predetermined specificity is at least about 90%, 91%, 92%, 93%, 94%, 95%, 96%, 97%, 98%, 99% or 100% specificity.

68. The method of any one of claims 62 to 67, wherein the increase in sensitivity of detection of the cancer in the plurality of cancers does not affect the specificity of detection of the cancer in the plurality of cancer.

69. The method of any one of claims 62 to 67, wherein the increase in sensitivity of detection of the cancer in the plurality of cancers does not reduce or substantially reduce the specificity of detection of the cancer in the plurality of cancer.

70. The method of claim 68 or 69, wherein the specificity of detection of the cancer in the plurality of cancers is at a plateau.

71. The method of any one of claims 42 to 70, wherein acquiring a value for the presence of one or more mutations comprises detecting the one or more mutations in the one or more driver genes.

72. The method of claim 71, wherein the one or more mutations comprise one or more driver gene mutations.

73. The method of any one of claims 42 to 72, wherein the one or more driver genes are chosen from: NRAS, CTNNB1, PIK3CA, FBXW7, APC, EGFR, BRAF, CDKN2A, PTEN, FGFR2, HRAS, KRAS, AKT1, TP53, PPP2R1A, or GNAS.

74. The method of any one of claims 42 to 73, wherein the presence of one or more mutations are evaluated in at least four driver genes chosen from: NRAS, CTNNB1, PIK3CA, FBXW7, APC, EGFR, BRAF, CDKN2A, PTEN, FGFR2, HRAS, KRAS, AKT1, TP53, PPP2R1A, or GNAS.

75. The method of any one of claims 42 to 74, wherein the presence of one or more mutations are evaluated in all sixteen of the following driver genes: NRAS, CTNNB1, PIK3CA, FBXW7, APC, EGFR, BRAF, CDKN2A, PTEN, FGFR2, HRAS, KRAS, AKT1, TP53, PPP2R1A, and GNAS.

76. The method of any one of claims 42 to 75, wherein acquiring a value for each of the plurality of protein biomarkers comprises detecting each of the plurality of protein biomarkers, e.g., chosen from: CA19-9, CEA, HGF, OPN, CA125, prolactin (PRL), TIMP-1, CA15-3, AFP or MPO.

77. The method of claim 76, wherein the plurality of protein biomarkers comprises at least four protein biomarkers.

78. The method of any one of claims 42 to 77, wherein acquiring a value for aneuploidy comprises detecting aneuploidy.

79. The method of any one of claims 42 to 78, wherein the plurality of cancers comprises at least four cancers.

80. The method of any one of claims 42 to 79, further comprising subjecting the subject to a radiologic scan, e.g., a PET-CT scan, of an organ or body region.

81. The method of claim 80, wherein the radiologic scanning of an organ or body region characterizes the cancer.

82. The method of claim 80, wherein the radiologic scanning of an organ or body region identifies the location of the cancer.

83. The method of any one of claims 80-82, wherein the radiologic scan is a PET-CT scan.

84. The method of any one of claims 80-83, wherein the radiologic scanning is performed after the subject is evaluated for the presence of each of a plurality of cancers.

85. The method of any one of claims 42-84, comprising administering to the subject one or more therapeutic interventions (e.g., surgery, adjuvant chemotherapy, neoadjuvant chemotherapy, radiation therapy, immunotherapy, targeted therapy, and/or an immune checkpoint inhibitor).

86. The method of any of claims 42-85, wherein the subject is asymptomatic for a cancer.

87. The method of any of claims 42-85, wherein the subject is asymptomatic for a cancer of the plurality of cancers.

88. The method of any of claims 42-85, wherein the subject is not known or determined to harbor a cancer cell.

89. The method of any of claims 42-85, wherein the subject has not been determined to have or diagnosed with a cancer.

90. The method of any of claims 42-85, wherein the subject has an early stage cancer, e.g., Stage I or Stage II.

91. A kit comprising:
(a) at least 2, 3, 4, 5, 6, 7, 8, 9 or 10 detection reagents, wherein a detection reagent mediates a readout that is a value of the level or presence of:
(i) one or more genetic biomarkers referred to herein;
(ii) one or more protein biomarkers referred to herein; and/or
(iii) the copy number or length of a genomic sequence disposed between at least two terminal repeated elements of a repeated element family (RE Family) referred to herein; and
(b) instructions for using said kit.

92. The kit of claim 91 wherein the detection reagent mediates a readout that is a value of the level or presence of aneuploidy in the genomic sequence.

93. A method of testing for the presence of cancer of a mammal comprising:
a) amplifying a plurality of chromosomal sequences in a DNA sample with a pair of primers complementary to the chromosomal sequences to form a plurality of amplicons;
b) determining at least a portion of the nucleic acid sequence of one or more of the plurality of amplicons;
c) mapping the sequenced amplicons to a reference genome;
d) dividing the DNA sample into a plurality of genomic intervals;
e) quantifying a plurality of features for the amplicons mapped to the genomic intervals;
f) comparing the plurality of features of amplicons in a first genomic interval with the plurality of features of amplicons in one or more different genomic intervals; and
g) determining the presence of cancer in the mammal when the plurality of features of amplicons in a first genomic interval is different from the plurality of features of amplicons in one or more different genomic intervals.

94. The method of claim 93, wherein at least 100,000 amplicons are formed in the step of amplifying.

95. The method of claim 93 or 94, wherein the cancer is a Stage I cancer.

96. The method of any one of claims 93 to 95, wherein the cancer is a liver cancer, an ovarian cancer, an esophageal cancer, a stomach cancer, a pancreatic cancer, a colorectal cancer, a lung cancer, a breast cancer, or a prostate cancer.

97. The method of any one of claims 93-96, further comprising determining the presence of aneuploidy when the plurality of features of amplicons in a first genomic interval is different from the plurality of features of amplicons in one or more different genomic intervals.

98. A method of detecting aneuploidy in a sample comprising low input DNA, using any of the methods disclosed herein.

99. The method of claim 98, wherein the sample comprises about 0.01 picogram (pg) to 500 pg DNA.

100. The method of claim 98 or 99, wherein the sample is a biological sample from a subject.

101. The method of any one of claims 98-100, wherein the sample comprises a liquid sample, a blood sample, a cell-free DNA sample (e.g., a circulating tumor DNA sample), a plasma sample, a serum sample; or a tissue sample.

102. The method of any one of claims 98-100 wherein the sample, e.g., biological sample, comprises cells (e.g., normal or cancer cells) and cell-free DNA.

103. A method of identifying or distinguishing a sample using any of the methods disclosed herein.

104. The method of claim 103, wherein the sample, e.g., first sample, from a subject, e.g., first subject, is distinguished from a second sample from a second subject.

105. The method of claim 103, wherein the sample is identified as being from a subject based on a polymorphism (e.g., a plurality of polymorphisms, e.g., common polymorphisms).

106. The method of claim 105, wherein the polymorphism, e.g., common polymorphism, is present in a repetitive element, e.g., as described herein.

107. The method of any one of claims 1-90 or 93 to 106, wherein the method is an in vitro method.


SEQUENCE LISTING (19 sequences)

SEQ ID NO: 1 - DNA, 57 bp, Artificial
  synthetic primer
  misc_feature (22)..(37): n is a, c, g, or t
cgacgtaaaacgacggccagtnnnnnnnnnnnnnnnnggtgaaaccccgtctctaca

SEQ ID NO: 2 - DNA, 56 bp, Artificial
  synthetic primer
  misc_feature (22)..(37): n is a, c, g, or t
cgacgtaaaacgacggccagtnnnnnnnnnnnnnnnnggtgaaaccccgtctctac

SEQ ID NO: 3 - DNA, 57 bp, Artificial
  synthetic primer
  misc_feature (22)..(37): n is a, c, g, or t
cgacgtaaaacgacggccagtnnnnnnnnnnnnnnnnggtgaaaccccgtctctact

SEQ ID NO: 4 - DNA, 59 bp, Artificial
  synthetic primer
  misc_feature (22)..(37): n is a, c, g, or t
cgacgtaaaacgacggccagtnnnnnnnnnnnnnnnncatgcctgtagtcccagctact

SEQ ID NO: 5 - DNA, 62 bp, Artificial
  synthetic primer
  misc_feature (22)..(37): n is a, c, g, or t
cgacgtaaaacgacggccagtnnnnnnnnnnnnnnnnatagtgaaaccccatctctacaaaa

SEQ ID NO: 6 - DNA, 58 bp, Artificial
  synthetic primer
  misc_feature (22)..(37): n is a, c, g, or t
cgacgtaaaacgacggccagtnnnnnnnnnnnnnnnnggtgaaaccccatctctacaa

SEQ ID NO: 7 - DNA, 61 bp, Artificial
  synthetic primer
  misc_feature (22)..(37): n is a, c, g, or t
cgacgtaaaacgacggccagtnnnnnnnnnnnnnnnnatagtgaaaccccatctctacaaa

SEQ ID NO: 8 - DNA, 55 bp, Artificial
  synthetic primer
  misc_feature (22)..(37): n is a, c, g, or t
cgacgtaaaacgacggccagtnnnnnnnnnnnnnnnngaggtgggaggattgctt

SEQ ID NO: 9 - DNA, 55 bp, Artificial
  synthetic primer
  misc_feature (22)..(37): n is a, c, g, or t
cgacgtaaaacgacggccagtnnnnnnnnnnnnnnnnaccagcctgggcaacata

SEQ ID NO: 10 - DNA, 49 bp, Artificial
  synthetic primer
cacacaggaaacagctatgaccatgcctcctaagtagctgggactacag

SEQ ID NO: 11 - DNA, 49 bp, Artificial; identical to SEQ ID NO: 10
  synthetic primer

SEQ ID NO: 12 - DNA, 49 bp, Artificial; identical to SEQ ID NO: 10
  synthetic primer

SEQ ID NO: 13 - skipped

SEQ ID NO: 14 - DNA, 60 bp, Artificial
  synthetic primer
cacacaggaaacagctatgaccatgtgcagtggcacgatcatagctcactgcagccttga

SEQ ID NO: 15 - DNA, 44 bp, Artificial
  synthetic primer
cacacaggaaacagctatgaccatgctcccgagtagctgggact

SEQ ID NO: 16 - DNA, 46 bp, Artificial
  synthetic primer
cacacaggaaacagctatgaccatgctcccgagtagctgggactac

SEQ ID NO: 17 - DNA, 45 bp, Artificial
  synthetic primer
cacacaggaaacagctatgaccatgcccgagtagctgggactaca

SEQ ID NO: 18 - DNA, 42 bp, Artificial
  synthetic primer
cacacaggaaacagctatgaccatgaggctggagtgcagtgg

SEQ ID NO: 19 - DNA, 42 bp, Artificial
  synthetic primer
cacacaggaaacagctatgaccatgccaccatgcctggctaa